(19) United States
(12) Patent Application Publication
(10) Pub. No.: US 2024/0307497 A1
(43) Pub. Date: Sep. 19, 2024

(54) ADIPONECTIN ALONE OR IN COMBINATION WITH EXTRACORPOREAL PHOTOPHERESIS (ECP) FOR IMMUNE RELATED ADVERSE EVENTS OF IMMUNE CHECKPOINT INHIBITORS

(71) Applicant: Mallinckrodt Pharmaceuticals Ireland Limited, Dublin 15 (IE)

(72) Inventor: Robert ZEISER, Freiburg (DE)

(73) Assignee: Mallinckrodt Pharmaceuticals Ireland Limited, Dublin 15 (IE)

(21) Appl. No.: 18/392,250

(22) Filed: Dec. 21, 2023

Related U.S. Application Data
(60) Provisional application No. 63/517,685, filed on Aug. 4, 2023.

(30) Foreign Application Priority Data
Dec. 21, 2022 (EP) 22215382.7

(51) Int. Cl. A61K 38/22; A61K 35/17; A61K 39/00; A61P 1/06; A61P 37/06; C07K 16/28

(52) U.S. Cl. CPC A61K 38/22; A61K 35/17; A61P 1/06; A61P 37/06; C07K 16/2818; C07K 16/2827; A61K 2039/505

(57) ABSTRACT

The disclosure relates to a substance adiponectin and/or adiponectin receptor agonist (ARA) for use in the treatment of immune checkpoint blockade (ICB)-induced immune related adverse events (irAEs) in a subject, wherein the subject is preferably receiving checkpoint blockade therapy as a cancer therapy. As well as to a combination medication comprising adiponectin and/or adiponectin receptor agonist (ARA) and a photosensitizing agent for combined use in the treatment of immune checkpoint blockade (ICB)-induced immune related adverse events (irAEs) in a subject, wherein the treatment further comprises the extracorporeal irradiation of a blood sample of the subject with ultraviolet A (UVA), preferably extracorporeal photopheresis (ECP) therapy.

### RELATED APPLICATIONS

This patent application claims priority to U.S. Provisional Patent Application No. 63/517,685, filed Aug. 4, 2023, and to European Patent Application No. EP 22215382.7, filed Dec. 21, 2022. The entire contents of each of the foregoing applications are incorporated herein by reference in their entireties.

### STATEMENT REGARDING SEQUENCE LISTING

The content of the electronically submitted sequence listing in XML format (Name 4789_0090002_SeqListing_ST26; Size: 64,656 bytes; and Date of Creation: Dec. 19, 2023) filed with the application is incorporated herein by reference in its entirety. The Sequence Listing is being submitted by Patent Center and is hereby incorporated by reference into the specification.

### FIELD

The disclosure relates to a substance adiponectin and/or adiponectin receptor agonist (ARA) for use in the treatment of immune checkpoint blockade (ICB)-induced immune related adverse events (irAEs) in a subject, wherein the subject is preferably receiving checkpoint blockade therapy as a cancer therapy. The disclosure further relates to a combination medication comprising adiponectin and/or adiponectin receptor agonist (ARA) and a photosensitizing agent for combined use in the treatment of immune checkpoint blockade (ICB)-induced immune related adverse events (irAEs) in a subject, wherein the treatment further comprises the extracorporeal irradiation of a blood sample of the subject with ultraviolet A (UVA), preferably extracorporeal photopheresis (ECP) therapy.

### BACKGROUND

Immune checkpoint blockade (ICB) has proven clinical benefit in the treatment of multiple cancers. Immune checkpoint blockade increases antitumor immunity by blocking negative regulators of immunity, such as cytotoxic T-lymphocyte antigen 4 (CTLA-4), programmed cell death-1 (PD-1) or programmed cell death ligand-1 (PD-L1). Immune-mediated tissue damage of an organ resulting from ICB-therapy is classified as immune-related adverse event (irAE). irAEs can affect any organ system and most commonly involves the gastrointestinal tract, lungs, endocrine glands, skin, and liver.

Treatment of irAEs includes cessation of ICB-therapy and/or administration of glucocorticosteroids, adapted to the severity of the irAEs. These measures incur the risk that lack of ICB-therapy and immunosuppression reduce anti-tumor immune responses. So far, no randomized trial has analyzed the impact of glucocorticosteroids on the anti-tumor immune effect in ICB treated patients and retrospective data are controversial.

Additionally, glucocorticosteroids cause major side-effects including hyperglycemia, fluid retention, psychological disorders, iatrogenic adrenal insufficiency if the glucocorticoids are tapered too fast and infectious complication.

Also, second line therapies such as TNF-antagonists may interfere with anti-tumor effects as TNF was shown to be required for anti-tumor effects in hematological malignancies (Schmaltz et al., 2003) and TNF-blockade for autoimmunity was connected to a numerically increased risk for lymphoproliferative cancers and non-melanoma skin cancers. A retrospective study that analyzed 790 patients with advanced melanoma being treated with ICB, reported a rate of serious infections of 13.5% in the subgroup of patients who received either glucocorticoids or infliximab (TNF antagonist). A recent clinical study showed that TNF-blockade along with immunotherapy was connected to a non-response to ICB in 50% of the patients.

In summary, patients developing severe irAEs (grade 3 and 4) after ICB are currently treated with a complete and durable interruption of the immunotherapy and the administration of glucocorticosteroids. Both interventions may have negative effects on the anti-tumor immune response. Currently no prospective randomized trial supports the use of glucocorticosteroids or second line therapies such as the frequently used approaches with mycophenolate mofetile (MMF), TNF antagonists or cyclosporine A. Novel approaches that are active against irAE are lacking.

Therefore, novel therapeutic approaches that interfere with irAEs without diminishing anti-tumor effects and which induce little systemic side effects are an unmet medical need.

### SUMMARY

In light of the prior art the technical problem underlying the present disclosure is to provide improved therapeutic strategies for the treatment of irAEs, especially during ICB therapy, which do not interfere with ICB mediated anti-tumor effects and cause less side effects than state of the art glucocorticoid treatment.

This problem is solved by the features disclosed herein, including the features of the independent claims and dependent claims.

The inventors found that both, the administration of adiponectin alone or the combined administration of adiponectin and/or adiponectin receptor agonist (ARA), and a photosensitizing agent in the context of extracorporeal photopheresis (ECP) therapy could successfully be used in the treatment of immune checkpoint blockade (ICB)-induced immune related adverse events (irAEs) in a subject.

The inventors have successfully applied extracorporeal photopheresis (ECP) in the treatment of ICB-induced colitis in a patient with metastatic melanoma before (Apostolova et al., 2020). Presently, when analyzing the mechanisms by which ECP reduced colitis after ICB, the inventors found that ECP caused adiponectin expression in the intestinal tract leading to arginase-1 (Arg-1) release by myeloid cells as well as reduced T cell activation and expansion of regulatory KLRG1⁺BTLA⁺CD47⁺TOX^{low} T cells. Adiponectin production was induced by apoptotic cells that were phagocytosed by macrophages in the intestinal tract. The inventors found that ECP reduced clinical and histological signs of ICB-induced colitis in mice as well as in vivo expansion of pathogenic luciferase-transgenic T cells infiltrating irAE target organs. The anti-tumor immunity in melanoma-bearing mice treated with anti-PD1 remained intact during ECP-treatment, while corticosteroids reduced anti-melanoma immune effects. Consistent with the findings in mice ECP induced adiponectin in blood and intestinal tissues of irAE patients.

The inventors identified ECP as a novel immunomodulatory therapy that controls ICB-induced irAE, such as colitis, by activating the adiponectin/Arg-1 axis and by direct effects on T cells and macrophages. Using genetic loss-of-function approaches (Adiponectin KO, Arginase-1 KO mice) and pharmacological interventions the inventors could show a functional role of the adiponectin/Arg-1 axis for the protective effects of ECP.

The inventors found that adiponectin and/or Adiponectin Receptor Agonist (ARA) administration enhances the activity of ECP in the treatment of ICB-induced irAE, which could be observed, e.g., as reduced intestinal inflammation and less weight loss during/after ICB therapy.

The disclosure therefore relates in a first aspect to a combination medication comprising a substance adiponectin and/or adiponectin receptor agonist (ARA) and a photosensitizing agent for combined use in the treatment of immune checkpoint blockade (ICB)-induced immune related adverse events (irAEs) in a subject, wherein the treatment (further) comprises the extracorporeal irradiation of a blood sample of the subject with ultraviolet A (UVA), preferably extracorporeal photopheresis (ECP) therapy.

In preferred embodiments the photosensitizing agent is administered ex vivo to the blood sample of the subject before the extracorporeal UVA-irradiation of the blood sample of the subject. In preferred embodiments the subject is or has been receiving checkpoint blockade therapy, for example, as a cancer therapy.

Unexpectedly, the inventors further found that also adiponectin and/or Adiponectin Receptor Agonist (ARA) administration alone also reduces and prevents ICB-induced irAEs.

The disclosure therefore relates in another aspect to adiponectin and/or Adiponectin Receptor Agonist (ARA) for use in the treatment or prevention of immune checkpoint blockade (ICB)-induced immune related adverse events (irAEs) in a subject. In preferred embodiments the subject is or has been receiving checkpoint blockade therapy, for example, as a cancer therapy.

ICB checkpoint blockade therapy commonly comprises administration of at least one antibody, preferably at least two antibodies, that are preferably selected from the group comprising cytotoxic T-lymphocyte antigen 4 (CTLA-4), programmed cell death-1 (PD-1) and/or programmed cell death ligand-1 (PD-L1) or Lymphocyte-activation gene 3 (LAG-3).

In embodiments of adiponectin and/or ARA for use according to the disclosure, Adiponectin and/or ARA is/are administered at least one hour before, during and/or at least one hour after administration of checkpoint blockade therapy.

In embodiments of adiponectin and/or ARA for use according to the disclosure, Adiponectin and/or ARA is/are administered at least 1 minute, 5, 10, 15, 20, 30, 45 or 60 minutes, or at least 1 hour, 2, 3, 4, 5, 6, 7, 8, 9, 10, 11, 12, 24, 36, or 48 hours, or at least 1, 2, 3, 4, 5, 6, 7, 14, 21, 28, 30 days or at least 1, 2, 3, 4, 5, 6, 7, 8, 9, 10, 11, 12, 13, 14, 15, 16, 20, 24, or 52 weeks after administration of checkpoint blockade therapy.

In embodiments of adiponectin and/or ARA for use according to the disclosure, Adiponectin and/or ARA is/are administered at least 1 minute, 5, 10, 15, 20, 30, 45 or 60 minutes, or at least 1 hour, 2, 3, 4, 5, 6, 7, 8, 9, 10, 11, 12, 24, 36, or 48 hours, or at least 1, 2, 3, 4, 5, 6, 7, 14, 21, 28, 30 days or at least 1, 2, 3, 4, 5, 6, 7, 8, 9, 10, 11, 12, 13, 14, 15, 16, 20, 24, or 52 weeks before administration of checkpoint blockade therapy.

In embodiments of adiponectin and/or ARA for use according to the disclosure, Adiponectin and/or ARA is/are administered at least once daily for at least 1, 2, 3, 4, 5, 6, 7, 14, 21, 28, 30 days or at least 1, 2, 3, 4, 5, 6, 7, 8, 9, 10, 11, 12, 13, 14, 15, 16, 20, 24, or 52 weeks during administration of checkpoint blockade therapy.

Preferably Adiponectin and/or ARA is/are administered at least once daily for at least 2 to 5 weeks during administration of checkpoint blockade therapy, more preferably for at least 3 weeks during administration of checkpoint blockade therapy.

In embodiments of adiponectin and/or ARA for use according to the disclosure, the subject suffers from cancer.

In embodiments of adiponectin and/or ARA for use according to the disclosure, the cancer is selected from the group comprising malignant melanoma, breast cancer or another cancer treatable by checkpoint blockade therapy, e.g., such as any cancer disclosed herein.

In embodiments of adiponectin and/or ARA for use according to the disclosure, the checkpoint blockade therapy comprises administration of at least one antibody, preferably at least two antibodies.

In embodiments of adiponectin and/or ARA for use according to the disclosure, the at least one antibody is selected from the group comprising cytotoxic T-lymphocyte antigen 4 (CTLA-4), programmed cell death-1 (PD-1) and/or programmed cell death ligand-1 (PD-L1) or Lymphocyte-activation gene 3 (LAG-3).

In embodiments of adiponectin and/or ARA for use according to the disclosure, irAEs can involve any organ, preferably the gastrointestinal tract, lungs, endocrine glands, skin, and/or liver.

In embodiments of adiponectin and/or ARA for use according to the disclosure, the irAE comprises an ICB-induced colitis.

In embodiments of adiponectin and/or ARA for use according to the disclosure, the administration of adiponectin and/or ARA reduces ICB-induced colitis.

In embodiments of adiponectin and/or ARA for use according to the disclosure, the administration of adiponectin and/or ARA does not interfere with the anti-tumor response induced by the checkpoint blockade therapy, which is preferably an anti-PD1 treatment, (at least partially) due to the tissue specific adiponectin induction.

In embodiments of adiponectin and/or ARA for use according to the disclosure, an anti-PD-1 induced anti-tumor immune response mediated by T cells in the tumor microenvironment is not impaired by the administration of Adiponectin and/or ARA, wherein the intact anti-tumor immune response is preferably proven by recall immunity experiments.

In another aspect the present disclosure relates to a combination medication for use in the treatment of immune checkpoint blockade (ICB)-induced immune related adverse events (irAEs) in a subject, comprising at least two of the following components:
a. adiponectin and/or Adiponectin Receptor Agonist (ARA), and
b. a photosensitizing agent,
   and wherein the treatment further comprises the treatment of a blood sample of said subject with extracorporeal ultraviolet A (UVA) irradiation, preferably extracorporeal photopheresis (ECP).

In preferred embodiments the photosensitizing agent is administered ex vivo to the blood sample before the treatment of the blood sample with extracorporeal ultraviolet A (UVA) irradiation, preferably extracorporeal photopheresis (ECP).

In embodiments the combination medication for use in the treatment of immune checkpoint blockade (ICB)-induced immune related adverse events (irAEs) in a subject, comprises the following components:
a. adiponectin and/or Adiponectin Receptor Agonist (ARA),
b. a photosensitizing agent, and
c. a blood sample of the subject and/or lymphocytes, preferably T cells, extracted therefrom,
   wherein the blood sample and/or lymphocytes extracted therefrom, has/have been subjected ex vivo to a treatment with the photosensitizing agent, and subsequent extracorporeal ultraviolet A (UVA) irradiation, preferably extracorporeal photopheresis (ECP).

In other words, in embodiments the combination medication for use in the treatment of immune checkpoint blockade (ICB)-induced immune related adverse events (irAEs) in a subject, comprises a substance adiponectin and/or adiponectin receptor agonist (ARA), a photosensitizing agent and a blood sample and/or lymphocytes, preferably T cells, comprised therein or extracted therefrom, wherein the blood sample and/or lymphocytes have been subjected to an extracorporeal treatment comprising (i) administration of the photosensitizing agent to the blood sample and/or lymphocytes, and subsequently (ii) an extracorporeal irradiation with ultraviolet A (UVA), preferably extracorporeal photopheresis (ECP).

In embodiments the combination medication for use in the treatment of immune checkpoint blockade (ICB)-induced immune related adverse events (irAEs) in a subject, comprises
a. adiponectin and/or adiponectin receptor agonist (ARA),
b. a photosensitizing agent, and
c. leukocytes, preferably (immunoregulatory) T cells, obtained from a method comprising the steps of
   (i) providing of a sample derived from an isolated blood sample of the subject,
   (ii) adding the photosensitizing agent to the sample, and
   (iii) subjecting the sample to UVA irradiation, preferably ECP treatment.

As shown in the examples, the positive effect and the efficacy of the administration of a sample that underwent ECP treatment according to the disclosure may be at least partially due to a modulation of the function of the leukocytes, specifically T cells, comprised in the blood sample, wherein said modulation is a result of the irradiation in presence of a photosensitizing agent. Therefore, the present disclosure also encompasses leukocytes, preferably lymphocytes, more preferably T cells, even more preferably immunomodulatory/immunoregulatory T cells, as combination medication with adiponectin and/or adiponectin receptor agonist (ARA) for the combined use in the treatment of and/or prevention of immune-related adverse events (irAE) in a subject that is receiving a checkpoint-inhibitor therapy. Preferably, the immunomodulatory T cells were generated by subjecting blood, the buffy coat, lymphocytes and/or MNCs cells from a person, preferably a subject that has received a checkpoint-inhibitor therapy and is suspected of developing or has developed irAE, to the ECP method described herein, and are subsequently used for treating a subject that has received a checkpoint-inhibitor therapy and is suspected of developing or has developed irAE, which is preferably the blood and/or cell donor.

In preferred embodiments the blood sample of the subject is subjected extracorporeal to centrifugation before the treatment with the photosensitizing agent and UVA irradiation, such that only the buffy coat, which preferably comprises lymphocytes and optionally other leukocytes and platelets, is subjected to extracorporeal treatment with the photosensitizing agent and UVA irradiation, and is preferably afterwards administered back to the subject.

In preferred embodiments the subject is or has been receiving checkpoint blockade therapy.

The disclosure further relates to a combination medication for use in the treatment of immune checkpoint blockade (ICB)-induced immune related adverse events (irAEs) in a subject, wherein the combination medication comprises
(i) adiponectin and/or adiponectin receptor agonist (ARA),
(ii) a photosensitizing agent, and
(iii) a closed extracorporeal photopheresis (ECP) system,
wherein the closed ECP system forms a closed circuit with the vascular system, preferably the venous system, of the subject.

In preferred embodiments in the closed ECP system a blood sample is received from a subject's vein through an intravenous line, whereinafter the buffy coat is separated from the residual blood by centrifugation within the ECP system, subsequently the residual blood is reintroduced into a subject's vein, while the buffy coat is mixed or combined within the ECP system with the photosensitizing agent and subjected to UVA irradiation, and finally the treated and irradiated buffy coat and/or lymphocytes derived therefrom is reinfused into a vein of the subject through an intravenous line.

In preferred embodiments the buffy coat comprises T cells. In preferred embodiments the reinfused buffy coat comprises immunoregulatory T cells.

In embodiments of the disclosure, the photosensitizing agent is a psoralen reagent, preferably 8-methoxypsoralen. Preferably herein the ‘ECP method’ or ‘ECP treatment’ of a blood sample, a fraction thereof or cells derived therefrom comprises the treatment or mixing of said blood sample, a fraction thereof or cells derived therefrom with a photosensitizing agent prior to its UVA irradiation. In preferred embodiments of the disclosure the photosensitizing agent is administered ex vivo/extracorporeal to a blood sample, or a fraction thereof, of the subject.

Aspects of the present disclosure are based on the finding that patients that have received a checkpoint-inhibitor therapy, for example, as a cancer treatment, which caused irAEs, can be effectively treated by administering adiponectin and/or adiponectin receptor agonist (ARA) and a photosensitizing agent and subsequently applying extracorporeal irradiation of a blood sample of the subject with ultraviolet A (UVA), preferably applying ECP. In preferred embodiments ECP basically refers to a method comprising adding extracorporeally a photosensitizing agent to a blood sample or blood-derived sample of an irAE patient, and subjecting the sample to extracorporeal UVA irradiation. It was found that preforming this combined treatment approach reduces both the occurrence of and the severity of irAEs in a subject, without impairing the anti-cancer effect of the ICB therapy.

In embodiments of a substance adiponectin and/or adiponectin receptor agonist (ARA) and a photosensitizing agent for combined use according to the disclosure, the subject is or has been receiving checkpoint blockade therapy.

In a further aspect, the disclosure relates to a combination medication for the use in the treatment and/or prevention of immune checkpoint blockade (ICB)-induced immune related adverse events (irAEs) in a subject, comprising adiponectin and/or adiponectin receptor agonist (ARA) and lymphocytes obtained from a method comprising the steps of
provision of a sample derived from an isolated blood sample of the subject,
adding a photosensitizing agent to the sample, and
subjecting the sample to UVA irradiation,
   wherein preferably the subject is or has been receiving checkpoint blockade therapy, for example, as a cancer therapy.

In preferred embodiments the lymphocytes comprise (immunoregulatory) KLRG1⁺BTLA⁺CD47⁺TOX^{low} T cells.

Hence, in preferred embodiments the combination medication for the use in the treatment and/or prevention of immune checkpoint blockade (ICB)-induced immune related adverse events (irAEs) in a subject, comprises adiponectin and/or adiponectin receptor agonist (ARA) and (immunoregulatory) T cells obtained from a method comprising the steps of
provision of a sample derived from an isolated blood sample of a subject,
adding a photosensitizing agent to the sample, and
subjecting the sample to UVA irradiation,
   wherein preferably the subject is or has been receiving checkpoint blockade therapy, for example, as a cancer therapy.

This aspect of the disclosure is based on the observation that T cells comprised in a blood-derived sample that underwent irradiation after adding a photosensitizing adopt an immunoregulatory phenotype that is particularly advantageous when using the resulting cells in the treatment and/or prevention of irAE in a subject in combination with the administration of adiponectin and/or adiponectin receptor agonist (ARA).

In preferred embodiments, the blood sample used for generating the immunoregulatory T cells of the disclosure is from a subject that has received a checkpoint-inhibitor therapy and is suspected of developing or has developed symptoms of immune-related adverse events (irAE). This embodiment is particularly advantageous, since the cells are autologous to said patient and there will be no adverse events that can occur upon transplantation of heterologous cells.

Preferably, the immunoregulatory T cells for use in such a treatment or prevention have been generated by adding a photosensitizing agent to a sample derived from a blood sample of a human subject and subjecting the sample to subsequent UVA irradiation. Adding a photosensitizing agent to the blood-derived sample and subjecting the sample to UVA irradiation preferably generates or induces (the formation of) immunoregulatory T cells in said sample.

In preferred embodiments the photosensitizing agent is 8-methoxypsoralen and/or the irradiation is UVA irradiation. In the context of the disclosure, irradiation is preferably performed by means of an extracorporeal photopheresis (ECP) system. Irradiation of the blood sample can be performed using any suitable system or irradiation device known to the skilled person.

The ECP treatment or ECP method according to the disclosure relates to a method comprising the steps of
provision of a sample derived from a blood sample of a subject that has received a checkpoint-inhibitor therapy and is suspected of developing or has developed symptoms of immune-related adverse events (irAE),
adding a photosensitizing agent to the sample, and
subjecting the sample to UVA irradiation, preferably extracorporeal photopheresis (ECP) therapy.

In preferred embodiments of the combination medication according to the disclosure, the adiponectin and/or ARA is administered before, during and/or after the ECP treatment of a sample of the subject, wherein the ECP treatment comprises extracorporeally (ex vivo) treating a blood sample of a patient with a photosensitizing agent and subjecting it to extracorporeal (ex vivo) UVA irradiation, and wherein the combination medication preferably further comprises administering said ECP-treated blood sample, a fraction thereof and/or lymphoid cells derived therefrom back to the subject.

In preferred embodiments the blood sample of the subject is subjected extracorporeally to centrifugation before the ECP treatment, such that only the buffy coat, which preferably comprises lymphocytes and optionally other leukocytes and platelets, is subjected to extracorporeal treatment with the photosensitizing agent and UVA irradiation, and is afterwards preferably administered (back) to the subject.

The combined ECP treatment of blood samples from the subject, such as in particular a blood sample comprising mononuclear cells (MNCs), leads to the generation of immunoregulatory T cells in said sample. In this context, “generation of” immunoregulatory T cells is understood as inducing or inducing the formation of such cells in the sample. In other words, cells that are comprised in the sample differentiate into or adopt a phenotype of immunoregulatory T cells.

It was found that the sample resulting from the ECP treatment according to the disclosure and in particular the induced immunoregulatory T cells comprised by the sample are useful for the treatment of irAE patients. It could be shown that administration of such a sample or cells comprised in such a sample, which underwent the method of the disclosure, to a subject that has received a checkpoint-inhibitor therapy in combination with adiponectin/ARA is effective in preventing the occurrence of irAE and even in the treatment of irAE that is already established in the subject. The combination medication comprising adiponectin and/or ARA and an ECP-treated blood sample of the subject, or lymphocytes extracted therefrom, showed even synergistic effects in preventing or treating ICB-induced irAE, when compared to the treatment of a subject with only one of the medications.

It was completely unexpected, that the therapy comprising administering adiponectin and/or ARA and using a sample or cells resulting from the ECP-method of the disclosure is even effective for irAE patients that are refractory to other immunosuppressive therapies, such as steroids or anti-TNF antibodies, and which continue to show symptoms of or still suffer from irAE after checkpoint-inhibitor treatment was discontinued.

Importantly, the ECP treatment according to the disclosure can be performed on a sample of the same subject that has received the checkpoint inhibitor therapy. Therefore, the cells or the sample resulting from the method of the disclosure can be administered to the same subject that served as a blood donor and therefore the resulting sample can represent an autologous cell therapy. In other embodiments the ECP treatment according to the disclosure can be performed on a sample of another subject, which is a compatible donor for the subject receiving ICB treatment, therefore the resulting sample can represent a heterologous cell therapy.

As used herein, the term “subject that is or has been receiving checkpoint-inhibitor therapy” includes subjects that are currently under ongoing checkpoint-inhibitor therapy, or subjects that have received a checkpoint-inhibitor therapy that was either discontinued, for example after irAE symptoms occurred, or completed.

In embodiments, the ECP treatment according to the disclosure is performed in vitro or ex vivo. As used herein, the terms in vivo and ex vivo are used synonymously. In the context of the present disclosure, the terms relate to a method that is performed on a blood-derived sample that has been removed from the human body, wherein the method of the disclosure is performed on the blood-derived sample out-side the human body. To this end, blood of a donor subject is taken out of the body, meaning out of the physiological circulatory system. As used herein, an “isolated blood sample” is a sample of blood (meaning a certain volume of blood) that is removed from the circulatory system of the donor to a location outside the body of the person. Such an isolated blood sample can be subjected or introduced into an extracorporeal photopheresis (ECP) system or an apheresis system for performing at least certain steps of the method of the disclosure. Therein, such a system can be an online system that is in a fluid connection with the blood circulatory system of the subject. In alternative embodiments, the method can be performed offline, wherein the blood-derived sample that is subjected to irradiation is disconnected from the circulatory system of the donor subject.

Accordingly, in embodiments, the ECP-treatment of the disclosure is an in vitro method. In embodiments of the method of the disclosure, the blood sample is an isolated blood sample. In embodiments, the method is an in vitro method and the blood sample is an isolated blood sample.

As used herein the term “blood sample” comprises all kinds of blood-derived samples, including blood-cell samples such as MNC samples that are generated from blood.

In embodiments of the disclosure, the subject (the blood donor and/or recipient of cells) shows symptoms of or suffers from irAE. In further embodiments, the subject has received immune checkpoint-inhibitor therapy (ICB), but the checkpoint-inhibitor therapy was either completed, or discontinued after symptoms and/or manifestation of irAE occurred in said subject. In embodiments, symptoms and/or manifestation of irAE occurred in the subject after the checkpoint-inhibitor therapy was discontinued. In embodiments, symptoms and/or manifestation of irAE were maintained after the checkpoint-inhibitor therapy was discontinued.

In embodiments of the disclosure, where the subject that has received a immune checkpoint-inhibitor therapy (ICB) and is suspected of developing or has developed symptoms of irAE serves as a blood donor, provision and/or isolation of the blood sample can occur at any of the time points described in the embodiments above. For example, sample isolation can occur prior to or after development of irAE symptoms. Furthermore, sample isolation can occur during checkpoint-inhibitor therapy or after discontinuation of said therapy.

In preferred embodiments of the disclosure, the sample resulting from the ECP-treatment according to the disclosure or the T cells for use according to the present disclosure are administered to the subject while check-point-inhibitor therapy is ongoing.

In preferred embodiments, sample isolation occurs during ongoing checkpoint-inhibitor therapy, either before or after occurrence of irAE symptoms. The sample or cells resulting from the method of the examples can be used preventively or therapeutically by administering the sample/cells to the subject during ongoing checkpoint-inhibitor therapy. Accordingly, in preferred embodiments of the disclosure, the subject that has received checkpoint-inhibitor therapy can receive immunoregulatory T cells, which are preferably autologous and have been generated by irradiating a blood-derived sample according to a method described herein, while checkpoint-inhibitor therapy is ongoing. Such embodiments are particularly advantageous, since checkpoint-inhibitor therapy, such as an anti-cancer checkpoint inhibitor therapy, can be maintained, while the patient receives cells of the disclosure and/or cells resulting from the method of the disclosure.

Accordingly, in such embodiments a checkpoint-inhibitor therapy can be administered to the subject for a longer period, since the administration of the irradiated cells either prevents the occurrence of irAE or ameliorates irAE so that the checkpoint-inhibitor therapy can be continued. Administration of such cells resulting from a method of the disclosure, in particular immunomodulatory T cells of the disclosure, does not interfere with the anti-cancer response of the subject to the checkpoint-inhibitor therapy. This represents an important advantage in comparison to known treatments/preventive measures of irAE, in particular administration of immunosuppressive drugs, such as corticosteroids. Performing ECP did not notably alter the anti-cancer effect of checkpoint-inhibitor therapy, while parallel administration of glucocorticoids (specific class of corticosteroids), in particular prednisolone, resulted in a worse outcome, indicating a reduced effectivity of the checkpoint-inhibitor treatment.

Furthermore, in embodiments where a (human) blood-derived sample that underwent a method of adding a photosensitizing agent to the sample and subjecting the sample to irradiation is used in the treatment and/or prevention of irAE in a subject that has received a checkpoint-inhibitor therapy, irrespective of the source of the blood-derived sample (autologous or heterologous), the administration of the sample or cells resulting from such a method can occur, for example, prior to or after development of irAE symptoms, and/or during the checkpoint-inhibitor therapy or after discontinuation of the checkpoint-inhibitor therapy.

In embodiments, the subject (blood-donor and/or of recipient of cells) suffers from cancer, such as malignant melanoma or another cancer treatable by checkpoint-inhibitor therapy.

In embodiments, the subject is receiving immunosuppressive drugs, such as steroids, corticosteroid, cyclosporine and/or anti-TNF antibodies (for example infliximab) and/or is refractory to immunosuppressive drugs.

In embodiments of the disclosure, the irAE (of a blood-donor and/or of recipient of cells) comprise symptoms of an autoimmune disease and/or is caused by an autoimmune reaction. In preferred embodiments, the irAE comprise or are autoimmune colitis.

In embodiments, the irAE comprise at least one irAE selected from the group comprising autoimmune colitis, autoimmune hepatitis, autoimmune thyroiditis and autoimmune dermatitis.

In embodiments, the irAE comprise at least one irAE selected from the group comprising immune checkpoint inhibitor-related colitis, immune checkpoint inhibitor-related hepatitis, immune checkpoint inhibitor-related thyroiditis and immune checkpoint inhibitor-related dermatitis.

Administration of cells that resulted from the methods described herein, such as immunoregulatory T cells, have a therapeutic effect on irAE patients that are refractory to immunosuppressive drugs that have been administered for treating the irAE symptoms. For such patients, there is no effective treatment available for ameliorating irAE symptoms and the present disclosure therefore represents a completely unexpected possibility of treating irAE in these patients.

In embodiments, the checkpoint-inhibitor therapy comprises administration of at least one of anti-CTLA4 antibodies and anti-PD-1 antibodies.

In embodiments, the immunoregulatory T cells for use in the treatment and/or prevention of irAE in a subject that has received a checkpoint-inhibitor therapy are autologous with respect to said subject. In alternative embodiment, the T cells may be heterologous with respect to said subject.

In embodiments, the combination medication comprising adiponectin and/or adiponectin receptor agonist (ARA) and lymphocytes, preferably (immunoregulatory) T cells, for use according to the present disclosure is administered upon occurrence of irAE symptoms. In embodiments, the cells are administered 1, 2, 3, 4, 5, 6 or 7 days or 1, 2, 3, 4, 5, 6, 7, 8, 9, 10, 12, 14, 16, 18, 20, 22, 24, 26, 28, 30, 35, 40, 45 or 50 weeks, preferably for at least 1-3 weeks, more preferably for at least 2 weeks after occurrence of irAE symptoms, and adiponectin and/or adiponectin receptor agonist (ARA) is administered for 1, 2, 3, 4, 5, 6 or 7 days or for 1, 2, 3, 4, 5, 6, 7, 8, 9, 10, 12, 14, 16, 18, 20, 22, 24, 26, 28, 30, 35, 40, 45 or 50 weeks, preferably for 1-5 weeks, more preferably for at least 3 weeks after occurrence of irAE symptoms. In embodiments, the combination medication comprising adiponectin and/or adiponectin receptor agonist (ARA) and immunoregulatory T cells are administered during checkpoint-inhibitor therapy is ongoing or after the checkpoint-inhibitor therapy was discontinued or completed.

In embodiments of the combination medication, the lymphocytes, preferably (immunoregulatory) T cells, are administered at least once to the subject, preferably at least twice, preferably on consecutive days. Accordingly, the subject may receive more than one dose of the lymphocytes/T cells, wherein preferably not more than one dose per day is administered. In further embodiments, the subject receives at least 2, 3, 4 or 5 dosages of the lymphocytes/T cells of the disclosure. In embodiments, the lymphocytes/the immunoregulatory T cells of the disclosure are administered to the subject at least every 8 weeks, preferably every 2-4 weeks. In embodiments the adiponectin and/or adiponectin receptor agonist (ARA) is administered before, during and/or after the administration of the lymphocytes/immunoregulatory T cells. In embodiments adiponectin is administered at least once, more preferably on two days, and at least once per day.

In embodiments of the combination medication, or a substance adiponectin and/or adiponectin receptor agonist (ARA) and a photosensitizing agent for combined use according to the disclosure, adiponectin and/or ARA and the photosensitizing agent are administered at least 30 minutes after administration of checkpoint blockade therapy.

In embodiments of the combination medication, or a substance adiponectin and/or adiponectin receptor agonist (ARA) and a photosensitizing agent for combined use according to the disclosure, the subject has been diagnosed with and/or is suffering from cancer.

In embodiments of the combination medication, or a substance adiponectin and/or adiponectin receptor agonist (ARA) and a photosensitizing agent for combined use according to the disclosure, the photosensitizing agent is a psoralen reagent, preferably 8-methoxypsoralen.

In embodiments of the combination medication, or a substance adiponectin and/or adiponectin receptor agonist (ARA) and a photosensitizing agent for combined use according to the disclosure, the wavelength of the UVA irradiation is from wavelength of 3200 to 4000 angstroms.

In embodiments of the combination medication, or a substance adiponectin and/or adiponectin receptor agonist (ARA) and a photosensitizing agent for combined use according to the disclosure, the subject suffers from cancer.

In embodiments of the combination medication, or a substance adiponectin and/or adiponectin receptor agonist (ARA) and a photosensitizing agent for (combined) use according to the disclosure, the cancer is selected from the group comprising malignant melanoma, breast cancer or another cancer treatable by checkpoint blockade therapy.

In embodiments of the combination medication, or a substance adiponectin and/or adiponectin receptor agonist (ARA) and a photosensitizing agent for (combined) use according to the disclosure, the checkpoint blockade therapy comprises administration of at least one antibody, preferably at least two antibodies.

In embodiments of the combination medication, or a substance adiponectin and/or adiponectin receptor agonist (ARA) and a photosensitizing agent for (combined) use according to the disclosure, the at least one antibody is selected from the group comprising cytotoxic T-lymphocyte antigen 4 (CTLA-4), programmed cell death-1 (PD-1) and/or programmed cell death ligand-1 (PD-L1) or Lymphocyte-activation gene 3 (LAG-3).

In embodiments of the combination medication, or a substance adiponectin and/or adiponectin receptor agonist (ARA) and a photosensitizing agent, for (combined) use according to the disclosure, irAEs can involve any organ, preferably the gastrointestinal tract, lungs, endocrine glands, skin, and liver.

In embodiments of the combination medication, or a substance adiponectin and/or adiponectin receptor agonist (ARA) and a photosensitizing agent, for (combined) use according to the disclosure, the irAE comprises an ICB induced colitis.

In embodiments of the combination medication, or a substance adiponectin and/or adiponectin receptor agonist (ARA) and a photosensitizing agent, for (combined) use according to the disclosure, said combination medication reduces ICB-induced colitis.

In embodiments of the combination medication, or a substance adiponectin and/or adiponectin receptor agonist (ARA) and a photosensitizing agent, for (combined) use according to the disclosure, said combination medication induces apoptosis in leukocytes, which are phagocytosed by intestinal macrophages thereby causing adiponectin production in the inflamed intestinal tract.

In embodiments of the combination medication, or a substance adiponectin and/or adiponectin receptor agonist (ARA) and a photosensitizing agent, for (combined) use according to the disclosure, said combination medication induces an increase of adiponectin, arginase-1 (Arg1) and tolerogenic KLRG1⁺BTLA⁺CD47⁺TOX^{low} T cells in inflamed intestines, but not in cancer tissue in said subject.

In embodiments of the combination medication, or a substance adiponectin and/or adiponectin receptor agonist (ARA) and a photosensitizing agent, for (combined) use according to the disclosure, said combination medication does not interfere with the anti-tumor response induced by anti-PD1 treatment due to the tissue specific adiponectin induction.

In embodiments of the combination medication, or a substance adiponectin and/or adiponectin receptor agonist (ARA) and a photosensitizing agent, for (combined) use according to the disclosure, an anti-PD-1 induced anti-tumor immune response mediated by T cells in the tumor microenvironment is not impaired by the said combination medication, wherein the intact anti-tumor immune response is proven by recall immunity experiments.

In another aspect the present disclosure relates to adiponectin and/or Adiponectin Receptor Agonist (ARA) for use in the treatment of a tumor patient, or a subject suffering from cancer, wherein said patient/subject has developed immune checkpoint blockade (ICB) induced immune related adverse events (irAEs), wherein said treatment comprises the combined administration of Adiponectin and/or ARA with an anti-tumor immunotherapy, wherein said immunotherapy comprises the administration of immunoregulatory T cells, and/or of blood samples.

In embodiments of adiponectin and/or ARA for use in the treatment of a tumor patient according to the disclosure, the immunoregulatory T cells and/or of blood samples are obtained from a method comprising the steps of
providing a sample derived from an isolated blood sample of said patient,
adding a photosensitizing agent to the sample, and subjecting the sample to irradiation.

In embodiments of adiponectin and/or ARA for use in the treatment of a tumor patient according to the disclosure, the photosensitizing agent is 8-methoxypsoralen and/or the irradiation is UVA irradiation.

In another aspect the present disclosure relates to a method of treating a subject that has received a checkpoint-inhibitor therapy and is suspected of developing or has developed symptoms of immune-related adverse events (irAE), the method comprising administering adiponectin and/or Adiponectin Receptor Agonist (ARA) at least once to the subject before, during or after subjecting said subject to an extracorporeal photopheresis (ECP) therapy, such as blood irradiation therapy by means of an ECP system.

In embodiments the extracorporeal photopheresis (ECP) therapy comprises the administration of a photosensitizing agent to a blood sample, or fragments thereof, of the subject before UVA irradiation of said blood sample, or fragments thereof.

Each optional or preferred feature of the disclosure that is disclosed or described in the context of one aspect of the disclosure is herewith also disclosed in the context of the other aspects of the disclosure described herein.

All features disclosed in the context of the ECP method for use in the treatment and/or prevention of immune-related adverse events (irAE) in a subject that has received a checkpoint-inhibitor therapy of the disclosure also relate to and are herewith disclosed also in the context of the in vitro method of the disclosure, the method of treatment of the disclosure and the lymphocytes and/or T cells for use in the treatment and/or prevention of immune-related adverse events (irAE) in a subject that has received a checkpoint-inhibitor therapy of the disclosure, and vice versa.

### BRIEF DESCRIPTION OF THE DRAWINGS

The disclosure is further described by the following figures. These are not intended to limit the scope of the disclosure but represent preferred embodiments of aspects of the disclosure provided for greater illustration of the disclosure described herein.

FIGS. 1a-1m shows that ECP-treatment reduced ICI-enhanced colitis in vivo by adiponectin induction.

Specifically, FIG. 1a shows a graph that depicts bodyweights normalized to bodyweight on do of the experiment. Bodyweights were taken daily. Groups include untreated mice (UT), animals with DSS-induced colitis treated with anti-PD1 or anti-PD1 plus ECP. Data represents the mean of the pooled weight of all animals of the indicated group. The arrows indicate when ECP was performed (d3 and d6). Data were pooled from 3-6 experiments.

FIG. 1b shows a scatter bar graph that shows mean (±s.e.m.) colon length of untreated animals, mice with DSS/anti-PD1-induced colitis and mice with DSS/anti-PD1-induced colitis treated with ECP, as indicated. The pooled values of two experiments are shown. P-value was calculated using one-way ANOVA.

FIG. 1c shows representative colons isolated from mice of the indicated group are shown. Colons were isolated on d8 of the experiment.

FIG. 1d shows representative histology images of colon samples from mice with DSS-induced colitis and anti-PD1 or anti-PD1 plus ECP-treatment. The arrows point towards neutrophils in the intestinal wall.

FIG. 1e and 1f show graphs that depict histopathological grading of neutrophil (FIG. 1e) and lymphocyte (FIG. 1f) infiltration into the colon (0=absent, 1=mild, 2=massive) of untreated (UT) mice or mice with DSS-induced colitis and anti-PD1 or anti-PD1 plus ECP-treatment. Each data point represents the value of an individual mouse. The experiment was done twice and the results (mean±s.e.m.) were pooled. P-values were calculated using the Kruskal-Wallis test.

FIG. 1g shows a relative gene expression, depicted as microarray heatmap (“Z-score intensity”) from RNA isolated from the colitis tissue of mice treated with DSS and anti-PD1 (n=4) or DSS and anti-PD1 plus ECP (n=5). Tile display shows the most significant differentially regulated genes as determined from a linear model-based approach. The scale represents the row-wise scaled levels of gene expression with red being highest and blue being lowest. The arrow points towards the most upregulated gene in the ECP group, Adipoq. The right column indicates the log 2 fold change between anti-PD1 plus ECP and anti-PD1. The asterisks highlight significant regulation.

FIG. 1h shows an Adipoq gene expression in the colon of mice that had DSS-induced colitis and were treated with anti-PD1 alone or anti-PD1 plus ECP. Gene expression data was measured by qPCR normalized to Hprt expression and is shown as fold change to anti-PD1 treatment. Results from three individual experiments were pooled (mean±s.e.m.). The p-value was calculated using an unpaired Student's t-test.

FIG. 1i shows a scatter bar graph that shows the mean (±s.e.m.) of adiponectin in serum samples of mice with DSS-induced colitis, treated with anti-PD1 alone or anti-PD1 plus ECP. Each data point represents an individual mouse. Results from nine experiments were pooled. The p-value was calculated using an unpaired Student's t-test.

FIG. 1j shows a scatter bar graph that depicts the colon length of mice with DSS/anti-PD1-induced colitis and ECP-treatment as indicated. WT: wildtype recipient, Adipoq^{-/-}: adiponectin-deficient recipient. ECP donors were WT for both groups. Results were pooled from two individual experiments (mean±s.e.m.). The p-value was calculated using an unpaired Student's t-test.

FIG. 1k shows a graph that shows bodyweights of mice treated with DSS, anti-PD1 and ECP, normalized to bodyweight at do of the experiment. WT: wildtype recipient, Adipoq^{-/-}: adiponectin deficient recipient. ECP donors were WT for both groups. Arrows indicate the timepoint of ECP transplantations. Data represents the mean of the pooled weight of all animals of the indicated group. Results were pooled from two individual experiments.

FIGS. 1l and 1m show scatter bar graphs that depict histopathological grading of neutrophil (FIG. 1l) and lymphocyte (FIG. 1m) infiltration into the colon of mice with DSS-induced colitis and anti-PD1 plus ECP-treatment. WT: wildtype recipient, Adipoq^{-/-}: adiponectin deficient recipient. ECP donors were WT for both groups. Results from two individual experiments were pooled (mean±s.e.m.), significance was calculated using a Mann-Whitney test.

FIGS. 2a-2u shows that phagocytosis of apoptotic leukocytes caused adiponectin expression in phagocytic cells.

Specifically, FIG. 2a shows representative flow cytometry plots of CellTrace Violet (CTV) in CD45.2⁺ macrophages. ECP-treated CTV stained CD45.1⁺ splenocytes were exposed to CD45.2⁺ bone marrow-derived macrophages for 48 h of co-culture.

FIG. 2b shows the quantification of cell trace violet positive CD45.2⁺ macrophages shown in FIG. 2a. Data were pooled from multiple experiments (mean±s.e.m.). P-value was calculated using an unpaired Student's t-test.

FIG. 2c shows representative IF images of BMDMs (CD11b⁺), DAPI, and ECP-treated splenocytes. The splenocytes were exposed to BMDMs for 24 h of co-culture. Scale bar: 20 μm. Arrows show cells that phagocytosed ECP-treated splenocytes.

FIG. 2d shows the quantification of macrophages with ECP-treated splenocytes as percent of single cells. Data were pooled from two experiments. The p-value was calculated using Wilcoxon test.

FIG. 2e-2h show scatter bar graphs of phosphorylated STAT6 (FIG. 2e), Arg1 (FIG. 2f), CD206 (FIG. 2g), CD301 (FIG. 2h) protein levels in CD45.2 BMDMs, either left untreated or co-cultured with ECP-treated CD45.1⁺ splenocytes for 48 h. Data were pooled from four experiments (mean±s.e.m.) and shown as fold change of MFI, compared to medium controls. Significance was calculated with a Wilcoxon test.

FIG. 2i shows a scatter bar graph that depicts Adipoq gene expression in CD45.2⁺ BMDMs (PrimeFlow RNA Assay), which were co-cultured with ECP-treated CD45.1⁺ splenocytes for 48 h. Data were pooled from two experiments (mean±s.e.m.) and depicted as fold change of MFI, compared to medium control. Significance was calculated with a Wilcoxon test.

FIG. 2j shows representative histograms of Adipoq RNA (PrimeFlow, gene expression analysis) in BMDMs only or BMDMs co-cultured with ECP-treated splenocytes for 48 h.

FIG. 2k shows a representative fluorescence image of the colitis tissue from melanoma-bearing mice with DSS/anti-PD1-induced colitis. Mice received VivoTrack680-stained ECP splenocytes or were untreated (control). Images were taken 12 h after transplant.

FIG. 2l shows a representative fluorescence image of the s.c. melanoma tissue from tumor-bearing mice with DSS/anti-PD1-induced colitis. Mice received VivoTrack680-stained ECP splenocytes or were untreated (control). Images were taken 12 h after transplant.

FIG. 2m shows a graph that depicts the quantification of tissue fluorescence signal (average radiant efficiency) from tissue samples shown in (FIGS. 2k and 2l) at 6 h and 12 h after transfer of ECP-treated cells. Significance was calculated using a Kruskal-Wallis test.

FIG. 2n shows a scatter bar graph that depicts CD45.2_VT680⁺ cells isolated from the lamina propria of the colon or from the subcutaneous melanoma tissue from tumor-bearing mice with DSS/anti-PD1-induced colitis. Mice received ECP-treated CD45.1⁺VT680⁺ splenocytes. Results (mean±s.e.m.) are pooled from two individual experiments, p-values were calculated with one-way ANOVA (*** is p<0.0001).

FIG. 2o shows the Arginase-1 protein level in CD45.2⁺CD11b⁺VT680⁺ cells isolated from the lamina propria of the colon from tumor-bearing mice with DSS/anti-PD1-induced colitis at 12 h after transfer of ECP-treated CD45.1⁺VT680⁺ splenocytes. Results (mean±s.e.m.) are pooled from two individual experiments, p-value was calculated with Wilcoxon test.

FIG. 2p shows representative histograms for Arg-1 of specimen described in FIG. 2o.

FIG. 2q-u show scatter bar graphs that show phosphorylated STAT6 (FIG. 2q), Arg1 (FIG. 2r), CD206 (FIG. 2s) and CD301 (FIG. 2t) protein levels or Adipoq gene expression (FIG. 2u) in CD45.2⁺ BMDMs co-cultured with ECP-treated CD45.1⁺ splenocytes for 48 h. BMDMs were treated with vehicle or the phagocytosis inhibitors Endosidin9 or PitStop2. Experiments were performed three times and data were pooled (mean±s.e.m.). Results are shown as fold change of MFI, compared to vehicle. Significance was calculated with the Friedman test.

FIGS. 2EA-2EK show that the immunosuppressive effects of ECP are reduced upon depletion of intestinal phagocytes.

Specifically, FIG. 2EA shows a scatter bar graph that depicts CD11b⁺F4/80⁺ macrophages as the percent of all immune cells in the colon wall of mice treated with DSS/anti-PD1/ECP. Mice either received vehicle (Encapsomes) or phagocyte depletion treatment (Clodrosomes). Data was pooled from two individual experiments (mean±s.e.m.). P-value was calculated with an unpaired Student's t-test.

FIG. 2EB shows bodyweights normalized to bodyweight on do of the experiment. All mice received DSS/anti-PD1/ECP-treatment. Mice were either treated with vehicle (Encapsomes) or phagocyte depletion treatment (Clodrosomes). Arrows indicate Encapsome and Clodrosome treatment or ECP transplantations. Data represents the mean of the pooled weight of all animals of the indicated group. Data were pooled from two experiments.

FIG. 2EC shows a scatter bar graph that shows mean (±s.e.m.) colon length of mice with DSS/anti-PD1-induced colitis. Mice received ECP combined with either vehicle or phagocyte depletion (Clodrosomes). Experiments were performed twice and p-value was calculated using an unpaired Student's 1-test.

FIG. 2ED and 2EE that show histopathological scoring of neutrophil (FIG. 2ED) and lymphocyte (FIG. 2EE) infiltration into the colon of mice with DSS/anti-PD1-induced colitis treated with ECP plus vehicle or phagocyte depletion. Scoring was done on d8 of the experiment. Each data point represents the value of an individual mouse. The results of two experiments were pooled (mean±s.e.m). The p-values were calculated using an unpaired Student's t-test.

FIG. 2EF shows a scatter bar graph that shows Ly6G⁺ neutrophils as the percent of all CD11b cells in the colon wall of mice treated with DSS/anti-PD1/ECP and either vehicle or phagocyte depletion (Clodrosomes). The results of two experiments were pooled (mean±s.e.m). The p-values were calculated using an unpaired Student's t-test.

FIG. 2EG shows a representative flow cytometry plot that shows neutrophils in the colon wall of DSS/anti-PD1/ECP-treated mice which received either vehicle or phagocyte-depleting Clodrosomes.

FIG. 2EH shows a scatter bar graph that shows adiponectin serum levels in mice treated with DSS/anti-PD1/ECP and either vehicle or phagocyte-depleting Clodrosomes. Data was pooled from two individual experiments (mean±s.e.m.). P-value was calculated with an unpaired Student's 1-test.

FIG. 2EI and 2EJ show graphs that show Adipoq (FIG. 2EI) or Arg1 (FIG. 2EJ) gene expression in colitis tissue of DSS/anti-PD1/ECP-treated mice which received either vehicle or phagocyte-depleting Clodrosomes. Gene expression was measured by qPCR and normalized to Hprt expression and is shown as fold change to vehicle treatment. Data from two individual experiments were pooled (mean±s.e.m.) and significance was calculated with an unpaired Student's 1-test.

FIG. 2EK shows a scatter bar graph that depicts CD45.2⁺VT680⁺ cells (mean±s.e.m.) isolated from the lamina propria of the colon tissue from tumor-bearing mice with DSS/anti-PD1-induced colitis. Mice received 2×10⁶ ECP-treated VT680⁺ MODE-K cells or 2×10⁶ ECP-treated CD45.1⁺VT680⁺ splenocytes 12 h before tissue harvest. P-value was calculated using Mann-Whitney test.

FIGS. 3a-31 show that scRNA seq based analysis of colonic immune cells revealed major differences in myeloid cells upon ECP-treatment.

Specifically, FIG. 3a shows a bar diagram that represents the frequency of intestinal immune cells based on their expression profile. Cells were isolated from the colon lamina propria of mice that received DSS/anti-PD1 treatment alone or combined with ECP. Two mice were analyzed per group.

FIG. 3b shows a UMAP visualization of immune cells isolated from the colon lamina propria of mice that received DSS/anti-PD1 treatment alone or combined with ECP. The different cell types were determined by Louvain clustering analysis and marker gene expression. Neutrophils and macrophages are highlighted. Two mice were analyzed per group.

FIGS. 3c and 3d show a UMAP visualization of calculated PPARγ (FIG. 3c) and STAT6 (FIG. 3d) transcription factor activity in immune cells isolated from the colon of mice that received DSS/anti-PD1 treatment alone or in combination with ECP. Clusters were described in FIG. 3b. Macrophage populations are marked. Transcription factor activity was calculated based on expression changes in downstream genes. Two mice were analyzed per group.

FIG. 3e shows a visualization of PPARγ and STAT6 transcription factor activity in the macrophage cluster shown in FIG. 3b. Macrophages from mice with DSS/anti-PD1 treatment alone or in combination with ECP are shown. Numbers show the calculated activity score, whereas the size of the circle represents the number of cells with detected activity. Two mice were analyzed per group.

FIG. 3f shows a graph that shows bodyweights normalized to do. Bodyweight was measured daily. Mice received DSS/anti-PD1 treatment combined with ECP, and were either treated with vehicle, PPARγ-inhibitor (GW9662), or STAT6-inhibitor (AS1517499). The arrows indicate when treatment with vehicle, PPARγ-inhibitor or STAT6-inhibitor was performed, or when mice received ECP transplantations. Each data point represents the mean of the pooled weight of all animals of the indicated group. Data was pooled from two experiments.

FIG. 3g shows a scatter bar graph that shows mean (±s.e.m.) colon length of mice with DSS/anti-PD1-induced colitis, combined with ECP transplantation. Mice were treated with vehicle, PPARγ-inhibitor or STAT6-inhibitor as indicated. Data from two experiments was pooled and significance was calculated using an ordinary one-way ANOVA.

FIGS. 3h and 3i show a histopathology scoring of neutrophil (FIG. 3h) and lymphocyte (FIG. 3i) infiltration into the colon of mice which received DSS/anti-PD1 treatment combined with ECP. Mice were treated with vehicle, PPARγ-inhibitor or STAT6-inhibitor as indicated. Scoring was done at d8. Two individual experiments were pooled (mean±s.e.m.). P-values were calculated by Kruskal-Wallis test.

FIG. 3j shows a graph that depicts adiponectin serum levels in mice that received DSS/anti-PD1 treatment combined with ECP, and which were additionally treated with vehicle, PPARγ-inhibitor or STAT6-inhibitor as indicated. Data from two experiments were pooled (mean±s.e.m.) and significance was calculated by Kruskal-Wallis test.

FIGS. 3k and 3l show graphs that show Adipoq (FIG. 3k) or Arg/(FIG. 3l) gene expression in colitis tissue of DSS/anti-PD1/ECP-treated mice which received either vehicle, PPARγ-inhibitor or STAT6-inhibitor as indicated. Gene expression was measured by qPCR and normalized to Hprt expression and shown as fold change to vehicle treatment. Data from two individual experiments was pooled (mean±s.e.m.) and significance was calculated with an ordinary one-way ANOVA or Kruskal-Wallis test.

FIGS. 3EA-3EE show that Adiponectin expression in vitro is reduced upon STAT6 and PPARγ blockade.

Specifically, FIGS. 3EA-3ED show scatter bar graphs that show Arg1 (FIG. 3EA), CD206 (FIG. 3EB) and CD301 (FIG. 3EC) protein levels or Adipoq gene expression (FIG. 3ED) in CD45.2 BMDMs co-cultured with ECP-treated CD45.1⁺ splenocytes for 48 h. BMDMs were treated with vehicle, STAT6-inhibitor (AS1517499) or PPARγ-inhibitor (T0070907). Data from three individual experiments was pooled (mean±s.e.m.). Results are shown as fold change of MFI compared to vehicle. Significance was calculated with the Friedman test.

FIG. 3EE shows violin plots that depict differential gene expression in the colonic macrophage cluster identified by scRNA Seq analysis. Cells isolated from DSS/anti-PD1 colitis mice with or without ECP-treatment are analyzed. Significance is indicated with p<0.01 (*), p<0.001 (**) and p<0.0001 (***).

FIGS. 4a-4p show that Adiponectin and Adiponectin-Receptor Agonist monotherapy as well as the combination with ECP reduced intestinal inflammation.

Specifically, FIG. 4a shows a graph that shows bodyweights normalized to initial bodyweight on do. Bodyweight was measured daily. Groups include animals with DSS/anti-PD1-induced colitis treated vehicle or Adiponectin receptor agonist (AdipoR Agonist; ARA). Data represents the mean of the pooled weight of all animals of the indicated group. Arrows indicate when treatment with vehicle or AdipoR Agonist was performed. Data were pooled from two experiments.

FIG. 4b shows a scatter bar graph that shows mean (±s.e.m.) colon length of mice with DSS/anti-PD1-induced colitis treated with vehicle or AdipoR Agonist as indicated. Data were pooled from two experiments. P-value was calculated using an unpaired Student's t-test.

FIGS. 4c and 4d show scatter bar graphs that depict histopathological grading of neutrophil (FIG. 4c) and lymphocyte (FIG. 4d) infiltration into the colon of mice with DSS/anti-PD1-induced colitis, treated with vehicle or AdipoR Agonist, as indicated. Results were pooled from two individual experiments (mean±s.e.m.). P-values were calculated using Mann-Whitney test.

FIG. 4e shows a graph that shows bodyweights normalized to initial bodyweight on do. Groups include animals with DSS/anti-PD1-induced colitis treated vehicle or recombinant Adiponectin. Data represents the mean of the pooled weight of all animals in the indicated group. The arrows indicate when treatment with vehicle or Adiponectin was performed. Data were pooled from two experiments.

FIG. 4f that shows a scatter bar graph that shows mean (±s.e.m.) colon length of mice with DSS/anti-PD1-induced colitis treated with vehicle or recombinant Adiponectin, as indicated. Data were pooled from two experiments. P-value was calculated using an unpaired Student's t-test.

FIGS. 4g and 4h show scatter bar graphs that depict histopathological grading of neutrophil (FIG. 4g) and lymphocyte (FIG. 4h) infiltration into the colon of mice with DSS/anti-PD1-induced colitis, treated with vehicle or recombinant Adiponectin, as indicated. Results were pooled from two individual experiments (mean±s.e.m.). P-values were calculated using Mann-Whitney test.

FIG. 4i shows a graph that shows bodyweights normalized to initial bodyweight on do. Groups include animals with DSS/anti-PD1-induced colitis treated with ECP in combination with vehicle or AdipoR Agonist. Data represents the mean of the pooled weight of all animals of the indicated group. The arrows indicate when treatment with vehicle or AdipoR Agonist and ECP was performed. Data were pooled from two experiments.

FIG. 4j shows a scatter bar graph that shows the mean (±s.e.m.) of the colon length of mice. Groups include animals with DSS/anti-PD1-induced colitis treated with ECP in combination with vehicle or AdipoR Agonist. Pooled values of two experiments are shown. P-value was calculated using an unpaired Student's t-test.

FIGS. 4k and 4l shows scatter bar graphs that depict histopathological grading of neutrophil (FIG. 4k) and lymphocyte (FIG. 4l) infiltration into the colon of mice with DSS/anti-PD1-induced colitis, treated with ECP in combination with vehicle or AdipoR Agonist. Results were pooled from two individual experiments (mean±s.e.m.). P-values were calculated using Mann-Whitney test.

FIG. 4m shows a graph that shows bodyweights normalized to initial bodyweight on d0. Groups include animals with DSS/anti-PD1-induced colitis treated with ECP in combination with vehicle or recombinant Adiponectin. Data represents the mean of the pooled weight of all animals from the indicated group. The arrows indicate when treatment with vehicle or Adiponectin (grey) and ECP (black) was performed. Data from two experiments were pooled.

FIG. 4n shows a scatter bar graph that shows the mean (±s.e.m.) of the colon length of mice. Groups include animals with DSS/anti-PD1-induced colitis treated with ECP in combination with vehicle or recombinant Adiponectin. Pooled values from two experiments are shown. P-value was calculated using an unpaired Student's t-test.

FIGS. 40 and 4p show scatter bar graphs that depict histopathological grading of neutrophil (FIG. 40) and lymphocyte (FIG. 4p) infiltration into the colon of mice with DSS/anti-PD1-induced colitis, treated with ECP in combination with vehicle or recombinant Adiponectin. Results were pooled from two individual experiments (mean±s.e.m.). P-values were calculated using the Mann-Whitney test.

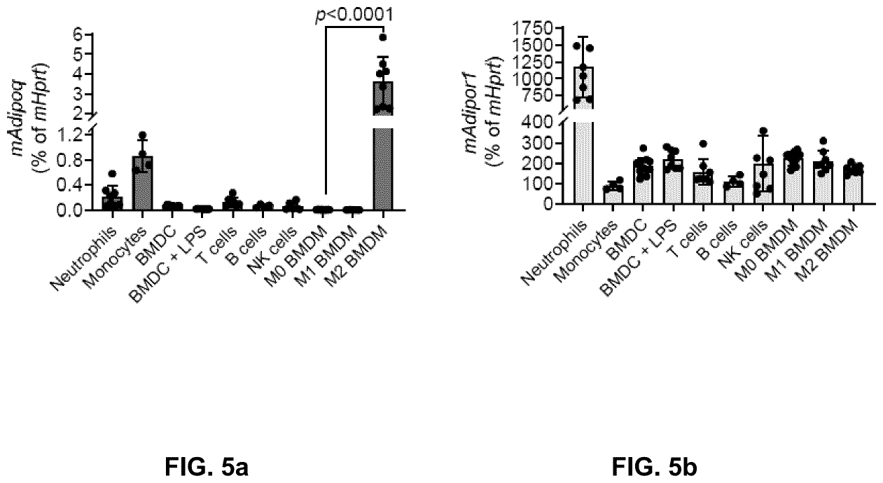

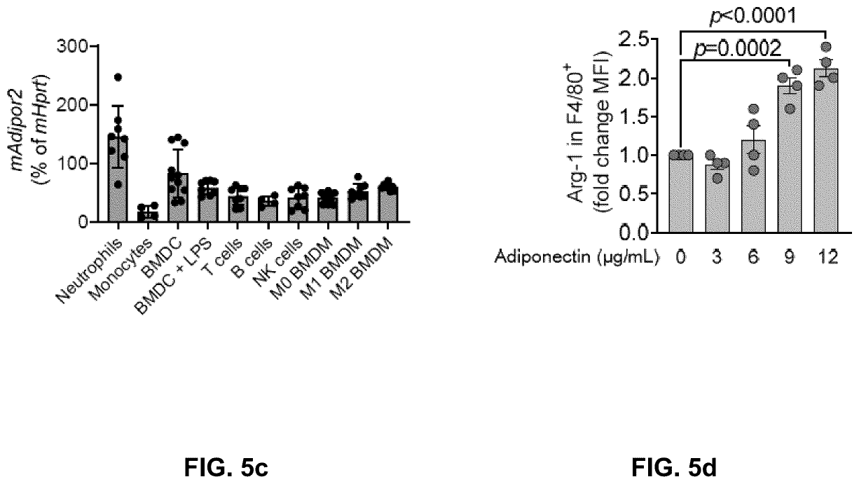

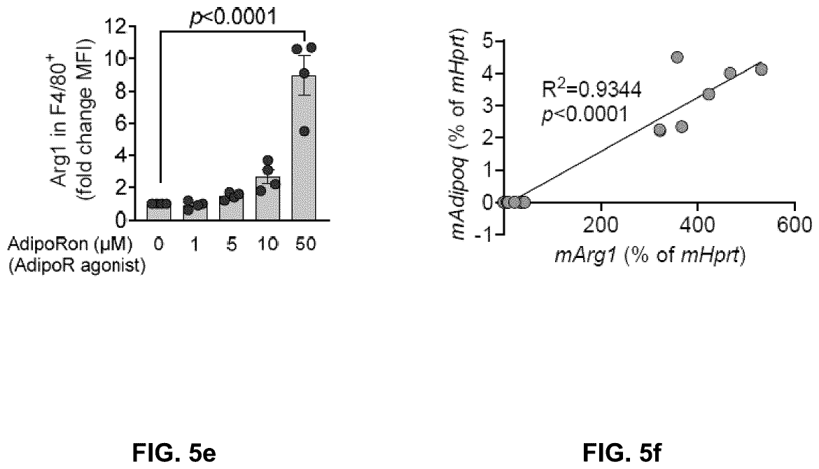

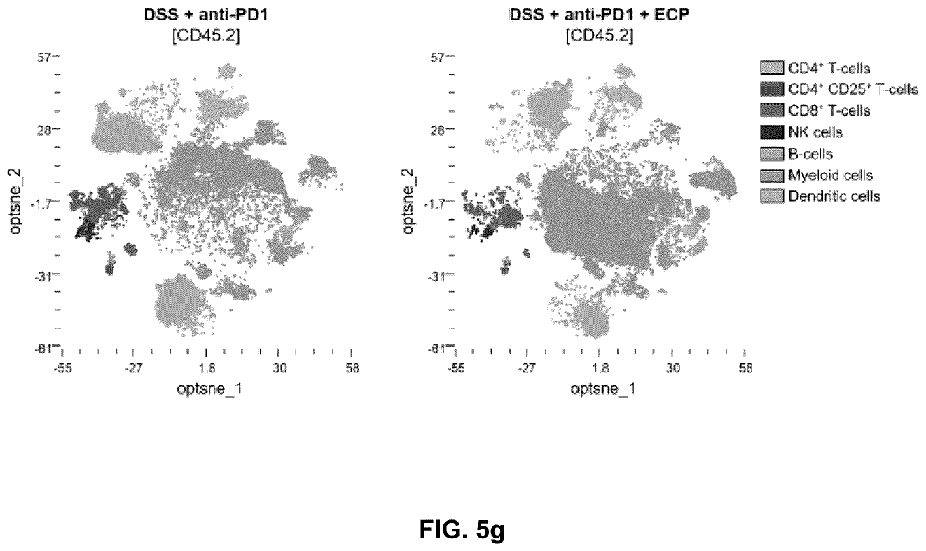

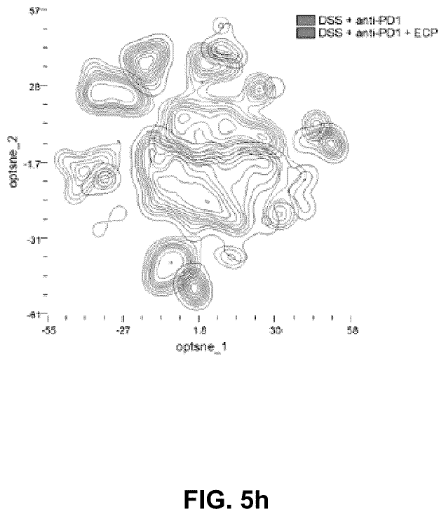

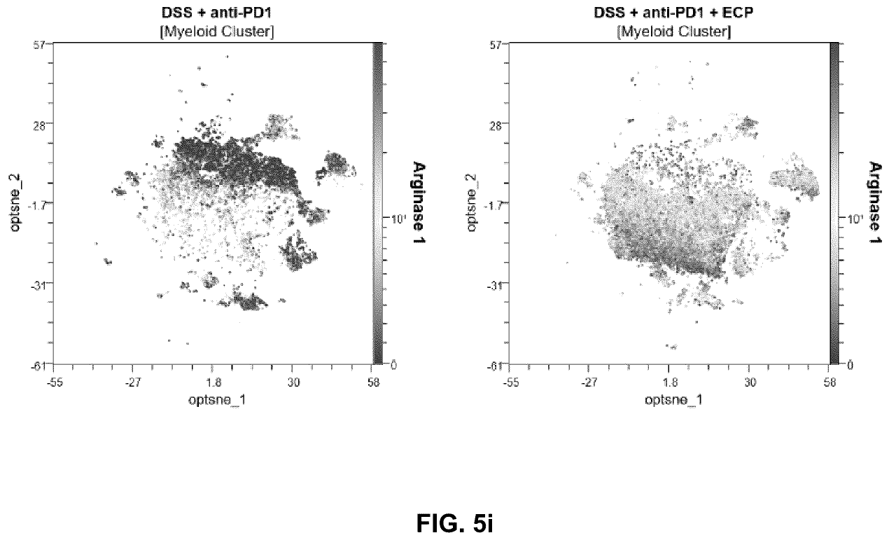

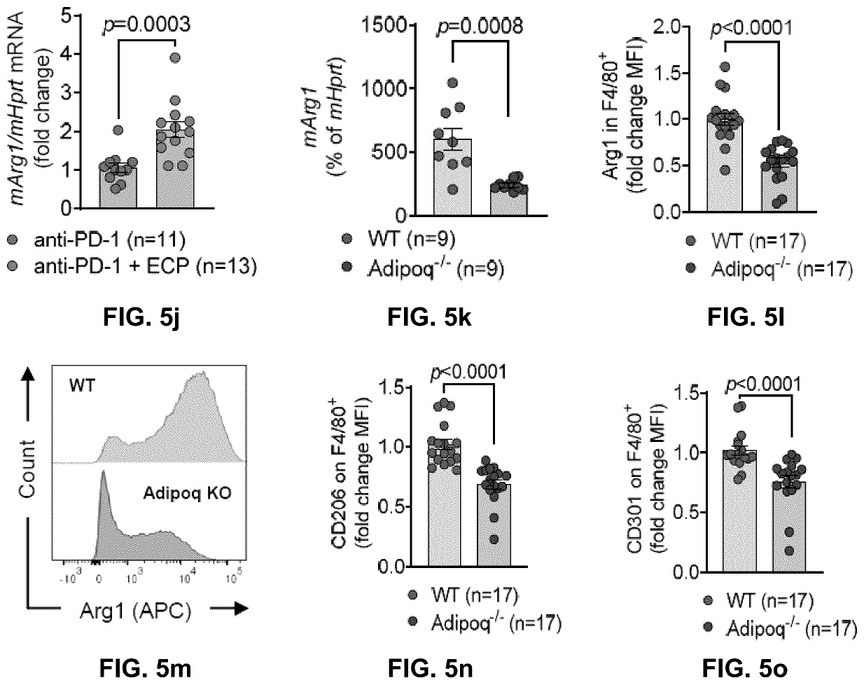

FIGS. 5a-5o show that ECP caused Arg1 upregulation in myeloid cells.

Specifically, FIGS. 5a-5c show scatter bar graphs that show the mean (±s.e.m.) of Adipoq (FIG. 5a), Adipor1 (FIG. 5b) and Adipor2 (FIG. 5c) gene expression in different cell types isolated from naïve mice. The pooled values of at least two individual experiments are shown (n=4-12 per cell type). Each data point represents a biological replicate. P-values were calculated using ordinary one-way ANOVA.

FIGS. 5d and 5e show scatter bar graphs that show the MFI of Arg1 in F4/80⁺ macrophages upon exposure to vehicle or increasing concentrations of recombinant Adiponectin (FIG. 5d) or AdipoR Agonist (FIG. 5e). The experiment was performed twice and the mean (±s.e.m.) is shown. Each data point shows a biological replicate (n=4). P-values were calculated using ordinary one-way ANOVA.

FIG. 5f shows a graph that shows the relative Adipoq expression as percent of Hprt (y-axis) and Arg1 expression as percent of Hprt (x-axis) in BMDMs. The pooled values of four experiments are shown (n=21). Correlation coefficient and p-value were calculated using regression analysis.

FIG. 5g show opt-SNE maps that display CD45.2⁺ recipient cells (ECP donor: CD45.1) from the colon lamina propria of mice with DSS-induced colitis, treated with anti-PD1 alone or anti-PD1 plus ECP. Immune cells were analyzed by CyTOF. Colors correspond to FlowSOM-guided clustering of cell populations, clusters were identified by signature marker expression.

FIG. 5h shows an overlay of opt-SNE maps shown in FIG. 5G. Maps display intestinal CD45.2⁺ recipient immune cells from mice with DSS-induced colitis treated with anti-PD1 alone or anti-PD1 plus ECP.

FIG. 5i shows an opt-SNE map displaying arginase-1 expression in the myeloid cluster of CD45.2⁺ recipient cells isolated from the colon lamina propria of mice with DSS-induced colitis, treated with anti-PD1 alone or anti-PD1 plus ECP.

FIG. 5j shows a scatter bar graph that depicts Arg1 gene expression in the colon of mice with DSS-induced colitis being treated with anti-PD1 alone or anti-PD1 plus ECP. Gene expression data was measured by qPCR normalized to Hprt expression and is shown as fold change to anti-PD1 treatment. Results from three individual experiments were pooled (mean±s.e.m.). P-value was calculated using an unpaired Student's 1-test.

FIG. 5k shows a scatter bar graph that displays mArg1 in % of mHprt expression in M2-polarized BMDMs generated from WT C57BL/6 or Adipoq^{-/-} mice. Results from three experiments are pooled (mean±s.e.m.). P-value was calculated using an unpaired Student's 1-test.

FIG. 5l shows Arg1 protein levels in M2-polarized F4/80⁺ BMDMs derived from WT or Adipoq^{-/-} mice. Data from four experiments is pooled (mean±s.e.m.) and shown as fold change of MFI compared to WT. Significance was calculated using an unpaired Student's 1-test.

FIG. 5m shows representative histograms of Arg1 protein in M2-polarized F4/80⁺ BMDMs derived from WT or Adipoq^{-/-} mice.

FIGS. 5n and 5o show scatter bar graphs that show CD206 (FIG. 5N) and CD301 (FIG. 5O) protein levels on M2-polarized F4/80⁺ BMDMs derived from WT or Adipoq-mice, as MFI fold change compared to WT. Data from four experiments is pooled (mean±s.e.m.). P-values were calculated using an unpaired Student's 1-test and Mann-Whitney test.

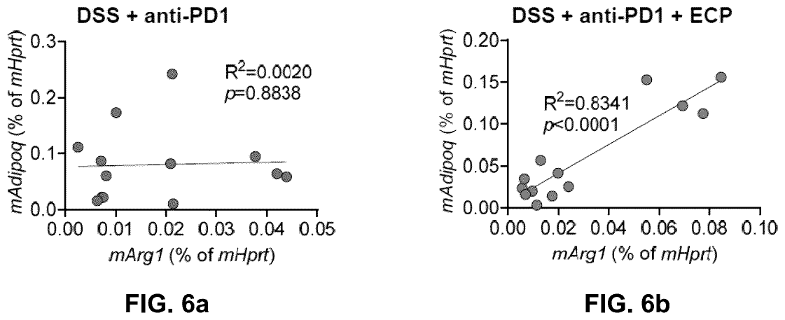

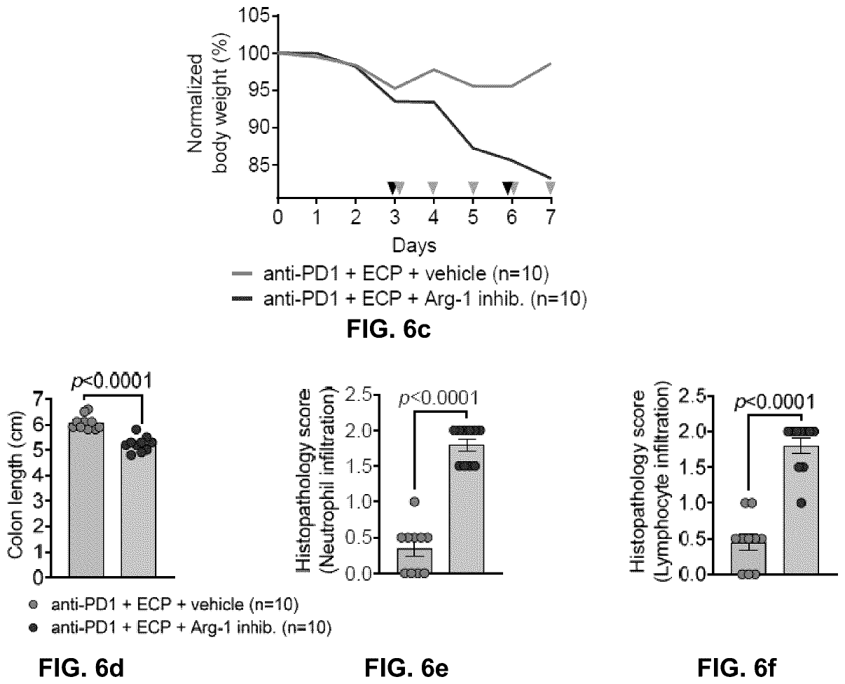

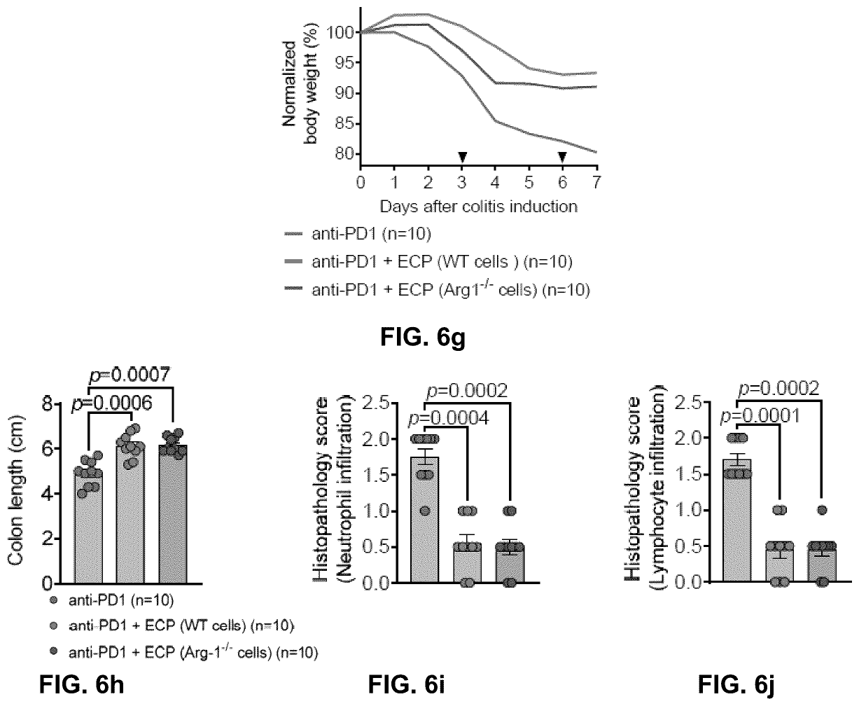

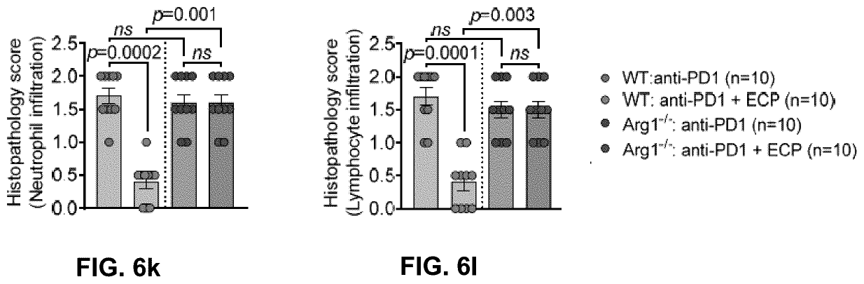

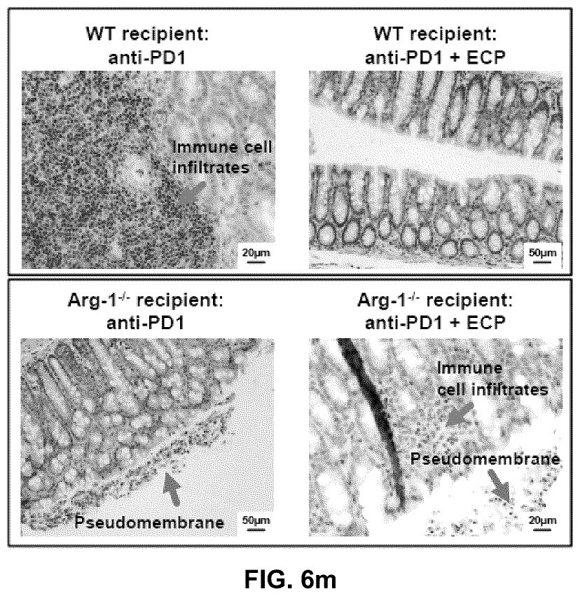

FIGS. 6a-6m show that Arg1 deficiency in the recipient compartment diminished protective ECP-effects.

Specifically, FIG. 6a shows a graph that displays the relative Adipoq expression as percent of Hprt (y-axis) and Arg1 expression as percent of Hprt (x-axis) in colon tissue of mice with DSS/anti-PD1-induced colitis. The pooled values of three experiments are shown (n=13). Correlation coefficient and p-value were calculated using regression analysis. No correlation was found.

FIG. 6b shows a graph that displays the relative Adipoq expression as percent of Hprt (y-axis), and Arg1 expression as percent of Hprt (x-axis) in colon tissue of mice with DSS/anti-PD1-induced colitis, treated with ECP. The pooled values of three experiments are shown (n=13). Correlation coefficient and p-value were calculated using regression analysis.

FIG. 6c shows a graph that shows bodyweights normalized to initial bodyweight on do. Groups include animals with DSS/anti-PD1-induced colitis treated with ECP and either vehicle or the Arg1-inhibitor CB1158. Data represents the mean pooled weight of all animals in the indicated group. The arrows indicate ECP transplantations and treatment with vehicle or Arg1-inhibitor. Data were pooled from two experiments.

FIG. 6d shows a scatter bar graph that shows mean (±s.e.m.) colon length of mice with DSS/anti-PD1-induced colitis treated with ECP and either vehicle or Arg1-inhibitor as indicated. Pooled values of two experiments are shown. P-value was calculated using an unpaired Student's 1-test.

FIGS. 6e and 6f show scatter bar graphs that depict histopathological grading of neutrophil (FIG. 6e) and lymphocyte (FIG. 6f) infiltration into the colon of mice with DSS/anti-PD1-induced colitis treated with ECP in combination with vehicle or Arg1-inhibitor (CB1158). Results were pooled from two individual experiments (mean±s.e.m.). P-values were calculated using a Mann-Whitney test.

FIG. 6g shows a graph that shows bodyweights normalized to initial bodyweight on do. Groups include animals with DSS-induced colitis treated with anti-PD1 alone or anti-PD1 plus ECP. When indicated, ECP-treated splenocytes were derived from WT or Arg1^{-/-} donor mice. Arrows indicate ECP transplantations. Data represents mean pooled weight of all animals in the indicated group. Data were pooled from two experiments.

FIG. 6h shows a scatter bar graph that shows mean (±s.e.m.) colon length of mice with DSS-induced colitis treated with anti-PD1 alone or anti-PD1 plus ECP. When indicated, ECP-treated splenocytes were derived from WT or Arg1^{-/-} donor mice. The pooled values from two experiment are shown. P-values were calculated using a Kruskal-Wallis test.

FIGS. 6i and 6j show scatter bar graphs that depict histopathological grading of neutrophil (FIG. 6i) and lymphocyte (FIG. 6j) infiltration into the colon of mice with DSS-induced colitis treated with anti-PD1 alone or anti-PD1 plus ECP. When indicated, ECP-treated splenocytes were derived from WT or Arg1^{-/-} donor mice. Results were pooled from two individual experiments (mean±s.e.m.). P-values were calculated using a Kruskal-Wallis test.

FIGS. 6k and 6l show scatter bar graphs that show histopathological grading of neutrophil (FIG. 6k) and lymphocyte (FIG. 6l) infiltration into the colon of mice with DSS-induced colitis treated with anti-PD1 alone or in combination with ECP. When indicated, the recipient mice were WT or Arg^{-/-}. ECP donor mice were WT. Results were pooled from two individual experiments (mean±s.e.m.). P-values were calculated using a Kruskal-Wallis test.

FIG. 6m shows representative H&E images of colon samples from mice with DSS-induced colitis treated with anti-PD1 alone or combined with ECP. When indicated, the recipient mice were WT or Arg1^{-/-}. FIG. 6m further includes arrows that point towards immune cell infiltrates in the colon wall and arrows that point towards a pseudomembrane on the colon mucosa.

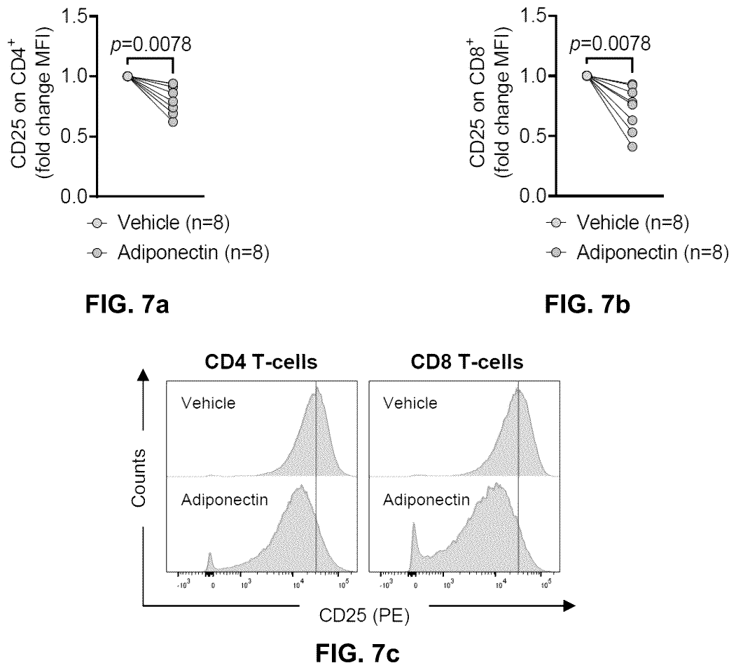

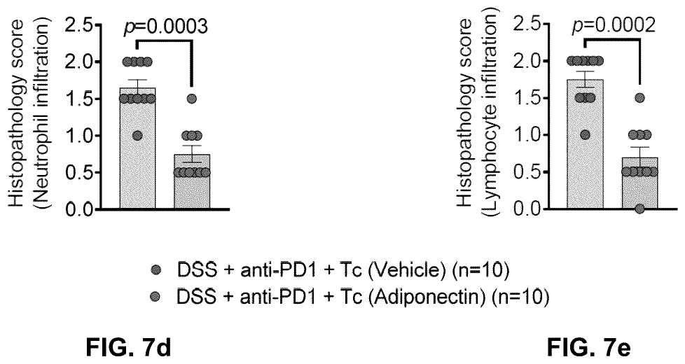

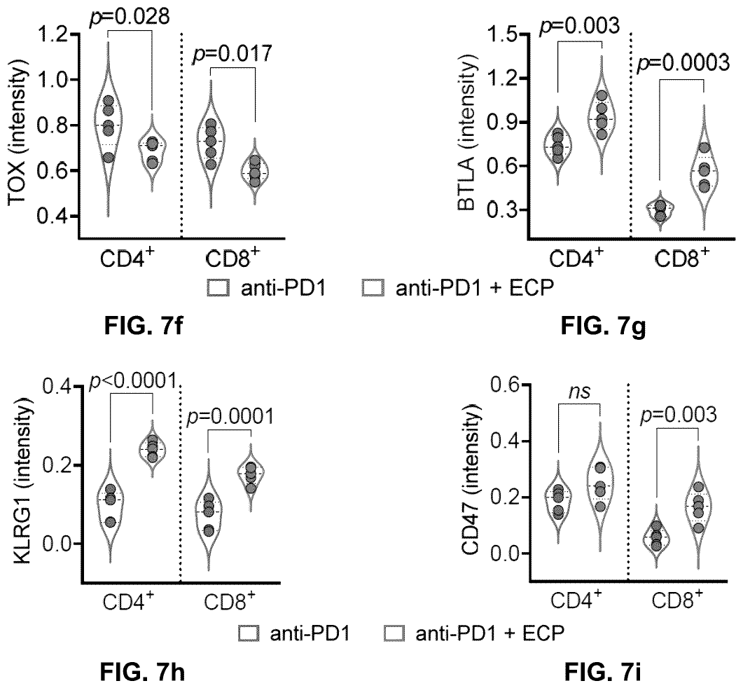

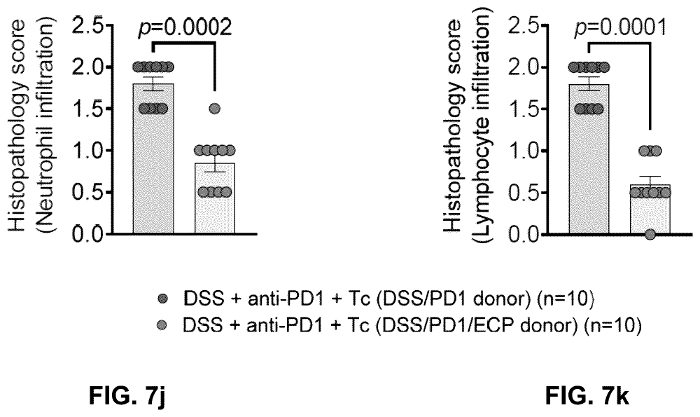

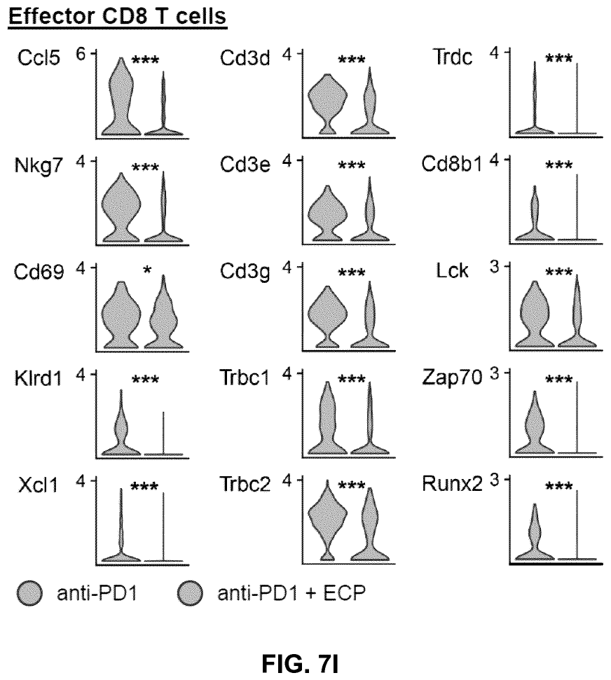

FIGS. 7a-7l show that ECP and adiponectin reduced pathogenic T-cell activation.

Specifically, FIGS. 7a and 7b show pan T-cells that were treated with vehicle or recombinant adiponectin in the presence of anti-CD3/CD28 activation beads. Graphs show CD25 expression on CD4⁺ (FIG. 7a) and CD8⁺ (FIG. 7b) T-cells after 72 h of culture. T-cells were adoptively transferred into recipients with DSS/anti-PD1-induced colitis. Data were pooled from two experiments. Significance was calculated using a Wilcoxon test.

FIG. 7c shows representative histograms of CD25 expression on CD4⁺ and CD8⁺ T-cells after 72 h treatment with vehicle or adiponectin in the presence of anti-CD3/CD28 activation beads.

FIGS. 7d and 7e show scatter bar graphs that show histopathological grading of neutrophil (FIG. 7d) and lymphocyte (FIG. 7e) infiltration into the colon of mice with DSS/anti-PD1-induced colitis, which received adoptive transfer of anti-CD3/CD28 activated T-cells treated with vehicle or adiponectin for 72 h. Results were pooled from two individual experiments (mean±s.e.m.). P-values were calculated using a Mann-Whitney test.

FIGS. 7f-7i show violin plots that display protein levels of TOX (FIG. 7f), BLTA (FIG. 7g), KLRG1 (FIG. 7h) and CD47 (FIG. 7i) in splenic CD4⁺ or CD8⁺ T-cells, analyzed by CyTOF. Gating on CD45.2⁺ recipient cells. Cells were isolated from mice with DSS-induced colitis treated with anti-PD1 alone or in combination with ECP. Data from one experiment (n=5).

FIGS. 7j and 7k show scatter bar graphs that show histopathological grading of neutrophil (FIG. 7j) and lymphocyte (FIG. 7k) infiltration into the colon of secondary recipients with DSS/anti-PD1-induced colitis which received adoptive transfer of splenic pan T-cells from primary donor. Donors had DSS-induced colitis and were either treated with anti-PD1 or anti-PD1 plus ECP. Results were pooled from two individual experiments (mean±s.e.m.). P-values were calculated using the Mann-Whitney test.

FIG. 7l shows violin plots that show expression of selected genes in the colonic effector CD8 T-cells cluster identified by scRNA Seq analysis. Cells isolated from DSS/anti-PD1 colitis mice with or without ECP-treatment are analyzed. Significance is indicated with p<0.01 (*), p<0.001 (**) and p<0.0001 (***).

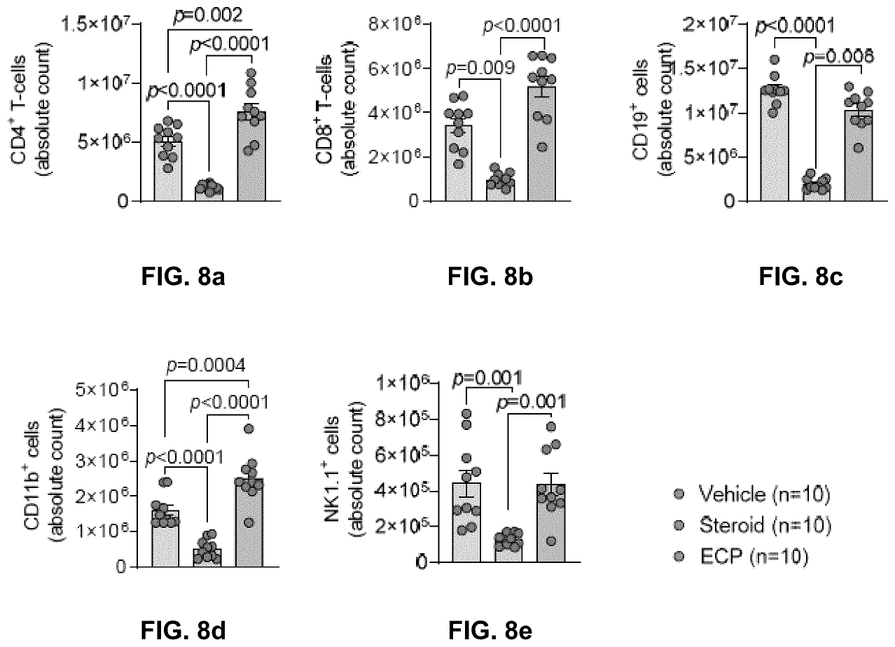

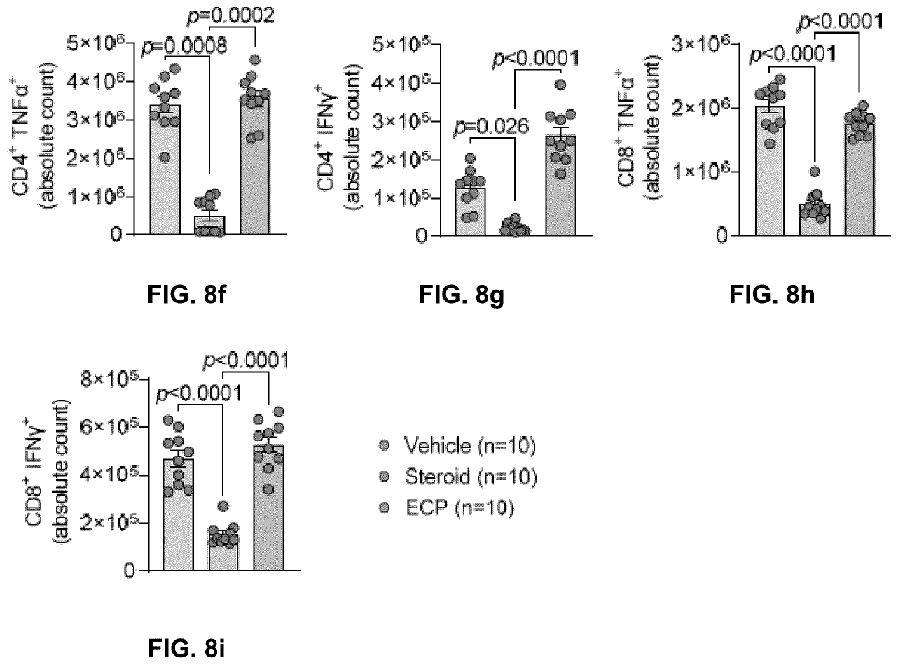

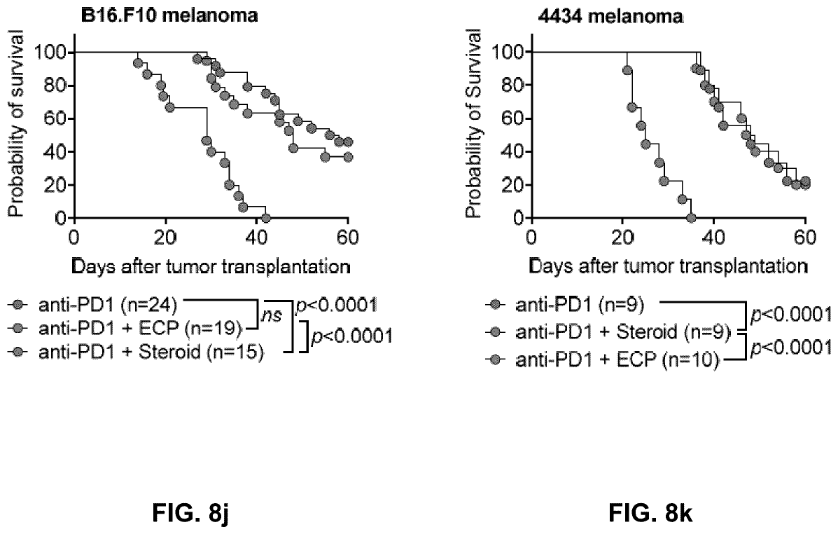

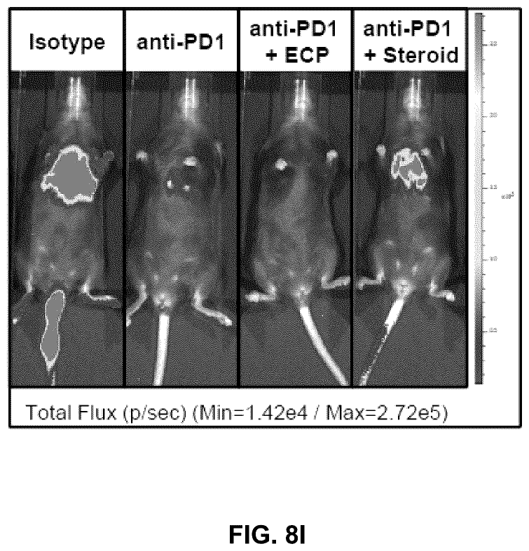

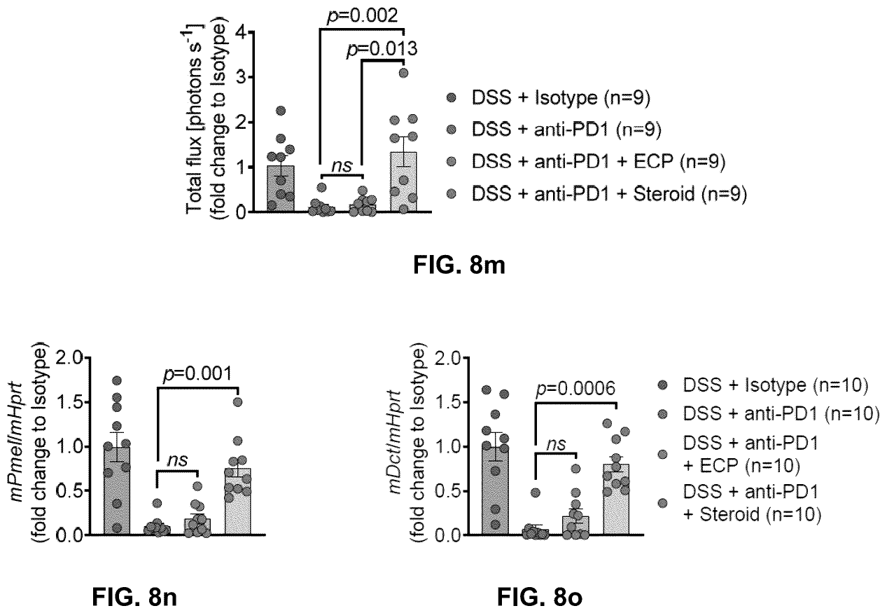

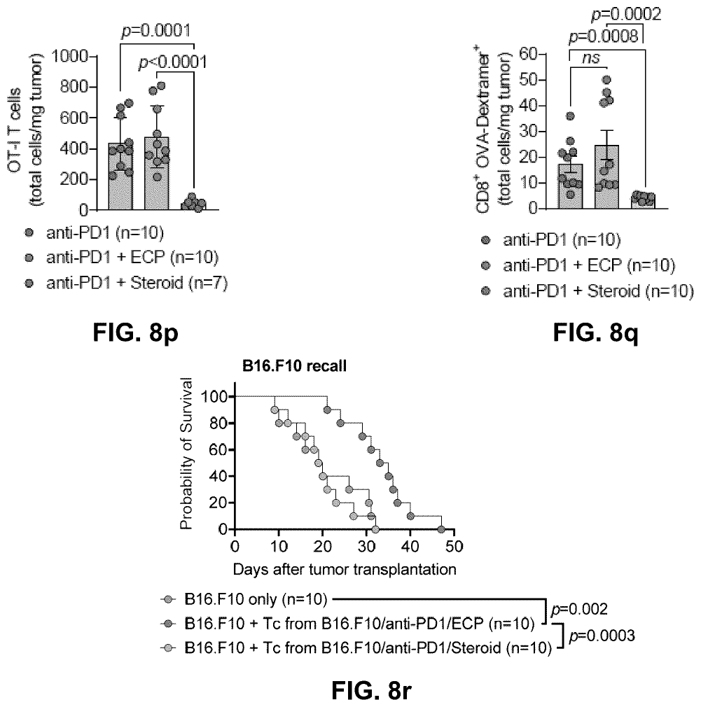

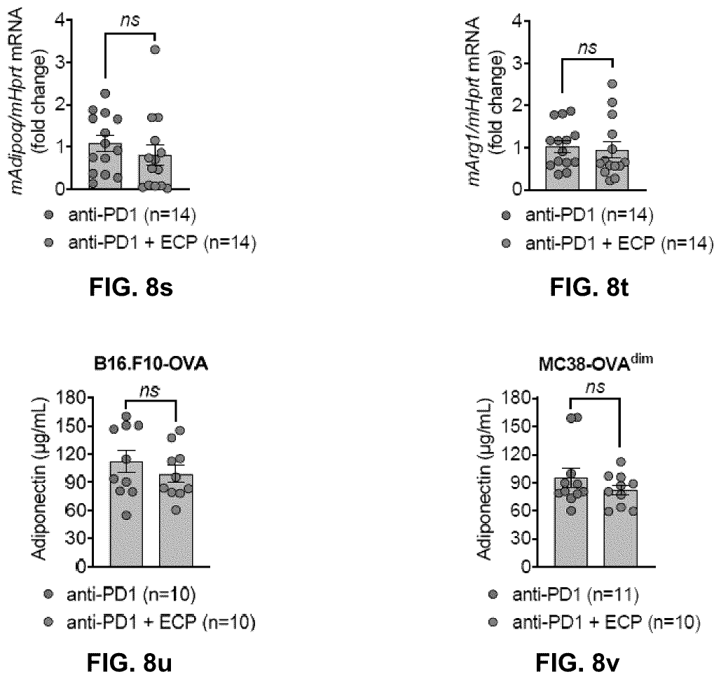

FIGS. 8a-8v show that the protective ECP-effect against irAE colitis allowed for anti-tumor immune responses.

Specifically, FIGS. 8a-8i show scatter bar graphs that show the mean (±s.e.m.) of the absolute counts of CD4⁺ (FIG. 8a), CD8⁺ (FIG. 8b), CD19⁺ (FIG. 8c), CD11b⁺ (FIG. 8d), NK1.1⁺ (FIG. 8e), CD4⁺TNF⁺ (FIG. 8f), CD4⁺INFγ⁺ (FIG. 8g), CD8⁺TNF⁺ (FIG. 8h) and CD8⁺INFγ⁺ (FIG. 8i) cells isolated from the spleens of mice treated with vehicle, prednisolone or ECP, as indicated. Mice received daily vehicle or prednisolone treatment for 50 days or eight weekly ECP transplantations from CD45.1⁺ donors. Pre-gating on CD45.2⁺ recipient cells. Each data point represents the value of an individual mouse. Significance was calculated using a Kruskal-Wallis test or an ordinary one-way ANOVA.

FIG. 8j shows a survival analysis of C57BL/6 mice i.v transplanted with B16.F10 melanoma cells (C57BL/6 background). All mice were treated with anti-PD1 and received ECP transplantations or steroid treatment as indicated. Data from three individual experiments were pooled. Significance was calculated by Mantel-Cox test.

FIG. 8k shows a survival analysis of C57BL/6 mice i.v. transplanted with BRAF^{V600E}-mutant 4434 melanoma cells (C57BL/6 background). All mice were treated with anti-PD1 and received ECP transplantation or steroid treatment, as indicated. Data from two individual experiment were pooled. Significance was calculated by Mantel-Cox test.

FIG. 8l shows representative bioluminescence images of mice at d23 after B16.F10^{luc/GFP} melanoma transfer. All mice received DSS additionally to induce colitis and were treated with isotype, anti-PD1, or anti-PD1 in combination with ECP or steroid as indicated. For BLI, the following parameters were kept constant for all groups: amount of luciferin injected, type of luciferin, exposure time and distance to camera.

FIG. 8m shows quantification of bioluminescence signals from mice shown and described in FIG. 8l. Fold change of total flux compared to isotype control is displayed. Data of two individual experiments was pooled (mean±s.e.m.). P-values were calculated using an ordinary one-way ANOVA.

FIGS. 8n and 80 show graphs that display Pmel (FIG. 8n) or Dct (FIG. 80) gene expression in the lungs of mice described in FIG. 8l. Gene expression was measured by qPCR and normalized to Hprt expression and shown as fold change to isotype treatment. Data from two individual experiments was pooled (mean±s.e.m.) and significance was calculated with a Kruskal-Wallis test.

FIG. 8p shows a scatter bar graph that shows the number of OT-I T-cells per mg tumor tissue. Mice with s.c. B16.F10-OVA tumors received adoptive transfer of OT-I T-cells and were treated with anti-PD1 alone or in combination with ECP or steroids. Tumors were digested on d16 after tumor cell transplantation to isolate TILs. Data of two individual experiments was pooled (mean±s.e.m.). P-values were calculated using an ordinary one-way ANOVA.

FIG. 8q shows a scatter bar graph that shows the number of OVA-specific CD8 T-cells per mg tumor tissue. Mice with s.c. MC38-OVA^{dim} tumors were treated with anti-PD1 alone or in combination with ECP or steroids. Tumors were digested on d16 after tumor cell transplantation to isolate TILs. Data of two individual experiments were pooled (mean±s.e.m.). P-values were calculated using the Kruskal-Wallis test.

FIG. 8r shows a survival analysis of C57BL/6 mice i.v. transplanted with B16.F10 melanoma cells (C57BL/6 background). Recipients were sublethally irradiated before tumor cell transplantation. If indicated, mice received adoptive transfer of T cells from B16.F10 melanoma-bearing mice treated with anti-PD1 plus or anti-PD1 plus steroids. Data from two individual experiments were pooled. Significance was calculated with the Mantel-Cox test.

FIGS. 8s and 8t show graphs that display Adipoq (FIG. 8s) or Arg (FIG. 8t) gene expression in the s.c. B16.F10-OVA tumor tissue of mice which received OT-I T-cells and were treated with anti-PD1 alone or in combination with ECP. Gene expression was measured by qPCR and normalized to Hprt expression and shown as fold change to anti-PD1 treatment. Data from three individual experiments was pooled (mean±s.e.m.) and significance was calculated using an unpaired Student's 1-test or Mann-Whitney test.

FIGS. 8u and 8v show scatter bar graphs that show adiponectin serum concentrations in mice with s.c. grown B16.F10-OVA tumors with OT-I T-cells transfer (FIG. 8u) or with s.c. grown MC38-OVA^{dim} tumors (FIG. 8v). Mice were treated with anti-PD1 alone or in combination with ECP. Data from three individual experiments were pooled (mean±s.e.m.) and significance was calculated using an unpaired Student's t-test or Mann-Whitney test.

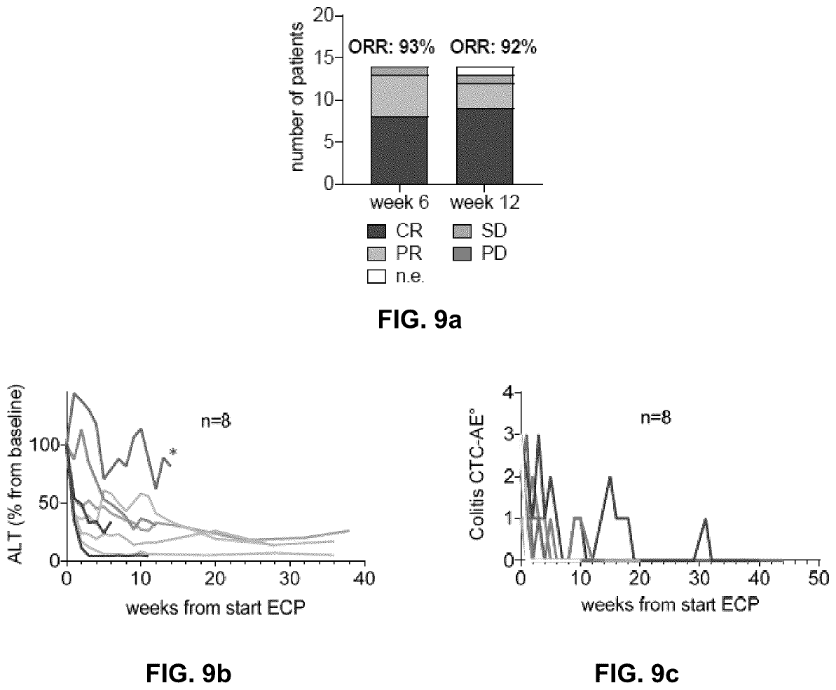

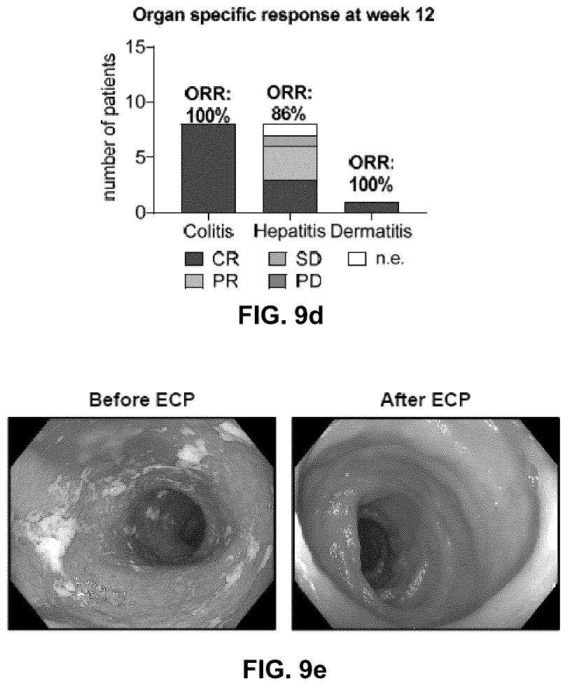

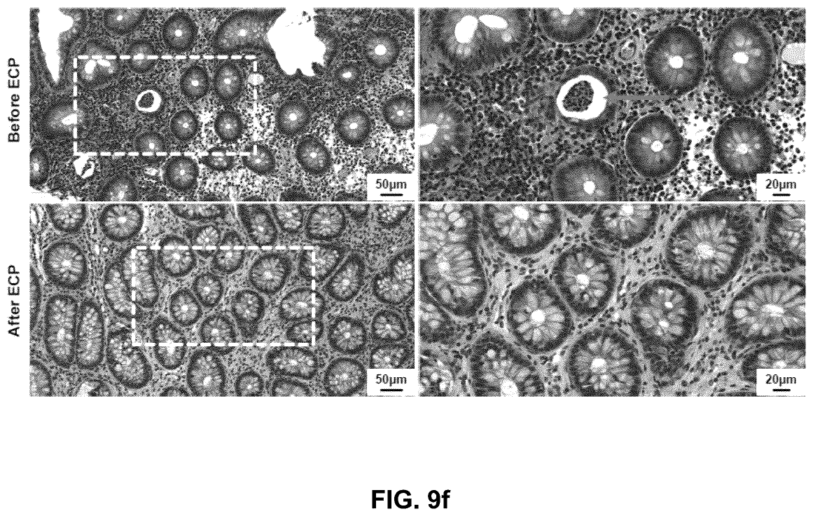

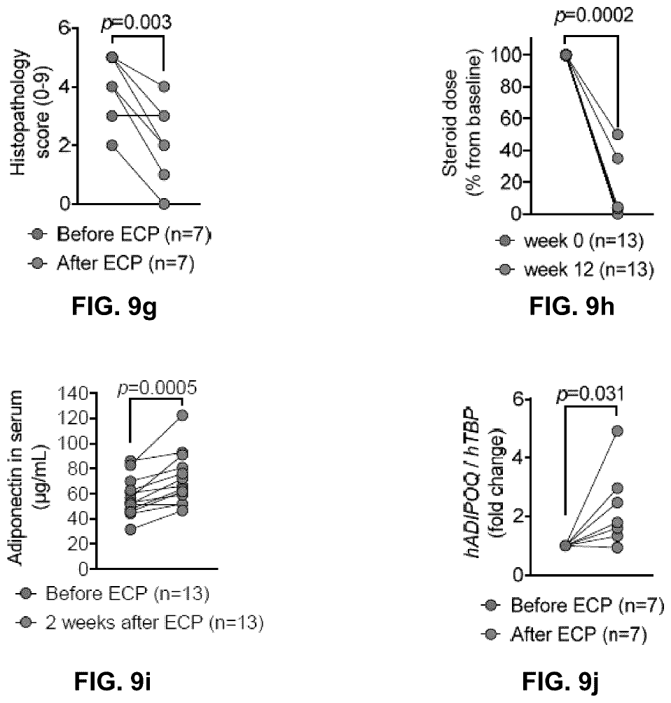

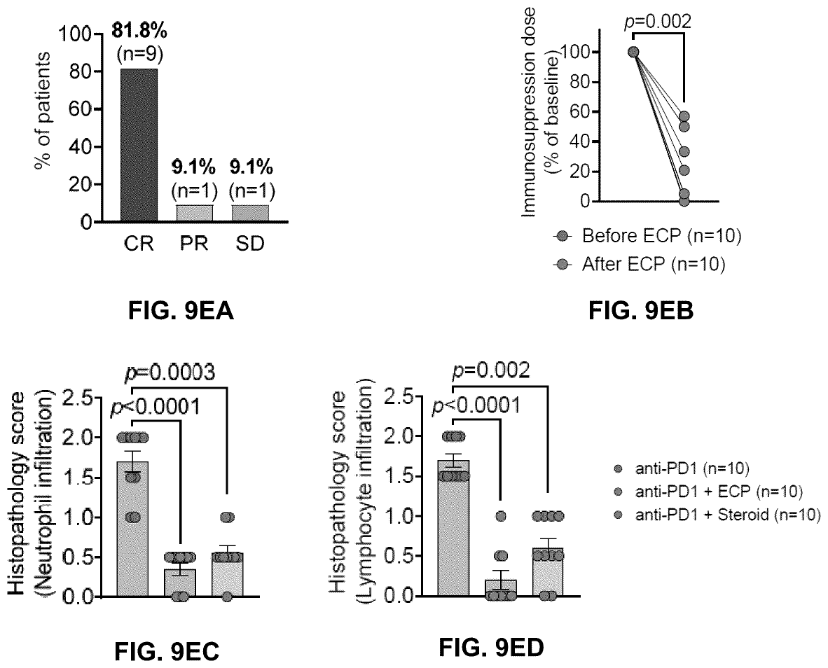

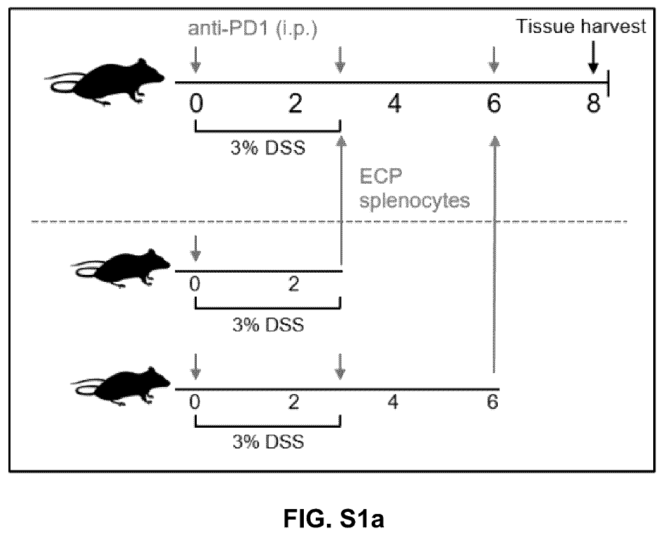

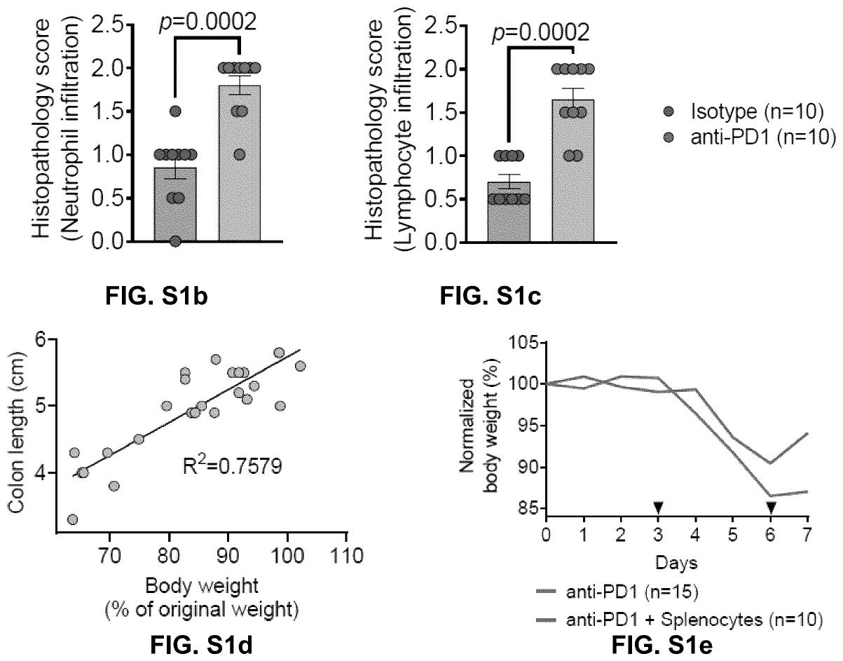

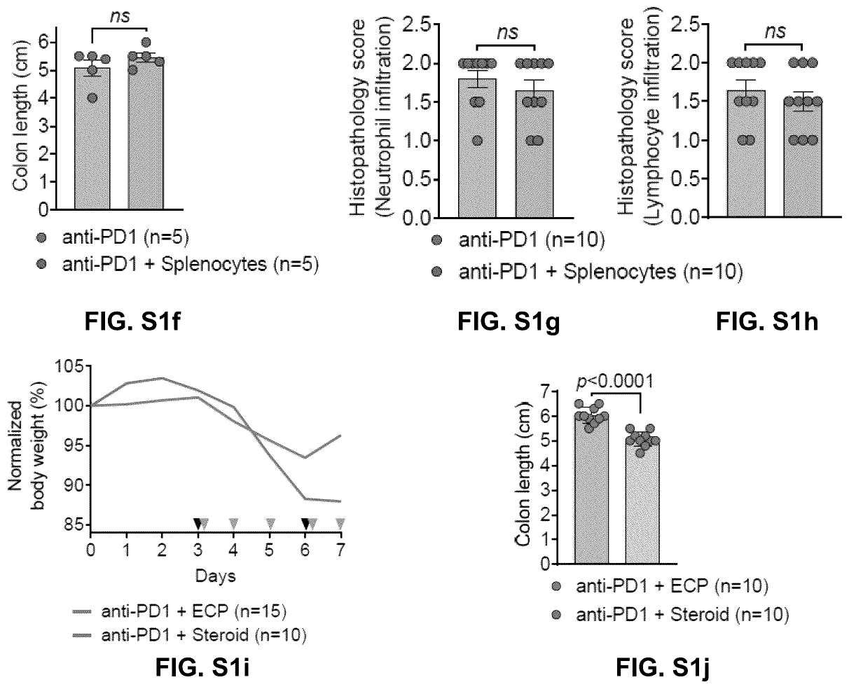

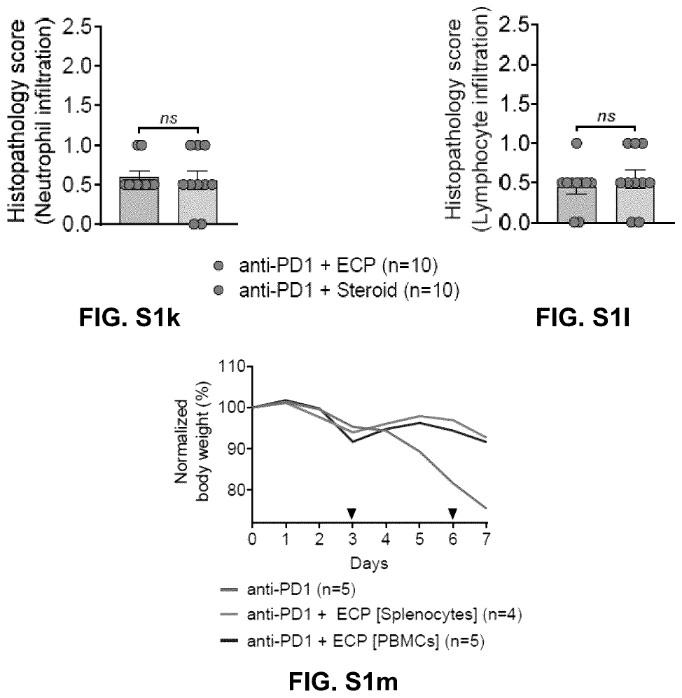

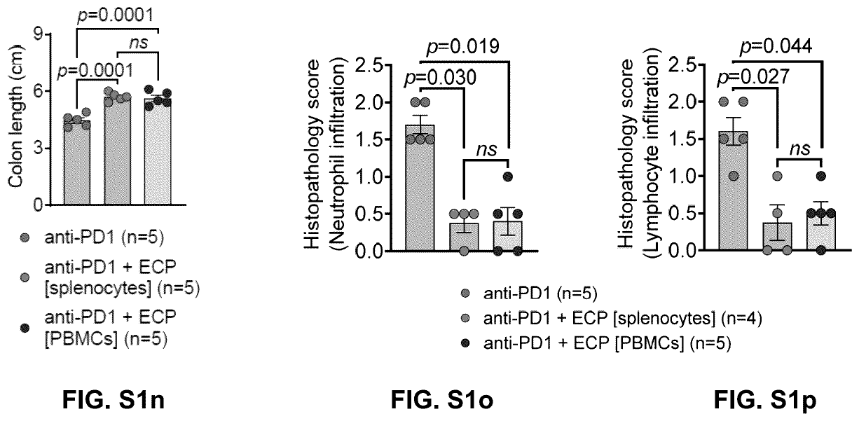

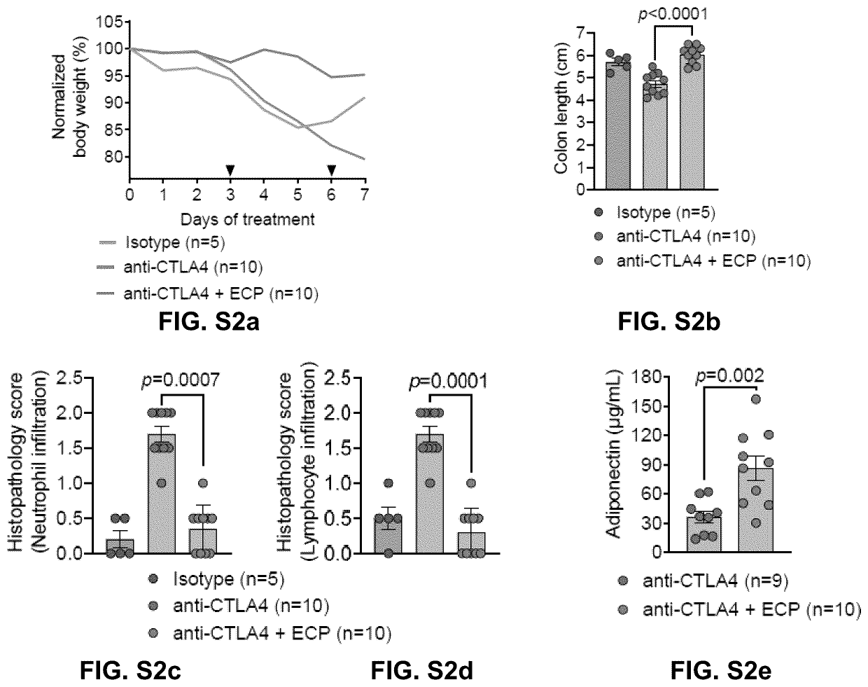

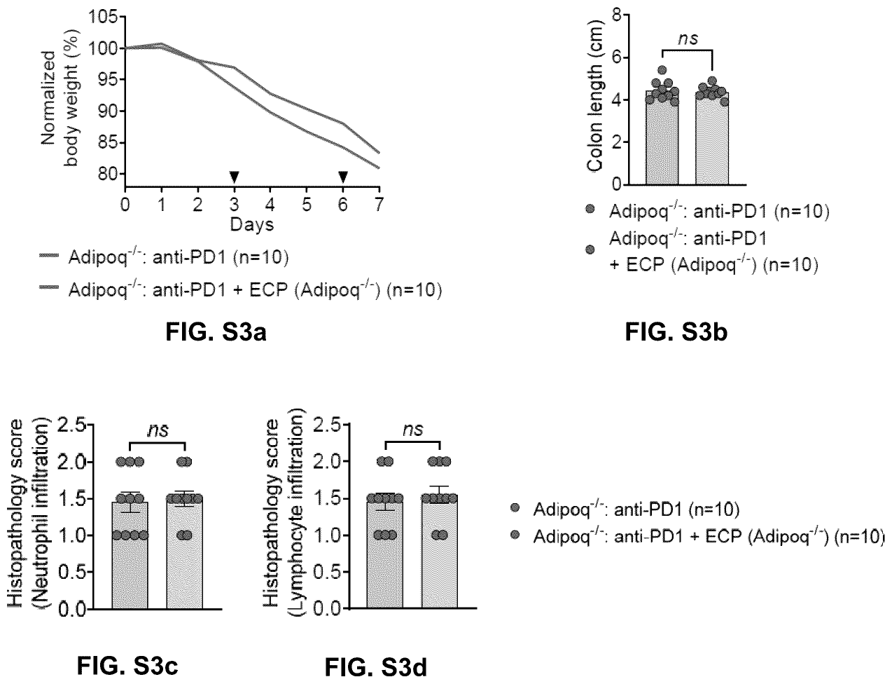

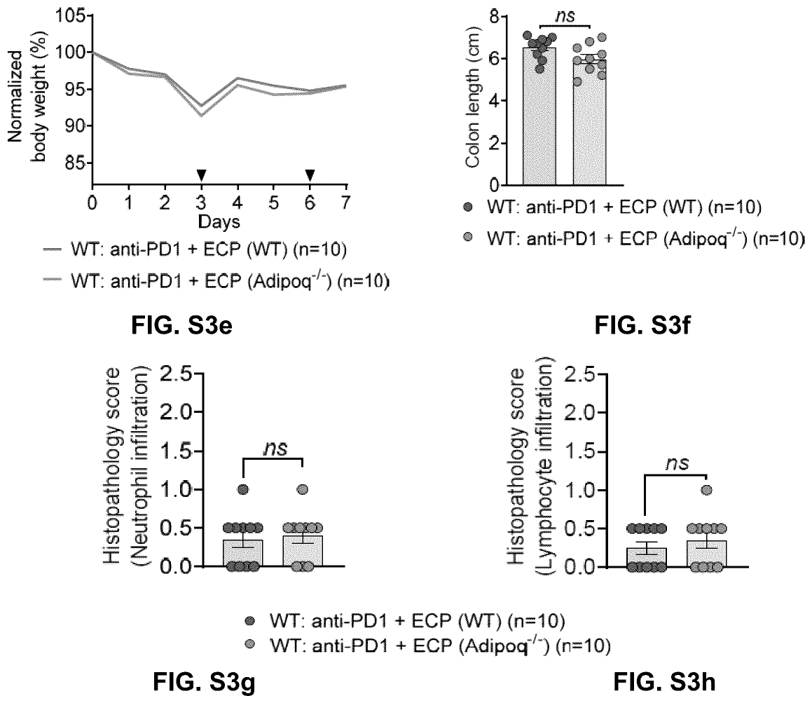

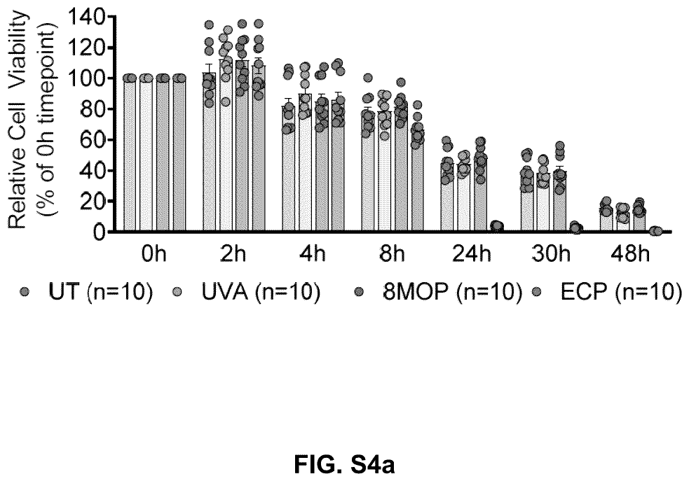

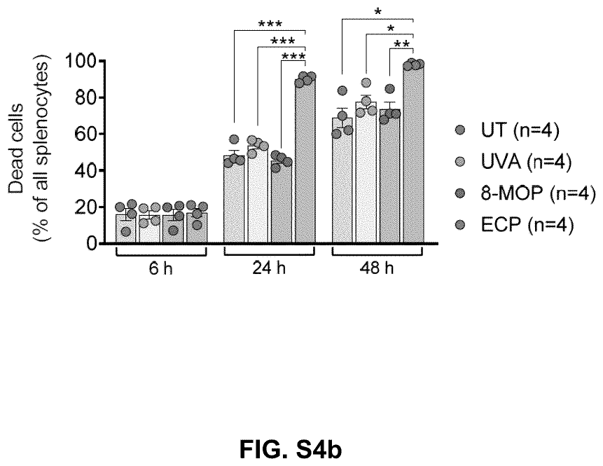

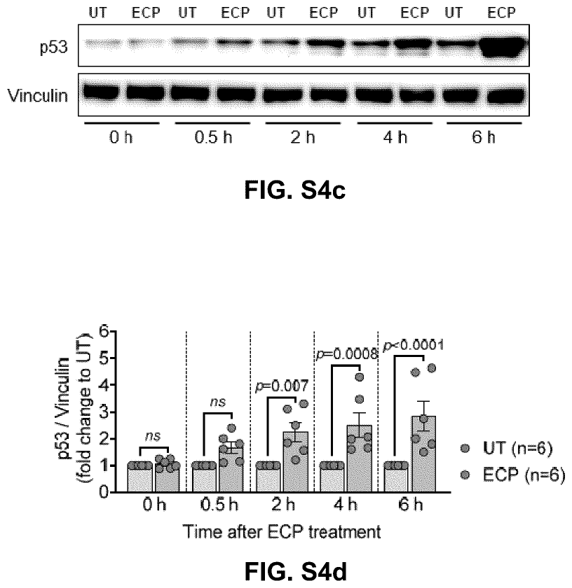

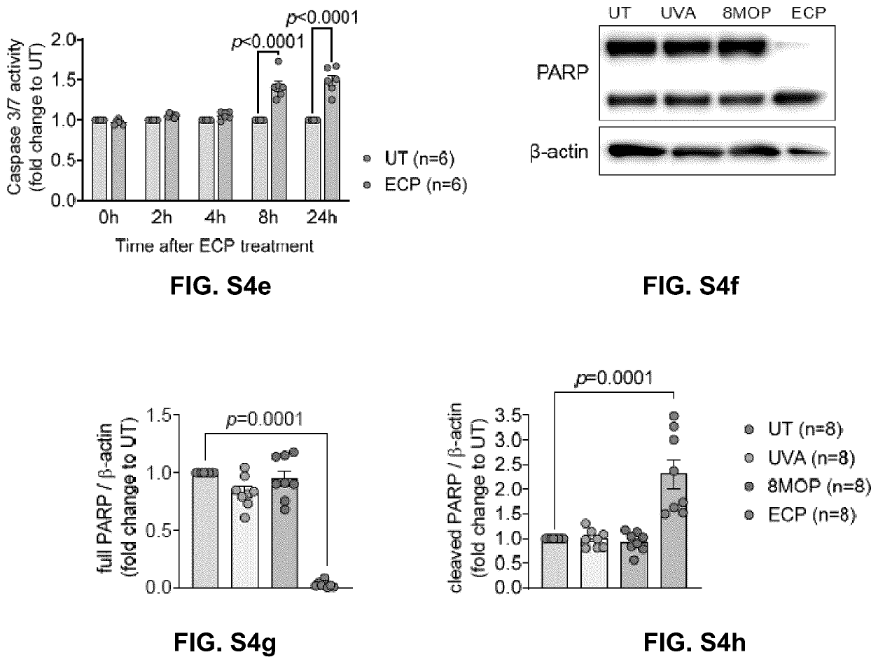

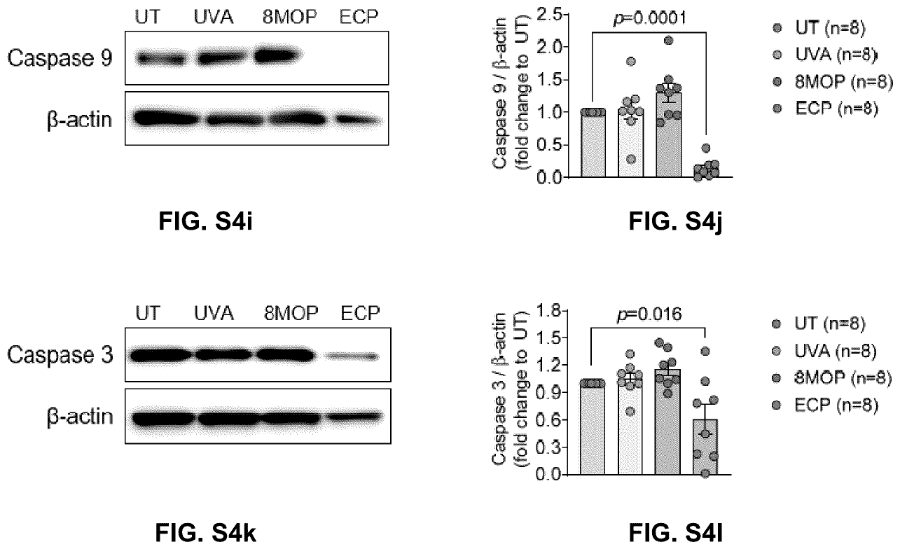

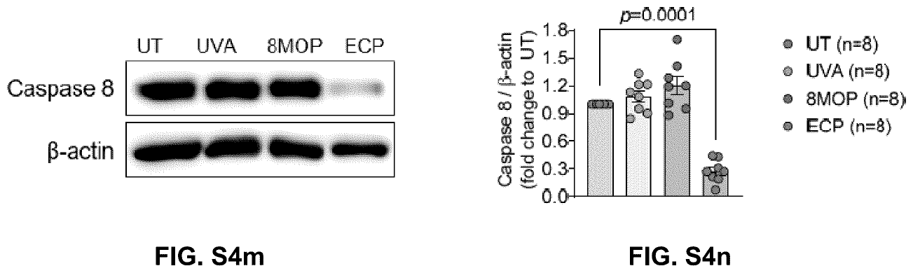

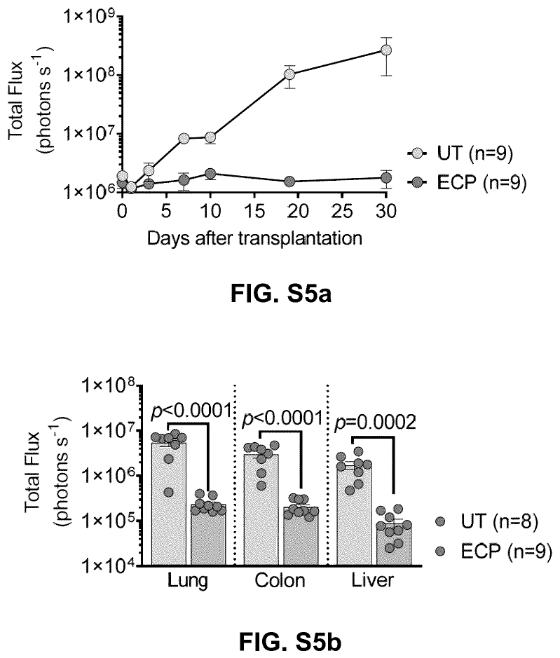

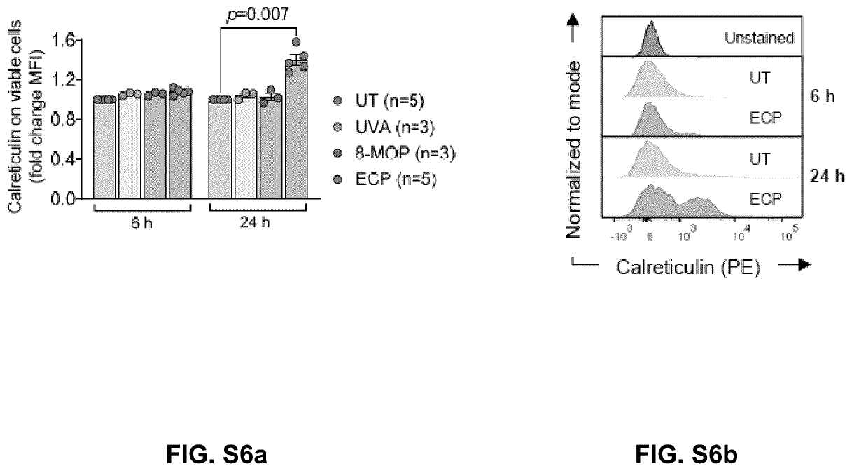

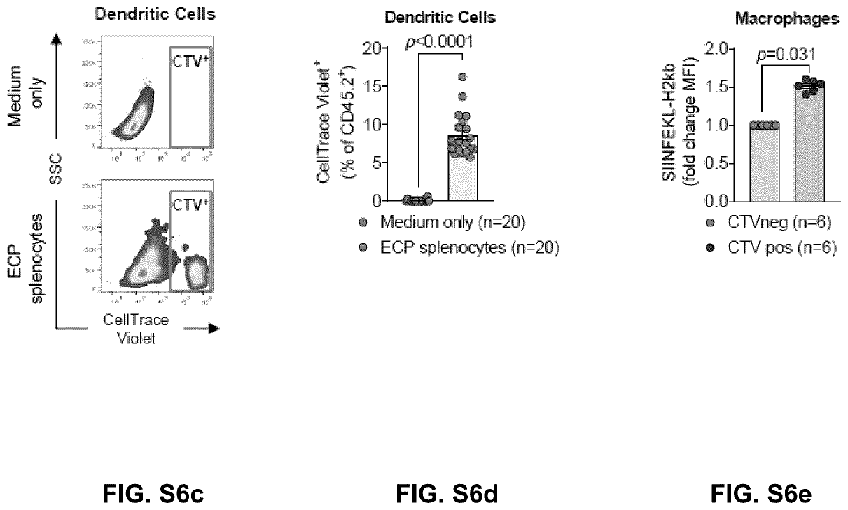

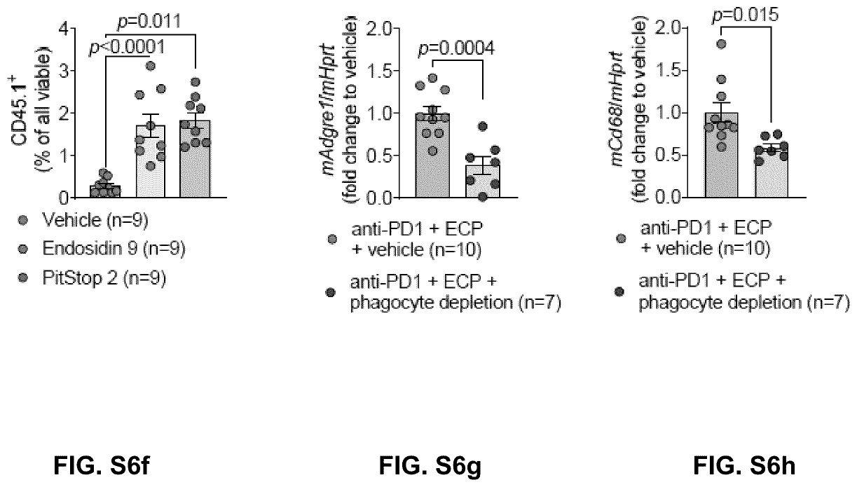

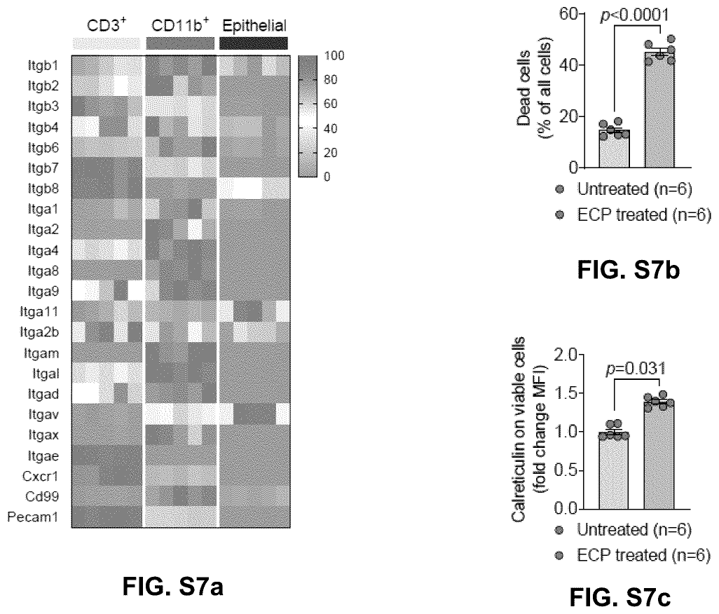

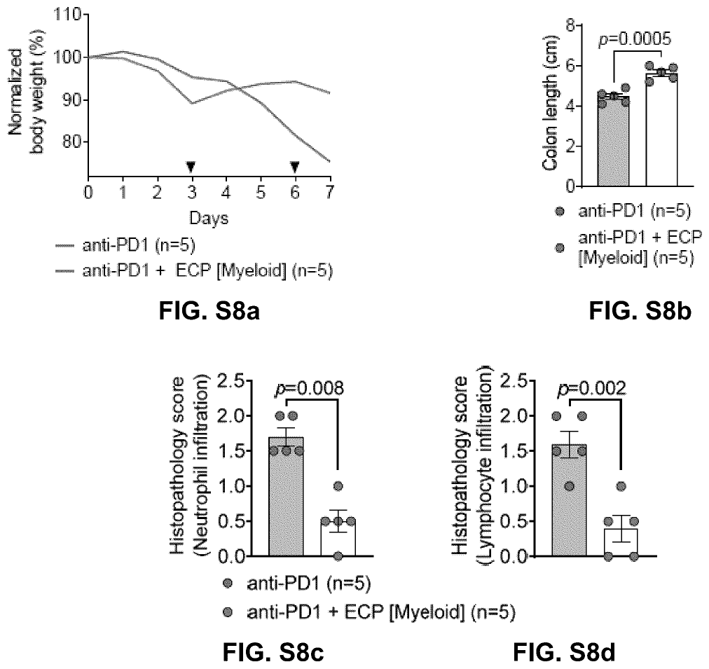

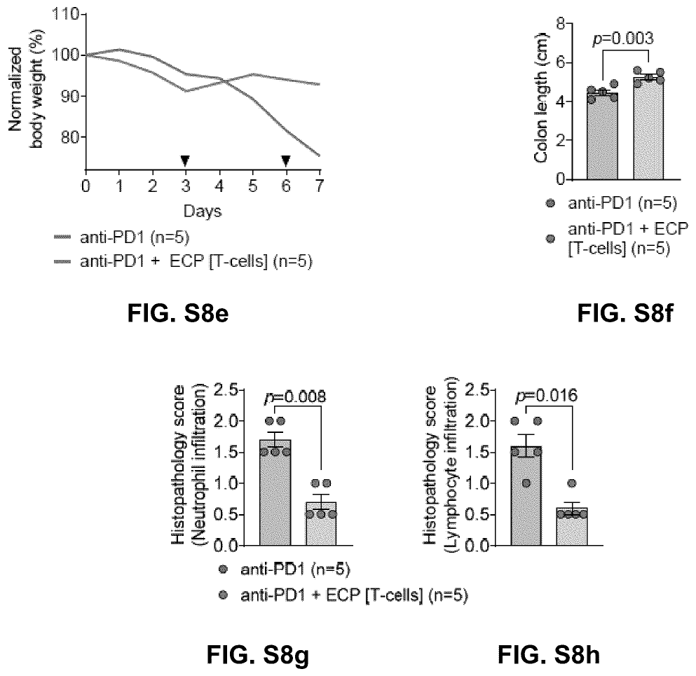

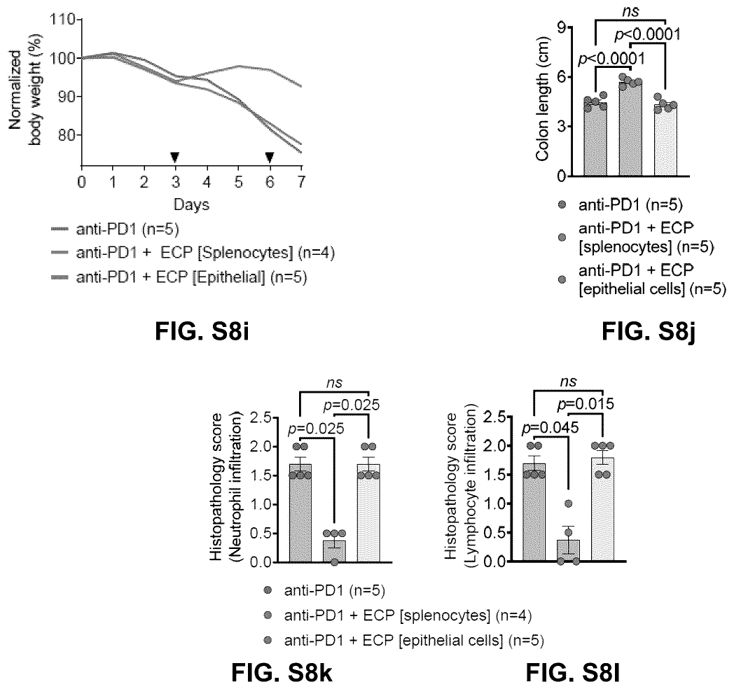

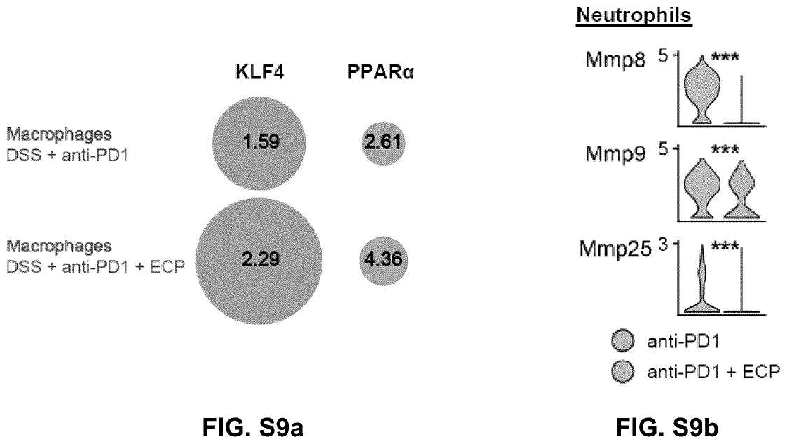

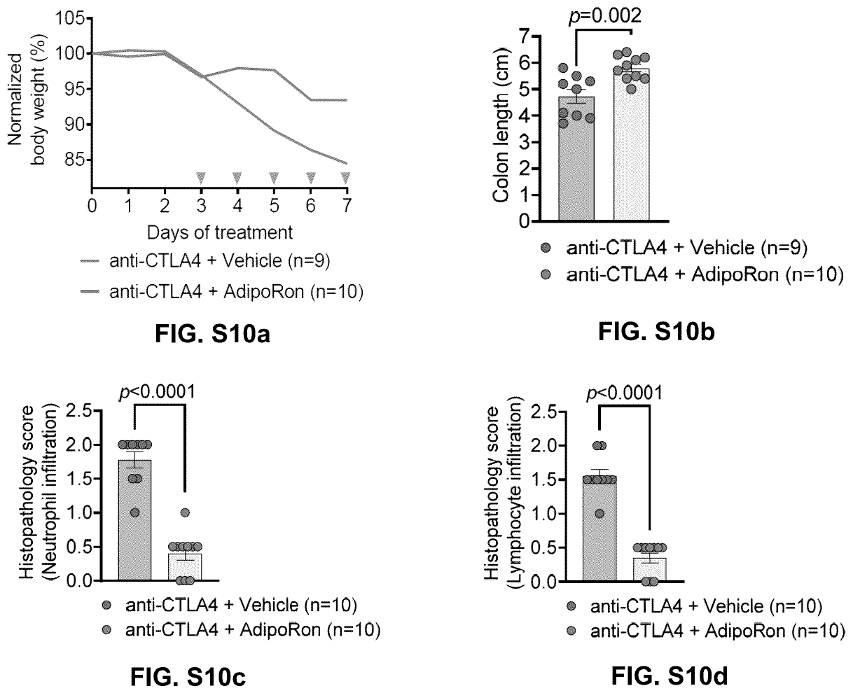

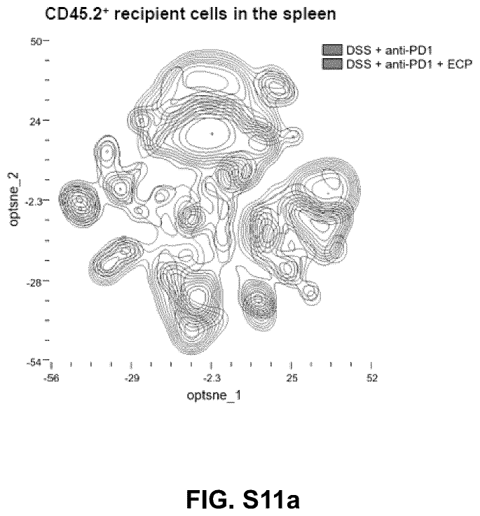

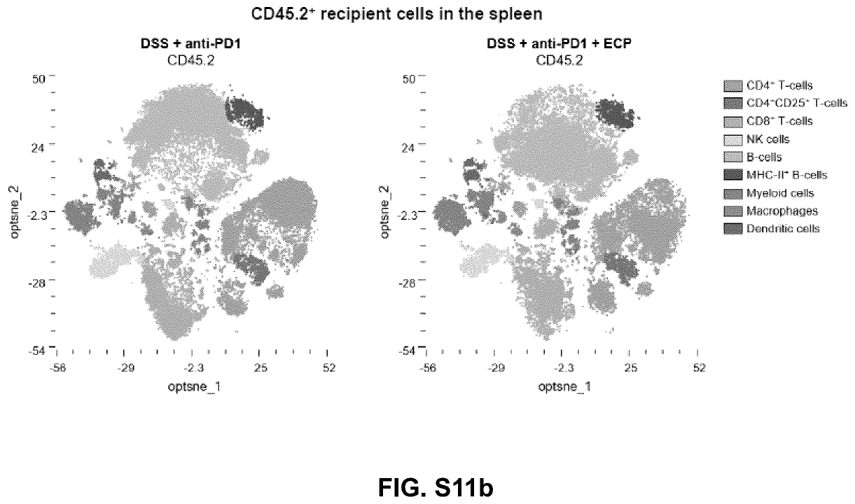

FIGS. 9a-9j show the clinical activity of ECP in patients with irAE.

Specifically, FIG. 9a shows a response assessment of immune-related adverse events (irAE) during/after ECP-treatment. The graph shows absolute numbers of patients with complete response (CR), partial response (PR), stable disease (SD) and progressive disease (PD) of n=14 patients at week 6 and week 12 included in the prospective clinical phase Ib/II trial. irAEs were graded according to CTC-AE V5.0. One patient was not eligible (n.e.) for response assessment at week 12 due to drop out from the study. CR was defined as complete resolution of irAE, PR as reduction of the initial CTC-AE grade of at least one organ, SD as unchanged CTC-AE grade and PD as an increase of the initial CTC-AE grade. All patients with irAE had not responded sufficiently to high dose glucocorticoid treatment for at least 72 h before start of the ECP-therapy. Overall response rates (ORR) at week 6 and 12 after start of the ECP-treatment are indicated above each bar.

FIG. 9b shows a time course of ALT levels of patients with ICI-induced hepatitis during ECP-treatment. Relative blood ALT levels of patients included in the prospective clinical phase Ib/II trial are represented as percentage of the baseline ALT level before start of the ECP-therapy as a surrogate parameter for irAE hepatitis severity. Each line represents an individual patient. ALT levels were assessed at weekly time points until week 12 of ECP-treatment followed by extension of the follow-up intervals. One patient (*line) had a very mild increase of ALT levels (CTC-AE Iº) in addition to his severe colitis before start of ECP. The ALT levels did not decrease much during ECP-therapy, as they were close to normal.

FIG. 9c shows a temporal evolution of ICI-induced colitis during ECP-treatment. Colitis severity of patients included in the prospective clinical phase Ib/II trial was graded according to CTC-AE V5.0 based on diarrhea frequency at weekly timepoints until week 12 of the ECP-therapy followed by an extension of the follow-up intervals. Each color represents an individual patient.

FIG. 9d shows an IrAE response assessment with respect to colitis, hepatitis and dermatitis at week 12 of ECP-treatment. Absolute number of patients with complete response (CR), partial response (PR), stable disease (SD) or progressive disease (PD) with the above described definitions are shown. ORR are indicated above each bar. One patient was not evaluated because of drop out from the study before week 12 (n.e.).

FIG. 9e shows representative endoscopic images of one patient with ICI-induced colitis before and after ECP-therapy. The images were captured before initiation of the ECP-therapy (before ECP) when the patient was under immunosuppression with high dose methylprednisolone and after 22 ECP-treatments (after ECP) and complete tapering of the immunosuppression. The patient was included in the real-world patient cohort and written informed consent for the publication of the endoscopic images was obtained.

FIG. 9f shows representative histological images of the colon mucosa of a patient with colitis before and after ECP (week 6). The arrow indicates a crypt abscess.

FIG. 9g shows a plot that shows severity of histopathological colitis before and after at least 8 ECP-treatments. Each data point represents an individual patient (n=7). P-value was calculated using a two-sided Student's paired 1-test.

FIG. 9h shows a graph that represents relative reduction of corticosteroid dosage after 12 weeks of ECP-therapy (week 12) compared to the first day of ECP-treatment of patients with irAE included in the prospective clinical phase Ib/II trial. Each dot represents an individual patient. One patient was not eligible for calculation of corticosteroid reduction due to drop out from the study before week 12. The p-value was calculated using a one sample Wilcoxon signed rank test.

FIG. 9i shows an Adiponectin concentration in the serum of patients before and 2 weeks after ECP-treatment. Each data point represents an individual patient. P-value was calculated using a two-sided Student's paired t-test.

FIG. 9j shows ADIPOQ gene expression in colitis tissue of irAE-colitis patients before and after ECP-therapy. Gene expression was measured by qPCR, normalized to TBP expression and shown as fold change relative to “before ECP”. Each dot represents an individual patient. The p-value was calculated using a one sample Wilcoxon signed rank test.

FIGS. 9EA-9ED show clinical activity of ECP in patients with irAE in the real-world patient cohort.

Specifically, FIG. 9EA shows an irAE response assessment in the retrospective real-world patient cohort after ECP-treatment. Relative amounts of the best irAE (colitis, arthralgia, hepatitis, dermatitis, mucositis) response to ECP-therapy is represented as a bar graph. Grading was performed according to CTC-AE V.5.0 in a retrospective analysis. Percentages of patients with complete remission (CR), defined as complete resolution of irAE, partial remission (PR), defined as decrease of initial irAE grade of at least one organ and stable disease (SD), defined as unchanged irAE grade, are indicated above each bar.

FIG. 9EB shows a reduction of immunosuppression dosage during ECP-treatment in patients with irAE. The relative changes of immunosuppression dosage before initiation (blue) and after end of ECP-therapy (red) is represented as percentage from baseline. The systemic immunosuppression of one patient was already fully tapered before start of the ECP-treatment, and the patient was still under ECP at the time point of analysis (not shown). Each data point represents an individual patient. The p-value was calculated using the Wilcoxon test.

FIGS. 9EC and 9ED show scatter bar graphs that show histopathological grading of neutrophil (FIG. 9EC) and lymphocyte (FIG. 9ED) infiltration into the colon of immunodeficient Rag2^{-/-}yc^{-/-} mice injected with human PBMCs and treated with human anti-PD1. If indicated, mice received ECP-treated human PBMCs or steroids. Analysis was done on d15 after first PBMC transfer. Results were pooled from two individual experiments (mean±s.e.m.). P-values were calculated using the Kruskal-Wallis test.

FIG. S1a-S1p show the ECP-treatment and ICI-colitis models.

Specifically, FIG. S1a shows the experimental setup of DSS/anti-PD1-induced colitis with ECP-treatment. DSS was given for 3 days, mice were treated with ICI on do, d3 and d6. ECP-treated splenocytes from donor mice were transplanted on d3 and d6. Unless otherwise indicated, serum and tissue samples were harvested on day 8 after start of DSS treatment.

FIG. S1b-S1c show graphs that depict histopathological grading of neutrophil (FIG. S1b) and lymphocyte (FIG. S1c) infiltration into the colon (0=absent, 1=mild, 2=massive) of mice with DSS-induced colitis and isotype or anti-PD1 treatment. Each data point represents the value of an individual mouse. The experiment was done twice and results (mean±s.e.m.) were pooled. P-values were calculated using a Mann-Whitney test.

FIG. S1d shows a graph that shows the colon length (y-axis) and bodyweight as % of initial weight (x-axis) of mice treated with DSS/anti-PD1 and DSS/anti-PD1/ECP. Colon length and bodyweight were measured on d8 after start of DSS treatment. The pooled values of five experiments are shown (n=25). Correlation coefficient was calculated using regression analysis.

FIG. S1e shows a graph that shows bodyweights normalized to initial bodyweight on do. Bodyweight was measured daily. Groups include animals with DSS-induced colitis treated with anti-PD1 alone or anti-PD1 and untreated donor-splenocytes. Each data point represents the mean of the pooled weight of all animals of the indicated group. Arrows indicate splenocyte transfer. Data from two experiments were pooled.

FIG. S1f shows a scatter bar graph that shows mean (±s.e.m.) colon length of mice with DSS-induced colitis, treated with anti-PD1 alone or anti-PD1 and untreated donor-splenocytes. P-value was calculated using an unpaired Student's t-test.

FIGS. S1g and S1h show graphs that depict histopathological grading of neutrophil (FIG. S1g) and lymphocyte (FIG. S1h) infiltration into the colon of mice with DSS-induced colitis treated with anti-PD1 alone or in combination with untreated donor-splenocytes. Each data point represents the value of an individual mouse. The experiment was done twice and results (mean±s.e.m.) were pooled. P-values were calculated using a Mann-Whitney test.

FIG. S1i shows a graph that shows bodyweights normalized to initial bodyweight on do. Bodyweight was measured daily. Groups include animals with DSS/anti-PD1-induced colitis treated with ECP or steroids. Each data point represents the mean pooled weight of all animals of the indicated group. Arrows indicate ECP (black) and steroid (grey) treatment. Data were pooled from two experiments.

FIG. S1j shows a scatter bar graph that shows mean (±s.e.m.) colon length of mice with DSS/anti-PD1-induced colitis, treated with ECP or steroids. Data were pooled from two experiments. P-value was calculated using an unpaired Student's t-test.

FIGS. S1k and S1l show graphs that depict histopathological grading of neutrophil (FIG. S1k) and lymphocyte (FIG. S1l) infiltration into the colon of mice with DSS/anti-PD1-induced colitis treated with ECP or steroids. The experiment was done twice and results (mean±s.e.m.) were pooled. P-values were calculated using Mann-Whitney test.

FIG. S1m shows a graph that depicts bodyweights normalized to bodyweight on do of the experiment. Bodyweights were taken daily. Groups include mice with DSS-induced colitis treated with anti-PD1, anti-PD1 plus 10⁷ ECP-treated splenocytes or anti-PD1 plus 10⁷ ECP-treated PBMCs. Data represents the mean of the pooled weight of all animals of the indicated group. The arrows indicate when ECP was performed (d3 and d6).

FIG. S1n shows a scatter bar graph that shows mean (±s.e.m.) colon length of mice with DSS/anti-PD1-induced colitis, mice with DSS/anti-PD1-induced colitis treated with ECP-treated splenocytes, and mice with DSS/anti-PD1-induced colitis treated with ECP-treated PBMCs, as indicated. P-values were calculated using one-way ANOVA.

FIGS. S1o and S1p show histopathological scoring of neutrophil (FIG. S1o) and lymphocyte (FIG. S1p) infiltration into the colon of mice with DSS/anti-PD1-induced colitis and mice with DSS/anti-PD1-induced colitis treated with ECP-splenocytes or ECP-PBMCs. Scoring was done on d8 of the experiment. Each data point represents the value of an individual mouse (mean±s.e.m). The p-values were calculated by Kruskal-Wallis test.

FIGS. S2a-S2e show that ECP-treatment is effective in anti-CTLA4-induced colitis.

Specifically, FIG. S2a shows a graph that shows bodyweights normalized to initial bodyweight on do. Bodyweight was measured daily. Groups include animals with DSS-induced colitis treated with isotype, anti-CTLA4, or anti-CTLA4 plus ECP. Each data point represents mean pooled weight of all animals of the indicated group. Arrows indicate ECP-treatment. Data were pooled from two experiments.

FIG. S2b shows a scatter bar graph that shows mean (±s.e.m.) colon length of mice with DSS-induced colitis treated with isotype, anti-CTLA4 or anti-CTLA4 plus ECP. Data were pooled from two experiments. P-value was calculated using an ordinary one-way ANOVA.

FIGS. S2c and S2d show graphs that depict histopathological grading of neutrophil (FIG. S2c) and lymphocyte (FIG. S2d) infiltration into the colon of mice with DSS-induced colitis treated with isotype, anti-CTLA4, or anti-CTLA4 plus ECP. The experiment was done twice and results (mean±s.e.m.) were pooled. P-values were calculated by Kruskal-Wallis test.

FIG. S2e shows a scatter plot that displays mean (±s.e.m.) adiponectin serum concentrations in mice with DSS-induced colitis treated either with anti-CTLA4 alone or in combination with ECP. Data were pooled from two experiments and p-value was calculated using an unpaired Student's 1-test.

FIG. S3a-S3h show that adiponectin deficiency in donor leukocytes did not reduce ECP efficacy.

Specifically, FIG. S3a shows a graph that shows bodyweights normalized to initial bodyweight on do. Bodyweight was measured daily. Groups include Adipoq^{-/-} animals with DSS-induced colitis treated with anti-PD1 alone or anti-PD1 plus ECP. Animals were adiponectin-deficient (Adipoq) in the donor and recipient compartment. Each data point represents mean pooled weight of all animals of the indicated group. Arrows indicate ECP-treatment. Data were pooled from two experiments.

FIG. S3b shows a scatter bar graph that shows mean (±s.e.m.) colon length of Adipoq-mice with DSS-induced colitis treated with anti-PD1 alone or anti-PD1 plus ECP. Animals were adiponectin-deficient in the donor and recipient compartments. Data were pooled from two experiments, p-value was calculated using an unpaired Student's t-test.

FIGS. S3c and S3d show graphs that depict histopathological grading of neutrophil (FIG. S3c) and lymphocyte (FIG. S3d) infiltration into the colon of Adipoq^{-/-} mice with DSS-induced colitis treated with anti-PD1 alone or anti-PD1 plus ECP. Animals were adiponectin-deficient in the donor and recipient compartments. The experiment was done twice and results (mean±s.e.m.) were pooled, p-values were calculated by Mann-Whitney test or unpaired Student's 1-test.

FIG. S3e shows a graph that shows bodyweights normalized to initial bodyweight on do. Bodyweight was measured daily. Groups include WT animals with DSS/anti-PD1-induced colitis treated ECP. Mice received ECP cells from WT donors or Adipoq^{-/-} donors as indicated. Each data point represents the mean of the pooled weight of all animals of the indicated group. Arrows indicate ECP-treatment. Data were pooled from two experiments.

FIG. S3f shows a scatter bar graph that shows mean (±s.e.m.) colon length of WT mice with DSS/anti-PD1-induced colitis treated with ECP. Mice received ECP cells from WT donors or Adipoq^{-/-} donors, as indicated. Data were pooled from two experiments and p-value was calculated using an unpaired Student's t-test.

FIGS. S3g and S3h show graphs that depict histopathological grading of neutrophil (FIG. S3g) and lymphocyte (FIG. S3h) infiltration into the colon of WT mice with DSS/anti-PD1-induced colitis treated with ECP. Mice received ECP cells from WT donors or Adipoq^{-/-} donors, as indicated. The experiment was done twice, results (mean±s.e.m.) were pooled and p-values were calculated by Mann-Whitney test.

FIGS. S4a-n show that ECP induced rapid apoptosis in leukocytes in vitro.

FIG. S4a shows a scatter bar graph that displays relative viability (% of 0 h timepoint) of murine splenocytes left untreated (UT) or treated with UVA or 8-MOP or ECP, as indicated. Cell viability was monitored using a luminescence-based viability assay. Data from three experiments was pooled (mean±s.e.m.).

FIG. S4b shows a graph that shows dead cells as percent of all analyzed murine splenocytes analyzed by flow cytometry at different timepoints after treatment. Splenocytes were left untreated (UT) or treated with UVA, 8-MOP, or ECP as indicated. P-values were calculated using a one-way ANOVA.

FIG. S4c shows a representative Western blot image of p53 and vinculin protein levels in murine splenocytes which were left untreated or underwent ECP-treatment. Analysis was done at different timepoints after treatment. Vinculin served as a loading control.

FIG. S4d shows a scatter bar graph that shows the p53 protein level in ECP-treated murine splenocytes as fold change to UT splenocytes at different timepoints after treatment. P53 was normalized to vinculin which served as a loading control. Each data point represents the value of an individual mouse. The p-values were calculated using two-way ANOVA.

FIG. S4e shows a graph that depicts Caspase 3/7 activity in ECP-treated murine splenocytes as fold change to UT splenocytes at different timepoints after treatment. The activity of caspases 3 and 7 was measured using a luminescence-based assay. Each data point represents the value of an individual mouse. The p-values were calculated using a two-way ANOVA.

FIGS. S4f-S4n show murine splenocytes that were left untreated (UT) or treated with UVA, 8-MOP, or ECP as indicated. Proteins were isolated at 24 h after treatment and analyzed by Western blotting; β-actin served as a loading control. Scatter bar graphs show protein levels of total protein or cleaved products. The abundances of PARP (FIGS. S4f, S4g, and S4h), Caspase 9 (FIGS. S4i and S4j), Caspase 3 (FIGS. S4k and S4l) and Caspase 8 (FIGS. S4m and S4n) were analyzed.

FIGS. S5a and S5b show that ECP-treated murine splenocytes undergoed cell death in vivo.

Specifically, FIG. S5a shows total flux (photons/s) in Rag2^{-/-}γc^{-/-} recipients after transplantation with splenocytes from C57BL/6^{luc} donor mice at multiple timepoints after transfer. C57BL/6^{luc} donor mice were transplanted with B16.F10 melanoma and treated with anti-PD1. Splenocytes were isolated on d9 and either left untreated (UT) or ECP-treated before transplantation.

FIG. S5b shows BLI signal quantification of ex vivo imaging of different organs isolated from Rag2^{-/-}γc^{-/-} recipients after transplantation with splenocytes from C57BL/6^{luc} donor mice. Analysis on d30 after transplantation. Donor mice were treated as described in (a). Data from two experiments are pooled (mean±s.e.m.). P-values were calculated using a two-way ANOVA.

FIGS. S6a-h show that ECP-treated splenocytes are phagocytosed by antigen-presenting cells.

Specifically, FIG. S6a shows calreticulin protein level on viable splenocytes that were untreated (UT) or exposed to UVA, 8-MOP, or ECP at various timepoints after treatment. Data is shown as fold change of MFI (mean±s.e.m.), relative to UT cells. P-values were calculated using a Kruskal-Wallis test.

FIG. S6b shows representative histograms for calreticulin on the surface of splenocytes that were untreated (UT) or exposed to ECP at 6 h and 24 h after treatment.

FIG. S6c shows representative flow cytometry plots that show CellTrace Violet (CTV) in CD45.2⁺ BMDCs. ECP-treated CTV stained CD45.1⁺ splenocytes were exposed to CD45.2⁺ BMDCs for 48 h.

FIG. S6d shows quantification of CTV⁺ CD45.2⁺ BMDCs as percent of all BMDCs. ECP-treated CTV stained CD45.1⁺ splenocytes were exposed to CD45.2⁺ BMDCs for 48 h. BMDCs only served as a control. Data were pooled from multiple experiments (mean±s.e.m.). P-value was calculated using a Wilcoxon test.

FIG. S6e shows analysis of SIINFEKL-H2 kb on CD45.2 BMDMs which were co-cultured for 48 h with ECP-treated CTV⁺CD45.1⁺ splenocytes loaded with SIINFEKL ovalbumin peptide. Fold change of MFI compared to CTV⁻CD45.2⁺ BMDMs is shown. BMDMs which phagocytosed ECP-treated cells became CTV positive. Data were pooled from two experiments (mean±s.e.m.). P-value was calculated using a Wilcoxon test.

FIG. S6f shows a scatter plot that displays CD45.1 ECP-treated splenocytes from co-cultures with CD45.2⁺ BMDMs. Macrophages were treated with vehicle or the phagocytosis inhibitors Endosidin 9 or PitStop 2. Viable CD45.1⁺ splenocytes represent non-phagocytosed cells. Data were pooled from two experiments (mean±s.e.m.). Significance was calculated using one-way ANOVA.

FIG. S6g and S6h show graphs that show Adgre1 (FIG. S6g) and Cd68 (FIG. S6h) gene expression in colitis tissue of DSS/anti-PD1/ECP-treated mice which either received vehicle or phagocyte-depleting Clodrosomes. Gene expression was measured by qPCR, normalized to Hprt expression and shown as fold change to vehicle treatment. Data from two individual experiments was pooled (mean±s.e.m.) and significance was calculated with an unpaired Student's 1-test or Wilcoxon test.

FIGS. S7a-S7c show that splenic immune cells had higher integrin expression compared to epithelial cells.

Specifically, FIG. S7a shows relative gene expression of molecules involved in transendothelial migration. RNA was isolated from FACS-sorted splenic cells from naïve mice or from the small intestinal epithelial cell line MODE-K (n=5 per group). Gene expression was measured by qPCR and normalized to Hprt expression. The scale represents the row-wise scaled levels of gene expression.

FIG. S7b shows a graph that shows dead MODE-K epithelial cells as percent of all analyzed cells, analyzed by flow cytometry at 24 h after treatment. Cells were left untreated or treated with ECP, data from two experiments is shown (mean±s.e.m.). P-value was calculated using a Student's 1-test.

FIG. S7c shows calreticulin protein level on viable MODE-K epithelial cells that were untreated or exposed to ECP. Calreticulin was measured at 24 h after treatment. Data from two experiments is shown as fold change of MFI (mean±s.e.m.), relative to untreated cells. P-value was calculated using a Wilcoxon test.

FIGS. S8a-l show that ECP-treated epithelial cells are not protective against ICI-induced colitis.

Specifically, FIGS. S8a and S8e show bodyweights normalized to the initial bodyweight on do. Bodyweight was taken daily. Mice with DSS/anti-PD1-induced colitis were transplanted with ECP-treated FACS-sorted splenic myeloid cells (FIG. S8a) or T-cells (FIG. S8e). Each data point represents mean pooled weight of all animals of the indicated group. Arrows indicate ECP-transplantations.

FIGS. S8b and S8f show scatter bar graph that shows mean colon length (±s.e.m.) of mice with DSS/anti-PD1-induced colitis, treated with ECP-exposed FASC-sorted splenic myeloid cells (FIG. S8b) or T-cells (FIG. S8f). P-values were calculated using an unpaired Student's 1-test.

FIGS. S8c and S8d show graphs that depict histopathological grading (mean±s.e.m.) of neutrophil (FIG. S8c) and lymphocyte (FIG. S8d) infiltration into the colon of mice with DSS/anti-PD-1-induced colitis, treated with ECP-exposed FACS-sorted myeloid cells. P-values were calculated by Mann-Whitney test or an unpaired Student's t-test.

FIGS. S8g and S8h show graphs that depict histopathological grading (mean±s.e.m.) of neutrophil (FIG. S8g) and lymphocyte (FIG. S8h) infiltration into the colon of mice with DSS/anti-PD-1-induced colitis treated with ECP-exposed FACS-sorted T-cells. P-values were calculated by Mann-Whitney test.

FIG. S8i shows bodyweights normalized to the initial bodyweight on do. Bodyweight was taken daily. Groups include mice with DSS-induced colitis treated with anti-PD1, anti-PD1 plus ECP-treated splenocytes (2×10⁶), or anti-PD1 plus ECP-treated MODE-K epithelial cells (2×10⁶). Each data point represents mean pooled weight of all animals of the indicated group. Arrows indicate ECP-transplantations.

FIG. S8j shows a scatter bar graph that shows mean colon length (±s.e.m.) of mice with DSS-induced colitis treated with anti-PD1, anti-PD1 plus ECP-exposed splenocytes (2×10⁶), or anti-PD1 plus ECP-exposed epithelial cells (2×10⁶). P-values were calculated using one-way ANOVA.

FIGS. S8k and S8l show graphs that depict histopathological grading (mean±s.e.m.) of neutrophil (FIG. S8k) and lymphocyte (FIG. S8l) infiltration into the colon of mice with DSS-induced colitis treated with anti-PD1 alone or in combination with ECP-exposed splenocytes (2×10⁶) or epithelial cells (2×10⁶). P-values were calculated using the Kruskal-Wallis test.

FIGS. S9a and S9b show that M2 Macrophage-related transcription factors are upregulated upon ECP.

Specifically, FIG. S9a shows a visualization of KLF4 and PPARα transcription factor activity in the colonic macrophage cluster identified by scRNA Seq. Macrophages from mice treated with DSS/anti-PD1 alone or in combination with ECP are shown. Numbers show the calculated activity score, whereas the size of the circle represents the number of cells with detected activity. Two mice were analyzed per group.

FIG. S9b shows violin plots depicting differential gene expression in the colonic neutrophil cluster identified by scRNA Seq analysis. Cells isolated from DSS/anti-PD1 colitis mice without or with ECP-treatment are analyzed. Significance is indicated with p<0.01 (*), p<0.001 (**) and p<0.0001 (***).

FIGS. S10a-S10d show that AdipoRon reduced anti-CTLA-4-induced colitis. Specifically, FIG. S10a shows bodyweights normalized to the initial bodyweight on do. Bodyweight was taken daily. Mice with DSS/anti-CTLA4-induced colitis were treated with vehicle or the adiponectin receptor agonist AdipoRon. Each data point represents mean pooled weight of all animals of the indicated group. Arrows indicate AdipoRon treatment. Data were pooled from two experiments.

FIG. S10b shows a scatter bar graph that shows mean colon length (±s.e.m.) of mice with DSS/anti-CTLA4-induced colitis treated with vehicle or AdipoRon. Data were pooled from two experiments, p-value was calculated using an unpaired Student's 1-test.

FIGS. S10c and S10d show graphs that depict histopathological grading of neutrophil (FIG. S10c) and lymphocyte (FIG. S10d) infiltration into the colon of mice with DSS/anti-CTLA4-induced colitis treated with vehicle or AdipoRon. The experiment was done twice, results (mean±s.e.m.) were pooled and p-values were calculated by Mann-Whitney test.

FIGS. S11a-S11c show that ECP caused changes in CD45.2+ cells residing in the spleen of mice with DSS/anti-PD1 induced colitis.

Specifically, FIG. S11a shows an overlay of opt-SNE maps displaying CD45.2⁺ recipient cells (ECP donor: CD45.1) in the spleen of mice with DSS-induced colitis treated with anti-PD1 alone or anti-PD1 plus ECP. Immune cells were analyzed by CyTOF.

FIG. S11b shows individual opt-SNE maps displaying CD45.2⁺ recipient cells (ECP donor: CD45.1) of mice with DSS-induced colitis treated with anti-PD1 alone or anti-PD1 plus ECP. Immune cells were analyzed by CyTOF. Colors correspond to FlowSOM-guided clustering of cell populations, clusters were identified by signature marker expression.

FIG. S11c show violin plots that show Arg1 protein levels in splenic CD45.2⁺CD11b⁺ recipient cells from mice with DSS-induced colitis, treated with anti-PD1 alone or anti-PD1 plus ECP. Analysis was by CyTOF, and an unpaired Student's t-test was used.

FIGS. S12a-S12e show that ECP rendered recipient myeloid cells tolerogenic in colitis mice.

Specifically, FIG. S12a shows a heatmap that displays protein levels of tolerogenic markers on splenic recipient CD45.2⁺CD11b⁺ myeloid cells isolated from mice with DSS-induced colitis treated with anti-PD1 alone or in combination with ECP. The scale represents the row-wise scaled level of marker expression, with red being highest and blue being lowest. Analysis by CyTOF.

FIGS. S12b-S12e show violin plots that show protein levels of CD39 (Figure S12b), PD-L1 (FIG. S12c), Tim3 (FIG. S12d) and B7-H4 (FIG. S12e) on splenic recipient CD45.2⁺CD11b⁺ myeloid cells isolated from mice DSS-induced colitis treated with anti-PD1 alone or in combination with ECP. Analysis was by CyTOF and an unpaired Student's t-test was used.

FIGS. S13a and S13b show that expression of intestinal stem cell genes increased after ECP in colitis mice.

Specifically, FIGS. S13a and S13b show graphs that show Asc12 (FIG. S12a) or Axin2 (FIG. S13b) gene expression in colitis tissue of mice with DSS-induced colitis treated with anti-PD1 alone or in combination with ECP. Gene expression was measured by qPCR and normalized to Hprt expression, and shown as fold change to anti-PD1 treatment. Data from three individual experiments was pooled (mean±s.e.m.) and significance was calculated with an unpaired Student's 1-test.

FIGS. S14a-S14q show that ECP increased tolerogenic T-cells in ICI-induced colitis.

Specifically, FIGS. S14a-S14c show pan T-cells that were treated with vehicle or recombinant adiponectin in the presence of anti-CD3/CD28 activation beads. Graphs show CD4⁺ (FIG. S14a) and CD8⁺ (FIG. S14b) T-cells as percent of all T-cells and FoxP3⁺ T_{reg} (FIG. S14c) as percent of CD4⁺ T-cells after 72 h of culture. Data were pooled from two experiments (mean±s.e.m.). Significance was calculated using a paired Student's 1-test.

FIGS. S14d-S14-f show absolute counts of splenic CD8 T-cells expressing BTLA (FIG. S14d), CD47 (FIG. S14e) or KLRG1 (FIG. S14f). Cells isolated from mice with DSS-induced colitis treated with anti-CTLA4 alone or in combination with ECP. Pre-gating on CD45.2⁺ recipient cells. Significance was calculated using a Mann-Whitney test. Mean (±s.e.m.) is shown.

FIGS. S14g and S14h show histopathological grading of neutrophil (Figure S14g) and lymphocyte (FIG. S14h) infiltration into the colon of mice with DSS-induced colitis, anti-PD1 or anti-PD1 plus ECP. Mice served as T-cell donors for adoptive transfer of T-cells into secondary colitis recipients. The experiment was done twice (mean±s.e.m.), and p-values were calculated by Mann-Whitney test.

FIGS. S14i-S14q show frequencies (mean±s.e.m.) of CD4 and CD8 T-cells (FIGS. S14i and S14n), KLRG1⁺ (FIGS. S14j and S140), CD47 (FIGS. S14k and S14p), BTLA (FIGS. S14l and S14q) T-cells, and FoxP3⁺ T_{reg} (FIG. S14m) in pan T-cells isolated from mice with DSS-induced colitis. Recipients were treated with anti-PD1 or anti-PD1 plus ECP. T-cells were isolated on d8 and adoptively transferred into secondary colitis recipients. Significance was calculated by Mann-Whitney or unpaired Student's 1-test.

FIGS. S15a-S15g show long-term steroid treatment reduced splenic immune cells.

Specifically, FIG. S15a shows spleen weight of mice treated long-term with vehicle or prednisolone for 50 days or eight weekly ECP transplantations. Spleens isolated on d50. Data of two experiments was pooled (mean±s.e.m.) and p-values were calculated using a One-way ANOVA.

FIGS. S15b-g show absolute counts of splenic CD45.2⁺ recipient monocytes (FIG. S15b), neutrophils (FIG. S15c), CD4⁺ FoxP3⁺ T_{reg} (FIG. S15d), pDCs (Figure S15e), cDC1 (FIG. S15f) and cDC2 (FIG. S15g) isolated from naïve mice treated with vehicle or prednisolone long-term for 50 days, or eight weekly ECP transplantations (CD45.1⁺ donors). Cells isolated on d50. Experiment was done twice (mean±s.e.m.), p-values were calculated by one-way ANOVA.

FIGS. S16a-S16m show that the protective ECP-effect did not reduce anti-tumor immune responses.

Specifically, FIG. S16a shows an experimental setup of intravenous B16.F10 melanoma model with anti-PD1, steroid, and ECP-treatment. If applicable, mice received transplantation of ECP-treated splenocytes from donor animals (arrows on days 13, 18, and 22). Arrows on days 1, 4, 8, 13, 16, and 22 indicate anti-PD1 and orange arrows from day 13 to day 22 indicate steroid treatment.

FIG. S16b shows a survival analysis of C57BL/6 mice i.v transplanted with B16.F10 melanoma cells (C57BL/6 background). Mice were treated with isotype or anti-PD1, and one group received anti-PD1 and untreated donor splenocytes. Data is from multiple experiments. Significance was calculated by Mantel-Cox test.

FIGS. S16c and S16d show histopathology scoring of neutrophil (FIG. S16c) and lymphocyte (FIG. S16d) infiltration into the colon of melanoma-bearing mice with DSS-induced colitis. Analysis on d23 after melanoma transplantation. Experiment was done as described in (a) and indicated in the methods section, with addition of 3% DSS treatment from d9-12. Experiment was done twice (mean±s.e.m.), p-values were calculated by Kruskal-Wallis test.

FIGS. S16e and S16f show expression mPmel (FIG. S16e) and mDct (Figure S16f) in % of mHprt expression in lungs of healthy C57BL/6 mice and B16.F10 melanoma cells (mean±s.e.m.). P-values were calculated with an unpaired Student's 1-test.

FIGS. S16g-S16j show protein levels of IFNγ (FIGS. S16g and S16i) in CD4⁺ and CD8⁺ splenic T-cells and CD107a (FIGS. S16h and S16j) on CD4⁺ and CD8⁺ splenic T-cells isolated from mice intravenously transplanted with B16.F10 melanoma. Mice were treated with anti-PD1 alone or anti-PD1 and ECP. Analysis on d23 after tumor transplantation. Splenocytes were re-stimulated before analysis. Significance was calculated by Mann-Whitney or unpaired Student's 1-test.

FIG. S16k shows spleen weight of mice bearing s.c. MC38-OVA^{dim} treated as indicated. Steroid treatment was 10 days. Spleens were isolated on d16 after tumor injection. Data of two experiments was pooled (mean±s.e.m.), p-values were calculated using a one-way ANOVA.

FIG. S16l shows a survival analysis of C57BL/6 mice i.v. transplanted with B16.F10 melanoma cells (C57BL/6 background). Recipients were sublethally irradiated before tumor cell transplantation. If indicated, mice received adoptive transfer of T-cells from B16.F10 melanoma-bearing mice treated with anti-PD1. Data from two individual experiments were pooled. Significance was calculated by Mantel-Cox test.

FIG. S16m shows a survival analysis of C57BL/6 mice i.v. transplanted with BRAF^{V600F}-mutant 4434 melanoma cells (C57BL/6 background). Recipients were sublethally irradiated before tumor cell transplantation. If indicated, mice received adoptive transfer of T-cells from B16.F10 melanoma-bearing mice treated with anti-PD1. The transferred T-cells have specificity against B16.F10 but not against 4434-BRAF^{V600E} Data from two individual experiments were pooled. Significance was calculated by Mantel-Cox test.

FIG. S17a-S17h show that ECP did not induce tolerogenic tumor-specific T-cells.

Specifically, FIGS. S17a-S17e show a total number of BTLA⁺ (FIG. S17a), CD47⁺ (FIG. S17b), KLRG1⁺ (FIG. S17c), PD-1 (FIG. S17d) or TIM-3 (Figure S17e) OT-I T-cells per tumor mass. Cells were isolated from mice (CD45.1) with s.c. B16.F10-OVA tumors, which received adoptive transfer of OT-I T-cells (CD45.2) and treatment with anti-PD1 or anti-PD1/ECP. Tumor-infiltrating T-cells were isolated on d16 after tumor transplantation. Data was pooled from two experiments (mean±s.e.m.). P-values were calculated by Mann-Whitney or unpaired Student's 1-test.

FIGS. S17f-h show a total number of BTLA⁺ (FIG. S17f), CD47⁺ (Figure S17g) and KLRG1⁺ (FIG. S17h) OVA-Dextramer CD8⁺ (tumor-specific) T-cells per tumor mass. Cells were isolated from mice with s.c. MC38-OVA^{dim} tumors, treated with anti-PD1 or anti-PD1/ECP (mean±s.e.m.). Tumor-infiltrating T-cells were isolated on d16 after tumor transplantation. P-values were calculated by Mann-Whitney or unpaired Student's 1-test.

FIGS. S18a-m show patient data of the prospective phase Ib/II clinical trial.

Specifically, FIG. S18a shows a scatter bar graph that shows mean weeks (±s.e.m.) of corticosteroid pretreatment before initiation of ECP therapy of n=14 patients included in the prospective clinical phase Ib/II trial (range 2-16 weeks). Each data point represents one patient.

FIG. S18b shows a scatter plot that displays numbers of ECP sessions (mean±s.e.m) performed per patient (range 9-30) of n=14 patients of the prospective clinical phase Ib/II trial. Each data point represents one patient. One patient had received only 9 ECP-treatments before treatment discontinuation due to disease progression, whereas one patient continued ECP therapy after 16 treatments due to relapse of colitis, which responded again to ECP.

FIG. S18c shows a tumor response assessment during ECP therapy within the prospective clinical phase Ib/II trial. The bar graph shows absolute numbers of patients with tumor responses before (week 0) and after 12 weeks of ECP-treatment (week 12). The bar shows absolute numbers of patients at week 0 and 12, respectively. One patient experienced a tumor progression and died shortly after week 6. This patient could not be evaluated for tumor response assessment at week 12 and is shown as not evaluable (n.e.). Tumor responses were classified as complete response (CR), partial response (PR), stable disease (SD) and progressive disease (PD) according to immune-related RECIST criteria based on radiological findings.

FIG. S18d shows an overall survival from start of ECP-therapy until death from any cause was estimated with the Kaplan Meier method. Median survival was not reached, median follow-up was 8 months, determined with the inverse Kaplan Meier method.

FIG. S18e shows an IrAE response assessment after 6 weeks of ECP therapy with respect to colitis, hepatitis and dermatitis. Absolute numbers of patients with complete response (CR), partial response (PR), stable disease (SD) and progressive disease (PD) are shown. ORRs in percent for each irAE are indicated above each bar.

FIGS. S18f-S18i show lymphocyte subpopulations as quantified by flow cytometry in the peripheral blood of patients before the first ECP-treatment and after the second ECP-treatment are shown. Lymphocyte counts are shown as percentage of peripheral blood leukocytes in patients included in the prospective clinical phase Ib/II trial. Each dot represents one patient. CD4⁺ (FIG. S18f) and CD8 T-cells (Figure S18g), NK cells (FIG. S18h), and B-cells (FIG. S18i) were analyzed individually. Significance was calculated using a paired Student's 1-test.

FIGS. S18j-S18m show lymphocyte subpopulations as quantified by flow cytometry at two different time points with intervals of at least 1 week between the 2 timepoints of samples collection while ECP therapy was ongoing in patients with irAE in the prospective clinical phase Ib/II trial. Relative lymphocyte counts of peripheral blood leukocytes are shown. Each dot represents one patient. CD4 (FIG. S18j) and CD8 T-cells (FIG. S18k), NK cells (FIG. S18l) and B-cells (FIG. S18m) were analyzed individually. Significance was calculated using a paired Student's 1-test.

FIGS. S19a-S19d show patient data of the real world patient cohort.

Specifically, FIG. S19a shows a scatter bar graph that shows mean weeks (+s.d.) of corticosteroid pretreatment before initiation of ECP therapy of n=7 patients receiving ECP for irAE at one center included in the real world patient cohort. Each dot represents one patient.

FIG. S19b shows a scatter bar graph that displays mean numbers of ECP sessions (±s.e.m) of n=7 patients with irAE included in the real world patient cohort and treated at one center. Each dot represents one patient.

FIG. S19c shows a time course of AST (grey dots) and ALT levels (orange squares) in U/l of a representative patient with ICI-induced hepatitis before (negative values) and after initiation of the ECP-treatment (positive values). Intervals of concomitantly applied systemic immunosuppression with corticosteroids and mycophenolate mofetil (MMF), as well as administration of infliximab are shown above the graph. In total, 7 ECPs were necessary for a complete remission of the immune-related hepatitis.

FIG. S19d shows a tumor response assessment of n=7 patients of the real world patient cohort before ECP and after termination of ECP-treatment. The tumor response was classified as complete response (CR), partial response (PR), stable disease (SD) and progressive disease (PD), according to immune-related RECIST criteria and based on radiological findings. The ECP-therapy in one patient was ongoing at the time of analysis (ongoing). This patient had a complete remission of his tumor at the data cut-off.

FIGS. S20a-S200 show that the protective ECP-effect was found in a humanized irAE model.

Specifically, FIG. S20a shows an experimental setup of the humanized irAE model with Rag2^{-/-}γc^{-/-} recipient mice. Recipients were injected i.p. with 10⁷ human healthy donor PBMCs and treated with human anti-PD1. If indicated, mice received ECP-treated human PBMCs or prednisolone as therapy. Organs were harvested on d15 after initial PBMC injection.

FIGS. S20b-S20g show scatter bar graphs that show histopathological grading of neutrophil (FIGS. S20b, S20d, and S20f) and lymphocyte (FIGS. S20c, S20e, and S20g) infiltration into the liver (FIGS. S20b and S20c), lung (FIGS. S20d and S20e), and skin (FIGS. S20f and S20g) of immunodeficient Rag2^{-/-}γc^{-/-} mice injected with human PBMCs and treated with human anti-PD1. If indicated, mice received ECP-treated human PBMCs or steroids. Analysis was done on d15 after first PBMC transfer. Results were pooled from two individual experiments (mean±s.e.m.). P-values were calculated using a Kruskal-Wallis test.

FIGS. S20h-S200 show scatter bar graphs that show histopathological grading of neutrophil (FIGS. S20h, S20j, S20l, and S20n) and lymphocyte (FIGS. S20i, S20k, S20m, and S20o) infiltration into the colon (FIGS. S20h and S20i), liver (FIGS. S20j and S20k), lung (FIGS. S20l and S20m), and skin (FIGS. S20n and S200) of immunodeficient Rag2^{-/-}γc^{-/-} mice left untreated or injected with human PBMCs and treated with vehicle or human anti-PD1. Analysis was done on d15 after first PBMC transfer. Results were pooled from two individual experiments (mean±s.e.m.). P-values were calculated using a Kruskal-Wallis test.

FIG. S21 shows a graphical abstract on the mechanism of action of ECP for ICI-induced colitis.

Specifically FIG. S21 shows that ECP induced apoptosis in leukocytes, which are phagocytosed by intestinal macrophages. The uptake induced a transcriptional program including STAT6- and PPARγ-activation, thereby enhancing adiponectin transcription. The intestinal macrophages were polarized towards an immunosuppressive phenotype, leading to Arg1 and adiponectin production. Consecutively, tolerogenic T-cells were increased in the inflamed intestines but not in melanoma tissue, thereby leaving anti-tumor immunity unaffected.

### DETAILED DESCRIPTION

All cited documents of the patent and non-patent literature are hereby incorporated by reference in their entirety.

The present disclosure is directed to adiponectin and/or adiponectin receptor agonist (ARA) or a combination medication comprising adiponectin and/or adiponectin receptor agonist (ARA), a photosensitizing agent and lymphocytes obtained from a method comprising the steps of (i) providing of a sample derived from an isolated blood sample of the subject, (ii) adding the photosensitizing agent to the sample, and (iii) subjecting the sample to UVA irradiation, preferably ECP treatment, for use in the treatment of immune checkpoint blockade (ICB)-induced immune related adverse events (irAEs) in a subject.

In preferred embodiments the photosensitizing agent is administered ex vivo to the blood sample of the subject before the extracorporeal UVA-irradiation of the blood sample of the subject. In preferred embodiments the subject is receiving checkpoint blockade therapy, for example, as a cancer therapy.

As used herein, the term “subject’ means a human or non-human animal selected for treatment or therapy. The subject or patient, such as the subject in need of treatment or prevention, may be an animal, a vertebrate animal, a mammal, a rodent (e.g. a guinea pig, a hamster, a rat, a mouse), a murine (e.g. a mouse), a canine (e.g. a dog), a feline (e.g. a cat), an equine (e.g. a horse), a primate, a simian (e.g. a monkey or ape), a monkey (e.g. a marmoset, a baboon), an ape (e. g. gorilla, chimpanzee, orangutan, gibbon), or a human. The meaning of the terms “animal”, “mammal”, etc. is well known in the art and can, for example, be deduced from Wehner und Gehring (1995; Thieme Verlag). The term subject may also refer to a “patient” and may be used herein interchangeably therewith. In the context of this disclosure, it is particularly envisaged that animals are to be treated which are economically, agronomically or scientifically important. Preferably, the subject/patient is a mammal. More preferably, the subject/patient is a human.

In embodiments of the present disclosure, the subject that has received a checkpoint-inhibitor therapy suffers from cancer, such as malignant melanoma or another cancer treatable by checkpoint-inhibitor therapy.

“Adiponectin” (also named Acrp30, GBP28, apM1, or AdipoQ) is a protein hormone and adipokine (cytokine secreted by adipose tissue), which is involved in regulating glucose levels as well as fatty acid breakdown. Adiponectin is known to bind to at least receptors, wherein two receptors comprise homology to G protein-coupled receptors, namely Adiponectin receptor 1 (AdipoR1), and Adiponectin receptor 2 (AdipoR2), and one receptor comprises similarity with the cadherin family, namely T-cadherin CDH13. Adiponectin receptors affect the downstream target AMP kinase, an important cellular metabolic rate control point. Expression of the receptors is correlated with insulin levels. Adiponectin commonly regulates a number of metabolic processes, such as glucose regulation and fatty acid oxidation, comprising glucose flux, decreased gluconeogenesis, increased glucose uptake, lipid catabolism, β-oxidation, triglyceride clearance, protection from endothelial dysfunction (important facet of atherosclerotic formation), insulin sensitivity, weight loss, control of energy metabolism, upregulation of uncoupling proteins, reduction of TNF-alpha, promotion of reverse cholesterol transport. Adiponectin is known to be able to automatically self-associate into larger structures, starting from three adiponectin molecules forming a homotrimer, followed by further self-association into hexamers or dodecamers. Monomeric (30-kDa) adiponectin is commonly not observed in the circulation and appears to be confined to adipocytes.

### Irradiation and Extracorporeal Photopheresis (ECP)

Photopheresis, or extracorporeal photopheresis or ECP, is a form of apheresis and photodynamic therapy in which blood is treated with a photosensitizing agent and subsequently irradiated with specified wavelengths of light to achieve an effect. For example, buffy coat (WBC+platelets) can be separated from whole blood, chemically treated with 8-methoxypsoralen (instilled into collection bag or given per os in advance), exposed to ultraviolet light (UVA), and returned to the patient. Activated 8-methoxypsoralen crosslinks DNA in exposed cells, ultimately resulting apoptosis of nucleated cells. The photochemically damaged T-cells returned to the patient appear to induce cytotoxic effects on T-cell formation.

Photopheresis involving 8-methoxypsoralen was first described in a 1987 New England Journal of Medicine publication (Edelson, R, et al. (1987). “Treatment of cutaneous T-cell lymphoma by extracorporeal photochemotherapy. Preliminary results”. New England Journal of Medicine. 316 (6): 297-303.). Photopheresis is currently standard therapy approved by the U.S. Food and Drug Administration (FDA) for cutaneous T-cell lymphoma. Evidence suggests that this treatment might be effective in the treatment of graft-versus-host disease. Photopheresis has also been used successfully in the treatment of epidermolysis bullosa acquisita when all other treatments have been ineffective.

ECP as used herein comprises blood irradiation therapy. In embodiments, ECP and ECP systems of the disclosure relates to blood irradiation therapy and systems for blood irradiation therapy. In embodiments of the disclosure, ECP relates to ECP with exception of blood irradiation therapy.

Blood irradiation therapy is a procedure in which the blood is exposed to low level red light (often laser light) for therapeutic reasons. Blood irradiation therapy can be administered in three ways. Extracorporeally, drawing blood out and irradiating it in a special cuvette. This method is used for the ultraviolet (UV) blood irradiation (UVBI) by UV lamps. The laser light is monochromatic, i.e., it has such a wavelength that allows you to bring light into the optical fiber and carry out irradiation intravenously through a catheter in a vein. This method is more simple and effective. Blood irradiation therapy is also administered externally through the skin on the projection of large blood vessels.

“Intravenous or intravascular laser blood irradiation (ILBI)” involves the in-vivo illumination of the blood by feeding low level laser light generated by a 1-3 mW helium-neon laser at a wavelength of 632.8 nm into a vascular channel, usually a vein in the forearm, under the assumption that any therapeutic effect will be circulated through the circulatory system. Most often wavelengths of 365, 405, 525 and 635 nm and power of 2.3 mW are used. The technique is widely used at present in Russia, less in Asia, and not extensively in other parts of the world. It is shown that ILBI improves blood flow and its transport activities, therefore, tissue trophism, has a positive effect on the immune system and cell metabolism. This issue is subject to skepticism. There have been some calls to increase research on this topic. Transcutaneous therapy applies laser light on unbroken skin in areas with large numbers of blood vessels (such as the forearm). Because of the skin acting as a barrier to the blood, absorbing low level laser energy, the power of the laser is often boosted to compensate. The problem can be solved by using pulsed matrix laser light sources. Extracorporeal irradiation is used only for ultraviolet blood irradiation, that involves drawing blood out through a vein and irradiating it outside of the body. Though promoted as a treatment for cancer, a 1952 review in the Journal of the American Medical Association and another review by the American Cancer Society in 1970 concluded the treatment was ineffective.

“Extracorporeal photopheresis (ECP)”, also known as “extracorporeal photoimmunotherapy” or “photochemotherapy”, is a leukapheresis-based therapy which was initially used in patients with cutaneous T-cell lymphoma (CTCL). Specifically, for the treatment of therapy refractory CTCL patients suffering from the leukemic variant, the Sezary Syndrome, ECP received FDA (United States Food and Drug Administration) approval in 1988. During ECP, whole blood of the patient is collected via a cubital vein, or a permanently implanted catheter, for separation of leucocytes from plasma and non-nucleated cells. With a specifically constructed device for this procedure, collected leukocytes, the so-called buffy coat, are then exposed to ultraviolet-A (UVA) irradiation in the presence of a photosensitizing agent, 8-methoxypsoralen prior to reinfusion to the patient.

Two basically different methods for performing ECP procedure have been described and comprised by the present disclosure. They differ in the device used for leukocyte collection and UVA irradiation: the “closed system” and the so called “open system.” The closed system is based on the original design by Edelson and coworkers and is the only FDA-approved system.

In a closed ECP system the buffy coat, comprising lymphocytes and platelets, is separated from the blood by centrifugation. While red blood cells and plasma are redirected into the vein of the subject (through an intravenous line), the separated buffy coat is mixed with a photosensitizing substance, preferably 8-methoxypsoralen, and is then subjected to ultraviolet A (UVA) irradiation (320-400 nm). The UVA radiation induces the incorporation of the photosensitizing substance into the DNA of the lymphocytes. Subsequently the treated buffy coat is reinfused into the patient through an intravenous line.

The open system is a system incorporating different separation instruments, mostly used outside the United States. Although ECP is a valid treatment method since 30 years and over 2 million of treatments have been performed, there are no reports about negative cytogenetic effects.

Indications for initiating ECP were continuously extended since its introduction. ECP treatments are generally well-tolerated by patients and there are almost no significant unwanted side effects. Taken together, ECP combines an excellent safety profile with efficacy.

Extracorporeal photopheresis (also sometimes referred to as extracorporeal photochemotherapy) is a process that includes: (1) collection of mononuclear cells (MNC) from a patient, (2) photoactivation treatment of the collected MNC cells; and (3) reinfusion of the treated cells (MNC) back to the patient. More specifically, ECP involves the extracorporeal exposure of peripheral blood mononuclear cells combined with a photoactive compound, such as 8-methoxypsoralen or “8-MOP” which is then photoactivated by ultraviolet light, followed by the reinfusion of the treated mononuclear cells. It is believed that the combination of 8-MOP and UV radiation causes apoptosis or programmed cell death of ECP-treated T-cells.

Although the precise mechanism of action in ECP treatment (in the different disease states) is not fully known, according to early theories, it was believed that photoactivation causes 8-MOP to irreversibly covalently bind to the DNA strands contained in the T-cell nucleus. When the photochemically damaged T-cells are reinfused, cytotoxic effects are induced. For example, a cytotoxic T-cell or “CD8+ cell” releases cytotoxins when exposed to infected or damaged cells or otherwise attacks cells carrying certain foreign or abnormal molecules on their surfaces. The cytotoxins target the damaged cell's membrane and enter the target cell, which eventually leads to apoptosis or programmed cell death of the targeted cell. In other words, after the treated mononuclear cells are returned to the body, the immune system recognizes the dying abnormal cells and begins to produce healthy lymphocytes (T-cells) to fight against those cells.

In addition to the above, it has also been theorized that extracorporeal photopheresis also induces monocytes (a type of mononuclear cell) to differentiate into dendritic cells capable of phagocytosing and processing the apoptotic T-cell antigens. When these activated dendritic cells are re-infused into systemic circulation, they may cause a systemic cytotoxic CD8+T-lymphocyte-mediated immune response to the processed apoptotic T-cell antigens like that described above. It will be appreciated that other possible mechanisms of action may be involved in achieving the benefits that have been observed from the ECP treatment of mononuclear cells and the subsequent benefits to patients undergoing ECP based therapies.

More recently, it has been postulated that ECP may result in an immune tolerant response in the patient. For example, in the case of graft versus-host disease, the infusion of apoptotic cells may stimulate regulatory T-cell generation, inhibit inflammatory cytokine production, cause the deletion of effective T-cells and result in other responses. See Peritt, “Potential Mechanisms of Photopheresis in Hematopoietic Stem Cell Transplantation,” Biology of Blood and Marrow Transplantation 12:7-12 (2006). While presently the theory of an immune tolerant response appears to be among the leading explanations, there exist other theories as to the mechanism of action of ECP relative to graft-versus-host disease, as well as other disease states.

Systems for performing ECP include, for example, the UVAR XTS Photopheresis System and the CellEx Photopheresis System available from Therakos, Inc., of Exton, Pa. Further details of performing ECP on the Therakos system can be found, for example, in U.S. Pat. No. 5,984,887.

There are currently two commonly used methods for performing photopheresis—online and offline systems and methods.

In online methods, a dedicated photopheresis device, such as the Therakos device mentioned above, is used to perform the entire therapy including reinfusion of treated MNCs. Such devices are “dedicated” photopheresis devices, designed only for performing photopheresis and cannot perform other collection protocols needed in a hospital or blood processing setting including, for example, multifunctional apheresis protocols for collection of platelets, plasma, RBCs, ganulocytes and/or perform plasma/RBC exchange protocols.

In offline photopheresis methods, a multifunctional apheresis device may be used to collect mononuclear cells. The collected MNCs, typically contained in one or more collection containers, are severed or otherwise separated from the tubing set used during collection, where they are later treated in a separate irradiation or UVA light device followed by manual reinfusion of the treated cells to a patient. However, during such offline methods, when the cells are transferred from the apheresis device to the irradiation device (which device may be located in another room or laboratory) communication with the donor must be severed and accordingly, the cells detached from the donor. Thus, additional traceability procedures are required to ensure that the treated MNC product is ultimately reinfused into the correct donor.

In embodiments ECP employs (a) apheresis with ex vivo collection of peripheral mononuclear cells, (b) photoactivation with exposure of leukocyte-enriched plasma to the photosensitizing agent 8-methoxypsoralen and ultraviolet A light, (c) reinfusion of such modified ECP-treated cells to the patient.

According to the disclosure, the expression “extracorporeal blood purification” refers preferably to the process of removing substances from body fluids through their clearance from flowing blood in a diverted circuit outside the patient's body (extracorporeal). Said substances may include endogenous toxins (i.e., uremic toxins), exogenous poisons (i.e., ethylene glycol or fungal toxin), administered drugs, viruses, bacteria, antibodies, metabolites and proteins (i.e., IMHA, myasthenia gravis), abnormal cells (i.e., leukemia), and excessive water. Therapeutic procedures include hemodialysis, including intermittent hemodialysis (HD, HDF, HF) and continuous renal replacement therapy (CRRT); hemoperfusion; plasma exchange and therapeutic apheresis. Such methods are known to a skilled person and the device of the disclosure can be incorporated accordingly.

The expression “blood” as used herein refers to whole blood which contains all components of the blood of an organism, including red cells, white cells, and platelets suspended in plasma. The expression “blood plasma” refers to the fluid, composed of about 92% water, 7% proteins such as albumin, gamma globulin, fibrinogen, complement factors, clotting factors, and 1% mineral salts, sugars, fats, electrolytes, hormones and vitamins which forms part of whole blood but no longer contains red and white cells and platelets. In the context of the present disclosure, the expression “blood plasma” or “plasma” refers to specific fractions of the above defined blood plasma in its standard meaning, such as, for example, blood serum.

In therapeutic apheresis whole blood can be treated or blood is separated into its component fractions, for example by centrifugation or by means of a plasma membrane or filter, and the fraction containing the solute which shall be removed, is specifically treated prior to return to the patient.

The present disclosure provides for an apheresis treatment in which whole blood or plasma (containing the target proteins) is removed from the patient's flowing blood and, after having been contacted with an apheresis device or matrix is returned to the patient.

### ICB Treatment

“Immune checkpoints” are an important part of the immune system, as they prevent an excessive immune response, which can lead to autoimmune reactions or generally damage healthy cells of the body. Immune checkpoints are effective when proteins on the surface of T cells (e.g., PD-1) recognize and bind to checkpoint proteins (e.g., PD-L1), on other cells, e.g., tumor cells. The binding of the checkpoint proteins to the partner proteins on T cells can result in T cell-inhibition and prevention of T cells killing the other cells. This mechanism can prevent auto-immune actions of T cells, but also the killing of tumor cells.

“Immune checkpoint blockade” (ICB) therapy, “checkpoint-inhibitor therapy” or “immune checkpoint inhibitors”, block (immune) checkpoint proteins from binding to their partner proteins on T cells, thereby preventing the inhibition of T cell-mediated killing of, e.g., cancer cells. Immune checkpoint blockade therapy can comprise a treatment with anticytotoxic T lymphocyte-associated antigen 4 (CTLA-4) (ipilimumab and tremelimumab), anti-programmed cell death receptor (PD-1) (nivolumab and pembrolizumab), or anti-PD-ligand (PD-L1) (durvalumab, atezolizumab, and avelumab) monoclonal antibodies. ICB uses the infiltration of immune cells into a tumor or tumor environment to initiate and/or revive an effective anti-tumor immune response. The PD-1/PD-L1 signaling pathway is considered crucial for the interactions between immune, stromal and tumor cells. Herein the terms “Immune checkpoint blockade” (ICB) therapy, “checkpoint-inhibitor therapy” or “immune checkpoint inhibitor” therapy may be used interchangeably.

“Immune checkpoint inhibitors” or “ICB” therapy can cause various side effects, which can depend on the patient health at treatment initiation, the type of cancer that is treated, its stage, the drug(s) administered and its/their dose. A group of side effects can be summarized as immune-related adverse events (irAEs).

Immune-Related Adverse Events (irAE)

As used herein, the term “Immune-related adverse events” (irAE) relates any side effect specific that specifically occur in the context of an immune checkpoint inhibitor treatment, for example in the context of a cancer therapy. IrAEs are unique and are different to adverse events occurring in the context of traditional cancer therapies, and typically have a delayed onset and prolonged duration. IrAEs can involve any organ or system. These effects are frequently low grade and are treatable and reversible; however, some adverse effects can be severe and lead to permanent disorders. Management is primarily based on corticosteroids and other immunomodulatory agents, which should be prescribed carefully to reduce the potential of short-term and long-term complications.

Frequent immune-related adverse events (irAEs) comprise gastrointestinal, endocrine, and dermatologic toxicities. Rare but more severe and potential fatal irAEs can comprise neurotoxicity, cardiotoxicity, and pulmonary toxicity. The observed cytotoxicity is commonly graded according to 4 grades of severity (presently according to the Common Terminology Criteria for Adverse Events, European Society for Medical Oncology guideline and American Society of Clinical Oncology guideline; see e.g., Brahmer et al., 2018, Haanen et al., 2018). Higher grades of irAE cytotoxicity are commonly treated with discontinuation of ICB treatment and/or initiation of glucocorticoid-treatment, e.g., comprising administration of prednisone.

In particular comprised by the term irAEs are symptoms of an autoimmune disease and autoimmune disease, such as (autoimmune) colitis, (autoimmune)hepatitis, (autoimmune)thyroiditis and (autoimmune)dermatitis.

The irAE due to administration of a checkpoint-inhibitor therapy in the present disclosure is not particularly limited. An irAE is to be understood as an adverse event that is presumed to be immune-related: irAE (see, for example, Drug Interview Form of OPDIVO® Intravenous Infusion 20 mg-100 mg, revised in April 2016 (version 9); Properties and Handling of Adverse Events of an Anti-CTLA-4 Antibody, Ipilimumab (YERVOY®), dated Aug. 24, 2015, issued by the Committee on Safety of New Drugs for Malignant Melanoma of the Japanese Dermatological Association).

Specific embodiments of the irAE of the present disclosure include interstitial lung disease, myasthenla gravis, myositis, colitis, type 1 diabetes mellitus, hepatic dysfunction (hepatic disorder), pulmonary disorder such as hepatitis (e.g., autoimmune pneumonia), pituitarism such as hypopituitarism or hypophysitis, thyroid dysfunction such as hypothyroidism, neuropathy, nephropathy, encephalitis, adrenal disorder such as adrenal insufficiency, severe skin disorder, venous thromboembolism, infusion reaction, psoriasis, psoriasiform rash, diarrhea (e.g., severe diarrhea), rheumatoid arthritis, uveitis, episcleritis, bursitis, exacerbation of radiodermatitis, chronic inflammatory demyelinating polyneuropathy (hereinafter also referred to as demyelinating polyneuropathy), biliary tract disorder, or nephritis, and pituitarism is preferable.

The immune-related adverse event (IrAE) has been commonly known to occur, for example, after 8 to 12 weeks after administration of ICB. The immune-related adverse event can be evaluated by “grade” or “IrAE evaluation”. Here, “irAE evaluation” is an index representing the seriousness of a disease, and is represented by 1 to 3. In the irAE evaluation, 1 represents a condition that “does not require additional therapeutic intervention due to irAE”, 2 represents a condition that “requires drug intervention, etc., due to irAE, but does not require hospitalization treatment or does not require interruption of treatment”, and 3 represents a condition that “requires drug intervention, etc., accompanied by hospitalization due to irAE and requires interruption of treatment”. The correspondence between “irAE evaluation” and “grade” varies depending on each disease.

### Immune Checkpoint Molecules and Checkpoint Modulators

In the context of the present disclosure, an immune checkpoint inhibitor is a drug that activates immune cells by modulating immune checkpoint molecules.

Immune checkpoint molecules are molecules in the immune system that either turn up a signal (co-stimulatory molecules) or turn down a signal provided to immune effector cells. Thus, immune checkpoint molecules can be subdivided into co-stimulatory checkpoint molecules or co-inhibitory checkpoint molecules. Co-stimulatory checkpoint molecules include co-stimulatory lymphocyte receptors, which are lymphocyte surface-receptors that can lead to an activation or stimulation of lymphocyte effector functions. Co-inhibitory checkpoint molecules include co-inhibitory lymphocyte receptors, which are lymphocyte surface-receptors that can lead to an inhibition of lymphocyte effector functions.

Co-stimulatory checkpoint molecules comprise, without limitation, HVEM, CD27, CD40, OX40, GITR, CD137, CD28 and ICOS.

In preferred embodiments of the present disclosure, the term co-stimulatory lymphocyte receptor does not refer to CD122.

HVEM (Herpesvirus entry mediator, CD270) is also known as tumor necrosis factor receptor superfamily member 14 (TNFRSF14) and is a receptor of the TNF-receptor superfamily, which can bind to BTLA.

CD27 supports antigen-specific expansion of naïve T cells and is vital for the generation of T cell memory and is also a memory marker of B cells. CD27's activity is governed by the transient availability of its ligand, CD70, on lymphocytes and dendritic cells. CD27 co-stimulation is known to suppress Th17 effector cell function. The agonistic monoclonal antibody CDX-1127/Varlilumab against CD27 has been shown to be effective in the context of T cell receptor stimulation in animal models.

CD28 is constitutively expressed on almost all human CD4+ T cells and on around half of all CD8 T cells. Binding of one of its two ligands CD80 and CD86, expressed for example on dendritic cells, prompts T cell expansion.

CD40 is expressed on a variety of immune system cells including antigen-presenting cells. The ligand of CD40 is called CD40L, also known as CD154, and is transiently expressed on the surface of activated CD4+ T cells, as its ligand. CD40 signaling is known to ‘license’ dendritic cells to mature and thereby trigger T-cell activation and differentiation.

4-1BB (CD137) is bound by CD137 ligand resulting in T-cell proliferation. CD137-mediated signaling is also known to protect T cells, and in particular, CD8+ T cells from activation-induced cell death. The fully human IgG2 agonistic monoclonal antibody Utomilumab (PF-05082566) targets 4-1BB to stimulate a more intense immune system attack on cancers.

OX40 (CD134) has OX40L (CD252) as its ligand. OX40 promotes the expansion of effector and memory T cells and is also known for its ability to suppress the differentiation and activity of T-regulatory cells. OX40 is being transiently expressed after T-cell receptor engagement, which is why it is only upregulated on the most recently antigen-activated T cells within inflammatory lesions, which is why OX40 is a valuable drug target. Agonistic anti-OX40 monoclonal antibodies have been shown to have clinical utility in advanced cancer. The pharma company AstraZeneca has three drugs in development targeting OX40: MEDI0562 is a humanized OX40 agonist; MEDI6469, murine OX40 agonist; and MEDI6383, an OX40 agonist.

GITR (Glucocorticoid-Induced TNFR family Related gene) prompts T cell expansion. The ligand for GITR (GITRL) is mainly expressed on antigen presenting cells. Antibodies to GITR have been shown to promote an anti-tumor response through loss of Treg lineage stability.

ICOS (Inducible T-cell costimulator, also called CD278) is expressed on activated T cells. Its ligand is ICOSL, expressed mainly on B cells and dendritic cells. The molecule seems is important in T cell effector function.

Co-inhibitory checkpoint molecules comprise, without limitation, A2AR, B7-H3, B7-H4, BTLA, CTLA-4, IDO, KIR, LAG3, PD-1, TIM-3, TIGIT and VISTA.

A2AR (Adenosine A2A receptor) is regarded as an important checkpoint in cancer therapy because adenosine in the immune microenvironment, leading to the activation of the A2a receptor, is negative immune feedback loop and the tumor microenvironment has relatively high concentrations of adenosine.

B7-H3, also called CD276, was originally understood to be a co-stimulatory molecule but is now regarded as co-inhibitory. MacroGenics is working on MGA271 (Enoblituzumab), which is an Fc-optimized monoclonal antibody that targets B7-H3.

B7-H4 (or VTCN1) is expressed by tumor cells and tumor-associated macrophages and plays a role in tumor evasion.

BTLA (B and T Lymphocyte Attenuator, also called CD272) is a co-inhibitory receptor, which has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA.

IDO (Indoleamine 2,3-dioxygenase) is a tryptophan catabolic enzyme with immune-inhibitory properties. Another important molecule is TDO, tryptophan 2,3-dioxygenase. IDO is known to suppress T and NK cells, generate and activate Tregs and myeloid-derived suppressor cells, and promote tumor angiogenesis.

KIR (Killer-cell Immunoglobulin-like Receptor) is a receptor for MHC Class I molecules on Natural Killer cells. Lirilumab is a monoclonal antibody to KIR.

LAG-3 (Lymphocyte Activation Gene-3) works to suppress an immune response by action to Tregs as well as direct effects on CD8+ T cells.

“Cytotoxic T-lymphocyte-associated protein 4” (CTLA-4, or CTLA4, or CD152-cluster of differentiation 152), is a protein receptor constitutively expressed in regulatory T cells, which functions as an immune checkpoint that can downregulate a T cell-mediated immune response. CTLA-4 can inactivate T cell effector function when bound to CD80 or CD86 on the surface of antigen-presenting cells. CTLA-4 is a regulatory molecule that represses T cell effector function after initial activation by costimulatory signals. Human monoclonal antibodies targeting CTLA-4 are used in ICB treatment and can increase T-cell function and anti-tumor immune response. Immune checkpoint blockade (ICB) therapy can comprise a treatment with anticytotoxic T lymphocyte-associated antigen 4 (CTLA-4) (ipilimumab and tremelimumab) monoclonal antibodies.

“Programmed cell death protein 1” (PD-1, or CD279-cluster of differentiation 279), is a cell surface protein/receptor of B and T cells, which plays a role in the regulation of auto-immune responses by promoting self-tolerance through suppression of T cell inflammatory activity. PD-1 has two ligands, PD-L1 and PD-L2. PD-1 is an immune checkpoint, which guards against autoimmunity through two mechanisms: by promoting apoptosis of antigen-specific T cells in lymph nodes, and by reducing apoptosis in regulatory T cells. An advantage of targeting PD-1 is that it can restore immune function in the tumor microenvironment. Immune checkpoint blockade (ICB) therapy can comprise a treatment with anti-programmed cell death receptor (PD-1) monoclonal antibodies, such as nivolumab (Opdivo-Bristol Myers Squibb), Pembrolizumab (Keytruda, MK-3475, Merck), Pidilizumab (CT-011, Cure Tech) and BMS-936559 (Bristol Myers Squibb). Both Atezolizumab (MPDL3280A, Roche) and Avelumab (Merck KGaA, Darmstadt, Germany & Pfizer) are monoclonal antibodies directed against PD-L1, the ligand of PD-1.

The transmembrane protein “programmed death-ligand 1” (PD-L1 or, CD274-cluster of differentiation 274, or B7-H1-B7 homolog 1) is encoded in human by the CD274 gene. IFN-γ stimulation induces PD-L1 expression on T cells, NK cells, macrophages, myeloid dendritic cells, B cells, epithelial cells, and vascular endothelial cells. Binding of PD-L1 to its receptor PD-1, which is present on activated T cells, B cells and myeloid cells, modulates activation or inhibition of the immune cells. The binding of PD-L1 to its receptor PD-1 on T cells initiates a signal that inhibits TCR-mediated activation of T cell proliferation und IL-2 production. Interaction of PD-1 with its ligand PD-L1 can prevent autoimmunity. PD-L1 can be highly expressed in a variety of cancers, particularly lung cancer. Immune checkpoint blockade (ICB) therapy can comprise a treatment with anti-PD-ligand (PD-L1) (durvalumab, atezolizumab, and avelumab) monoclonal antibodies.

TIM-3 (T-cell Immunoglobulin domain and Mucin domain 3) expresses on activated human CD4+ T cells and regulates Th1 and Th17 cytokines. TIM-3 acts as a negative regulator of Th1/Th17 function by triggering cell death upon interaction with its ligand, galectin-9.

VISTA (V-domain Ig suppressor of T cell activation) is a protein that is primarily expressed on hematopoietic cells so that consistent expression of VISTA on leukocytes within tumors may allow VISTA blockade to be effective across a broad range of solid tumors.

TIGIT (T cell immunoreceptor with Ig and ITIM domains, also called WUCAM and Vstm3) is an immune receptor present on some T cells and Natural Killer Cells and regulates T cell mediated immunity. TIGIT could bind to CD155 on DCs and macrophages with high affinity and to CD112 with lower affinity.

Co-inhibitory lymphocyte receptors of the present disclosure comprise PD-1, CTLA-4, TIM-3, LAG-3, TIGIT, BTLA or VISTA. Co-stimulatory lymphocyte receptors of the present disclosure comprise OX40, 4-1BB, GITR, CD27, HVEM, CD28 or CD40.

An inhibitor of a receptor prevents the generation of a signal by the respective receptor. Accordingly, an inhibitor of a co-inhibitory lymphocyte receptor is a molecule that prevents the activation of the respective receptor and thereby prevents the generation of an inhibitory signal. Conversely, an activator of a receptor induces the generation of a signal by the respective receptor and an activator of a co-stimulatory lymphocyte receptor leads to the generation of a stimulatory signal.

Checkpoint modulators are molecules that interfere with the activity of immune checkpoint molecules, either by stimulating or inhibiting the activity of immune checkpoint molecules.

Soluble checkpoint modulators are molecules that may be able to freely diffuse and, for example, are not bound to a cell membrane or do not remain intracellular.

Checkpoint inhibitors in the sense of the disclosure comprise lymphocyte-stimulating checkpoint modulators, which are molecules that lead to an activation of lymphocytes, preferably effector T cells, either through activation of a co-stimulatory checkpoint molecule, or through inhibition of a co-inhibitory checkpoint molecules. Furthermore, soluble lymphocyte-stimulating checkpoint modulators include molecules that interfere with the activation of membrane bound immune checkpoint molecules, such as a soluble form of the respective immune checkpoint molecule.

Checkpoint modulators can be naturally occurring molecules or engineered molecules with the respective function interfering with or modulating the activity of an immune checkpoint molecule. Checkpoint modulators include, for example, antibodies or antibody-fragments activity directed against immune checkpoint molecule with agonistic or antagonistic, and ligands or modified ligands of immune checkpoint molecules.

The present disclosure encompasses both treatment and prophylactic treatment of a subject. A “prophylactic” treatment is a treatment administered to a subject who does not exhibit signs of a disease or exhibits only early signs for the purpose of decreasing the risk of developing pathology. Prophylactic treatment is administered to prevent the progression, acceleration, escalation and/or occurrence of a disease.

“Glucocorticosteroids”, which are further called “glucocorticoids”, “corticosteroids” or simply “steroids”, are the presently most effective anti-inflammatory substances used in the treatment of chronic inflammatory, auto-immune and immune diseases. Glucocorticoids induce immunosuppression, which majorly comprises the decreases in the function and numbers of lymphocytes, of both B cells and T cells. Glucocorticoids are lipophilic hormones, which can be subclassified into glucocorticoids produced in the zona fasciculata of the adrenal cortex, mineralocorticoids produced in the zona glomerulosa, and sex hormones produced in the zona reticularis and to a great extent in the gonads. A variety of synthetic glucocorticoids are available for therapeutic use. Examples of glucocorticoids are cortisol (hydrocortisone), cortisone, prednisone, prednisolone, methylprednisolone and dexamethasone.

### Immune Cells

Immune cells as described herein relate to biological cells involved in the immune response in a subject. Immune cells are preferably selected from T Cells, B Cells, Dendritic Cells, Granulocytes, Innate Lymphoid Cells (ILCs), Megakaryocytes, Monocytes/Macrophages, Natural Killer (NK) Cells, Platelets, Red Blood Cells (RBCs) and/or Thymocytes.

The term “immune cells” comprises the MNCs comprised in blood, which may also be called peripheral blood mononuclear cell (PBMC). PBMCs comprise any peripheral blood cell having a round nucleus and consist mainly of lymphocytes (T cells, B cells, NK cells) and monocytes, whereas erythrocytes and platelets have no nuclei, and granulocytes (neutrophils, basophils, and eosinophils) have multi-lobed nuclei. In humans, lymphocytes (lymphoid cells) make up the majority of the PBMC population, followed by monocytes, and only a small percentage of dendritic cells. These cells can be extracted from whole blood using ficoll, a hydrophilic polysaccharide that separates layers of blood, and “density gradient centrifugation”, which will separate the blood into a top layer of plasma, followed by a layer of PBMCs and a bottom fraction of polymorphonuclear cells (such as neutrophils and eosinophils) and erythrocytes. The polymorphonuclear cells can be further isolated by lysing the red blood cells. Basophils are sometimes found in both the denser and the PBMC fractions.

In preferred embodiments of the present disclosure leukocytes may particularly refer to lymphocytes, wherein leukocytes or lymphocytes may preferably comprise or refer to T cells.

A “buffy coat” can be isolated from blood plasma and erythrocytes by centrifugation. The buffy coat can also be referred to as the fraction of an anticoagulated blood sample that contains most of the white blood cells (leucocytes) and platelets after centrifugation. It is rich in a number of immune cells including platelets and leukocytes (white blood cells), such as lymphocytes, granulocytes, monocytes, and macrophages. Buffy coats can be used for enrichment of high numbers of leukocyte subset from peripheral blood mononuclear cells (PBMCs). PBMCs are individual lymphocytes and monocytes that can be separated from the residual whole blood sample through a process called density gradient centrifugation (see above).

“T cells” or T lymphocytes are a type of lymphocyte (a subtype of white blood cell) that plays a central role in cell-mediated immunity. They can be distinguished from other lymphocytes, such as B cells and natural killer cells, by the presence of a T-cell receptor on the cell surface. The several subsets of T cells each have a distinct function. T cell subtypes include, without limitation, T helper type 1 (Th1) cells, T helper type 2 (Th2) cells, T helper type 9 (Th9) cells, T helper type 17 (Th17) cells, T helper type 22 (Th22) cells, Follicular helper T (Tfh) cells, Regulatory T (Treg) cells, Natural killer T (NKT) cells, Gamma delta T cells, CD8+ cytotoxic T lymphocytes (CTLs). Further non-limiting embodiments of T cells include thymocytes, immature T lymphocytes, mature T lymphocytes, resting T lymphocytes, cytokine-induced killer cells (CIK cells) or activated T lymphocytes. Cytokine-induced killer (CIK) cells are typically CD3- and CD56-positive, non-major histocompatibility complex (MHC)-restricted, natural killer (NK)-like T lymphocytes. The T cell can be a CD4+ T cell, a cytotoxic T cell (CTL; CD8+ T cell), CD4+CD8+ T cell, CD4 CD8 T cell, or any other subset of T cells.

“Natural killer (NK) cells” are cytotoxic innate effector cells analogous to the cytotoxic T cells of the adaptive immune system. NK cells are a group of innate immune cells that show spontaneous cytolytic activity against stressed cells, e.g., cancer cells or virus-infected cells. Upon activation, NK cells can secrete cytokines such as interferon-γ (IFN-γ), tumor necrosis factor-α (TNF-α), granulocyte macrophage colony-stimulating factor (GM-CSF), and chemokines (CCL1, CCL2, CCL3, CCL4, CCL5, and CXCL8) that can modulate the function of other innate and adaptive immune cells. NK cells in human are commonly identified as CD3 CD56 cells. They are distributed throughout the blood, organs, and lymphoid tissue and make up around 15% of the peripheral blood lymphocytes. NK cells play a role in tumor surveillance and the rapid elimination of virus-infected cells. They do not require the missing “self” signal of MHC Class I and can recognize stressed cells in the absence of antibodies, allowing them to react much more quickly than the adaptive immune system. Natural killer (NK) cells play critical roles in host immunity against cancer. In response, cancers develop mechanisms to escape NK cell attack or induce defective NK cells (Cheng M et al. Cell Mol Immunol. 2013 May; 10(3):230-52. doi: 10.1038/cmi.2013.10. Epub 2013 Apr. 22.).

The present disclosure in particular relates to immunoregulatory T cells (which can also be referred to as immunomodulatory T cells) for use in the treatment and/or prevention of immune-related adverse events (irAE) in a subject that has received a checkpoint-inhibitor therapy, preferably as a cellular therapy.

In embodiments, generation or induction of immunoregulatory T cells in a sample can be measured by comparing the monocyte and T cell phenotype and population distribution in a sample before and after performing the method of the disclosure, for example by flow cytometry, gene expression and/or mass spectrometry analysis of protein abundance. For example, a shift from Arginase-1^{low} to Arginase-1^{high} monocytes after irradiation is indicative in of the induction of an immunoregulatory phenotype. Furthermore, a decrease in the expression of GM-CSF, IFN-γ, TNF and/or IL-2 in the T cell population would also indicate induction of immunoregulatory T cells. A skilled person is aware of suitable methods and protocols of identifying different monocyte and T cell populations within a sample. For example, in a blood-derived sample, the total monocytes and T cell population may be defined and identified, preferably by flow cytometry, as tolerogenic KLRG1⁺BTLA⁺CD47⁺TOX^{low} T cell population.

Further characteristics and definitions of immunoregulatory T cells are well established in the art and are subject of multiple research and review articles that are known or can be identified by a skilled person.

The term “immunomodulatory functions” relates to functions or properties of molecules or cells that induce a changes or modulation in the function, action or status of any component of the immune system.

Cellular therapies typically involve the administration of immune cells isolated from the blood of the patient. Cell types that can be used in this way are, without limitation, natural killer cells, lymphokine-activated killer cells, cytotoxic T cells, monocytes, macrophages, granulocytes and dendritic cells. Dendritic cell therapy provokes anti-tumor responses by causing dendritic cells to present tumor antigens. Dendritic cells present antigens to lymphocytes, which activates them, priming them to kill other cells that present the antigen.

### Cancer

In the context of the present disclosure, the term “cancer” relates to the treatment of all kinds of cancer, independent of whether the cancer is associated with the formation of a solid tumor or whether the cancer cells do not form a solid tumor, as it is the case for certain leukemias.

Cancer comprises a group of diseases that can affect any part of the body and is caused by abnormal cell growth and proliferation. These proliferating cells have the potential to invade the surrounding tissue and/or to spread to other parts of the body where they form metastasis. Worldwide, there were 14 million new cases of cancer and 8.2 million cancer related deaths in 2012 (World Cancer Report 2014). The majority of cancers is caused by environmental signals involving tobacco use, obesity and infections among others, while around 5-10% are genetic cases. Cancers can be classified into subcategories based on the cell of origin. The most common subcategories are carcinomas from epithelial cells, sarcomas from connective tissue and lymphomas and leukemias from hematopoietic cells. Cancer is associated with a high variety of local and systemic symptoms and cannot be cured in many cases. In light of the high number of new cancer patients and cancer related deaths novel treatment strategies are required.

Cancer according to the present disclosure refers to all types of cancer or neoplasm or malignant tumors found in mammals, including leukemias, sarcomas, melanomas and carcinomas. Either solid tumors and/or liquid tumors (such as leukemia or lymphoma) may be treated.

Melanomas include, but are not limited to include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

Leukemias include, but are not limited to acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Sarcomas include, but are not limited to a chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Carcinomas include, but are not limited to acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma exulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticurn, carcinoma telangiectodes, transitional cell carcinoma, carcinoma tuberosum, tuberous carcinoma, verrucous carcinoma, and carcinoma villosum.

Additional cancers include, but are not limited to Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, cervical cancer, endometrial cancer, adrenal cortical cancer, and prostate cancer.

In some embodiments, “tumor” shall include, without limitation, a prostate tumor, a pancreatic tumor, a squamous cell carcinoma, a breast tumor, a melanoma, a basal cell carcinoma, a hepatocellular carcinoma, a choloangiocellular carcinoma, testicular cancer, a neuroblastoma, a glioma or a malignant astrocytic tumor such as glioblastma multiforme, a colorectal tumor, an endometrial carcinoma, a lung carcinoma, an ovarian tumor, a cervical tumor, an osteosarcoma, a rhabdo/leiomyosarcoma, a synovial sarcoma, an angiosarcoma, an Ewing sarcoma/PNET and a malignant lymphoma. These include primary tumors as well as metastatic tumors (both vascularized and non-vascularized).

### Therapeutic Application of the Disclosure

### Pharmaceutical Combination or Combination Medicine

According to the present disclosure, a “pharmaceutical combination” or “combination medicine” is the combined presence of adiponectin and/or adiponectin receptor agonist (ARA), and a photosensitizing agent and/or lymphoid cells obtained by ECP-treatment according to the disclosure, in proximity to one another. In one embodiment, the combination is suitable for combined administration.

In one embodiment, the pharmaceutical combination or combination medication as described herein is characterized in that adiponectin and/or adiponectin receptor agonist (ARA) is in a pharmaceutical composition in admixture with a pharmaceutically acceptable carrier, and the photosensitizing agent is in a separate pharmaceutical composition in admixture with a pharmaceutically acceptable carrier, and the blood sample or lymphoid cells are in a separate pharmaceutical composition in admixture with a pharmaceutically acceptable carrier. The combination medication or pharmaceutical combination of the present disclosure can therefore in some embodiments relate to the presence of two or three or even more separate compositions or dosage forms in proximity to each other. The agents in combination are not required to be present in a single composition.

### Combined Administration

According to the present disclosure, the term “combined administration”, otherwise known as co-administration or joint treatment, encompasses in some embodiments the administration of separate formulations of the compounds described herein, whereby treatment may occur within minutes of each other, in the same hour, on the same day, in the same week or in the same month as one another. Alternating administration of two agents is considered as one embodiment of combined administration. Staggered administration is encompassed by the term combined administration, whereby one agent may be administered, followed by the later administration of a second agent, optionally followed by administration of the first agent, again, and so forth. Simultaneous administration of multiple agents is considered as one embodiment of combined administration. Simultaneous administration encompasses in some embodiments, for example the taking of multiple compositions comprising the multiple agents at the same time, e.g., orally by ingesting separate tablets simultaneously. A combination medicament, such as a single formulation comprising multiple agents disclosed herein, and optionally additional anti-cancer and/or immuno-suppressing (e.g., steroids) or immuno-modulating medicaments, may also be used in order to co-administer the various components in a single administration or dosage.

In the context of the present disclosure combined administration or combined use may comprise in preferred embodiments the administration of lymphocytes to the subject and/or the administration of adiponectin and/or adiponectin receptor agonist (ARA) to the subject and the ex vivo/extracorporeal administration of a photosensitizing agent to a sample/cells of the subject.

A combined therapy or combined administration of one agent may precede or follow treatment with the other agent to be combined, by intervals ranging from minutes to weeks. In embodiments where the second agent and the first agent are administered separately, one would generally ensure that a significant period of time did not expire between the time of each delivery, such that the first and second agents would still be able to exert an advantageously combined synergistic effect on a treatment site. In such instances, it is contemplated that one would contact the subject with both modalities within about 12-96 hours of each other and, in embodiments preferably, within about 6-48 hours of each other. In some situations, it may be desirable to extend the time period for treatment significantly, however, where several days (2, 3, 4, 5, 6 or 7) to several weeks (1, 2, 3, 4, 5, 6, 7, 8, 9, 10, 11 or 12) lapse between the respective administrations.

In embodiments, the therapeutic agent or combination medication is administered from day 1, 2, 3, 4, 5, 6 or day 7 of the occurrence of irAE symptoms, or for 1, 2, 3, 4, 5, 6, 7, 8, 9, 10, 12, 14, 16, 18, 20, 22, 24, 26, 28, 30, 35, 40, 45 or 50 weeks, or from day 1 to at least 3 weeks, after occurrence of irAE symptoms. In embodiments, the therapeutic agent or combination medication is administered between 1-5 days before the administration of the ICB therapy, or for 1, 2, 3, 4, 5, 6 or 7 days or 1, 2, 3, 4, 5, 6, 7, 8, 9, 10, 12, 14, 16, 18, 20, 22, 24, 26, 28, 30, 35, 40, 45 or 50 weeks before the administration of the ICB therapy. In preferred embodiments, the therapeutic agent or combination medication is administered between 1-14 days, more preferable for 1-5 days before the administration of the ICB therapy.

In the meaning of the disclosure, any form of administration of the multiple agents described herein is encompassed by combined administration, such that a beneficial additional therapeutic effect, preferably a synergistic effect, is achieved through the combined administration of the two medications, and preferably two agents and ECP.

It is understood that substituents and substitution patterns of the compounds described herein can be selected by one of ordinary skill in the art to provide compounds that are chemically stable and that can be readily synthesized by techniques known in the art and further by the methods set forth in this disclosure.

Another aspect of the disclosure includes “pharmaceutical compositions” prepared for administration to a subject and which include a “therapeutically effective amount” of one or more of the compounds disclosed herein. In certain embodiments, the pharmaceutical compositions are useful for treating immune checkpoint blockade (ICB)-induced immune related adverse events (irAEs) in a subject. The therapeutically effective amount of a disclosed compound (e.g., agent, substance, blood sample or cells) will depend on the route of administration, the species of subject and the physical characteristics of the subject being treated. Specific factors that can be taken into account include disease severity and stage, weight, diet and concurrent medications. The relationship of these factors to determining a therapeutically effective amount of the disclosed compounds is understood by those of skill in the art.

Pharmaceutical compositions for administration to a subject can include at least one further pharmaceutically acceptable additive such as carriers, thickeners, diluents, buffers, preservatives, surface active agents and the like in addition to the molecule of choice. Pharmaceutical compositions can also include one or more additional active ingredients such as antimicrobial agents, anti-inflammatory agents, anesthetics, and the like. The pharmaceutically acceptable carriers useful for these formulations are conventional. Remington's Pharmaceutical Sciences, by E. W. Martin, Mack Publishing Co., Easton, PA, 19th Edition (1995), describes compositions and formulations suitable for pharmaceutical delivery of the compounds herein disclosed.

In general, the nature of the carrier will depend on the particular mode of administration being employed. For instance, parenteral formulations usually contain injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The pharmaceutical compositions can be administered by intramuscular, intraocular, subcutaneous, intravenous, intra-arterial, intra-articular, intraperitoneal, intrathecal, intracerebroventricular, or parenteral routes. Optionally, compositions can be administered to subjects by a variety of mucosal administration modes, including by oral, rectal, intraocular, intranasal, intrapulmonary, or transdermal delivery, or by topical delivery to other surfaces.

The compositions according to the disclosure can alternatively contain as pharmaceutically acceptable carrier substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

In accordance with the treatment methods according to the disclosure, the compound can be delivered to a subject in a manner consistent with conventional methodologies associated with management of the disorder for which treatment or prevention is sought. In accordance with the disclosure herein, a prophylactically or therapeutically effective amount of the compound and/or other biologically active agent is administered to a subject in need of such treatment for a time and under conditions sufficient to prevent, inhibit, and/or ameliorate a selected disease or condition or one or more symptom(s) thereof.

“Administration of and “administering a” compound should be understood to mean providing a compound, a prodrug of a compound, a combination of at least two compounds or a pharmaceutical composition as described herein. The compound(s) or composition(s) can be administered by another person to the subject (e.g., intravenously) or it can be self-administered by the subject (e.g., tablets).

Dosage can be varied by the attending clinician to maintain a desired concentration at a target site (for example, the lungs or systemic circulation). Higher or lower concentrations can be selected based on the mode of delivery, for example, intravenous or subcutaneous delivery, trans-epidermal, rectal, oral, pulmonary, or intranasal delivery. Dosage can also be adjusted based on the release rate of the administered formulation, for example, of an intrapulmonary spray versus powder, sustained release oral versus injected particulate or transdermal delivery formulations, and so forth.

The present disclosure also relates to a method of treatment of subjects suffering from the medical conditions disclosed herein. The method of treatment comprises preferably the administration of a therapeutically effective amount of a compound disclosed herein to a subject in need thereof.

A “therapeutically effective amount” refers to a quantity of a specified compound sufficient to achieve a desired effect in a subject being treated with said compound. For example, this may be the amount of a compound disclosed herein useful in treating a condition of immune checkpoint blockade (ICB)-induced immune related adverse events (irAEs) in a subject. The therapeutically effective amount or diagnostically effective amount of a compound will be dependent on the subject being treated, the severity of the affliction, and the manner of administration of the therapeutic composition. Dosage regimens can be adjusted to provide an optimum prophylactic or therapeutic response. A therapeutically effective amount is also one in which any toxic or detrimental side effects of the compound and/or other biologically active agent is outweighed in clinical terms by therapeutically beneficial effects.

A non-limiting range for a therapeutically effective amount of a compound adiponectin or ARA and/or other biologically active agent within the methods and formulations of the disclosure is about 0.001 mg/kg body weight to 50 mg/kg body weight, 0.01 mg/kg body weight to about 20 mg/kg body weight, such as about 0.05 mg kg to about 5 mg/kg body weight, or about 0.2 mg/kg to about 2 mg/kg body weight, or about 1*10\hat{ }4 cells/kg body weight.

A non-limiting range for a therapeutically effective amount of tolerogenic T cells about 1*10\hat{ }3 cells/kg body weight, about 5*10\hat{ }3 cells/kg body weight, about 1*10\hat{ }4 cells/kg body weight, about 5*10\hat{ }4 cells/kg body weight, about 1*10\hat{ }5 cells/kg body weight, about 5*10\hat{ }5 cells/kg body weight, about 1*10\hat{ }6 cells/kg body weight, about 5*10\hat{ }6 cells/kg body weight, about 1*10\hat{ }7 cells/kg body weight, about 5*10\hat{ }7 cells/kg body weight, or about 1*10\hat{ }8 cells/kg body weight or more.

A non-limiting range for a therapeutically effective amount of the photosensitizing agent, preferably 8-Methoxyproralen, is between 1-10 mg/ml sample, preferably about 2 mg/ml sample.

As used herein, the term “approximately” or “about” is used to describe and account for small variations. For example, the term may refer to less than or equal to 10, such as less than or equal down to 1, when appropriate, also the term may refer to more than or equal to 10, such as more than or equal up to 100 or more, when appropriate. It is to be understood that range format is used for the sake of simplicity and brevity and is to be flexibly understood to include numeric values expressly stated as boundaries of a range, encompassing each numeric value and sub-ranges.

The instant disclosure also includes kits, packages and multi-container units containing the herein described pharmaceutical compositions, active ingredients, and/or means for administering the same for use in the prevention and treatment of diseases and other conditions in mammalian subjects.

With the above context, the following consecutively numbered embodiments provide further specific aspects of the disclosure:
1. Adiponectin and/or Adiponectin Receptor Agonist (ARA) for use in the treatment or prevention of immune checkpoint blockade (ICB)-induced immune related adverse events (irAEs) in a subject.
2. Adiponectin and/or ARA for use according to embodiment 1, wherein the subject is receiving checkpoint blockade therapy.
3. Adiponectin and/or ARA for use according to any one of the preceding embodiments, wherein Adiponectin and/or ARA is administered at least 1 hour before, during and/or at least one hour after administration of checkpoint blockade therapy.
4. Adiponectin and/or ARA for use according to any one of the preceding embodiments, wherein the checkpoint blockade therapy comprises administration of at least one antibody, preferably at least two antibodies.
5. Adiponectin and/or ARA for use according to any one of the preceding embodiments, wherein the at least one antibody is selected from the group comprising cytotoxic T-lymphocyte antigen 4 (CTLA-4), programmed cell death-1 (PD-1) and/or programmed cell death ligand-1 (PD-L1) or Lymphocyte-activation gene 3 (LAG-3).
6. Adiponectin and/or ARA for use according to any one of the preceding embodiments, wherein irAEs can involve any organ, preferably the gastrointestinal tract, lungs, endocrine glands, skin, and liver.
7. Adiponectin and/or ARA for use according to any one of the preceding embodiments, wherein the irAE comprise an ICB-induced colitis.
8. Adiponectin and/or ARA for use according to any one of the preceding embodiments, wherein the administration of Adiponectin and/or ARA reduces ICB-induced colitis.
9. Adiponectin and/or ARA for use according to any one of the preceding embodiments, wherein the administration of Adiponectin and/or ARA does not interfere with the anti-tumor response induced by the checkpoint blockade therapy, which is preferably an anti-PDItreatment, due to the tissue specific adiponectin induction.
10. Combination medication for use in the treatment of immune checkpoint blockade (ICB)-induced immune related adverse events (irAEs) in a subject, comprising at least two of the following components:
   a. a photosensitizing agent, and
   b. Adiponectin and/or Adiponectin Receptor Agonist (ARA), and wherein a blood sample of said subject is subjected to ultraviolet A (UVA) irradiation extracorporeally, preferably to extracorporeal photopheresis (ECP) therapy
11. The combination medication for use according to embodiment 10, wherein the photosensitizing agent is a psoralen reagent, preferably 8-methoxypsoralen.
12. The combination medication for use according to embodiments 10 to 11, wherein the wavelength of the UVA irradiation is from wavelength of 3200 to 4000 angstroms.
13. The combination medication for use according to embodiments 11 to 12, wherein the subject suffers from cancer.
14. The combination medication for use according to embodiments 10 to 13, wherein the checkpoint blockade therapy comprises administration of at least one antibody, preferably at least two antibodies.
15. The combination medication for use according to embodiments 10 to 14, wherein the at least one antibody is selected from the group comprising cytotoxic T-lymphocyte antigen 4 (CTLA-4), programmed cell death-1 (PD-1) and/or programmed cell death ligand-1 (PD-L1) or Lymphocyte-activation gene 3 (LAG-3).
16. The combination medication for use according to embodiments 10 to 15, wherein irAEs can involve any organ, preferably the gastrointestinal tract, lungs, endocrine glands, skin, and liver.
17. The combination medication for use according to embodiments 10 to 16, wherein the irAE comprises an ICB induced colitis.
18. The combination medication for use according to embodiments 10 to 17, wherein the said combination medication induces an increase of adiponectin, arginase-1 (Arg1) and tolerogenic KLRG1⁺BTLA⁺CD47⁺TOX^{low} T cells in inflamed intestines, but not in cancer tissue in said subject.
19. Adiponectin and/or Adiponectin Receptor Agonist (ARA) for use in the treatment of a tumor patient (or: subject suffering from cancer), wherein said patient has developed immune checkpoint blockade (ICB) induced immune related adverse events (irAEs), wherein said treatment comprises the combined administration of Adiponectin and/or ARA with an anti-tumor immunotherapy, wherein said immunotherapy comprises the administration of immunoregulatory T cells (or more general: blood samples).
20. Adiponectin and/or ARA for use in the treatment of a tumor patient according to the preceding embodiment, wherein the immunoregulatory T cells (or: blood samples)
   are obtained from a method comprising the steps of
   providing a sample derived from an isolated blood sample of said patient,
   adding a photosensitizing agent to the sample, and subjecting the sample to irradiation.
21. Adiponectin and/or ARA for use in the treatment of a tumor patient according to the embodiments 19 and 20 wherein the photosensitizing agent is 8-methoxypsoralen and/or the irradiation is UVA irradiation.
22. A method of treating or preventing immune checkpoint blockade (ICB)-induced immune related adverse events (irAEs) comprising administering Adiponectin and/or Adiponectin Receptor Agonist (ARA) in a subject, wherein the subject is receiving checkpoint blockade therapy.
23. The method of embodiment 22, wherein Adiponectin and/or ARA is administered at least one hour before, concurrently with, and/or at least one hour after administration of the checkpoint blockade therapy.
24. The method of embodiment 22 or 23, wherein the checkpoint blockade therapy comprises administration of at least one antibody.
25. The method of embodiment 24, wherein the at least one antibody is selected from the group consisting of cytotoxic T-lymphocyte antigen 4 (CTLA-4), programmed cell death-1 (PD-1), programmed cell death ligand-1 (PD-L1), and Lymphocyte-activation gene 3 (LAG-3).
26. The method of any one of embodiments 22 or 23, wherein the checkpoint blockade therapy comprises administration of two or more antibodies.
27. The method of embodiment 26, wherein at least one of the two or more antibodies is selected from the group consisting of cytotoxic T-lymphocyte antigen 4 (CTLA-4), programmed cell death-1 (PD-1), programmed cell death ligand-1 (PD-L1), and Lymphocyte-activation gene 3 (LAG-3).
28. The method of embodiment 26 or 27, wherein the two or more antibodies are selected from the group consisting of cytotoxic T-lymphocyte antigen 4 (CTLA-4), programmed cell death-1 (PD-1), programmed cell death ligand-1 (PD-L1), and Lymphocyte-activation gene 3 (LAG-3).
29. The method of any one of embodiments 22-28, wherein the irAEs involve in any organ.
30. The method of embodiment 29, wherein the organ is selected from the group consisting of gastrointestinal tract, lungs, endocrine glands, skin, and liver.
31. The method of any one of embodiments 22-30, wherein the irAEs comprise an ICB-induced colitis.
32. The method of any one of embodiments 22-31, wherein the administration of Adiponectin and/or ARA reduces ICB-induced colitis.
33. The method of any one of embodiments 22-32, wherein the administration of Adiponectin and/or ARA does not interfere with an anti-tumor response induced by the checkpoint blockade therapy due to the tissue specific adiponectin induction.
34. The method of any one of embodiments 22-33, wherein the checkpoint blockade therapy is an anti-PD1 treatment.
35. A method of treating immune checkpoint blockade (ICB)-induced immune related adverse events (irAEs) in a subject comprising administering components a and b to the subject:
   (a) a photosensitizing agent, and
   (b) Adiponectin and/or Adiponectin Receptor Agonist (ARA), and wherein a blood sample of the subject is subjected to ultraviolet A (UVA) irradiation extracorporeally.
36. The method of embodiment 35, wherein the ultraviolet A (UVA) irradiation is extracorporeal photopheresis (ECP) therapy.
37. The method of embodiment 35 or 36, wherein the photosensitizing agent is a psoralen reagent.
38. The method of embodiment 37, wherein the psoralen reagent is 8-methoxypsoralen.
39. The method of any one of embodiments 35-38, wherein a wavelength of the UVA irradiation is from wavelength of 3200 to 4000 angstroms.
40. The method of any one of embodiments 35-39, wherein the subject suffers from cancer.
41. The method of embodiment 40, wherein the cancer is malignant melanoma or breast cancer.
42. The method of any one of embodiments 35-41, wherein the subject is receiving checkpoint blockade therapy.
43. The method of embodiment 42, wherein the checkpoint blockade therapy comprises administration of at least one antibody.
44. The method of embodiment 43, wherein the at least one antibody is selected from the group consisting of cytotoxic T-lymphocyte antigen 4 (CTLA-4), programmed cell death-1 (PD-1), programmed cell death ligand-1 (PD-L1), and Lymphocyte-activation gene 3 (LAG-3).
45. The method of embodiment 42, wherein the checkpoint blockade therapy comprises administration of two or more antibodies.
46. The method of embodiment 45, wherein at least one of the two or more antibodies is selected from the group consisting of cytotoxic T-lymphocyte antigen 4 (CTLA-4), programmed cell death-1 (PD-1), programmed cell death ligand-1 (PD-L1), and Lymphocyte-activation gene 3 (LAG-3).
47. The method of embodiment 45 or 46, wherein the two or more antibodies are selected from the group consisting of cytotoxic T-lymphocyte antigen 4 (CTLA-4), programmed cell death-1 (PD-1), programmed cell death ligand-1 (PD-L1), and Lymphocyte-activation gene 3 (LAG-3).
48. The method of any one of embodiments 35-47, wherein the irAEs involve in any organ.
49. The method of embodiment 48, wherein the organ is selected from the group consisting of gastrointestinal tract, lungs, endocrine glands, skin, and liver.
50. The method of any one of embodiments 35-49, wherein the irAEs comprise an ICB-induced colitis.
51. The method of any one of embodiments 35-50, wherein the administration of Adiponectin and/or ARA reduces ICB-induced colitis.
52. The method of any one of embodiments 35-51, wherein the administration of Adiponectin and/or ARA does not interfere with an anti-tumor response induced by the checkpoint blockade therapy due to the tissue specific adiponectin induction.
53. The method of embodiment 51, wherein the checkpoint blockade therapy is an anti-PD1 treatment.
54. The method of any one of embodiments 35-53, wherein the treatment induces an increase of adiponectin, arginase-1 (Arg1) and tolerogenic KLRG1⁺BTLA⁺CD47⁺TOX^{low} T cells in inflamed intestines, but not in cancer tissue in said subject.
55. A method of treating a patient with cancer comprising administering a combination of Adiponectin and/or Adiponectin Receptor Agonist (ARA) with an anti-tumor immunotherapy, wherein the immunotherapy comprises administration of immunoregulatory T cells, and the patient has immune checkpoint blockade (ICB) induced immune related adverse events (irAEs).
56. The method of embodiment 55, wherein the immunoregulatory T cells are prepared by:
   (a) obtaining a sample derived from an isolated blood sample of the patient,
   (b) adding a photosensitizing agent to the sample, and
   (c) subjecting the sample to irradiation.
57. The method of embodiment 56, wherein the photosensitizing agent is 8-methoxypsoralen.
58. The method of embodiment 56 or 57, wherein the irradiation is ultraviolet A (UVA) irradiation.
59. The method of any one of embodiments 55-58, wherein the patient has a cancer that is malignant melanoma or breast cancer.

### EXAMPLES

The disclosure is further described by the following examples. These are not intended to limit the scope of the disclosure but represent preferred embodiments of aspects of the disclosure provided for greater illustration of the disclosure described herein.

### Example 1

### Methods

Evaluation of irAE Response of Patients

The severity of irAE was evaluated by a clinical assessment of the symptoms, and carried out before the initiation of ECP as well as at every patient visit after initiation of the therapy. irAE severity and response definitions were established based on the ASCO guidelines 23.

Grading of irAE was performed according to the Common Terminology Criteria for Adverse Events (CTC-AE v5). Complete response (CR) was defined as the complete resolution of all irAE-related symptoms. Partial response (PR) was defined as the reduction of at least 1 grade according to CTC-AE in at least one irAE organ manifestation. Stable disease (SD) was defined as no change in the CTC-AE grades of all irAE. Progressive disease (PD) was defined as the increase of at least 1 grade according to CTC-AE in at least one irAE organ manifestation. CR, PR and PD were diagnosed only if the symptom severity remained stable for at least 5 consecutive days.

The full analysis data set (FAS) and the safety set (SAF) included all the patients who received at least one administration of ECP. Descriptive statistics was employed for reporting continuous variables and frequency and percentages of discrete variables. Exact two-sided 95% confidence intervals for ORR were calculated, based on the binomial distribution. Overall survival (OS) and progression free survival (PFS) were defined as time from start of treatment with ECP until death (OS)/death or disease progression (PFS), treating observation times where the event of interest did not occur as censored. Analyses of OS and PFS were performed with the Kaplan Meier method.

### Cell Lines

The B16.F10 murine melanoma cell line (C57BL/6 background) was provided by Hanspeter Pircher (Freiburg University, Germany). Luciferase transgenic B16.F10^{luc/GFP} cells were established as described in Mastroianni, J., et al., “miR-146a Controls Immune Response in the Melanoma Microenvironment,” Cancer Res 79, 183-195 (2019). The BRAF-mutant 4434 murine melanoma cell line was established C57BL/6 LSL-Braf^{V600E}; Tyr:: CreERT^{+/o} mice (Dhomen, N., et al., “Oncogenic Braf induces melanocyte senescence and melanoma in mice.” Cancer Cell 15, 294-303 (2009)) and kindly provided by Richard Marais (Cancer Research UK Manchester Institute, Manchester, UK). The MC38 and MC38^{OVA} cell lines were kindly provided by Karen Dixon and Vijay Kuchroo (Harvard Medical School, US). All cell lines were cultured in DMEM high glucose medium (Gibco), supplemented with 10% FCS (Sigma) and 1% Penicillin/streptomycin (Gibco) at 37° C. and 5% CO2 in a humidified atmosphere. Medium for the MC38 line was additionally supplemented with 1% sodium pyruvate (Gibco). B16.F10luc/GFP cells were cultured with 2 mg/mL of the neomycin analogue Geneticin (G-418 sulfate: Genaxxon) for two passages after thawing. The immortalized small intestinal epithelial cell line MODE-K26 was cultured in RPMI 1640 (Gibco) supplemented with 10% FCS (Sigma), 1% Penicillin/streptomycin (Gibco), 1 mM sodium pyruvate (Gibco), 10 mM HEPES (Gibco), 0.1 mM MEM non-essential amino acids (Gibco) and 50 μM 2-Mercaptoethanol (Gibco) at 37° C. and 5% CO2 in a humidified atmosphere. Cells were tested repeatedly for mycoplasma contamination and found to be negative.

### ECP-Treatment, Mouse Models

For modeling ECP transplantation in vivo, splenocytes of donor mice were used. Donor animals were treated identically to the respective recipient groups. Spleens were harvested, meshed and erythrocytes were lysed. Splenocytes were resuspended to 3-5×106 cells/mL in RPMI, supplemented with 10% FCS and 1% P/S, and treated with 200 ng/mL 8-methoxypsoralen (8-MOP; Uvadex©, Therakos) at 37° C. and 5% CO2 for 30 min. Following incubation, the cells were exposed to UVA light (2 J/cm2) in a calibrated BS-02 irradiation chamber, operated in dose-controlled mode (Opsytec Dr. Gröbel). ECP-treated splenocytes were resuspended in PBS and 107 cells were injected intravenously into recipient mice. Where indicated, PBMCs were treated with ECP comparably to splenocytes and 107 PBMCs were injected intravenously into recipient mice. PBMCs from mouse EDTA-blood were isolated using Ficoll-Paque PREMIUM 1.084 (Cytiva). For ECP-treatment of epithelial cells, 6×106 MODE-K cells were seeded onto 10 cm dishes one day before ECP-treatment. The next day, cells were treated with 8-methoxypsoralen and UVA light in a calibrated BS-02 irradiation chamber. Cells were washed with PBS and 2×106 ECP-treated MODE-K cells were injected intravenously into recipient mice.

### DSS-Induced Colitis Model

For the induction of acute colitis, mice received 3% Dextran Sodium Sulfate (DSS colitis grade, 36-50 kDa; MP Biomedicals) in drinking water from day 0 to day 3, followed by normal drinking water. Mice were injected i.p. with 0.2 mg immune checkpoint inhibitor (ICI) or the respective isotype controls on days 0, 3 and 6 of the experiment. Recipients were transplanted i.v. with 107 ECP-treated donor splenocytes on days 3 and 6. In some experiments, steroid, adiponectin or AdipoRon were applied daily from days 3-7. Arginase-1 inhibitor was applied twice per day from days 4-7. STAT6 or PPARγ inhibitors were applied from day 4-7. Mice were injected with Clodrosomes on days 1, 3, 5 and 7. If not indicated differently, mice were sacrificed in day 8 by retroorbital bleeding to harvest serum and organs which were harvested for histopathological scoring and gene expression analysis.

### ECP-Treatment Using Enriched Immune Cells

Colitis was induced as described using 3% DSS and ICI-treatment. Where indicated, ECP-treated splenocytes were FACS-sorted for T-cells (CD45+CD3+; 3×106) or myeloid cells (CD45+CD3-CD19-CD11b+; 1×106) before intravenous injection into recipient mice. Antibodies for sorting are summarized in Table S8. Table S8 is shown below.

TABLE S8
Antibodies for FACS-sorting

| Antigen | Clone | Isotype | Dilution | Vendor | Fluorochrome |
|---|---|---|---|---|---|
| CD45 | 30-F11 | Rat IgG2b, κ | 1:200 | Biolegend | Pacific Blue |
| CD3 | 14A2 | Rat IgG2b, κ | 1:200 | Biolegend | Alexa Fluor |
| | | | | | 488 |
| CD11b | M1/70 | Rat IgG2b, κ | 1:100 | BioLegend | PE |

### Hematopoietic Chimera Generation

To generate mice lacking Arginase-1 in the hematopoietic compartment, WT C57BL/6 recipients were transplanted on day-30 with 5×106 Arg1−/− or C57BL/6 WT bone marrow (BM) cells i.v. in the tail vein after 12 Gy (2×6 Gy) total body irradiation (TBI). Mice were used as ECP recipients or donors in DSS-induced colitis experiments.

### B16.F10 Intravenous/Metastatic Tumor Model

C57BL/6 mice were intravenously injected with 10 000 B16.F10luc/GFP melanoma cells in the tail vein and treated with 0.2 mg ICI or the respective isotype control on day 1, 4, 8, 16 and 22. ECP-treated splenocytes were transplanted (107; i.v.) on days 13, 18, and 22, and daily steroids were given (i.p.) from days 13-22. Survival of the mice was monitored until day 60.

### 4434 Intravenous Tumor Model

C57BL/6 recipients were transplanted intravenously with 2×106 4434 melanoma cells in the tail vein and the treatment schedules were comparable as described for the B16.F10 (i.v.) model.

### MC38-OVAdim Subcutaneous Tumor Model

C57BL/6 recipients were transplanted subcutaneously with 1×106 MC38-OVAdim or 0.5×106 MC38 cells into the shorn right flank. Recipients were treated i.p. with 0.2 mg ICI on days 1, 4, 8, 11, 15. ECP-treated splenocytes were transplanted (107; i.v.) on days 6, 11 and 15, daily steroids were given (i.p.) from days 6-15. On day 16, serum samples were harvested by retroorbital bleeding, the mice were euthanized and tumors were resected for further studies.

B16.F10-OVA Subcutaneous Model with Adoptive T-Cell Transfer

CD45.1 recipients were transplanted subcutaneously with 1×106 B16.F10-OVA cells into the shorn right flank and treated i.p. with 0.2 mg ICI on day 1, 4, 8, 11, 15. ECP-treated splenocytes were transplanted (107; i.v.) on days 6, 11 and 15, daily steroids were given (i.p.) from days 6-15. OT-I splenocytes (CD45.2) were activated on day 2 at a density of 2×106 cells/mL in the presence of 100 U/mL rmIL-2 (Peprotech) and 0.1 μg/mL OVA257-264 peptide (Sigma) in RPMI supplemented with 10% FCS, 1% P/S, 55 μM beta-Mercaptoethanol (Gibco) and 4 mM glutamine (Gibco). Splenocytes were washed on day 4 and replated with 100 U/mL rmIL-2 at 2×106 cells/mL. On day 5, OT-I splenocytes were washed and 5×106 cells were injected intravenously into the tail vein of tumor bearing mice. On day 16, serum samples were harvested by retroorbital bleeding, the mice were euthanized and tumors were resected for further studies.

### Combined Colitis and B16.F10 Tumor Model

C57BL/6 mice were intravenously injected with 10 000 B16.F10luc/GFP melanoma cells in the tail vein and received 3% Dextran Sodium Sulfate (DSS colitis grade, 36-50 kDa; MP Biomedicals) in drinking water from day 9-12, followed by normal drinking water. Mice were treated with 0.2 mg ICI or the respective isotype control on days 1, 4, 8, 16 and 22. ECP-treated splenocytes were transplanted (107; i.v.) on days 13, 18 and 22, daily steroid was given (i.p.) from days 13-22. Mice were imaged with BLI on day 23, organs and serum were harvested on day 24.

### Recall Immunity Experiment

For evaluation of recall immunity, T-cell donor mice were challenged with 104 B16.F10 melanoma cells (i.v.) and treated with anti-PD1 (days 1, 3, 6, 9, 12), ECP (days 3, 7, 12) or steroids (days 3-12). Secondary recipients were sub-lethally irradiated with 6 Gy TBI and injected i.v. with 104 B16.F10 or 2×106 4434 melanoma cells. Splenic T-cells were enriched from T-cell donors on day 13 using the Pan T-cell Isolation Kit II (mouse, Miltenyi) and 1×105 T-cells were transplanted i.v. into secondary recipients on day 3 following tumor injection. Untreated melanoma mice served as control.

### In Vivo Migration Analysis

To analyze tissue-specific migration and phagocytosis of ECP-treated cells, mice were fed with an alfalfa-free diet (D10012Gi AIN-93, ResearchDiets) throughout the experiment to reduce background fluorescence. Recipients were transplanted subcutaneously with 1×106 B16.F10 melanoma cells into the shorn right flank. Mice were treated with 0.2 mg anti-PD1 on days 1, 5, 8 and 11, and received 3% DSS (colitis grade, 36-50 kDa; MP Biomedicals) in drinking water from days 5-8. Donor splenocytes were isolated on day 12, subjected to ECP-treatment and stained with VivoTrack 680 (PerkinElmer) according to manufacturer's instructions. Where indicated, MODE-K small intestinal epithelial cells were subjected to ECP-treatment and VivoTrack 680 staining. Cells were resuspended in PBS and 107 ECP-treated VivoTrack 680 stained splenocytes were transplanted intravenously. For experiments with epithelial cell transfer, 2×106 MODE-K cells or splenocytes were transplanted intravenously. Recipients were euthanized at defined timepoints post-transplantation, and tumors and colitis tissue were harvested for ex vivo fluorescence imaging (FLI). FLI was done using an IVIS Lumina III (PerkinElmer). Following this, colitis and tumor tissues were processed into single cell suspensions as described.

### Adoptive Transfer of Adiponectin-Treated T-Cells

Splenic T-cells were isolated from C57BL/6 donors using Pan T-cell isolation kit II (Miltenyi) and activated using anti-CD3/CD28 beads (ThermoScientific) in the presence of 30 U/mL rmIL2 (Peprotech). T-cells were treated with 9 μg/mL recombinant murine adiponectin (GenScript) or vehicle for 72 h. C57BL/6 recipients received 1% DSS (MP Biomedicals) in drinking water from day 0 to day 3, followed by normal drinking water to induce mild colitis. Mice were injected i.p. with 0.2 mg anti-PD1 on day 0, day 3 and day 6 of the experiment. On day 4, activation beads were removed, T-cells were washed and 5×106 T-cells were intravenously injected into colitis animals. Colitis tissue was harvested on day 8. T-cells were stained for CD25 surface expression to determine activation.

Adoptive Transfer of T-Cells from Colitis Donors

C57BL/6 donor mice received 3% DSS in drinking water and anti-PD1 treatment according to the schedule described before. Splenic T-cells for the donor cohort were isolated on day 8 using Pan T-cell isolation kit II (Miltenyi). The C57BL/6 recipient cohort received 1% DSS (MP Biomedicals) in drinking water from days 0 to 3, followed by normal drinking water to induce mild colitis. Mice were injected i.p. with 0.2 mg anti-PD1 on days 0, 3 and 6 of the experiment. On day 4, the mice were injected i.v. with splenic T-cells from the donor animals. Colitis tissue of the recipient cohort was harvested on day 8.

Xenograft-Induced irAE Model

Xenograft-induced irAE was induced as described in Perez-Ruiz, E., et al., “Prophylactic TNF blockade uncouples efficacy and toxicity in dual CTLA-4 and PD-1 immunotherapy.” Nature 569, 428-432 (2019). In brief, Rag2−/−γc−/− were injected i.p. with 107 human PBMCs in 200 μL PBS. Healthy donor PBMCs were isolated by density-gradient separation (Lymphocyte separation medium, Anprotec). On days 4, 7, 10, and 13, mice were treated i.p. with 200 μg Pembrolizumab and transplanted i.v. with 107 ECP-treated human PBMCs, depending on the treatment cohort. Steroids were applied i.p. daily from days 4-13. The mice were sacrificed on day 15 for serum and organ harvest.

### Long-Term Steroid and ECP-Treatment

WT C57BL/6 (CD45.2) mice were injected daily with steroids (1 mg/kg bodyweight) or vehicle for 50 days, or received 8 weekly ECP transplantations. Splenocytes for ECP transplantations were isolated from naïve CD45.1 donors. Recipient mice were sacrificed on day 50 and spleens were processed for FACS analysis.

### In Vivo Apoptosis Analysis

For a better understanding of whether ECP-treated splenocytes also undergo apoptosis in vivo, C57BL/6luc mice received B16.F10 melanoma (i.v.) and 0.2 mg anti-PD1 (d1, d4, d8). Splenocytes were isolated and either ECP-treated or left untreated, and 10⁷ splenocytes were injected intravenously into Rag2^{-/-}γc^{-/-} recipients. Expansion of cells was monitored by BLI. Recipients were sacrificed on day 30 and colon, lung, liver and spleen underwent ex vivo BLI.

### In Vivo Treatments

Treatment timepoints are indicated in the figures and methods. ICI treatment consisted of either 0.2 mg anti-PD1 (mouse, clone RMP1-14; Ichorbio) or 0.2 mg anti-CTLA4 (mouse, clone 9D9; Ichorbio) per injection intraperitoneally. Controls were Rat IgG2a Isotype Control (clone 1-1, Ichorbio) and Mouse IgG2b Isotype Control (clone MPC-11, Ichorbio), respectively. Pembrolizumab (Keytruda, anti-human PD1; MSD) was injected i.p. at 0.2 mg. Steroids (Prednisolone, Solu-Decortin H; Merck) were dissolved in PBS applied i.p. at 1 mg/kg bodyweight. Adiponectin/Acrp30 (GenScript) was reconstituted in H2O, diluted in PBS and applied i.p. at 4 μg per injection. AdipoRon (MedChemExpress) was prepared in DMSO/corn oil (5%/95%) and given orally at 50 mg/kg bodyweight. The Arginase-1 inhibitor CB-1158 (Numidargistat) dihydrochloride (MedChemExpress) was given orally in H₂O twice per day at 100 mg/kg bodyweight. The STAT6-inhibitor AS1517499 (MedChemExpress) and the PPARγ-inhibitor GW9662 (MedChemExpress) were dissolved in DMSO/PEG300/Tween80/PBS (10%/40%/5%/45%) and applied i.p. at 10 mg/kg and 1 mg/kg, respectively. Phagocytes were depleted using the Standard Macrophage Depletion Kit (Encapsula Nano Sciences) by intravenous injection of 0.2 mL Clodronate-containing liposomes (Clodrosome) or control liposomes (Encapsome). Macrophage depletion was confirmed by FACS and qPCR.

Histopathological Analysis of irAE Target Organs

Colon, liver, skin and lung were harvested, formalin-fixed and paraffin embedded. Sections were stained with H&E and scored by an experienced pathologist, based on histopathological characterization of human irAEs, including lymphocyte and neutrophil infiltration, crypt abscesses, and apoptotic cells on a scale from 0 (absent) to 2 (massive). Scoring was done blinded to the experimental group.

Histopathology Scoring of irAE in Colon Patient Tissue

Intestinal biopsy specimens (small intestine, large intestine) were prepared according to standardized procedures for formalin-fixed and paraffin-embedded tissues at the Institute of Surgical Pathology, Freiburg. In brief, 2 μm sections were generated and H&E stains were performed. Scoring of respective specimens was relied on the following parameters: eosinophilia, apoptosis, ulcer, cryptitis or crypt abscess formation, lympho-plasmocytic infiltration and signs of regeneration, focal or diffuse disease presentation (each parameter evaluated for absence (0) or presence (1)). A sum score (0-9) for each individual sample was calculated in a blinded fashion by an experienced pathologist.

### In Vivo Bioluminescence Imaging (BLI)

Luciferin (D-luciferin, potassium salt (S)-4,5-Dihydro-2-(6-hydroxy-2-benzothiazolyl)-4-thiazolecarboxylic acid potassium salt; Biosynth) was dissolved in H₂O and injected i.p. at a concentration of 150 μg/g body weight. After 10 minutes, the mice were imaged using an IVIS Lumina III in vivo imaging system (PerkinElmer) with an exposure time of 2 minutes. The luciferase signal was quantified in photons per second per mouse. Acquisition, analysis, and visualization of BLI were performed using Living Image Software (PerkinElmer).

### Intestinal Leukocyte Isolation

Intestinal leukocytes were isolated using the lamina propria dissociation kit (Miltenyi). In brief, colon samples were harvested and Peyer's patches removed. Samples were flushed with PBS to remove feces, opened longitudinally and cut into 5-10 mm segments. Epithelial cells were removed using pre-digestion solution. Colon tissue was transferred into gentleMACS C Tubes (Miltenyi) containing the enzyme mix and digested on a gentleMACS Octo Dissociator with Heater (Miltenyi) using the appropriate program. Cells were washed with PBS and applied on a 100 μm strainer before further downstream applications.

### Tumor Digestion

Single cell suspensions were obtained from B16.F10 tumor tissue after digestion with 1 mg/mL collagenase IA (Sigma) and 50 μg/mL DNase I (Sigma) in DMEM medium at 37° C. and 1,400 rpm for 1 h. Hematopoietic cells were enriched by density gradient centrifugation with 70% (3 mL), 40% (4 mL) and 30% (3 mL) Percoll (Cytiva), where the latter contained the digested tumor. Enriched cells were collected after centrifugation (680 g, acc 3, no brake, RT) above the bottom phase. MC38 tumors were digested with 20 μg/mL Liberase™ (Roche) and 50 μg/mL DNase I (Sigma) in DMEM medium at 37° C. and 1,000 rpm for 30 min. Immune cells were enriched by density gradient centrifugation with 30% Percoll (Cytiva) for 20 min at 800 g (no brake).

### Mass Cytometry (CyTOF)

For multidimensional protein analysis, C57BL/6 (CD45.2) mice were treated with DSS and anti-PD1 to induce colitis, as described, whereas one group received ECP splenocytes from CD45.1 donors. Organs were harvested on day 8 for analysis. Spleens were meshed and underwent erythrocyte lysis; colon tissue was digested as described and 3×106 cells were used for staining. Dead cells were stained with 2.5 UM MM-DOTA 139La for 5 min at RT, followed by surface protein staining for 30 min at RT. Cells were fixed in 1.6% PFA in PBS for 5 min at RT and permeabilized using the Transcription Factor Staining Buffer Set (eBioscience). Intracellular proteins were labelled for 60 min at RT, followed by secondary antibodies for 30 min at RT. Cells were stained with 125 nM Cell-ID Intercalator-Ir (Fluidigm) in 4% PFA in PBS o/n at 4° C. Data were acquired on a Helios CyTOF (Fluidigm) and processed using FlowJo v10 (TreeStar) and OMIQ (Dotmatics) softwares. Files were cleaned manually by exclusion of doublets and dead cells. Cells were scaled using Arcsinh from −5 to 12000 and pre-gated on CD45.2 recipient cells, followed by subsampling on 23000 (colon) or 8000 (spleen) cells. Dimension reduction was run to create opt-SNE plots. Different populations were generated by FlowSOM clustering with Elbow and Consensus metaclustering. Clusters were identified based on marker expression. Violin plots show median marker intensity in specified clusters. Heatmaps were generated on medians with Euclidean row distance. All antibodies used for CyTOF are summarized in Table S9 (shown below).

TABLE S9
CyTOF antibodies

| Marker | Channel | Clone | Dilution | Isotype |
|---|---|---|---|---|
| anti-PE | 165 Ho | n/a | 1:200 | n/a |
| Arg1-PE | n/a | AlexF5 | 1:100 | Rat IgG2a, κ |
| B7-H3 | 175 Lu | MIH35 | 1:50 | Rat IgG2a, κ |
| B7-H4 | 161 Dy | HMH4-5G1 | 1:100 | Armenian Hamster IgG |
| B220 | 176 Yb | RA3-6B2 | 1:200 | Rat IgG2a, κ |
| BTLA | 143 Nd | 6A6 | 1:100 | Armenian Hamster IgG |
| CXCR5 | 153 Eu | L138D7 | 1:100 | Rat IgG2b, κ |
| CD3e | 152 Sm | 145-2C11 | 1:200 | Armenian Hamster IgG |
| CD4 | 172 Yb | RM4-5 | 1:800 | Rat IgG2a, κ |
| CD8a | 168 Er | 53-6.7 | 1:800 | Rat IgG2a, κ |
| CD11b | 148 Nd | M1/70 | 1:200 | Rat IgG2b, κ |
| [MAC1] | | | | |
| CD11c | 142 Nd | N418 | 1:400 | Armenian Hamster IgG |
| CD19 | 149 Dm | 6D5 | 1:400 | Rat IgG2a, κ |
| CD25 | 151 Eu | 3C7 | 1:100 | Rat IgG2b, κ |
| CD39 | 163 Dy | 5F2 | 1:100 | Mouse IgG1, κ |
| CD44 | 171 Yb | IM7 | 1:800 | Rat IgG2b, κ |
| CD45.1 | 147 Sm | A20 | 1:200 | Mouse (A.SW) IgG2a, κ |
| CD45.2 | 111 Cd | 104 | 1:100 | Mouse (SJL) IgG2a, κ |
| CD47 | 144 Nd | Miap301 | 1:200 | Rat IgG2a, κ |
| CD62L | 160 Gd | MEL-14 | 1:800 | Rat IgG2a, κ |
| CD69 | 145 Nd | H1.2F3 | 1:100 | Armenian Hamster IgG |
| CD127 | 150 Nd | A7R34 | 1:200 | Rat IgG2a, κ |
| CD206 | 115 In | C068C2 | 1:100 | Rat IgG2a, κ |
| CTLA-4 | 159 Tb | 9H10 | 1:100 | Syrian Hamster IgG |
| Eomes | 158 Gd | Dan11mag | 1:100 | Rat IgG2a, κ |
| F4/80 | 113 Cd | BM8 | 1:100 | Rat IgG2a, κ |
| FOXP3 | 112 Cd | FJK-16s | 1:100 | Rat IgG2a, κ |
| KLRG1 | 157 Gd | 2F1/KLRG1 | 1:200 | Syrian Hamster IgG |
| Lag-3 | 167 Er | C9B7W | 1:50 | Rat IgG1, κ |
| Ly108 | 156 Gd | 13G3-19D | 1:400 | Mouse IgG2a, κ |
| Ly6G/C | 141 Pr | RB6-8C5 | 1:200 | Rat IgG2b, κ |
| [Gr1] | | | | |
| MHC II | 114 Cd | AF6-120.1 | 1:200 | Mouse (BALB/c) IgG2a, κ |
| NK1.1 | 170 Er | PK136 | 1:200 | Mouse IgG2a, κ |
| PD-1 | 173 Yb | RMP1-30 | 1:100 | Rat IgG2b, κ |
| PD-L1 | 162 Dy | 10F.9G2 | 1:200 | Rat IgG2b, κ |
| PD-L2 | 164 Dy | TY25 | 1:200 | Rat IgG2a, κ |
| T-bet | 155 Gd | 4B10 | 1:100 | Mouse IgG1, κ |
| TCRβ | 169 Tm | H57-597 | 1:400 | Armenian Hamster IgG |
| TER-119 | 154 Sm | TER119 | 1:100 | Rat IgG2b, κ |
| Tim3 | 146 Nd | RMT3-23 | 1:50 | Rat IgG2a, κ |
| TOX | 174 Yb | REA473 | 1:50 | recombinant human IgG1 |
| Vista | 166 Er | MIH63 | 1:100 | Rat IgG2a, κ |

RNA Isolation, Reverse Transcription and qPCR

Total RNA from tissues was isolated using QIAzol lysis reagent, RNA from cells was isolated using RNeasy Mini or Micro Kit. The RNeasy FFPE Kit was used to isolate RNA from formalin-fixed paraffin-embedded patient specimens (all QIAGEN). Up to 1 μg of total RNA was reverse transcribed into cDNA using the High-Capacity cDNA Reverse Transcription Kit (ThermoScientific), 15 ng of Yeast RNA (ThermoScientific) were spiked into RNA samples from FFPE-tissue. Quantitative PCR (qPCR) was done using LightCycler 480 SYBR Green I Master mix (Roche) with 10-40 ng of cDNA and 5 μM forward and reverse primer on a LightCycler 480 machine (Roche). Primers (Table S10) were obtained from Eurofins Genomics (Germany). Gene expression was calculated relative to reference genes or relative to control using the 2-44CT method. Table S10 is shown below.

S10: Primer sequences

| Gene | Forward | reverse |
|---|---|---|
| hADIPOQ | 5′-TATGATGGCTCCACTGGTA-3′ | 5′-GAGCATAGCCTTGTCCTTCT-3′ |
| hTBP | 5′-GCCCGAAACGCCGAATAT-3′ | 5′-CCGTGGTTCGTGGCTCTCT-3′ |
| mAdgre1 | 5′-CGTGTTGTTGGTGGCACTGTGA-3′ | 5′-CCACATCAGTGTTCCAGGAGAC-3′ |
| mAdipoq | 5′-CTCCACCCAAGGGAACTTGT-3′ | 5′-AGGACCAAGAAGACCTGCAT-3′ |
| mAdipor1 | 5′-TCTGCCTCAGTTTCTCCTGGCT-3′ | 5′-GTAATAGAGCCAGGGAACGAAGC-3′ |
| mAdipor2 | 5′-ACACAGAGACGGGCAACATT-3′ | 5′-GAAGAGTCGGGAGACCCCTT-3′ |
| mAsc12 | 5′-CCTATGCCTTACCCATGCT-3′ | 5′-TTTCCAAGTCCTGATGCTG-3′ |
| mArg1 | 5′-GGAATCTGCATGGGCAACCTGTGT-3′ | 5′-AGGGTCTACGTCTCGCAAGCCA-3′ |
| mAxin2 | 5′-GAAAGCCGCCATAGTCTGGA-3′ | 5′-CTCTTCATAGCTGCCGGAGG-3′ |
| mCd68 | 5′-GGCGGTGGAATACAATGTGTCC-3′ | 5′-AGCAGGTCAAGGTGAACAGCTG-3′ |
| mCd99 | 5′-GCGAGTGACGACTTCAACCTGG-3′ | 5′-CGTCCTCCAGGTCGAAGCCTC-3′ |
| mCxcr1 | 5′-TACGATCAGCGCCTCAATGCCA-3′ | 5′-AGCAGGAAACCAGCCACTAGCT-3′ |
| mDct | 5′-GCAAGATTGCCTGTCTCTCCAG-3′ | 5′-CTTGAGAGTCCAGTGTTCCGTC-3′ |
| mHprt | 5′-AGCCTAAGATGAGCGCAAGT-3′ | 5′-TTACTAGGCAGATGGCCACA-3′ |
| mItga1 | 5′-GGCAGTGGCAAGACCATAAGGA-3′ | 5′-CATCTCTCCGTGGATAGACTGG-3′ |
| mItga2 | 5′-TACAGACGTGCTCCTGGTAGGT-3′ | 5′-CCGAGCATTTCCAGTGCCTTCT-3′ |
| mItga2b | 5′-GGTGCTGACAATGTGTTGGAGC-3′ | 5′-GGTGCTGACAATGTGTTGGAGC-3′ |
| mItga4 | 5′-GCAAAGAGGTCCCAGGCTACAT-3′ | 5′-CCTGTAATCACGTCAGAAGTCCC-3′ |
| mItga8 | 5′-CCGATTTGCTGTTCCTCGCCTT-3′ | 5′-GACCTGAGCAATGGCAGTGATG-3′ |
| mItga9 | 5′-ACAGCACCTCAGTGTCACGATG-3′ | 5′-TTCTGTGCCACATCAGCCGTCA-3′ |
| m1tga11 | 5′-TGGAGATGTCGCAGACTGGCTT-3′ | 5′-GAGGAATCACCTTGCCAGCACT-3′ |
| mItgad | 5′-GCTTAGGAGTCTGCCTTTGCTG-3′ | 5′-GTCAGGTGAACCTTTGCGGACA-3′ |
| mItgae | 5′-GAAGTGGAACGGAGGGTCCTTT-3′ | 5′-GTCTGGAACTCGTAGGTGACCT-3′ |
| mItga1 | 5′-CATGCAGCCTATCCTGAGACCT-3′ | 5′-GGTCAGGTTTGCCTCACACTTC-3′ |
| mItgam | 5′-TACTTCGGGCAGTCTCTGAGTG-3′ | 5′-ATGGTTGCCTCCAGTCTCAGCA-3′ |
| mItgav | 5′-GTGTGAGGAACTGGTCGCCTAT-3′ | 5′-CCGTTCTCTGGTCCAACCGATA-3′ |
| mItgax | 5′-TGCCAGGATGACCTTAGTGTCG-3′ | 5′-CAGAGTGACTGTGGTTCCGTAG-3′ |
| mItgb1 | 5′-CTCCAGAAGGTGGCTTTGATGC-3′ | 5′-GTGAAACCCAGCATCCGTGGAA-3′ |
| mItgb2 | 5′-CTTTCCGAGAGCAACATCCAGC-3′ | 5′-GTTGCTGGAGTCGTCAGACAGT-3º |
| mItgb3 | 5′-GTGAGTGCGATGACTTCTCCTG-3′ | 5′-CAGGTGTCAGTGCGTGTAGTAC-3′ |
| mItgb4 | 5′-ACACAAGCTCCAGCAGACGAAG-3′ | 5′-TCCACCTGCTTCTCTGTCAGCT-3′ |
| mItgb6 | 5′-ACTCATTCCTGGAGCAACCGTG-3′ | 5′-GCTGTGAAAGACAGGTTGAGTCC-3′ |
| mItgb7 | 5′-TGATGTCCGAGTCAACCAGACG-3′ | 5′-ACTCCTCTGAGAAGCCAAGAGC-3′ |
| mItgb8 | 5′-ATGCTTCAGGCTGCCGTCTGTG-3′ | 5′-CAGTTTCCGTCATTCGGCACCA-3′ |
| mPecam1 | 5′-CCAAAGCCAGTAGCATCATGGTC-3′ | 5′-GGATGGTGAAGTTGGCTACAGG-3′ |
| mPme1 | 5′-GGAAGTGACTGTCTACCACCGA-3′ | 5′-CTCAGGAAGTGCTTGGTCTCTC-3′ |

### Microarray Analysis

Microarray analysis was done from the colon tissue of mice with DSS-induced colitis treated with anti-PD1 alone or anti-PD1 plus ECP. Tissue was harvested, flushed, washed in ice-cold PBS and homogenized for total RNA using the miRNeasy Mini Kit (Qiagen). RNA quality was assessed using an Agilent 2200 TapeStation (Agilent Technologies) and RIN was above 8.8 for all samples. 250 ng of total RNA were processed to fragmented and labeled ds-cDNA using the GeneChip™ WT PLUS Kit (Applied Biosystems), hybridized on Clariom S mouse microarrays (Applied Biosystems) using the GeneChip™ Hybridization, Wash, and Stain Kit (Applied Biosystems) and scanned according to the manufacturer's instructions. Microarray analysis was performed as described in Hamarsheh, S., et al., “Immune modulatory effects of oncogenic KRAS in cancer.” Nat Commun. 11, 5439 (2020). Briefly, the arrays were normalized via robust multichip averaging as implemented in the R/Bioconductor oligo package and gene level annotation was retrieved from pd.clariom.s.mouse R/Bioconductor package. A linear model-based approach, limma R/Bioconductor package, was used to identify regulated genes between DSS/anti-PD1 and DSS/anti-PD1/ECP. An adjusted (Benjamini-Hochberg) p-value below 0.05 was considered to be significant.

Single Cell RNA Sequencing (scRNA Seq)

WT C57BL/6 mice were treated with DSS and ICI according to the previously described schedule to induce colitis, whereas one group received ECP splenocytes (n=2 per group). On day 8, the colon was harvested and digested to isolate intestinal leukocytes. Dead cells were removed using the Dead Cell Removal Kit (Miltenyi), followed by immune cell enrichment using CD45 MicroBeads (Miltenyi). Cells were washed and counted in 0.04% BSA/PBS according to the 10× Genomics protocol and 10 000 CD45+ cells were encapsulated into droplets with barcoded Gel Beads using the Chromium Controller Single-Cell Instrument (10× Genomics). Libraries were prepared using Chromium Next GEM Single Cell 3′ Reagent Kits v3.1 (10× Genomics). Generated scRNA Seq libraries were pooled and sequenced on a NovaSeq 6000 (Illumina) at 44658 mean reads per cell. scRNA fastq files were processed with 10× Genomics Cell Ranger 6.1.2, using “cellranger count—no-bam-nosecondary” command. The mouse reference transcriptome was downloaded from the pre-built Cell Ranger reference package (mm10 GENCODE vM23/Ensembl 98 2020-A). Other parameters were set as default. Downstream analysis was performed with the Seurat (v4.3.0) R/Bioconductor package. Single cells were discarded if they were expressing less than 200 genes or more than 4000 genes, or if their percentage of mitochondrial reads was higher than 10%. Genes quantified in less than 3 cells were filtered-out. Read counts were normalized with a scale factor of 10 000. Normalized data were scaled and centered, whereas percentage of mitochondrial content was regressed out. The Principal Component Analysis on the top 3000 variable features was then computed. A nearest neighbor graph was constructed on the top 20 principal components and Louvain clustering was performed with a resolution set as 0.8. Finally Uniform Manifold Approximation and Projection (UMAP) was used to visualize the remaining cells on 2 dimensions. Cells types were annotated using the sctype R scripts using custom immune system marker gene lists (Table S11). Cluster/cell type specific marker genes were found using the “FindConservedMarkers” function with the following parameters: test.use=“MAST”, min.pct=0.25, logfc.threshold=0.25, min.cells.group=10, only.pos=TRUE. Table S11 is shown below.

TABLE S11
Modified scType immune system marker genes

| | geneSymbolmore1 | geneSymbolmore2 |
|---|---|---|
| Cell Name | (positive marker) | (negative marker) |
| CD4+ Treg | Foxp3, CD4, CD3e, Izkf2, Tnfrsf4, Tnfrsf9, Tnfrsf18, Il10, Ctla4 | |
| Pro-B cells | CD27, CD220, IgD, CD24, PTPRC, PAX5, CD24, CD38, | |
| | CD79A, DNTT, C10orf10, VPREB1, ARPP21, CD99, IGLL1, | |
| | CD9, CD79B, TCL1A, IGLL5, HLA-DQA1, HLA-DQB1, | |
| | VPREB3, IGLL5 | |
| Pre-B cells | CD19, CD220, CD27, IgD, CD24, PTPRC, PAX5, CD24, CD38, | |
| | CD79A, NSMCE1, PCDH9, ACSM3, CCDC191, TCL1A, | |
| | CD79B, TCL1A, IGLL5, HLA-DQA1, HLA-DQB1, VPREB3, | |
| | IGLL5 | |
| Naive B cells | CD19, IgD, CD38, CD24, CD20, MS4A1, PTPRC, PAX5, CD24, | |
| | CD38, CD79A, JCHAIN, SSR4, FKBP11, SEC11C, DERL3, | |
| | PRDX4, IGLL5, CD79B, TCL1A, IGLL5, HLA-DQA1, HLA- | |
| | DQB1, CD138, CD38, VPREB3, IGLL5 | |
| Memory B cells | CD19, CD27, IgD, CD38, CD24, CD20, MS4A1, PTPRC, PAX5, | |
| | CD24, CD38, CD79A, JCHAIN, SSR4, FKBP11, SEC11C, | |
| | DERL3, PRDX4, IGLL5, CD79B, TCL1A, IGLL5, HLA-DQA1, | |
| | HLA-DQB1, CD138, CD38, CD27, VPREB3, IGLL5, CD40 | |
| Plasma B cells | CD19, CD27, IgD, CD38, CD24, CD20, MS4A1, PTPRC, PAX5, | CD20, MS4A1 |
| | CD24, CD38, CD79A, JCHAIN, SSR4, FKBP11, SEC11C, | |
| | DERL3, PRDX4, IGLL5, CD79B, TCL1A, IGLL5, HLA-DQA1, | |
| | HLA-DQB1, CD138, CD38, VPREB3, IGLL5 | |
| Naive CD8+ T | CD8, CD2, CD3D, CD3E, CD3G, CD3Z, CD45RA, CD62L, | CD25, CD44, CD69, |
| cells | CD27, CD127, CCR7, CD45, CD8A, CD8B, CCR6, CD11b, | HLA-DRA, CD95 |
| | CD30, CD6, CTLA4, IL2RA, PTPRC, SELL, CCR7, GNLY, | |
| | Trac, Ltb, Cd52, Trbc2, Shisa5, Lck, Thy1, Dapl1 | |
| Naive CD4+ T | CD4, CD2, CD3D, CD3E, CD3G, CD3Z, CD45RA, CD62L, | CD25, CD44, CD69, |
| cells | CD27, CD127, CCR7, CD45, CCR6, CD11b, CD30, CD6, | HLA-DRA, CD95 |
| | CTLA4, IL2RA, PTPRC, SELL, CCR7, Trac, Ltb, Cd52, Trbc2, | |
| | Shisa5, Lck, Thy1, Dapl1 | |
| Memory CD8+ T | CD8, CD2, CD3D, CD3E, CD3G, CD3Z, CD25, CD45RA, | CD19 |
| cells | CD62L, CD27, CD127, CCR7, CD45, CD8A, CD8B, CCR6, | |
| | CD30, CD45RO, CD6, CTLA4, IL2RA, G ZMB, SELL, CCR7, | |
| | GNLY, S100A4, Trac, Ltb, Cd52, Trbc2, Shisa5, Lck, Thy1, | |
| | Dapl1 | |
| Memory CD4+ T | CD4, CD2, CD3D, CD3E, CD3G, CD3Z, CD25, CD45RA, | CD19 |
| cells | CD62L, CD27, CD127, FOXP3, CCR7, CD45, CCR6, CD30, | |
| | CD45RO, CD6, CTLA4, IL2RA, GZMB, SELL, CCR7, S100A4, | |
| | Trac, Ltb, Cd52, Trbc2, Shisa5, Lck, Thy1, Dapl1 | |
| Effector CD8+ T | CD8, CD2, CD3D, CD3E, CD3G, CD3Z, CD25, CD45RA, | SELL, CCR7, CD19 |
| cells | CD62L, CD27, CD127, CCR7, CD45, CD8A, CD8B, CCR6, | |
| | CD11b, CD30, CD6, CTLA4, IL2RA, GZMB, PTPRC, GNLY, | |
| | Trac, Ltb, Cd52, Trbc2, Shisa5, Lck, Thy1, Dapl1, IFNG | |
| Effector CD4+ T | CD4, CD2, CD3D, CD3E, CD3G, CD3Z, CD25, CD45RA, | SELL, CCR7, CD19 |
| cells | CD62L, CD27, CD127, FOXP3, CCR7, CD45, CCR6, CD11b, | |
| | CD30, CD6, CTLA4, IL2RA, GZMB, PTPRC, Trac, Ltb, Cd52, | |
| | Trbc2, Shisa5, Lck, Thy1, Dapl1, IFNG | |
| T Helper 1 cells | CD4, CD3D, CD3E, CD3G, CD3Z, CXCR1, IFNG, Tbet, IL2 | IL4, IL17 |
| T Helper 2 cells | CD4, CD3D, CD3E, CD3G, CD3Z, CCR4, IL4, STAT6, GATA3 | IL17, IL2 |
| T Helper 17 cells | CD4, CD3D, CD3E, CD3G, CD3Z, CCR4, IL17, RORgt, STAT3 | IL4, IL2 |
| γδ-T cells | CD2, CD3D, CD3E, CD3G, CD3Z, CD25, CD45RA, CD62L, | SELL, CCR7, CD19 |
| | CD27, CD127, CCR7, CD45, CCR6, CD11b, CD30, CD6, | |
| | CTLA4, IL2RA, GZMB, PTPRC, TRDV2, TRGV9, TRGC1, | |
| | Trac, Ltb, Cd52, Trbc2, Shisa5, Lck, Thy1, Dapl1, TRG, TRD | |
| Platelets | CD41, CD42b, CD61, CD31, PPBP, PF4, GNG11, SDPR, CLU, | |
| | CD41, CD110 | |
| CD8+ NKT-like | CD8, CD56, CD2, CD16, CD94, CD3D, CD3E, CD3G, CD3Z, | |
| cells | NKp46, CD11b, CD161, CD314, CD69, NKG7, CD122, | |
| | NKG2D, GZMB, GZMA, GZMM, GNLY, COX6A2, ZMAT4, | |
| | KIR2DL4 | |
| CD4+ NKT-like | CD4, CD56, CD2, CD16, CD94, CD3D, CD3E, CD3G, CD3Z, | |
| cells | NKp46, CD11b, CD161, CD314, CD69, NKG7, CD122, | |
| | NKG2D, GZMB, GZMA, GZMM, COX6A2, ZMAT4, KIR2DL4 | |
| Natural killer | CD56, CD2, CD16, CD94, NKp46, CD11b, CD161, CD314, | CD3D, CD3E, CD3G, |
| cells | CD69, NKG7, CD122, NKG2D, GZMB, GZMA, GZMM, | CD3Z, CD4, CD8 |
| | FCGR3A, GNLY, COX6A2, ZMAT4, KIR2DL4, NKG7 | |
| Eosinophils | CD11b, CD193, CD123, CD125, CD15, SIGLEC8, CLC, | |
| | GATA1, CEBPE, SEMG1, ALOX15, CCL23, PRSS41, PRSS33, | |
| | THBS4, FOXI1 | |
| Neutrophils | CD66b, CD11b, CD15, CD16, CXCL8, FCGR3B, MNDA, | |
| | CXCR2, MPO, ELANE, PRTN3, MPO, AZU1, LYZ, S100A8, | |
| | S100A9, PI3, CHI3L1, ANXA3, CXCL1, TGM3, BTNL3, | |
| | C4BPA, MMP9, CD24, BPI, LTF, GCA, Camp, Ngp, Chil3, Ltf | |
| Basophils | CD63, CD203c, CD123, CLC, MS4A3, TCN1, CPA3, HDC, | |
| | GATA2, MS4A2, IL4, GCSAML, GATA2, TPSAB1 | |
| Mast cells | CD117, CD203c, CD25, KIT, SLC18A2, CD33, CD32, FCER1A, | |
| | TPSD1, HPGDS | |
| Classical | CD14, CD11b, CD68, HLA-DR, CD33, CD11c, CD123, CD15, | CD56, CD16 |
| Monocytes | CD3D, CD3E, CD3G, CD3Z, CD66b, VCAN, S100A12, | |
| | CXCL8, S100A8, S100A9, LYZ, CST3, Elane1, LY6C1 | |
| Non-classical | CD14, CD16, CD11b, CD68, HLA-DR, CD33, CD11c, CD123, | CD56 |
| monocytes | CD15, CD3D, CD3E, CD3G, CD3Z, CD66b, FCGR3A, | |
| | CDKN1C, LST1, FCER1G, MS4A7, RHOC, S100A8, S100A9, | |
| | CST3, C1QC, Elane1, CX3CR1, TREML4, CD43 | |
| Intermediate | CD14, CD16, CD11b, CD68, HLA-DR, CD33, CD11c, CD123, | CD56 |
| monocytes | CD15, CD3D, CD3E, CD3G, CD3Z, CD66b, IL1B, S100A8, | |
| | S100A9, CST3, C1QC, Elane1, LY6C1, CD209 | |
| Macrophages | ADGRE1, CD68, CD163, CD14, CD11b, CD80, CD86, CD16, | CD56 |
| | CD64, CCL18, CD115, CD11c, CD32, HLA-DR, MRC1, MSR1, | |
| | GCA, Pf4 | |
| M1 Macrophages | ADGRE1, CD68, NOS2, TNF, CSF2, IFNG, IL6 | ARG1 |
| M2 Macrophages | ADGRE1, CD68, ARG1, CD206, CD301, CSF1R, IL10, IL4 | NOS2 |
| Megakaryocyte | CD61, CD41, CD42b, CD41a, CD42a, CXCR4, CD110 | |
| Endothelial | PECAM1, CD34, KDR, CDH5, PROM1, PDPN, TEK, FLT1, | |
| | VCAM1, PTPRC, VWF, ENG, MCAM, ICAM1, FLT4, SELE | |
| Erythroid-like and | PTPRC, GYPA, RUVBL1, TFRC, FOLR1, CD36, ITGA4, HBB, | |
| erythroid | CD235a, HBD, CA1 | |
| precursor cells | | |
| HSC/MPP cells | CD105, CD34, CD44, CD73, CD45, CD29, STRO-1, NANOG, | PTPRC, CD38 |
| | SOX2, CD133, CD166, CD146, CD31, Nestin, OCT4, CD117, | |
| | KDR, CXCL8, AVP, CRHBP, ALDH1A1, CD49, CD90, CD69, | |
| | CD24, CD38, CD45RA, Keratin-19, ASPM, CD10, CD123, | |
| | ABCG2, CD135, CD49f, EpCAM, Keratin-7, SCA-1, CD14, | |
| | CD150, CD271, HLA-DR | |
| Progenitor cells | CD105, CD34, CD44, CD73, CD45, CD29, STRO-1, NANOG, | |
| | SOX2, CD133, CD166, CD146, CD31, Nestin, OCT4, CD117, | |
| | KDR, AVP, CRHBP, ALDH1A1, STMN1, CD38, PTPRC, | |
| | CD135 | |
| Myeloid | ITGAX, CD83, CD1C, NRP1, CLEC4C, CD86, IL3RA, CD80, | |
| Dendritic cells | CD1A, ITGAX, CD40, HLA-DQA1, CD11c, HLA-DR, HLA- | |
| | DPB1, HLA-DPA1, CLEC10A, CST3, GPR31, ODF3L1, PRB2, | |
| | CD207, ARSE, CLEC141, MRC, EBLN1, CRIP3 | |
| Plasmacytoid | ITGAX, CD83, CD1C, NRP1, CLEC4C, CD86, IL3RA, CD80, | |
| Dendritic cells | CD1A, ITGAX, CD40, HLA-DQA1, CD11c, HLA-DR, HLA- | |
| | DPB1, HLA-DPA1, CLEC10A, CST3, TPM2, LRRC26, ASIP, | |
| | GPM6B, KRT5, NTM, SCT, SHD, KCNA5, SCARA5, EPHA2, | |
| | MYMX, CD317, CD45R, LY6C, Siglec-H | |
| Granulocytes | CD203c, CD15, CD11b, CD63, CD66b, CD123, CD16, CD33, | |
| | CD117, CD45, Fc-epsilon RI-alpha, CD125, CD13, CD14, | |
| | CD25, CD44, CD69, CD9, HLA-DR, CCR3, CD116, CD11c, | |
| | CD193, CD24, CD32, CD43, CXCL8, FCGR3B, MNDA, | |
| | SIGLEC8, AZU1, MPO, CTSG, LYZ | |
| ISG expressing | IFIT1, IFIT2, IFIT3, IFIT5, ISG15, CCL3, CCL4, CCL3L3, | |
| immune cells | RSAD2, OASL, CXCL10, IFI15, ISG20 | |

To quantify transcription factor and signaling pathway activity, the decoupler R package was used with Dorothea and Progeny annotations respectively. The regulatory activity was calculated for every single cell by using wmean. Differences of activity between different cell type and/or condition was assessed by Wilcoxon test.

### Measurement of Adiponectin in Murine Serum

For quantification of adiponectin concentrations in murine serum specimen, blood was harvested by retroorbital bleeding and serum was isolated by centrifugation. Adiponectin concentrations were measured using Adiponectin Mouse ELISA Kit (ThermoFisher) at 20,000-fold dilution.

### Measurement of Adiponectin in Patient Serum

Adiponectin concentrations in patient serum samples were measured using the Human Adiponectin ProQuantum Immunoassay Kit (ThermoFisher) in 384-well format at 1,000-fold dilution.

### Isolation of Immune Cell Populations

Immune cells were isolated for gene expression analysis from bone marrow using Neutrophil Isolation Kit or Monocyte Isolation Kit, or from the spleen using NK cell Isolation Kit, Pan B-cell Isolation Kit or Pan T-cell Isolation Kit II (all for mouse; Miltenyi).

### Generation and Culture of Bone Marrow Derived Macrophages (BMDMs)

BMDMs were generated from bone marrow after erythrocyte lysis. Cells were cultured in RPMI supplemented with 10% FCS, 1% P/S and 20 ng/mL rmM-CSF (Peprotech). Culture medium was replaced on days 5 and 6. Cells were used on day 7 with a purity >95%, confirmed by surface CD11b and F4/80 expression. BMDMs (M0) were polarized for 16 h into M1 with 50 ng/mL rmIFNγ (Peprotech) and 20 ng/mL LPS (Sigma), or into M2 with 20 ng/mL rmIL-4 (Peprotech). If indicated, BMDMs were treated with increasing concentrations of recombinant adiponectin/Acrp30 (GenScript) or AdipoRon (MedChemExpress) for 48 h.

### Generation and Culture of Bone Marrow Derived Dendritic Cells (BMDCs)

BMDCs were generated and cultured from bone marrow as described in Stickel, N., et al., “MicroRNA-146a reduces MHC-II expression via targeting JAK/STAT-signaling in dendritic cells after stem cell transplantation.” Leukemia 31, 2732-2741 (2017). Cells were used on day 7 with a purity >90%, confirmed by CD11c surface expression. LPS (Sigma) stimulation was done with 100 ng/ml for 16 h.

### In Vitro Phagocytosis Analysis

WT C57BL/6 (CD45.2) bone marrow cells were generated into BMDMs and BMDCs for 7 days and seeded in 6-well plates at 10⁶ cells/well. The next day, splenocytes from CD45.1 mice were ECP-treated as described, stained with CellTrace Violet (CTV; ThermoScientific) and 2×10⁶ CTV⁺ splenocytes were added into the BMDM and BMDC cultures. Plates were centrifuged at 300×g for 2 min and cultured for 48 h before further FACS or PrimeFlow analysis. Phagocytosis was analyzed by FACS using congenic markers and CTV signal. If indicated, endocytosis was blocked by pre-incubation of BMDCs with 50 μM Endosidin9 (Sigma) or 50 μM PitStop2 (Sigma) for 1 h before addition of splenocytes. The STAT6 inhibitor AS1517499 (MedChemExpress) or the PPARγ inhibitor T0070907 (MedChemExpress) were added at 500 nM or 1 μM, respectively, into co-cultures 1 h after adding splenocytes.

### Flow Cytometry (FACS)

Spleens were isolated, meshed and erythrocytes were lysed. Single cell suspensions of tissues were prepared as described. Viability was analyzed by either Zombie NIR fixable dye (BioLegend) or LIVE/DEAD Fixable Aqua (Invitrogen) staining. Fc receptors were blocked with anti-mouse CD16/CD32 (BioLegend) for 10 min at 4° C., followed by surface staining with fluorochrome-conjugated antibodies for 30 min at 4° C. Cells were fixed and permeabilized for 40-60 min at 4° C. using the Fixation/Permeabilization Kit (BD Biosciences) or Transcription Factor Staining Buffer Set (eBioscience) for staining of cytoplasmatic proteins and cytokines or intranuclear proteins, respectively. For intracellular cytokine staining, cells were stimulated in the presence of Cell Stimulation Cocktail with protein transport inhibitor (eBioscience) for 4 h before surface and intracellular cytokine staining. Antibody against CD107α was added during stimulation. Calreticulin was stained for 20 min with an unconjugated antibody, followed by 20 min staining with a fluorochrome-conjugated anti-rabbit secondary antibody. Antigen-specific CD8 T-cells were analyzed after H-2Kb/OVA²⁵⁷⁻²⁶⁴ dextramer staining (Immudex). To determine protein phosphorylation, cells were surface stained, incubated with Lyse/Fix (BD Biosciences) for 10 min at 37° C., and permeabilized with ice-cold Phosflow Perm Buffer III (BD Biosciences) for 30 min at 4° C. Phosphorylated STAT6 (pY641) was stained for 30 min at 4° C. If total cell counts were needed, 123count eBeads Counting Beads (Invitrogen) were added directly before acquisition. All data were acquired on a BD LSR Fortessa (BD Biosciences) and analyzed with FlowJo v10 (TreeStar). For analysis of immune cells in patient blood, 50 μL EDTA blood were incubated with 5 μL of the respective antibody for 30 min, followed by addition of 2.5 mL Lyse/Fix (BD Biosciences). Samples were incubated for 10 min, before resuspension in FACS Flow (BD Biosciences). For some patients, analysis was done using the BD Multitest 6-Color TBNK together with BD Trucount counting Tubes according to manufacturer's instructions (BD Biosciences). Data was acquired with a Canto II flow cytometer (BD Biosciences) and analyzed using FACSDiva Software V6 (BD Biosciences). Antibodies used for Flow cytometry are summarized in Table S12 (shown below).

TABLE S12
Flow cytometry antibodies

| | | | |
|---|---|---|---|
| Antigen | Clone | Isotype | Dilution |

Antibodies for murine samples

| | | | |
|---|---|---|---|
| anti-rabbit (PE) | Polyclonal | Donkey Ig | 1:50 |
| Arginase 1 | A1exF5 | Rat IgG2a, κ | 1:100 |
| BTLA | 6A6 | Armenian hamster IgG | 1:100 |
| Calreticulin | Polyclonal | Rabbit Ig | 1:200 |
| CD3 | 17A2 | Rat IgG2b, κ | 1:200 |
| CD4 | GK1.5 | Rat IgG2b, κ | 1:200 |
| CD8 | 53-6.7 | Rat IgG2a, κ | 1:200 |
| CD11b | M1/70 | Rat IgG2b, κ | 1:200 |
| CD11c | N418 | Armenian Hamster IgG | 1:100 |
| CD19 | 6D5 | Rat IgG2a, κ | 1:200 |
| CD25 | PC61 | Rat IgG1, λ | 1:150 |
| CD45 | 30-F11 | Rat IgG2b, κ | 1:200 |
| CD45.1 | A20 | Mouse (A.SW) IgG2a, κ | 1:200 |
| CD45.2 | 104 | Mouse (SJL) IgG2a, κ | 1:200 |
| CD47 | miap301 | Rat IgG2a, κ | 1:100 |
| CD86 | GL-1 | Rat IgG2a, κ | 1:100 |
| CD107a | 1D4B | Rat IgG2a, κ | 1:100 |
| CD115 | AFS98 | Rat IgG2a, κ | 1:100 |
| CD206 | C068C2 | Rat IgG2a, κ | 1:150 |
| CD301 | LOM-14 | Rat IgG2b, κ | 1:150 |
| Cx3Cr1 | Polyclonal | Goat IgG | 1:100 |
| F4/80 | BM8 | Rat IgG2a, κ | 1:100 |
| FoxP3 | FJK-16s | Rat IgG2a, κ | 1:100 |
| Gr-1 | RB6-8C5 | Rat IgG2b, κ | 1:200 |
| IFNγ | XMG1.2 | Rat IgG1, κ | 1:100 |
| KLRG1 | 2F1/KLRG1 | Syrian Hamster IgG | 1:50 |
| Ly6C | HK1.4 | Rat IgG2c, κ | 1:200 |
| Ly6G | 1A8 | Rat IgG2a, κ | 1:200 |
| MHCII (I-Ab) | AF6-120.1 | Mouse (BALB/c) IgG2a, κ | 1:400 |
| NK1.1 | PK136 | Mouse IgG2a, κ | 1:100 |
| PD-1 | 29F.1A12 | Rat IgG2a, κ | 1:100 |
| SIINFEKL-H2kb | 25-D1.16 | Mouse IgG1, κ | 1:50 |
| STAT6 (pY641) | 18/P-Stat6 | Mouse IgG2a | 1:5 |
| Tim3 | RMT3-23 | Rat IgG2a, κ | 1:100 |
| TNFα | MP6-XT22 | Rat IgG1, κ | 1:100 |
| TOX | REA473 | recombinant human IgG1 | 1:50 |

Antibodies for human samples

| | | | |
|---|---|---|---|
| CD3 | SK7 | Mouse IgG1, κ | 1:10 |
| CD4 | SK3 | Mouse IgG1, κ | 1:10 |
| CD8 | SK1 | Mouse IgG1, κ | 1:10 |
| CD16 | B73.1 | Mouse IgG1, κ | 1:10 |
| CD19 | HIB 19 | Mouse IgG1, κ | 1:10 |
| CD19 | SJ25C1 | Mouse IgG1, κ | 1:10 |
| CD56 | MY31 | Mouse IgG1, κ | 1:10 |
| CD56 | NCAM16.2 | Mouse IgG2b, κ | 1:10 |

### Prime Flow™ RNA Assay

The PrimeFlow™ RNA Assay (Invitrogen) was performed according to the manufacturer's instructions to detect gene expression at the single cell level by FACS. An Adipoq-specific AlexaFluor 647 labeled probe was used for the hybridization (assay ID: VB1-17726-PF, type1; Invitrogen).

### Phagocytosis Confocal Imaging

BMDMs were cultured from C57BL/6 mice as described and 10⁵ cells were seeded into Chamber Slides (8-chamber, Nunc; ThermoScientific). The next day, splenocytes from C57BL/6 mice were ECP-treated, stained with 5 μM Cell Proliferation Dye eFluor670 (AxF647; eBioscience) and 10⁶ splenocytes were added onto BMDMs. Co-cultures were incubated for 24 h, washed with PBS, stained for CD11b surface expression (Table S13) and fixed with 4% PFA (Merck). The slides were mounted with ProLong Diamond mounting medium with DAPI (Invitrogen). Immunofluorescence images were taken at a LSM710 confocal microscope (Zeiss). For quantification, slides were scanned on a ScanR (Olympus) microscope and AxF488+AxF647+ cells were counted using the ScanR analysis software (Olympus). Table S13 is shown below.

TABLE S13
Antibodies for confocal imaging

| Antigen | Fluorochrome | Isotype | Clone | Dilution |
|---|---|---|---|---|
| CD11b | Alexa Fluor 488 | Rat IgG2b, κ | M1/70 | 1:100 |

### Western Blot

Cells were lysed with RIPA lysis buffer (Santa Cruz Biotechnology) supplemented with Phosphatase Inhibitor Cocktail 2 (Sigma). Protein concentrations were determined with Pierce BCA protein assay (ThermoScientific). Proteins were separated on SDS-PAGE gradient gels (Bis-Tris, 4-12%; ThermoScientific), then transferred onto Nitrocellulose membranes (Merck). Membranes were blotted for proteins with specific antibodies, and imaged using the INTAS ECL Chemocam Imager after incubation with a chemiluminescent substrate (Advansta). Western blots were quantified using LabImage 1D software. Antibodies and dilutions are summarized in Table S14 (shown below).

TABLE S14
Western blot antibodies

| Antigen | Isotype/Source | Clone | Dilution |
|---|---|---|---|
| Mouse Caspase 3 | Rabbit | Polyclonal | 1:1000 |
| Mouse Caspase 8 | Rabbit IgG | D35G2 | 1:1000 |
| Mouse Caspase 9 | Mouse IgG1 | C9 | 1:1000 |
| Mouse p53 | Rabbit IgG | D2H90 | 1:1000 |
| Mouse PARP | Rabbit | Polyclonal | 1:1000 |

### Viability and Caspase 3/7 Activity Assays

For analysis of cell viability and caspase 3/7 activity following ECP-treatment, WT C57BL/6 splenocytes were left either untreated, treated with only 8-MOP or UVA light, or the combination thereof (ECP). Viability was analyzed using the CellTiter-Glo 2.0 Cell Viability assay (Promega), where viability correlated with luminescence signal. Caspase activity was measured by increased luminescence signal using the Caspase-Glo 3/7 assay system (Promega).

### DSS Colitis Model

For the induction of colitis, C57BL/6 WT, Adipoq^{-/-} or Arg1-deficient chimera mice received 3% Dextran Sodium Sulfate (DSS colitis grade, 36-50 kDa; MP Biomedicals) in drinking water from day 0 to day 3, followed by normal drinking water. Mice were injected (i.p.) with 0.2 mg immune checkpoint blockade (ICB) or the respective isotype controls on day 0, day 3 and day 6 of the experiment. Recipients were transplanted (i.v.) with 10⁷ ECP treated donor splenocytes on day 3 and day 6. In some experiments, steroid, Adiponectin and AdipoRon were applied daily from day 3-7. Arginase-1 inhibitor was applied twice per day from day 4-7. Serum was harvested on day 8 by retroorbital bleeding under ketamine/xylazine anesthesia, followed by harvest of large intestine and spleen.

### Immunohistochemistry and Evaluation of Adiponectin Staining

For immunohistochemistry (IHC), 2 μm sections of FFPE-tissue were generated, de-paraffinized, rehydrated and underwent established heat-induced antigen retrieval (HIAR-steam cooker, 20 minutes, pH9). HIAR was followed by peroxidase blocking for 15 minutes using 1% H₂O₂. Blocking of sections was performed in 5% NGS in PBS for 30 minutes, followed by primary antibody incubation in blocking solution for 1 hour (primary antibody adiponectin, ThermoFisher, #710179, Lot:2052429, 1:400 dilution). HRP-linked secondary antibodies (Dako) were applied for 30 minutes in blocking solution. Visualization and staining was performed using the DAB+Substrate Chromogen System (Dako) including counterstaining with Haematoxylin. After dehydration slides were mounted using Entellan.

### Microscopy and Scoring of Adiponectin Staining

For analysis of immunohistochemistry (IHC) and histology, an inverted Zeiss Axio Imager microscope (Zeiss Imager.M1) equipped with an Axiocam 506 color camera and equipped with 10×, 20×, 40× objective was used. Quantifications of adiponectin positive cells within the lamina propria were performed on digitalized images (randomly captured at 40× magnification). Positive and negative cells in hotspot regions were assigned using Fiji ImageJ v1.52. Adiponectin positive cells were expressed as either densities (in patient samples) or positive cells per hotspot region (in murine samples).

### MC38^{OVA} Subcutaneous Tumor Model

WT C57BL/6 recipients were transplanted subcutaneously with 1×10⁶ MC38^{OVA} or 0.5×10⁶ MC38 (control only) cells into the shorn right flank. Recipients were treated with 0.2 mg ICB on day 1, 4, 8, 11, 15. ECP treated splenocytes were transplanted (10⁷; i.v.) on day 6, 11 and 15, daily steroid was given (i.p.) from day 6-15. On day 16, serum samples were harvested by retroorbital bleeding, the mice were euthanized and tumors and spleen were resected for further studies.

B16.F10^{OVA} Subcutaneous Model with Adoptive T Cell Transfer

CD45.1 recipients were transplanted subcutaneously with 1×10⁶ B16.F10^{OVA} cells into the shorn right flank. Recipients were treated with 0.2 mg ICB on day 1, 4, 8, 11, 15. ECP treated splenocytes were transplanted (10⁷; i.v.) on day 6, 11 and 15, daily steroid was given (i.p.) from day 6-15. OT-I splenocytes (CD45.2) were activated on day 2 at a density of 2×10⁶ cells/mL in the presence of 100 U/mL rmIL-2 (Peprotech) and 0.1 μg/mL OVA257-264 peptide (Sigma) in RPMI supplemented with 10% FCS, 1% P/S, 55 μM beta-Mercaptoethanol (Gibco) and 4 mM glutamine (Gibco). Splenocytes were washed on day 4 and replated with 100 U/mL rmIL-2 at 2×10⁶ cells/mL. On day 5, OT-I splenocytes were washed and 5×10⁶ cells were injected intravenously into the tail vein of tumor bearing mice. On day 16, serum samples were harvested by retroorbital bleeding, the mice were euthanized and tumors and spleen were resected for further studies.

### ALT and AST Activity Assays

The ALT and AST activity assays on serum harvested from mice with xenograft-induced irAE were performed according to manufacturer's instructions (Sigma).

### Results

### ECP Reduced Immune Related Colitis Via Adiponectin Expression

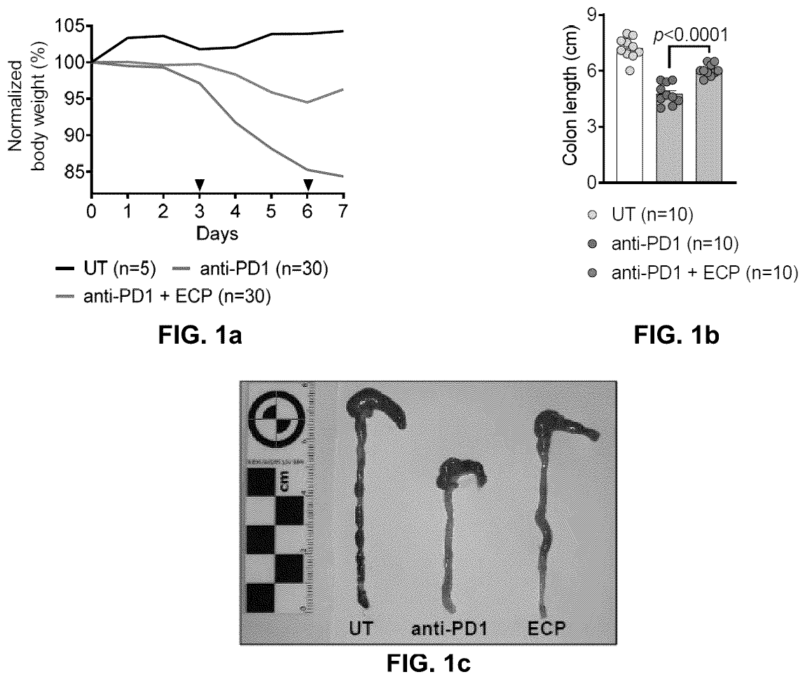

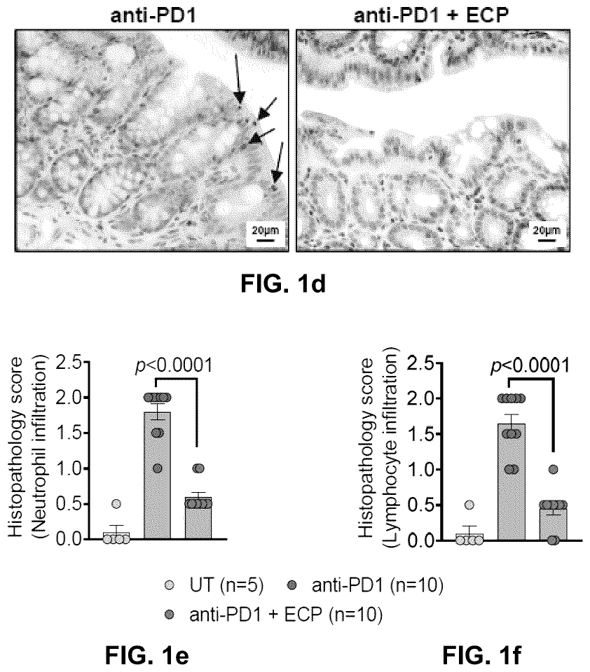

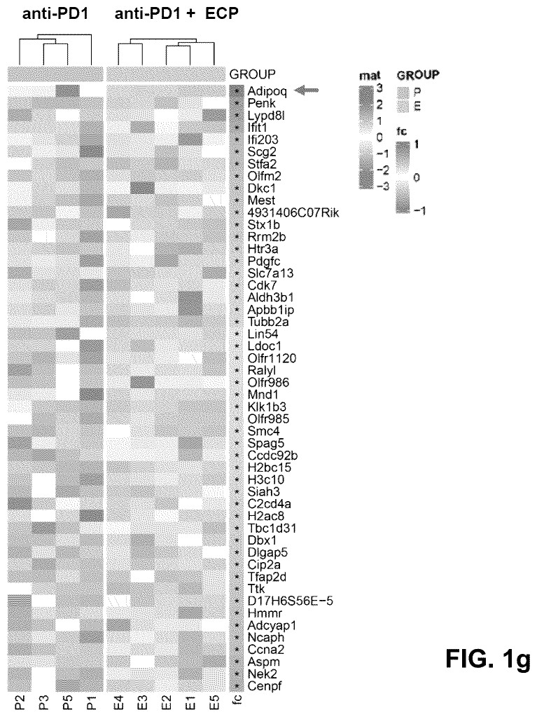

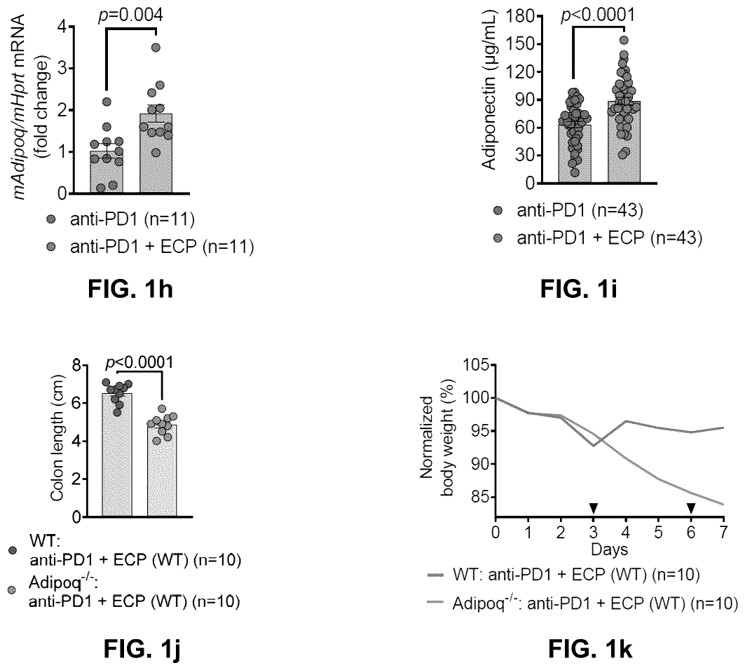

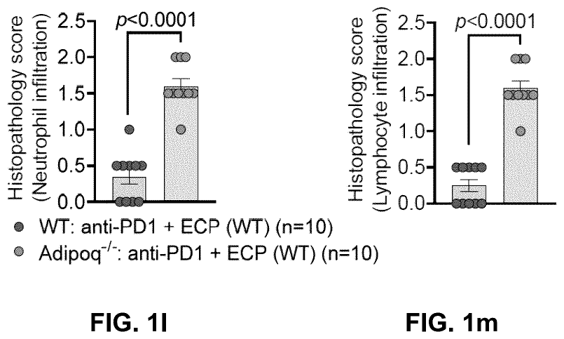

To test ECP in a controlled in vivo irAE-colitis model, mice were treated with 3% DSS and anti-PD1 to induce colitis (FIGS. Sla). Anti-PD1 treatment increased colitis severity compared to the isotype (FIGS. S1b and S1c) indicating ICI-induced colitis. DSS and anti-PD1 treatment reduced the body weight of the mice consistent with the development of colitis (FIG. 1a). Transfer of ECP-treated donor splenocytes reduced weight loss (FIG. 1a) and prevented inflammation-induced colon shortening compared to the group treated with anti-PD1 alone (FIGS. 1b and 1c). Colon length was reported as a surrogate parameter for the severity of ICI-induced colitis and colon length and bodyweight were found to correlate (FIG. S1d). In agreement with reduced colitis severity, reduced infiltration of neutrophil granulocytes (neutrophils) and lymphocytes in the colon wall of mice treated with ECP compared to the group treated with anti-PD1 was observed (FIGS. 1d-f). Transfer of untreated splenocytes was not protective (FIGS. S1e-S1h) and improvement of colitis by ECP was comparable to steroid treatment (FIGS. S1i-S1l). Comparable to splenocytes the use of PBMCs for the ECP-procedure induced protection from colitis (FIGS. S1m-S1p), indicating the comparability to the application of ECP in patients, where PBMCs are used. Transplantation of ECP-treated splenocytes showed similar results with DSS/anti-CTLA4-induced colitis (FIGS. S2a-S2d), which is frequently applied in combination with anti-PD1 as a treatment in metastatic melanoma.

Transcriptional changes of cells residing in the intestinal wall were investigated and it was found that Adipoq (adiponectin) to be the most significantly upregulated gene in colitis mice treated with anti-PD1/ECP compared to anti-PD1 alone (FIGS. 1g and 1h). Furthermore, adiponectin serum concentrations were increased upon ECP-treatment in DSS/anti-PD1-colitis mice (FIG. 1i). The same pattern was seen in the colitis model using anti-CTLA4 (FIG. S2e). Adipoq^{-/-} recipients were not protected by ECP (FIGS. 1j-m), which is comparable to the situation when ECP-donor and recipients were Adipoq^{-/-}(FIGS. S3a-S3d). Adiponectin-deficiency in ECP-treated splenocytes was not relevant for immunosuppressive ECP-effects (FIGS. S3e-S3h). These findings indicated that ECP reduces ICI-induced colitis in mice by causing production of adiponectin in recipient cells.

Adiponectin was Induced by Apoptotic Leukocytes that Accumulate in Inflamed Colon Tissue

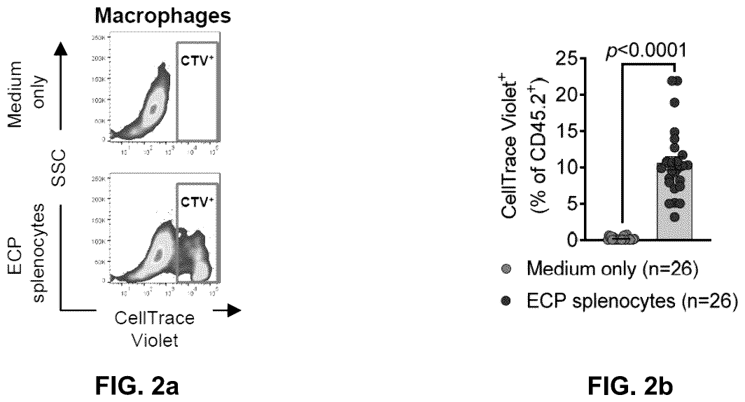

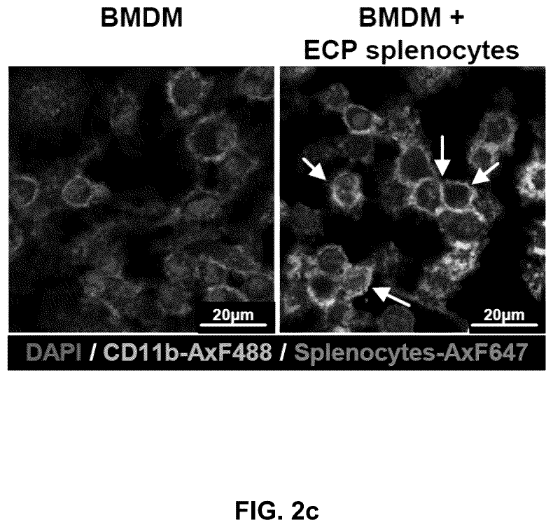

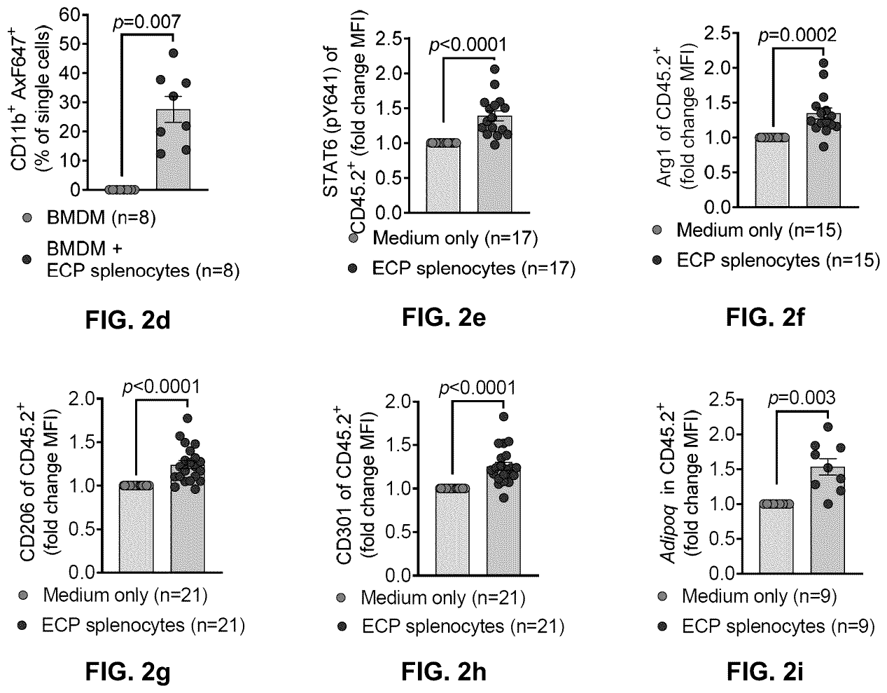

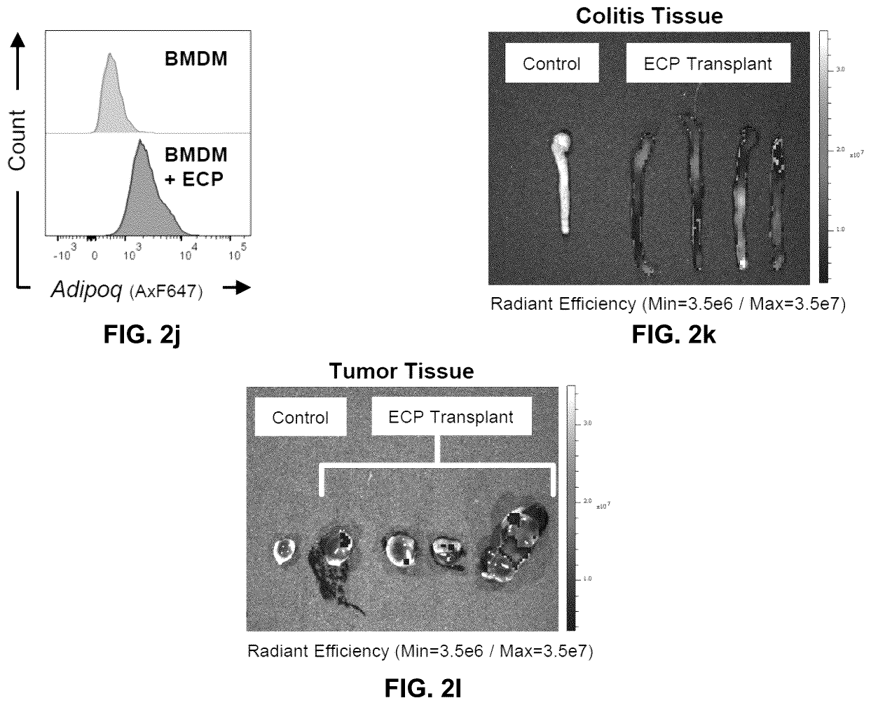

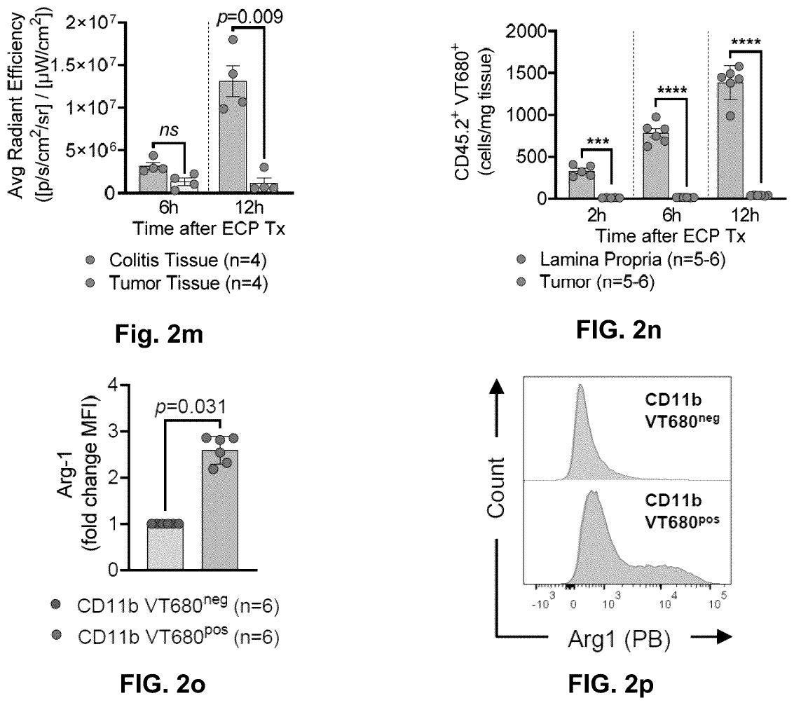

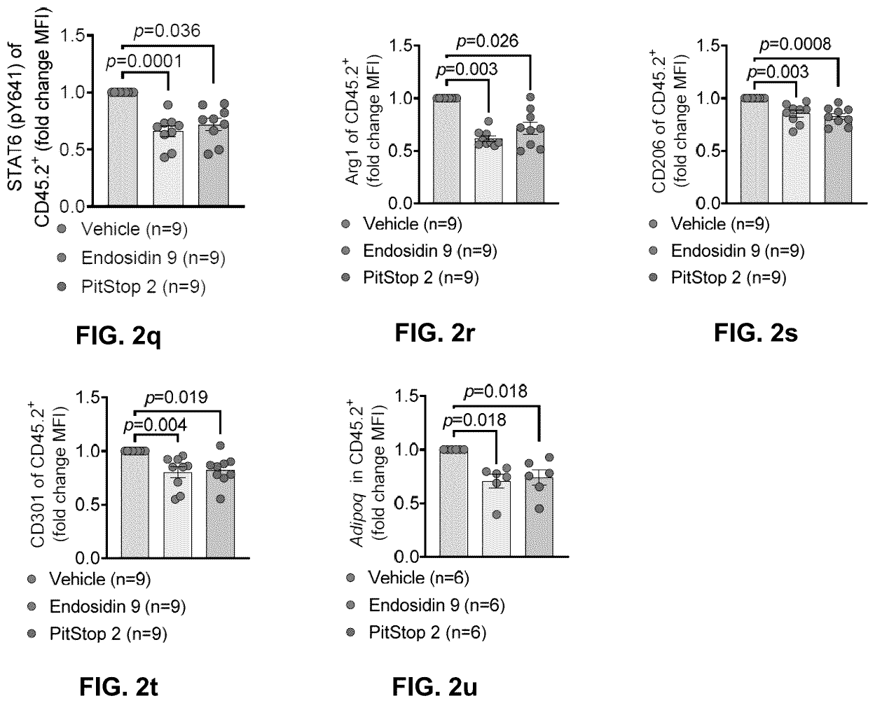

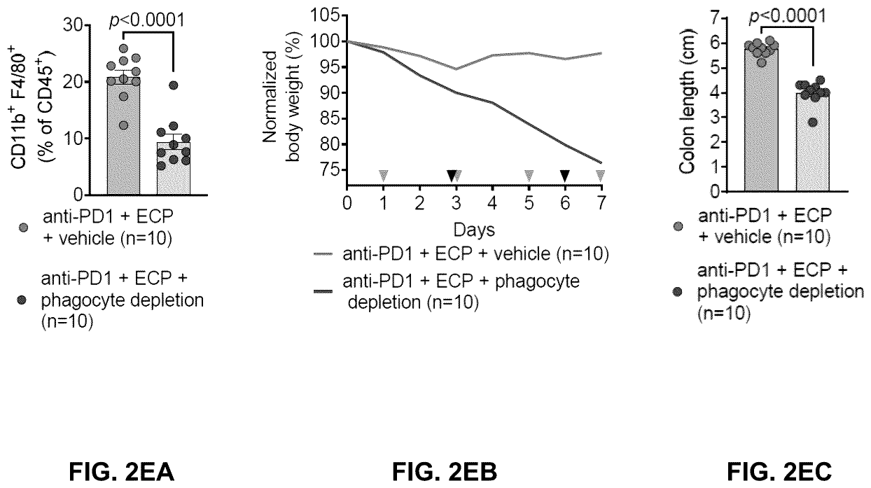

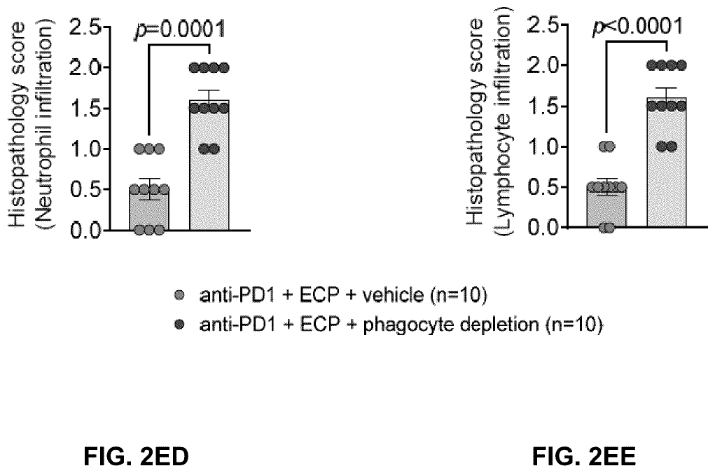

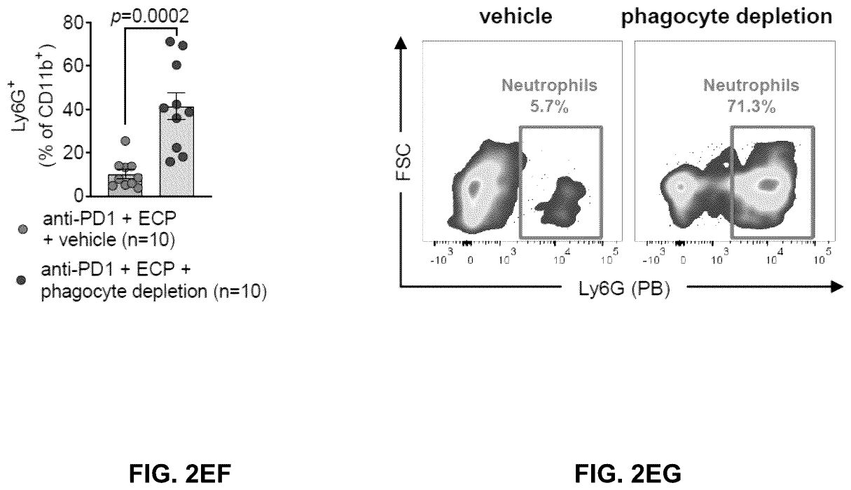

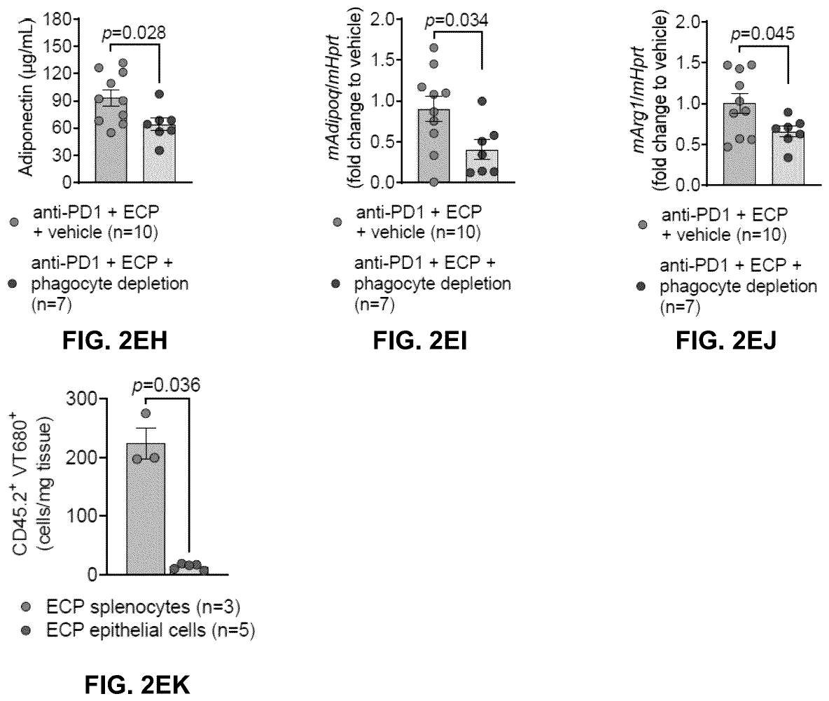

ECP-treated leukocytes underwent rapid apoptosis in vitro, and only the combination of ultraviolet A light (UVA) and 8-Methoxypsoralen (8-MOP), which is ECP, induced cell death (FIGS. S4a and S4b). Apoptosis was indicated by p53-stabilization with enhanced caspase activity and cleavage of caspases-3/8/9 and PARP (FIGS. S4c-S4n). Findings were verified in vivo by transfer of luciferase-transgenic splenocytes from melanoma-bearing donors into Rag2^{-/-}γc^{-/-} mice, whereas only viable cells convert luciferin and emit light. BLI-signal was decreased upon transfer of ECP-treated splenocytes (FIGS. S5a and S5b). To understand how ECP mediates adiponectin induction, ECP-treated leukocytes (CD45.1⁺) were exposed to bone marrow-derived macrophages (BMDM, CD45.2⁺), which have been shown to regulate intestinal inflammation. ECP-treated leukocytes were internalized by BMDMs (FIGS. 2a-d) consistent with high Calreticulin surface levels acting as an engulfment signal and stimulating clearance of dying cells by phagocytes (FIGS. S6a and S6b). Results were reproducible when using bone marrow-derived dendritic cells (BMDC) as phagocytes (FIGS. S6c and S6d). Phagocytosis of cells was confirmed when using SIINFEKL peptide-loaded leukocytes co-cultured with BMDMs where SIINFEKL was found on CellTrace Violet⁺ (CTV⁺) BMDMs after co-culture (FIG. S6e). Mechanistically, phagocytosis of ECP-treated splenocytes coincided with STAT6-activation in macrophages (FIG. 2e) which is known to drive their polarization towards an immunosuppressive phenotype (M2-like) and could explain the protective effect of ECP in the intestine. In agreement with these results, co-culture with ECP-treated splenocytes induced Arg1, CD206 and CD301 in macrophages in vitro (FIGS. 2f-h) indicative of an anti-inflammatory phenotype. Moreover, Adipoq gene expression increased in BMDMs upon co-culture with ECP-treated splenocytes (FIGS. 2i and 2j).

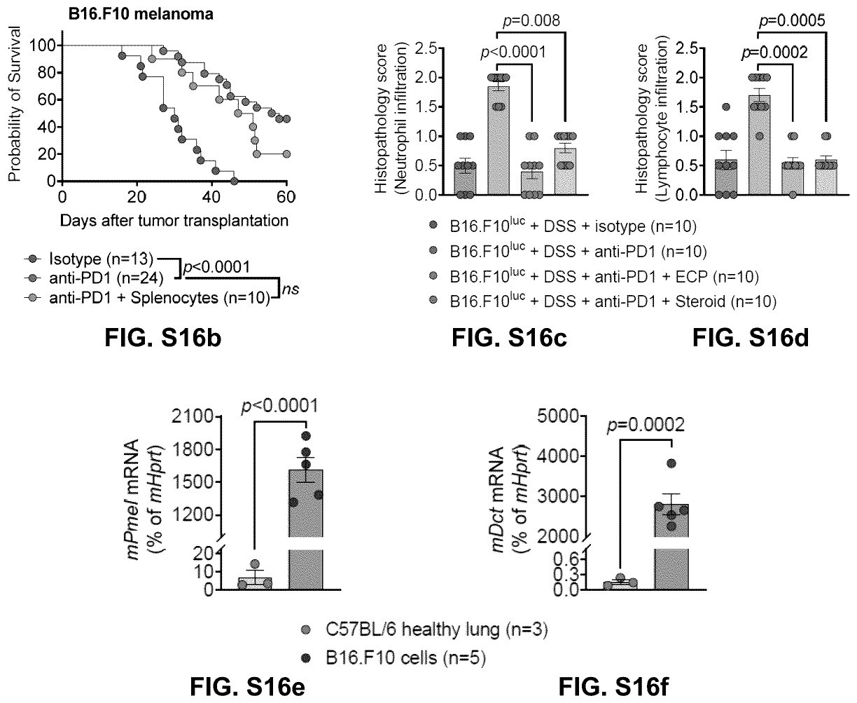

To understand if ECP-treated leukocytes reached the intestinal tract, VivoTrack680-stained (VT680) CD45.1 ECP-treated donor cells transplanted into CD45.2⁺ recipients were used. High fluorescence signal was seen in colitis 12 h post-transplantation, but not in B16.F10-melanoma tissue (FIGS. 2k-2m). VT680⁺CD45.2⁺ recipient cells were detectable primarily in the colon of colitis/melanoma mice after transfer of ECP-treated CD45.1⁺VT680⁺ leukocytes (FIG. 2n) indicating phagocytes with engulfed VT680⁺-labelled donor cells in the inflamed intestine. The VT680 recipient myeloid cells showed high arginase-1 expression 12 h after ECP transplantation indicating their M2-like polarization after uptake of ECP-treated leukocytes (FIGS. 20 and 2p). Blocking the uptake of ECP-treated leukocytes in vitro (FIG. S6f) reduced STAT6 phosphorylation, M2-like polarization and adiponectin induction in BMDMs (FIGS. 2q-2u). Depleting colonic phagocytic cells in DSS/anti-PD1-induced colitis in vivo using clodronate-loaded liposomes (FIGS. 2EA, S6g, and S6h) abrogated the protective effects of the ECP (FIGS. 2EB-2EE), increased infiltration of inflammatory neutrophils (FIGS. 2EF and 2EG), and diminished protective adiponectin production and Arg1 expression (FIGS. 2EH-2EJ).

To test whether protection against ICI-colitis was dependent on the capacity of the pre-apoptotic ECP-exposed cells to migrate to inflamed tissue, myeloid cells (CD45⁺CD11b⁺), T-cells (CD45⁺CD3⁺), and epithelial cells were compared. Epithelial cells served as a control as they lack most of the molecules which are important for transendothelial migration (FIG. S7a). Epithelial cells also underwent apoptosis and upregulated surface calreticulin after ECP-treatment (FIGS. S7b and S7c). While ECP-exposed immune cells were protective, ECP-treated epithelial cells were not (FIGS. S8a-S8l) indicating that the capacity to migrate into the inflamed colon is required for anti-inflammatory ECP-effects (FIG. 2EK).

These findings indicate that ECP-induced apoptotic leukocytes accumulate in the colitis tissue where they are phagocytosed by intestinal macrophages. This causes local anti-inflammatory polarization and adiponectin production. Conversely, phagocytosis of ECP-treated CD45.1⁺VT680⁺ leukocytes was not detected in the tumor microenvironment.

### ECP Enhanced STAT6 and PPARγ Activity in Colonic Macrophages

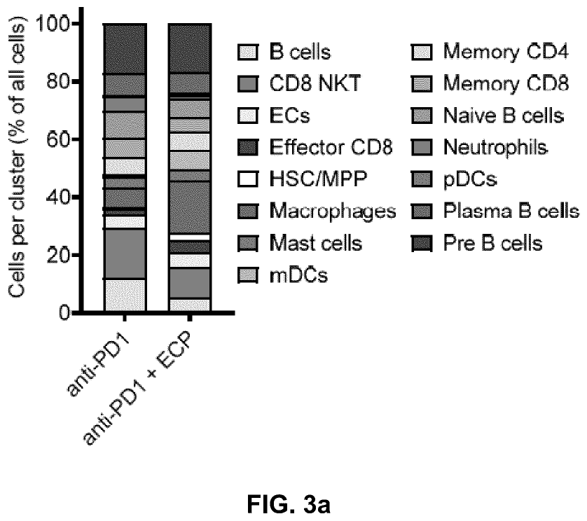

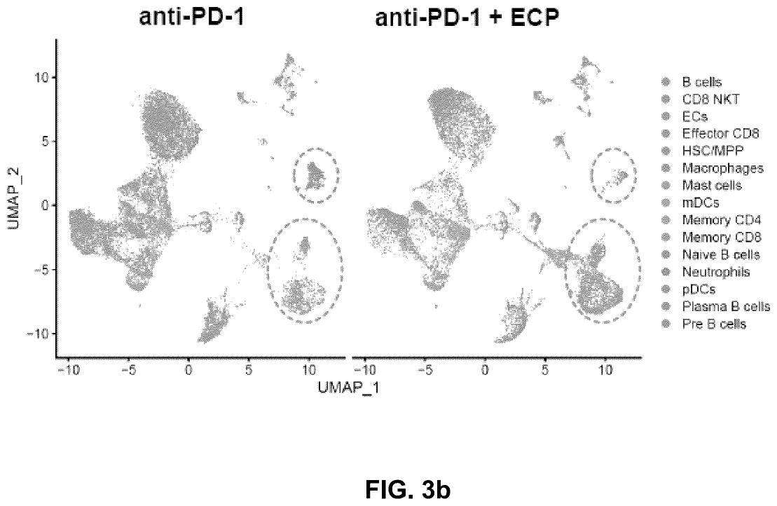

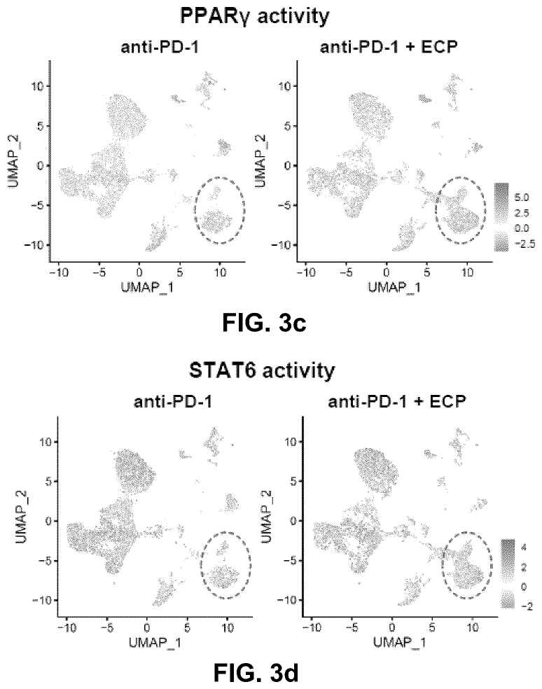

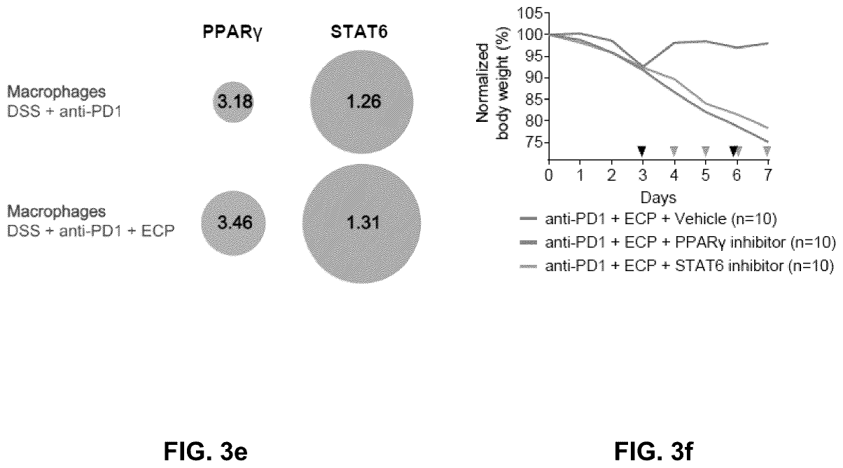

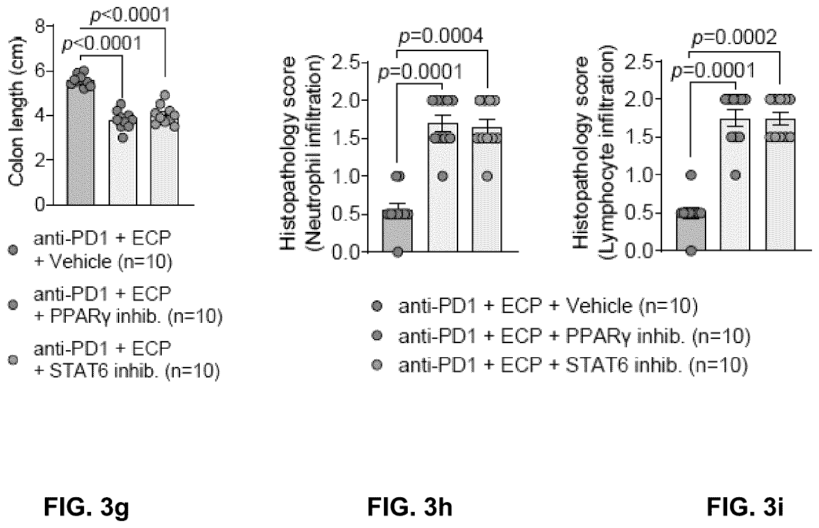

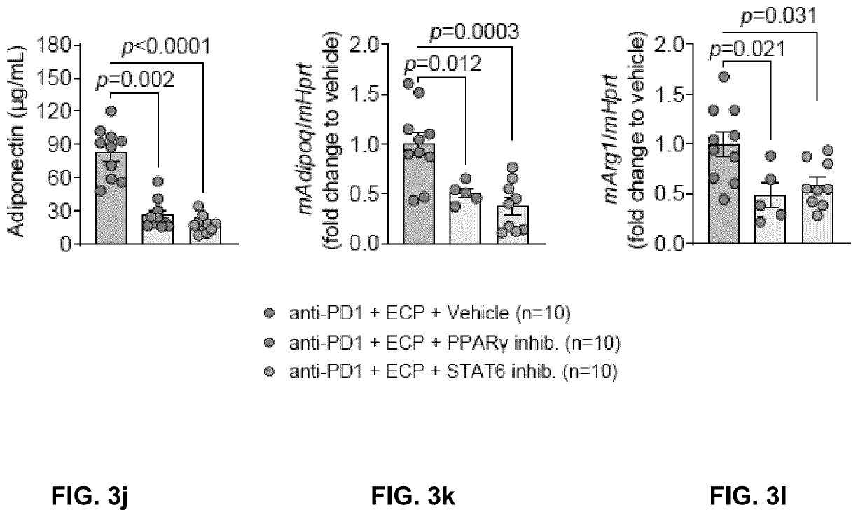

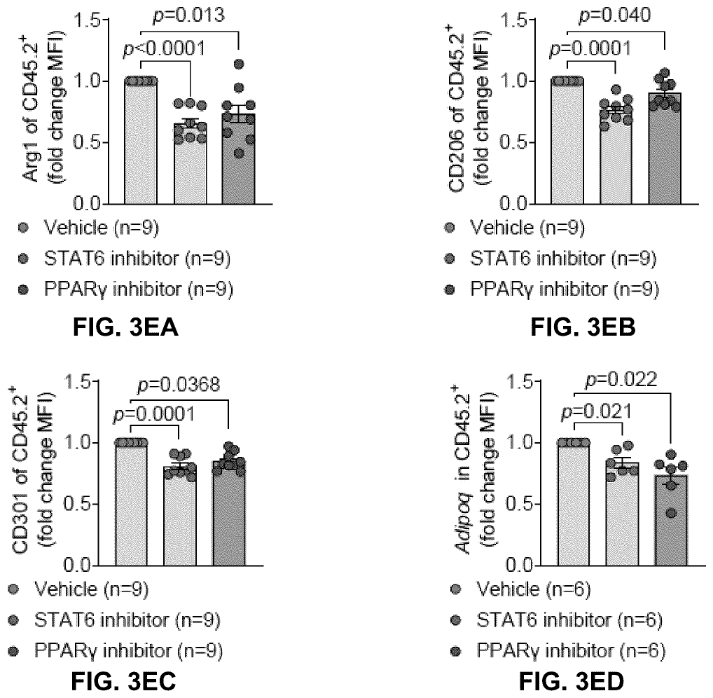

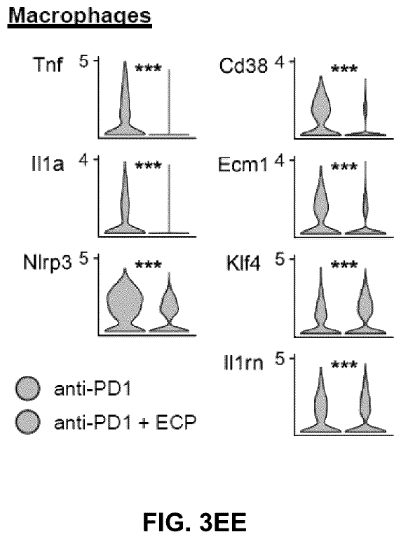

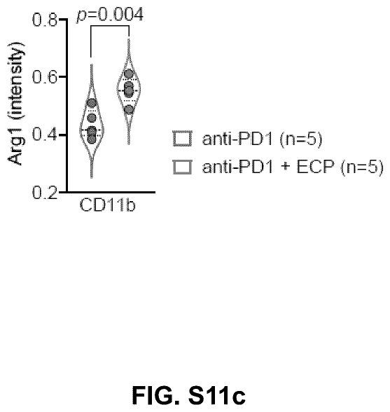

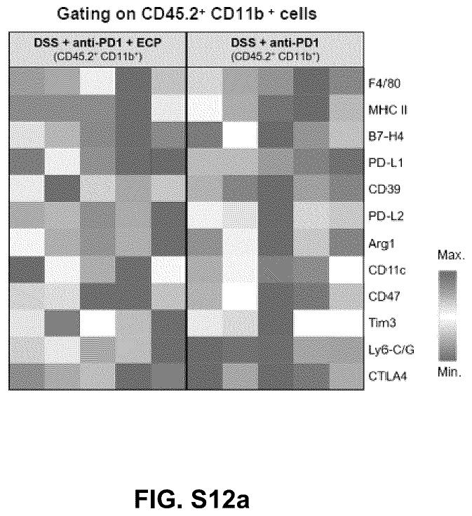

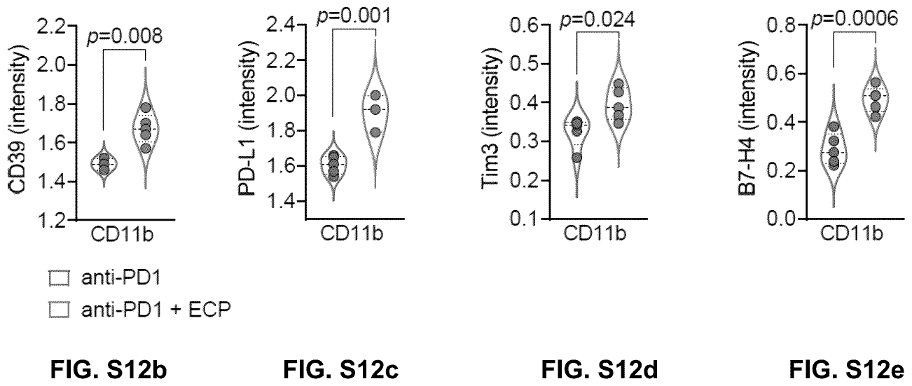

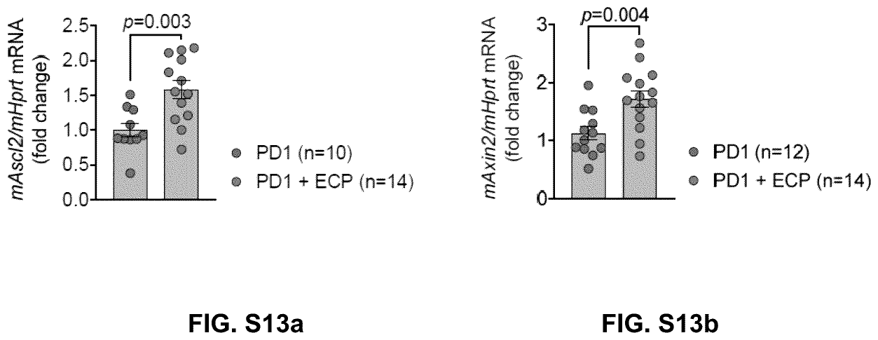

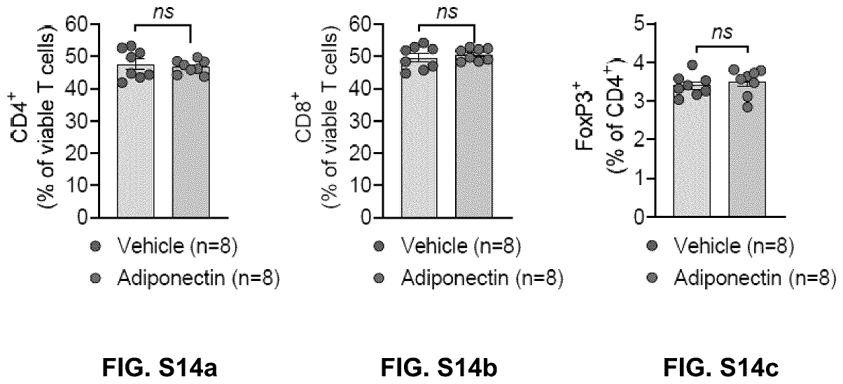

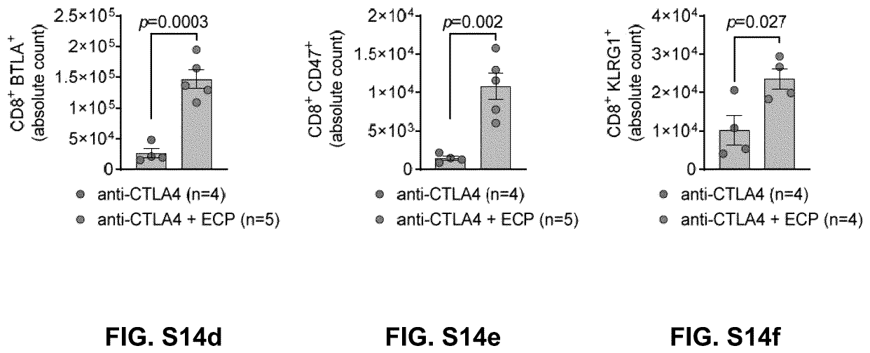

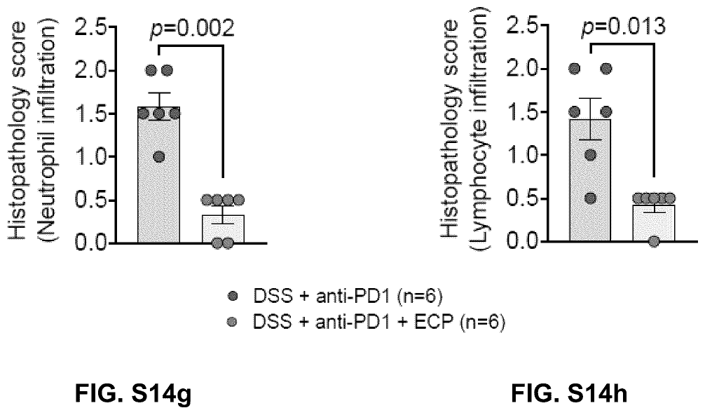

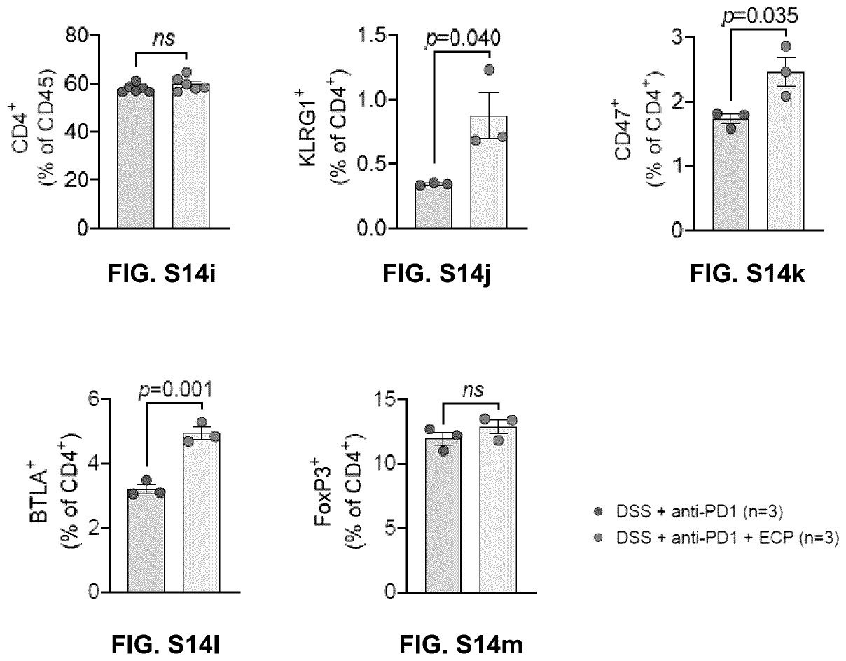

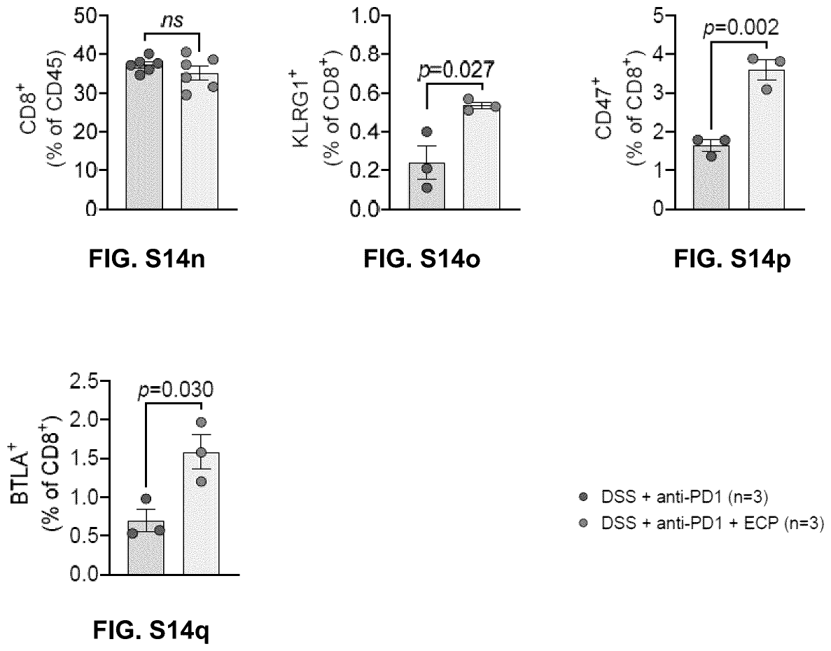

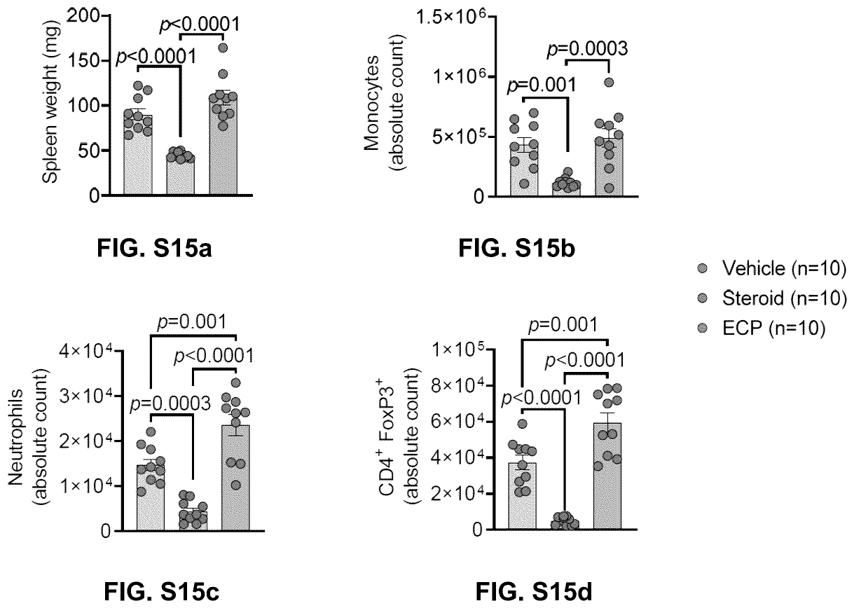

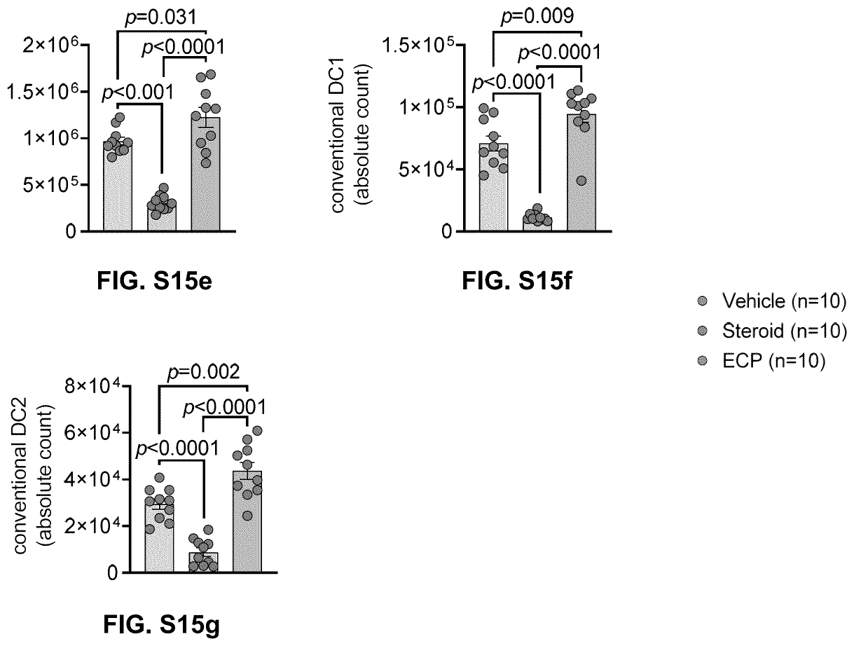

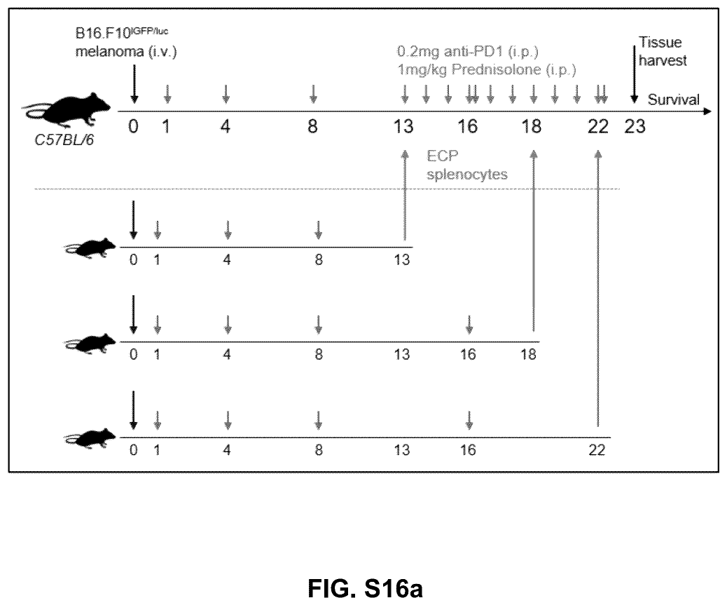

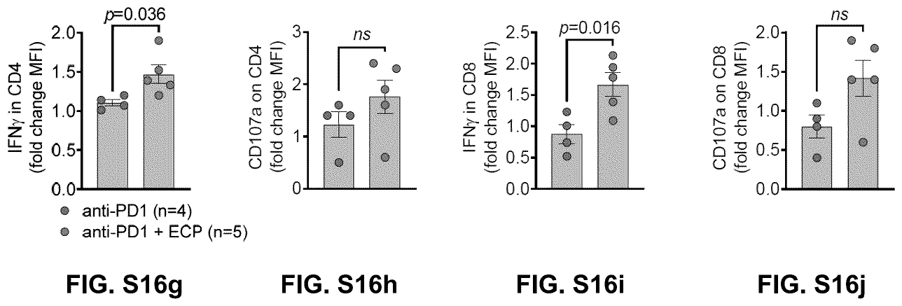

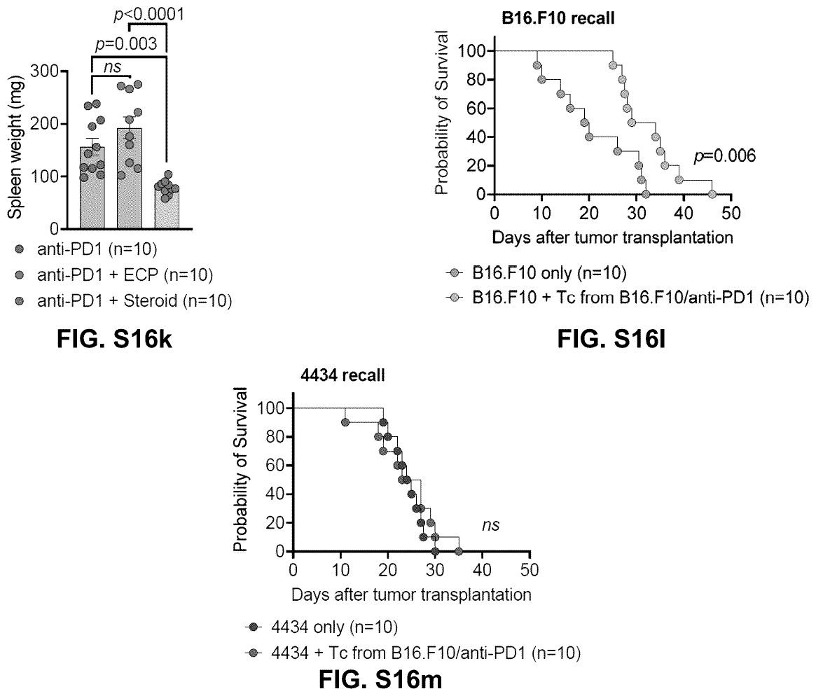

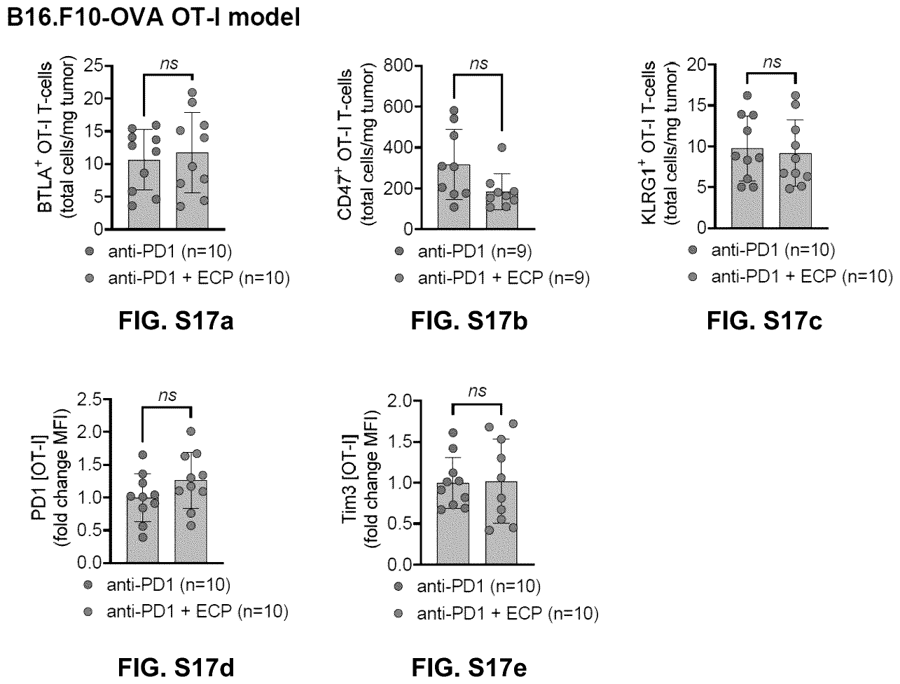

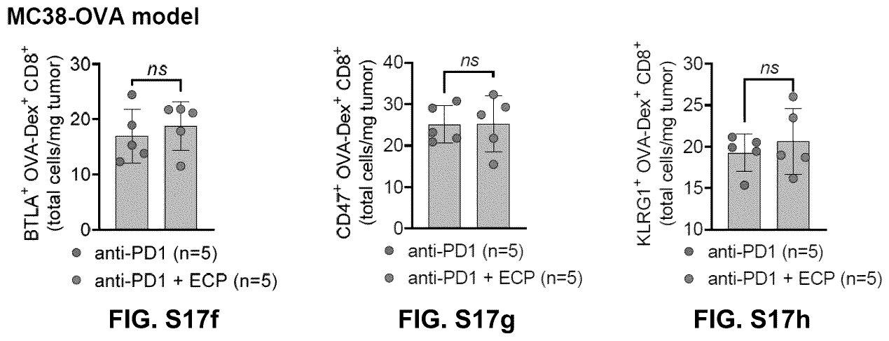

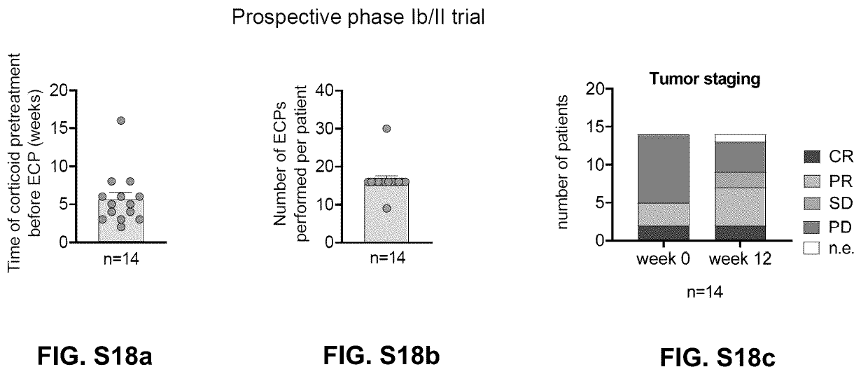

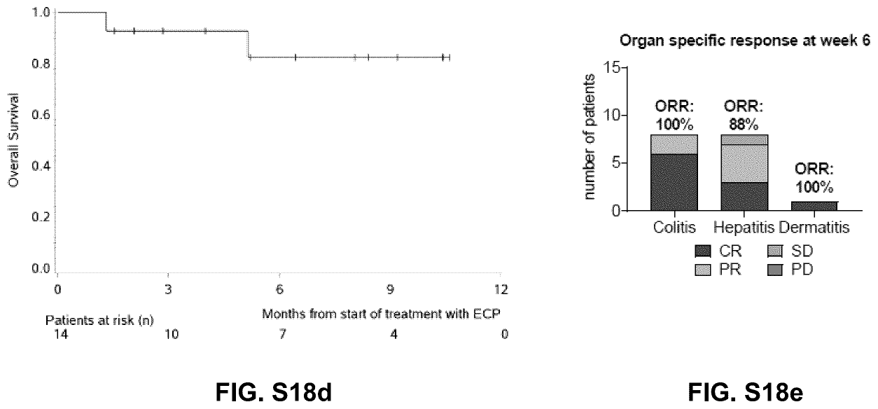

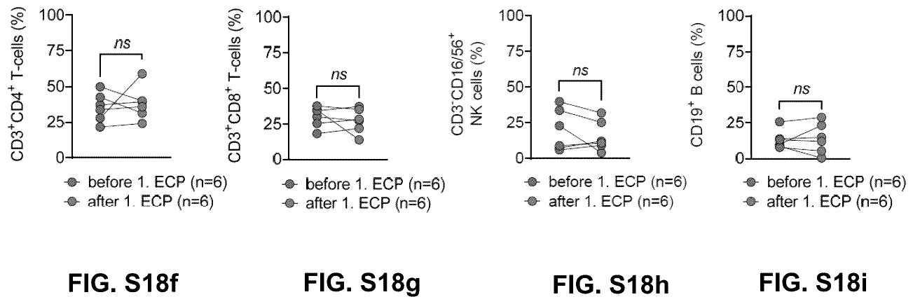

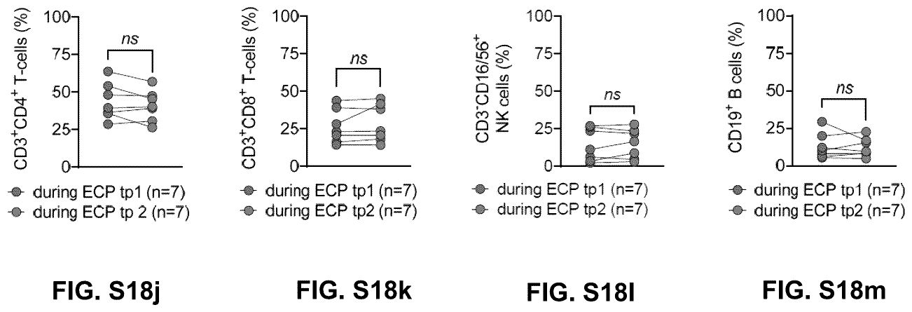

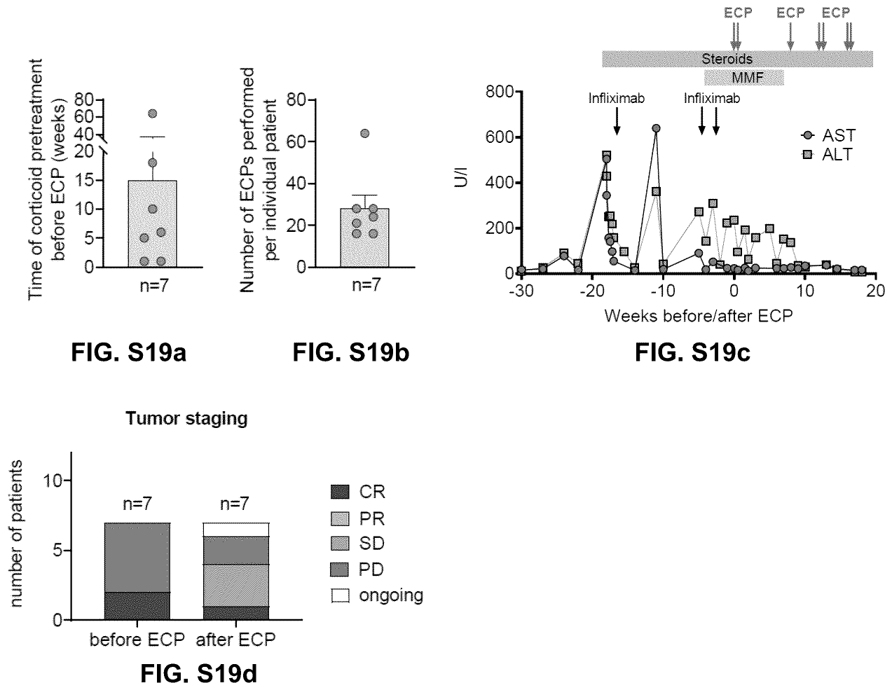

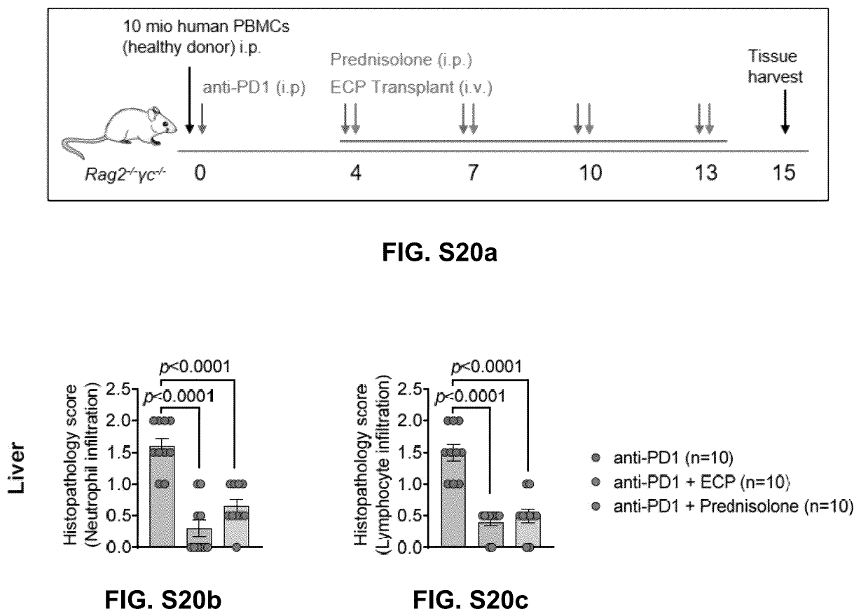

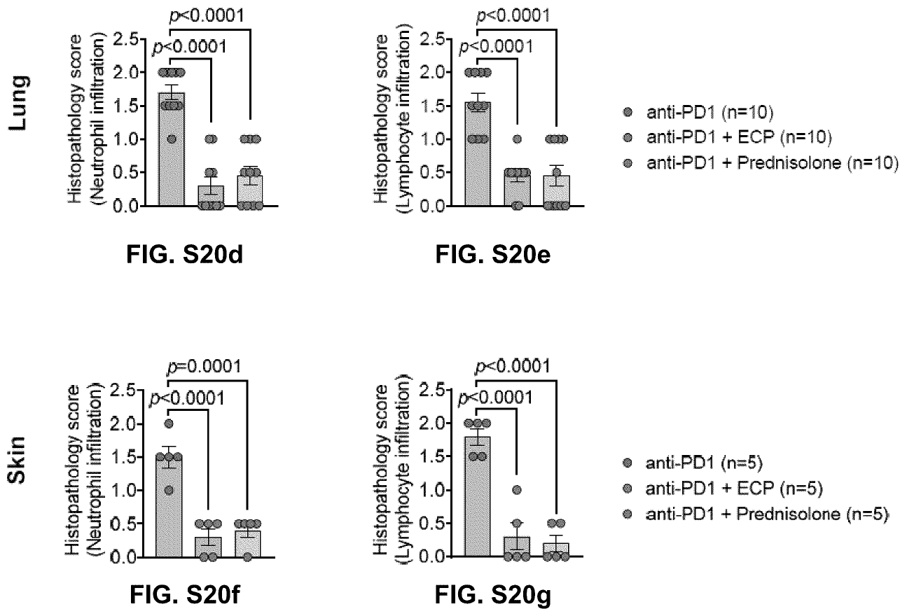

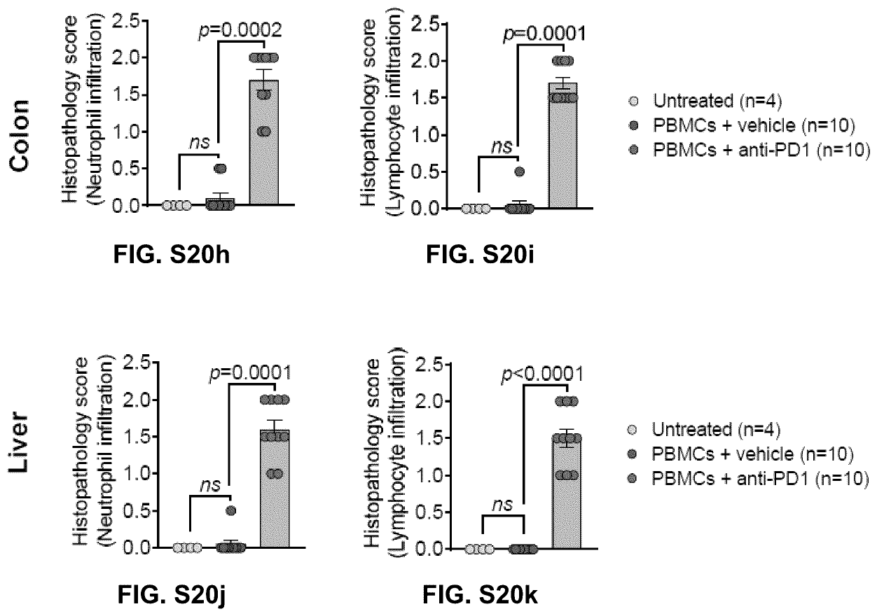

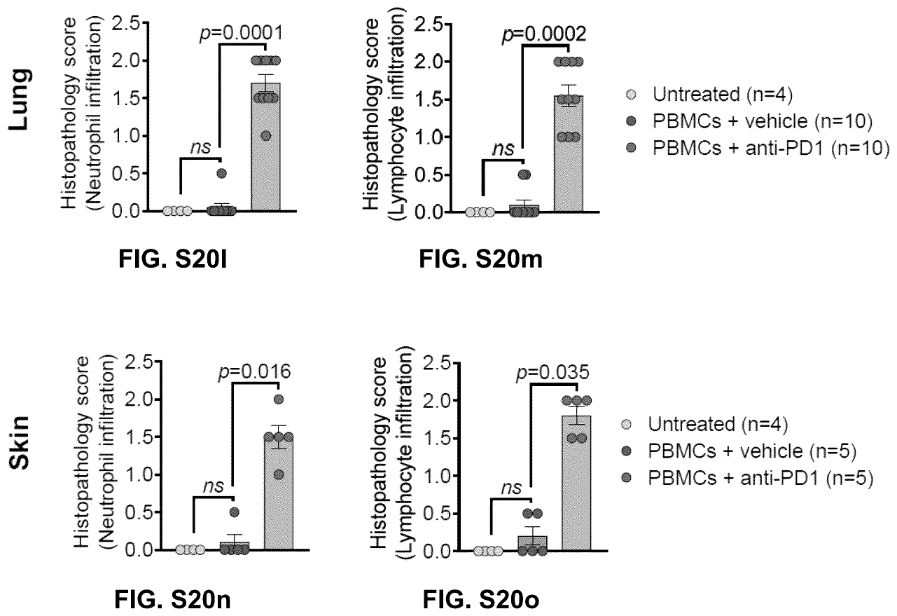

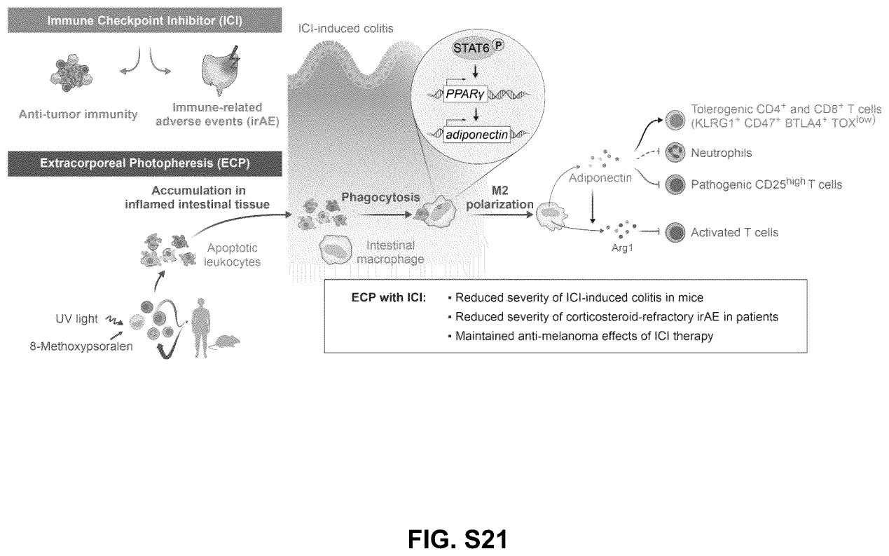

Using scRNA-seq, intestinal immune cells from anti-PD1 and anti-PD1/ECP-treated colitis mice were profiled, identifying major changes in colonic myeloid cells upon ECP-treatment (FIGS. 3a and 3b). Macrophages were enriched whereas neutrophils were decreased in ECP-treated mice (FIGS. 3a and 3b) which is in agreement with the known contribution of neutrophils to colitis. Analyzing transcription factor activities, an increase in cells with higher STAT6- and PPARγ-activity in colonic macrophages after ECP-treatment was observed (FIGS. 3c-3e), both of which are important in polarizing macrophages towards an anti-inflammatory phenotype. In addition, KLF4 and PPARα, which both promote an anti-inflammatory state, showed increased activity in macrophages after ECP-treatment (FIG. S9a). Supporting the importance of PPARγ and STAT6 for the immunosuppressive effects of ECP, inhibiting either in colitis mice abrogated protective ECP-effects (FIGS. 3f-3i), as well as the beneficial increase of adiponectin and Arg1 (FIGS. 3j-31). Consistent with these results, reduced anti-inflammatory polarization and lower Adipoq expression was seen in BMDMs co-cultured with ECP-treated splenocytes upon PPARγ and STAT6 inhibition (FIGS. 3EA-3ED). Further scRNA-seq analysis confirmed a transition from pro-inflammatory towards M2-like macrophages (FIGS. 3EA-3EE, Table S1), whereas neutrophils expressed lower levels of tissue-damaging matrix-metalloproteinases in ECP-treated mice (FIG. S9b, Table S1). Table S1 is shown below.

TABLE S1
Selected genes from scRNA Seq analysis clusters

| | | | Average log2FC |
|---|---|---|---|
| Cluster | Gene | p-value | (ECP vs. PD1) |
| Macrophages | Tnf | 1.1795076E−45 | −1.7686296 |
| Macrophages | Il1a | 3.5416712E−45 | −1.0363769 |
| Macrophages | Nlrp3 | 1.3725282E−31 | −0.8513514 |
| Macrophages | Cd38 | 1.8800933E−57 | −0.8628562 |
| Macrophages | Ecm1 | 1.0058807E−31 | −0.8013273 |
| Macrophages | Klf4 | 4.0671276E−18 | 0.6183515 |
| Macrophages | Il1rn | 0.0000527 | 0.1927991 |
| Effector CD8 | Ccl5 | 4.7100595E−20 | −2.1335190 |
| Effector CD8 | Nkg7 | 6.0202030E−25 | −0.9356569 |
| Effector CD8 | Cd69 | 0.0087711 | −0.2935040 |
| Effector CD8 | Klrd1 | 3.2300536E−09 | −0.7160694 |
| Effector CD8 | Xcl1 | 7.7039557E−06 | −0.5977766 |
| Effector CD8 | Cd3d | 3.8608101E−28 | −0.8851795 |
| Effector CD8 | Cd3e | 3.6186499E−20 | −0.5115299 |
| Effector CD8 | Cd3g | 8.5443695E−24 | −0.7762495 |
| Effector CD8 | Trbc1 | 2.3385067E−07 | −0.6708627 |
| Effector CD8 | Trbc2 | 1.5832261E−25 | −0.8181384 |
| Effector CD8 | Trdc | 8.8529013E−11 | −0.9759705 |
| Effector CD8 | Cd8b1 | 2.8437599E−07 | −0.3965417 |
| Effector CD8 | Lck | 4.1864085E−10 | −0.4579562 |
| Effector CD8 | Zap70 | 2.7644297E−12 | −0.4277690 |
| Effector CD8 | Runx2 | 1.1366398E−10 | −0.4546475 |
| Neutrophils | Mmp8 | 1.8265359E−68 | −3.0645733 |
| Neutrophils | Mmp9 | 1.3991089E−14 | −0.5670902 |
| Neutrophils | Mmp25 | 8.5590298E−12 | −0.8144944 |

scRNA-seq based analysis and functional inhibitor-based studies indicate that ECP induces a regulatory PPARγ- and STAT6-dependent program in macrophages leading to an alternatively activated macrophage phenotype and adiponectin production.

### Adiponectin Reduced Intestinal Inflammation

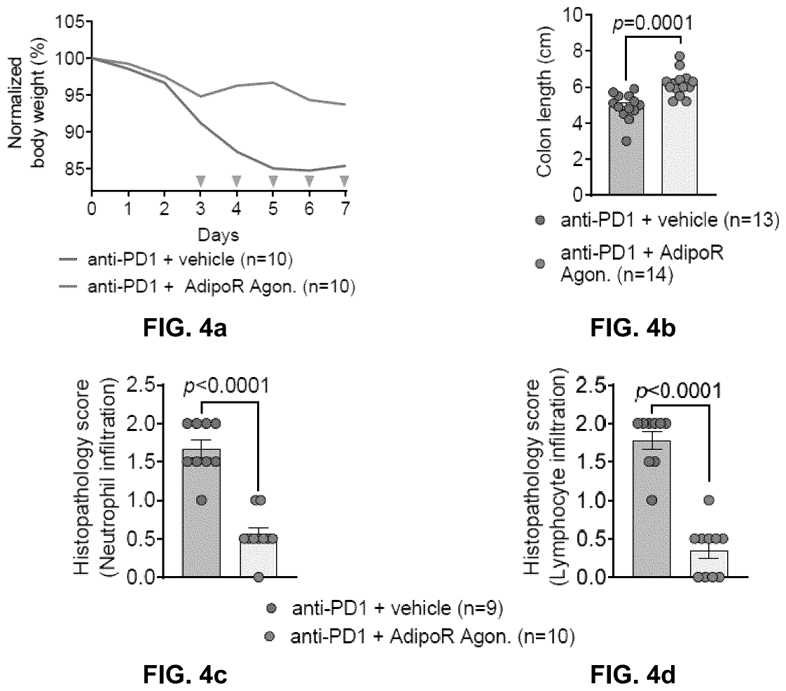

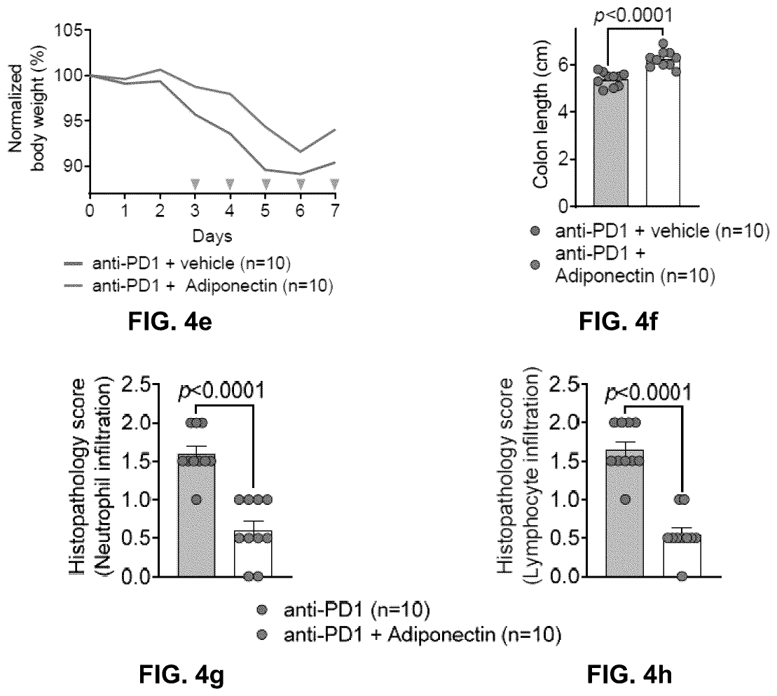

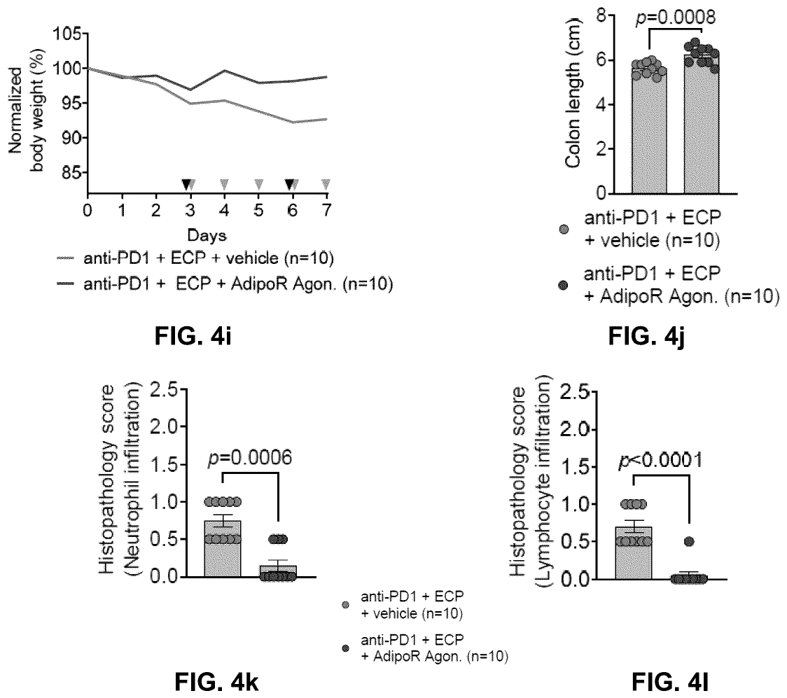

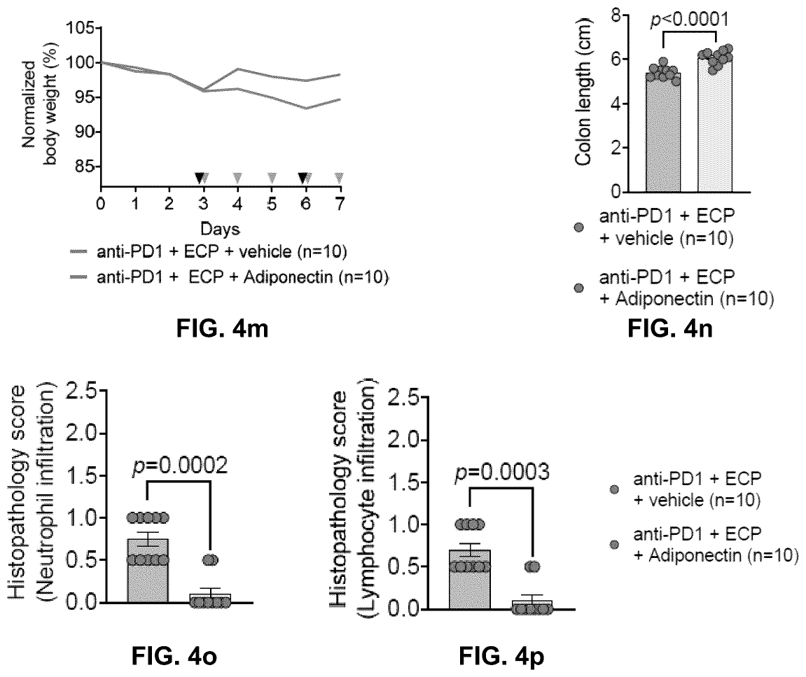

The importance of adiponectin in resolving ICI-therapy-induced colitis was confirmed by application of an adiponectin receptor agonist (AdipoRon, ARA) or recombinant adiponectin in mice with DSS/anti-PD1-induced colitis (FIGS. 4a-h). Both treatments synergized with ECP to reduce ICI-induced colonic inflammation (FIGS. 4i-p). ARA-treatment was not only effective in anti-PD1-mediated colitis, but also in DSS/anti-CTLA4-induced intestinal inflammation (FIGS. S10a-S10d). This data showed that adiponectin signaling can reduce the severity of colitis induced by ICI-therapy and that it acts synergistically with ECP.

### Adiponectin and ECP Elevated Arg1 in Myeloid Cells

The profiling of various immune cells isolated from naïve mice revealed particularly high Adipoq expression in monocytes and M2-polarized macrophages (FIG. 5a), whereas the Adipor1 and Adipor2 receptors were expressed by multiple cells with particularly high expression seen in neutrophils (FIGS. 5b and 5c). The influence of adiponectin on BMDMs in vitro was evaluated and it was found that an upregulation of immunosuppressive Arg1 upon exposure to adiponectin or ARA (FIGS. 5d and 5e), whereas the expression of Adipoq and Arg1 was highly correlative in BMDMs (FIG. 5f).

Using CyTOF-based analysis of immune cells isolated from colitis mice, it was confirmed that major changes in the recipient's myeloid cells derived from the lamina propria and spleen upon ECP-treatment (FIGS. 5g, 5h, S11a, and S11b). Arginase-1 was among the most prominently upregulated markers in ECP-treated colitis mice (FIGS. 5i and S11c). Consistently, Arg1 expression was upregulated in colitis tissue upon ECP-therapy (FIG. 5j). In addition, the immunosuppressive molecules CD39, PD-L1, Tim3, and B7-H4 were increased in the splenic myeloid subsets of colitis mice after ECP-treatment (FIGS. S12a-S12e). Higher expression of intestinal stem cell-related genes Ascl2 and Axin2 indicate increased intestinal repair (FIGS. S13a and S13b). The connection between adiponectin and arginase-1 was further studied and it was found that there was reduced Arg1 expression in M2-polarized Adipoq^{-/-}-BMDMs compared to WT (FIGS. 5k-5m). This is consistent with the lower levels of M2-like markers CD206 and CD301 (FIGS. 5n and 5o).

These findings indicated a direct link between adiponectin and higher Arg1 expression and anti-inflammatory polarization in mice with ICI-induced colitis.

### Arg1 Blockade Abrogated Protective ECP Effects

Arg1 and Adipoq expression in the colon of mice with DSS-induced colitis was compared and a positive correlation when mice received anti-PD1/ECP-treatment, but not with anti-PD1 alone, was found (FIGS. 6a and 6b). In agreement with Arg1 playing a functional role after ECP, Arg1-inhibition reduced protective ECP-effects in DSS/anti-PD1-induced colitis (FIGS. 6c-6f). Using Arg1^{-/-} mice, its functional role in reducing ICI-induced colitis only in ECP-recipients was confirmed, but not in the donor compartment (FIGS. 6g-6m). These findings indicate that Arg1 is required in the recipient tissues to allow for the full protective ECP-effect, while it is not required in leukocytes undergoing ECP.

### Adiponectin and ECP Reduce T-Cell Activation

The impact of adiponectin and ECP on T-cells was studied by activating them in vitro with anti-CD3/CD28-beads in the presence of adiponectin. This reduced the expression of CD25, the most prominent T-cell activation marker, on CD4⁺ and CD8⁺ T-cells (FIGS. 7a-7c) but no effect on FoxP3 expression and CD4⁺/CD8⁺ T-cell frequencies was observed (FIGS. S14a-S14c). The functional role of CD25-reduction upon adiponectin-treatment was proven by adoptively transferring vehicle- and adiponectin-treated T-cells into DSS/anti-PD1-treated mice. Colitis severity was lower in mice receiving adiponectin-treated T-cells compared to vehicle (FIGS. 7d and 7e). Splenic T-cells from DSS/anti-PD1 colitis mice by CyTOF was profiled and reduced TOX and enhanced B- and T-lymphocyte attenuator (BTLA), killer cell lectin-like receptor G1 (KLRG1) and CD47 expression on T-cells upon ECP-treatment were observed (FIGS. 7f-7i). Results were confirmed in anti-CTLA4-induced colitis (FIGS. S14d-S14f). Findings indicated reduced T-cell exhaustion and cytotoxicity upon ECP-therapy, as these markers are connected to reduced proliferative capacity and lower cytokine production. Transferring splenic T-cells from DSS/anti-PD1 or DSS/anti-PD1/ECP donors (FIGS. S14g and S14h) into secondary colitis recipients confirmed their tolerogenic phenotype upon ECP-treatment (FIGS. 7j and 7k). Transferred T-cells were enriched for CD4⁺KLRG1⁺CD47 BTLA⁺TOX^{low} T-cells, with no differences in T_{reg}, CD4⁺ and CD8⁺ frequencies (FIGS. S14i-S14q). These findings supported the concept of ECP and adiponectin affecting recipient T-cells, causing tolerogenic CD4 CD25^{low} T-cells in vitro and CD4 KLRG1⁺CD47⁺BTLA TOX^{low} T-cells in vivo. Consistently, scRNA-seq confirmed reduced T-cell activation in colonic lamina propria-derived effector T-cells after ECP-treatment (FIG. 7l, Table S1).

Immunosuppressive ECP-Effects Against irAEs Allowed for Anti-Tumor Immunity

Naïve mice were treated with daily steroids or weekly ECP-transplantations for 50 days to understand whether immunomodulatory ECP-effects influence recipient immunity. Long-term steroids are the standard first-line therapy for patients with severe ICI-induced colitis. Steroids diminished all major and effector splenic immune cell populations, whereas ECP-treatment showed no reduction compared to vehicle (FIGS. 8a-8i and S15a-S15g). These results were confirmed using a metastatic B16.F10-melanoma model, where additional steroid application, but not ECP-treatment, reduced survival compared to anti-PD1-therapy alone (FIGS. S16 and 8j). Transfer of untreated splenocytes did not affect survival of tumor-bearing mice (FIG. S16b). These findings were reproduced using 4344-BRAF^{V600E} mutant melanoma cells as a second model (FIG. 8k). Results were supported by an additional model in which DSS/anti-PD1-mediated colitis was induced in melanoma-bearing mice. Although colitis was similarly reduced between the steroid and ECP-treated groups (FIGS. S16c and S16d), the steroid-treated mice showed higher tumor burden, which was identified by luminescence imaging and expression of melanoma-specific transcripts (FIGS. S16e and S16f) in the lungs (FIGS. 8l-8o). Control of tumor growth was comparable between anti-PD1 and ECP-treated groups (FIGS. 8l-8o). Consistent with functional anti-melanoma immunity, the cytotoxic T-cells were not attenuated upon ECP-treatment of melanoma-bearing colitis mice (FIGS. S16g-S16j). Using OVA-transgenic tumors, a reduction of adoptively-transferred OT-I T-cells and endogenous CD8⁺OVA-Dextramer⁺ T-cells in the microenvironment of s.c. B16.F10-OVA melanoma and MC38-OVA^{dim} colorectal tumors upon steroid treatment was confirmed. In contrast, ECP-treatment did not affect tumor-specific T-cells (FIGS. 8p, 8q, and S16k). In support of the findings, T-cells isolated from B16.F10 melanoma-bearing anti-PD1 and anti-PD1/ECP-treated mice showed recall-immunity when transplanted into secondary B16.F10 melanoma-bearing recipients, whereas this was not the case when T-cells from anti-PD1/steroid treated donors were used (FIGS. 8r, S16l, and S16m). To understand why anti-tumor immunity remained intact upon ECP, Adipoq and Arg1 in the tumor microenvironment were analyzed. Neither local expression nor systemic levels of adiponectin were increased upon ECP-treatment (FIGS. 8s-8v). Furthermore, the analysis of tumor-specific T-cells revealed no elevated frequencies of tolerogenic T-cells in the tumor microenvironment (FIGS. S17a-S17h).

These findings indicated that ECP does not interfere with anti-PD1-induced anti-tumor immunity. The immune response remained intact due to the tissue specific effect of ECP induced by phagocytosis of apoptotic leukocytes accumulating in the inflamed colon but not in the tumor microenvironment (FIGS. 2k-2n).

### Example 2

### Patients

Key eligibility criteria included a minimum age of 18 years and steroid-refractory irAEs affecting the intestinal tract, liver, skin or lungs. Patients were eligible if they had received treatment with an anti-PD1, anti-PD-L1, an anti-CTLA4 antibody, or any combination of these for any type of malignancy in the last 24 months before screening Patients should have clinical and/or histological evidence of immune-related adverse events as follows:

Colitis: Diarrhea with increase of ≥4 stools over baseline; No improvement after 72 h treatment with at least 1 mg/kg BW/day prednisolone equivalent;

Hepatitis: Alanine aminotransferase and/or aspartate aminotransferase ≥3×ULN if baseline was normal; or ≥3× baseline if baseline was abnormal; No improvement after 72 h treatment with at least 1 mg/kg BW/day prednisolone equivalent;

Pneumonitis: Radiographic changes and new symptoms such as cough, dyspnea or chest pain; No improvement after 72 h treatment with 1 mg/kg BW/day prednisolone equivalent;

Dermatitis: Skin erythema, maculopapular or pustulopapular rash covering ≥30% of the body surface area; No improvement after 72 h treatment with at least 1 mg/kg BW/day prednisolone equivalent;

Maximum of one additional (second line) therapy after steroid treatment before ECP starts (e.g. infliximab for colitis).

An Eastern Cooperative Oncology Group (ECOG) performance status of 0 to 2.

### Clinical ECP-Treatment of Patients Treated in the Prospective Phase Ib/II Trial

ECP was performed on a Therakos CellEx photopheresis system. Methoxsalen (Uvadex©) was administered as a photo-sensitizing agent and 1500 ml blood were processed during each procedure. For the first 4 weeks, two procedures were performed on consecutive days each week. After week 4, the intervals were increased and the 2 days of ECP were performed every second week. Response was assessed on weeks 6 and 12 after start of ECP.

### Assessment of Response and Safety

irAE severity and response definitions were established based on the ASCO guidelines. Patients were monitored for clinical or laboratory signs of irAE at each visit. Adverse events (AE) and serious adverse events (SAE) were scored according to the Common Terminology Criteria for Adverse Events (CTCAE), version 5.0. Relationship to ECP was assessed for each AE/SAE by the investigator.

### End Points

The primary endpoint was the rate of treatment-related adverse events (AEs) and serious adverse events (SAEs) in patients treated with ECP for immune-checkpoint inhibitor-induced colitis, pneumonitis, hepatitis or dermatitis. A positive result from the study is defined as ≤50% of the patients developing a treatment-related SAE. The key secondary endpoint was objective response rate (ORR) to treatment after 6 and 12 weeks of ECP-therapy. The response criteria for each organ are described in the study protocol. Additional secondary endpoints were time to response, discontinuation of other immunosuppressive therapies and the incidence of tumor progression or relapse.

### Patient Cohort Analysis

The retrospective irAE-patient cohort analysis included 11 patients treated with ECP for irAE after informed consent. Inclusion criteria were: previous treatment with anti-CTLA4, anti-PD1, anti-PD-L1 antibodies, or a combination of these; clinical diagnosis of irAE; at least one previous therapy line for irAE prior to off-label treatment with ECP. All patients were informed about the off-label use of ECP as part of the pilot trial and gave their written informed consent prior to the first ECP procedure. The analysis of the clinical data was approved by the institutional review board. All procedures involving the analysis of peripheral blood were approved by the local Ethics Committees. Patients gave their written informed consent for the use of biological material for research.

### Clinical ECP-Treatment of Patients in the Real-World Patient Cohort

At all four centers, ECP was performed on a Therakos CellEx photopheresis system. Methoxsalen (Uvadex©) was administered as a photo-sensitizing agent and 1500 ml blood were processed during each procedure. At three centers, two procedures were commonly performed on consecutive days. The frequency and total number of ECP procedures were determined on an individual basis for each patient. At one center, one ECP procedure was performed every 2-3 days for a total of 14 days.

### Mice

Mice were used between 6 and 14 weeks of age, 15-25 mg of weight, and only female or male donor/recipient pairs were used.

### Statistical Analysis of the Clinical Data

Clinical data was managed and analyzed using Prism 9 (GraphPad), and SAS 9.4. Descriptive statistics were used to report continuous variables, as well as the frequency and percentages of discrete variables. P-values for the comparison of paired samples were calculated using the two-tailed paired Student's t-test.

### Statistical Analysis of Mouse and In Vitro Experiments

For the sample size in the murine survival experiments, a power analysis was performed. A sample size of at least n=10 per group was determined by 80% power to reach a statistical significance of 0.05 to detect an effect size of at least 1.06. Differences in animal survival (Kaplan-Meier survival curves) were analyzed by Mantel Cox test. The experiments were performed in a non-blinded fashion. All data were tested for normality applying the Kolmogorov-Smirnov test. For statistical analysis of two groups, an unpaired or paired two-tailed Student's 1-test was applied, dependent on the study design. If the data did not meet the criteria of normality, the Mann-Whitney test or Wilcoxon test was applied for unpaired or paired analyses, respectively. For comparison of more than 2 groups, the Kruskal-Wallis-Test (no matching or pairing) or the Friedman test (paired) with Dunn's multiple testing (if non-parametric testing was suggested) was used. An ordinary (no matching or pairing) or repeated measures (paired) one-way ANOVA with Tukey's multiple comparison in the case of normally distributed data was performed. Statistical analysis was performed using GraphPad Prism 9 (GraphPad). Data are presented as mean±standard error of the mean (s.e.m). A p-value<0.05 was considered significant.

Clinical Activity of ECP in Patients with irAE

Based on the pre-clinical findings, the clinical efficacy and safety of ECP in a prospective multicenter phase Ib/II trial were evaluated. As predefined in the study protocol, 14 patients were included, and the demographic and clinical characteristics are summarized in Table S2. All patients received 1-2 previous treatments for irAE and were corticosteroid-refractory. This was defined as the absence of symptom improvement after at least one week of 1 mg/kg BW steroid equivalent or rebound of symptoms upon tapering of immunosuppression. Patients received corticosteroid therapy for a median of 5 weeks prior to ECP (range 2-15 weeks) (FIG. S18a), irAE severity grades 3-4 were present in 57% before initiation of ECP (Table S2). At study inclusion, patients presented with colitis, hepatitis and dermatitis or combinations thereof. Underlying diseases included skin melanoma, uveal melanoma, non-small cell lung cancer (NSCLC) or thyroid cancer (Table S2). Patients received a median of 16 ECP-procedures (Figure S18b) and were monitored for AEs during ECP-treatment (Table S3). No SAE was considered directly related to ECP. Tumor staging before and after ECP indicated no evidence for increased progressive disease (PD) rates after ECP-treatment (FIG. S18c). At the time point of analysis, 2 of the 14 patients had died due to tumor progression. Both patients already had PD of the tumor at study inclusion. Median survival was not reached, median follow-up was 8 months, determined with the inverse Kaplan Meier method (FIG. S18d). Response to ECP-treatment was defined as complete resolution (CR) or improvement of irAE according to CTCAE grading (PR). Overall response rates of the irAEs (ORR) were 93% at week 6 (all 14 patients reached this time point) and 92% at week 12 (13 patients reached this time point, 1 died before week 12 due to tumor progression) (FIG. 9a). Individual patient responses with regard to hepatitis, colitis and dermatitis severity are shown (FIGS. 9b-9g and S18e). One patient had a relapse of the colitis after week 12 but responded again to ECP without the need for corticosteroid doses >0.5 mg/kg BW prednisone equivalent (FIG. 9c). At week 12 the CR rate was 100% for ICI-colitis patients (FIG. 9d). Steroids could be reduced after ECP-treatment in all patients (FIG. 9h). Two patients receiving ECP were re-challenged with ICI after resolution of irAEs without evidence of irAE relapse. The frequencies of major immune cell-populations remained stable when analyzed before, during or after ECP-therapy (FIGS. S18f-S18m). Confirming pre-clinical data, adiponectin serum levels and colonic ADIPOQ expression increased after ECP-treatment (FIGS. 9i and 9j). Tables S2 and S3 are shown below.

TABLE S2
Demographic and clinical characteristics of the patient
treated in the prospective phase Ib/II trial

| Variable | Value | |
|---|---|---|
| Total number of patients | 14 | |
| Age | [years] | |
| median | 59.5 | |
| range | 19-79 | |
| Sex | [absolute number (%)] | |
| female | 9 | (64) |
| male | 5 | (36) |
| Malignant disease | [absolute number (%)] | |
| melanoma of the skin | 9 | (64) |
| uveal melanoma | 1 | (7) |
| NSCLC | 3 | (21) |
| thyroid carcinoma | 1 | (7) |
| Primary immune checkpoint inhibitor treatment | [absolute number (%)] | |
| double immune checkpoint inhibitor therapy | 7 | (50) |
| (ipilimumab/nivolumab) | | |
| sequentially nivolumab and ipilimumab | 1 | (7) |
| nivolumab | 2 | (14) |
| pembrolizumab | 3 | (21) |
| unknown | 1 | (7) |
| irAE type at start of ECP (some pts had multiple | [absolute number (%)] | |
| irAE) | | |
| Colitis | 8 | (57) |
| Hepatitis | 8 | (57) |
| Dermatitis | 1 | (7) |
| leading irAE severity at ECP start | [absolute number (%)] | |
| Colitis | | |
| G1 | 0 | (0) |
| G2 | 4 | (57.1) |
| G3 | 3 | (42.9) |
| G4 | 0 | (0) |
| | Range: 2-3 | |
| | Median: 2.5 | |
| Hepatitis | | |
| G1 | 1 | (16.7) |
| G2 | 1 | (16.7) |
| G3 | 2 | (33.3) |
| G4 | 2 | (33.3) |
| | Range: 1-4 | |
| | Median: 3.0 | |
| Dermatitis | | |
| G1 | 0 | (0) |
| G2 | 0 | (0) |
| G3 | 0 | (0) |
| G4 | 1 | (100) |
| Immunosuppressive therapy prior to ECP | [absolute number (%)] | |
| Corticosteroid | 14 | (100) |
| MMF | 2 | (14.3) |

TABLE S3
Adverse events in patients treated in the prospective phase Ib/II trial

| | | | | | ongoing/ |
|---|---|---|---|---|---|
| | No. of | CTC-AE grade | Relation to | | resolved |
| AE | events | (no. of events) | ECP likely | SAE | (no. of events) |
| iron deficiency | 2 | II (1), | no | no | ongoing (2) |
| | | unknown (1) | | | |
| photosensitivity | 2 | I (2) | yes | no | resolved (2) |
| rash | 2 | I (2) | no | no | resolved (2) |
| fatigue | 2 | I (2) | no | no | resolved (2) |
| increased body | 2 | I (2) | no | no | resolved |
| temperature | | | | | |
| dry eye | 2 | I (1), II (1) | no | no | resolved (1), ongoing |
| | | | | | (1) |
| gastrointestinal infection | 1 | III | no | yes | resolved |
| bronchitis | 1 | II | no | no | resolved |
| thrombosis | 1 | II | no | no | resolved |
| disorientation | 1 | II | no | no | resolved |
| unsteady gait | 1 | II | no | no | ongoing |
| SARS-CoV2 infection | 1 | II | no | yes | resolved |
| nausea | 1 | II | no | no | resolved |
| blood stream infection | 1 | II | no | no | resolved |
| (most probably | | | | | |
| contamination from skin) | | | | | |
| urinary tract infection | 1 | II | no | no | resolved |
| upper respiratory infection | 1 | I | no | no | resolved |
| rhinorrhea | 1 | I | no | no | resolved |
| throat complaints | 1 | I | no | no | resolved |
| multiple naevi of the skin | 1 | I | no | no | ongoing |
| urge to urinate | 1 | I | no | no | resolved |
| cough | 1 | I | no | no | resolved |
| exanthema | 1 | I | no | no | ongoing |

In addition to the prospective trial, real-world data from 11 irAE-patients was collected. Patients received corticosteroid monotherapy or combinations with mycophenolate mofetil (MMF), Cyclosporine A (CSA) or infliximab (Table S4; Figure S19a). At ECP-initiation, patients presented with colitis, hepatitis, dermatitis, arthritis, or combinations thereof. Underlying diseases included skin melanoma, uveal melanoma, renal cell carcinoma and NSCLC (Table S4). Patients received 21 ECP-procedures (median) until achievement of best response (FIG. S19b), were monitored for AEs during ECP-treatment and no SAE could be directly linked to ECP (Table S5). Nine of 11 patients (81.8%) reached CR, defined as the absence of any irAE-symptoms, while one patient achieved PR (decrease ≥1 grade in ≥1 organ-manifestation) and one patient had SD (FIG. 9EA). The clinical course of a representative hepatitis patient is shown in FIG. S19c. Steroids were reduced in all patients after ECP-treatment (FIG. 9EB). Five patients were re-challenged with ICI after irAE-resolution. Two patients did not show irAE-relapse, whereas 3 relapsed from initial irAE, leading to ICI-termination or a switch from double to single ICI treatment. Tumor staging indicated no increased progressive disease-rates after ECP-therapy (FIG. S19d). In summary, ECP appeared to be safe and effective against ICI-induced irAEs with no unexpected toxicities. Tables S4 and S5 are shown below.

TABLE S4
Demographic and clinical characteristics
of the real world patient cohort

| Variable | Value | |
|---|---|---|
| Total number of patients | 11 | |
| Age | [years] | |
| median | 66 | |
| range | 31-74 | |
| Sex | [absolute number (%)] | |
| female | 3 | (27.3) |
| male | 8 | (72.3) |
| Malignant disease | [absolute number (%)] | |
| melanoma of the skin | 8 | (72.3) |
| uveal melanoma | 1 | (9.1) |
| renal cell carcinoma | 1 | (9.1) |
| HNSCC | 1 | (9.1) |
| Primary immune checkpoint inhibitor treatment | [absolute number (%)] | |
| ipilimumab/nivolumab | 6 | (54.5) |
| pembrolizumab/tyrosine kinase inhibitor | 1 | (9.1) |
| pembrolizumab, ipilimumab/nivolumab | 1 | (9.1) |
| ipilimumab/nivolumab, ipilimumab mono, | 1 | (9.1) |
| nivolumab mono | | |
| nivolumab, ipilimumab/nivolumab | 1 | (9.1) |
| pembrolizumab | 1 | (9.1) |
| irAE type | [absolute number (%)] | |
| Colitis | 9 | (81.8) |
| Hepatitis | 2 | (18.2) |
| Dermatitis | 1 | (9.1) |
| Arthritis | 1 | (9.1) |
| Stomatitis | 1 | (9.1) |
| leading irAE severity at ECP start | [absolute number | |
| | (% of colitis/hepatitis/ | |
| | dermatitis)] | |
| Colitis | | |
| G1 | 1 | (11.1) |
| G2 | 2 | (22.2) |
| G3 | 6 | (66.7) |
| G4 | 0 | |
| | Range: 1-3 | |
| | Median: 3.0 | |
| Hepatitis | | |
| G1 | 0 | |
| G2 | 0 | |
| G3 | 0 | |
| G4 | 1 | (100) |
| Dermatitis | | |
| G1 | 0 | |
| G2 | 1 | (100) |
| G3 | 0 | |
| G4 | 0 | |
| Immunosuppressive therapy prior to ECP | [absolute number (%)] | |
| Corticosteroid | 11 | (100) |
| MMF | 2 | (18.2) |
| Infliximab | 8 | (72.7) |
| CSA | 1 | (9.1) |

TABLE S5
Adverse events in the real world patient cohort

| | | | | | ongoing/ |
|---|---|---|---|---|---|
| | No. of | CTC-AE grade | Relation to | | resolved |
| AE | events | (no. of events) | ECP likely | SAE | (no. of events) |
| local bacterial/fungal | 5 | I (2), II (3) | No | no | resolved (5) |
| infection | | | | | |
| leukocytosis/ | 5 | I (5) | No | no | resolved (4) |
| neutrophilia | | | | | ongoing (1) |
| anemia | 4 | II (4) | Yes | no | resolved (1) |
| | | | | | ongoing (3) |
| increased creatinine | 4 | I (2), II (2) | No | no | resolved (2) |
| | | | | | ongoing (2) |
| increase of bile duct or liver | 2 | I (1), III (1) | No | no | resolved (1) |
| enzymes | | | | | ongoing (1) |
| systemic (bacterial) | 3 | III (3) | No | yes | resolved (3) |
| infection/sepsis | | | | | |
| cataract | 2 | III (2) | No | yes | resolved (2) |
| hyperlipidemia | 2 | I (2) | No | no | unknown (2) |
| drug eruption | 2 | II (1), III (1) | No | no | resolved (2) |
| arthralgia | 2 | I (2) | No | no | resolved (1) |
| | | | | | ongoing (1) |
| abdominal pain | 2 | I (1), II (1) | No | no | unknown (2) |
| increased lipase | 2 | I (1), II (1) | No | no | resolved (1) |
| | | | | | unknown (1) |
| hypokalemia | 1 | IV | No | yes | resolved |
| pneumonitis | 1 | III | No | yes | resolved |
| acute limb ischemia | 1 | III | No | yes | resolved |
| aseptic bone necrosis | 1 | III | No | yes | resolved |
| secondary adrenal | 1 | II | No | no | resolved |
| insufficiency | | | | | |
| hypotension | 1 | II | No | no | resolved |
| nausea | 1 | II | No | no | unknown |
| edema | 1 | I | No | no | resolved |
| hypercalcemia | 1 | I | No | no | resolved |
| hyperuricemia | 1 | I | No | no | resolved |
| hypalbuminemia | 1 | II | No | no | ongoing |
| hypocalcemia | 1 | II | Yes | no | resolved |
| thrombocytopenia | 1 | I | No | no | resolved |
| diarrhea | 1 | III | No | no | resolved |
| anal fissure | 1 | II | No | no | resolved |
| osteoporosis | 1 | II | No | no | ongoing |
| biceps tendon rupture | 1 | II | No | no | resolved |
| glaucoma | 1 | I | No | no | ongoing |
| restless legs syndrome | 1 | I | No | no | resolved |
| muscle cramps | 1 | I | No | no | resolved |
| COVID19 infection | 1 | I | No | no | resolved |
| rash | 1 | I | No | no | resolved |
| vascular access complication | 1 | III | No | no | resolved |
| (thrombosis) | | | | | |
| hyponatremia | 1 | II | No | no | ongoing |
| weight loss | 1 | I | No | no | unknown |
| fatigue | 1 | I | No | no | unknown |
| urinary incontinence | 1 | I | No | no | unknown |
| vitiligo | 1 | I | No | no | unknown |
| alopecia | 1 | I | No | no | unknown |
| pruritus | 1 | I | No | no | unknown |

In a humanized irAE-model, Rag2^{-/-}γc^{-/-} mice received healthy-donor PBMCs and anti-PD1 (FIG. S20a). Application of ECP resolved histopathological signs of irAEs in major target organs, comparable to steroid treatment (FIGS. 9EC, 9ED, and S20b-S20g). irAEs were low to absent in mice receiving PBMCs only, confirming ICI-mediated inflammation (FIGS. S20h-S200).

These findings indicated that ECP has activity in patients with ICI-induced colitis who failed to respond to steroids or other immunosuppressive agents in a prospective clinical trial and a real-world setting, and this was confirmed in a humanized irAE-mouse model.

### Discussion

Patients developing severe grade 3 or 4 irAEs after ICB are currently treated with glucocorticosteroids and long-term interruption of the immunotherapy. Both interventions can negatively influence anti-tumor immunity, as suggested by retrospective analyses. Additionally, it is desirable to avoid glucocorticosteroids because of the systemic side-effects including hyperglycemia, fluid retention, psychological disorders, and infectious complications. Currently, no prospective randomized trial supports the use of glucocorticosteroids or second line therapies such as mycophenolate mofetile (MMF), TNF antagonists or cyclosporine A. Novel approaches that counteract irAEs without strong systemic immunosuppression are lacking. ECP activity in patients with severe chronic graft-versus-host-disease (cGVHD), which was refractory to ruxolitinib, has been previously reported. In addition, a patient with irAE colitis who was successfully treated with ECP has also been previously reported. However, the molecular mechanisms underlying ECP-induced immunomodulation remained unclear.

A model of DSS/ICI-induced colitis was employed to investigate the mechanisms of ECP-induced resolution of irAE-colitis. Established ICI-sensitive melanoma models was used to understand the impact of ECP on anti-tumor responses after anti-PD1-based immunotherapy. Using an unbiased microarray approach, ECP-induced adiponectin expression was discovered in the intestinal tract, which has never before been connected to the mode of action of ECP. Adiponectin was originally identified in adipocytes, but later studies also revealed its expression in non-adipose cells. Increased adipose tissue mass is associated with the development of autoimmune diseases and certain adipokines such as adiponectin, which are likely produced to counterbalance adipose tissue-related inflammation. Obese patients have lower adiponectin serum levels, suggesting this lack may contribute to the body's inability to counteract inflammation in obesity. Consistently, adiponectin was seen to decrease in patients with type-2 diabetes, highlighting its role as an anti-inflammatory molecule. Adiponectin binds to its two receptors AdipoR1 and AdipoR2, which are highly expressed on myeloid cells. It can polarize macrophages towards a tolerogenic phenotype, seen by elevated IL-10 production and reduced expression of IFNγ and TNF. Overexpression of adiponectin in macrophages in mice caused decreased serum levels of proinflammatory MCP-1 and TNF when mice were fed with a high-fat diet. Similarly, Adipoq^{-/-} mice showed increased levels of TNF in adipose tissue and blood. Furthermore, adiponectin treatment reduced TNF-production and disease activity in mice with nonalcoholic fatty liver disease. Consistent with its anti-inflammatory role, adiponectin negatively regulated pro-inflammatory IL-33 signaling in ILC2 residing in adipose tissue. Adiponectin itself is negatively regulated by inflammatory mediators, and anti-IL-6R inhibition increased systemic adiponectin levels in RA patients.

The study showed that adiponectin is induced by phagocytic uptake of apoptotic leukocytes in inflamed tissues. Leukocytes were rendered apoptotic by ECP-treatment and cleared by phagocytic cells residing in the intestinal wall of colitis-mice. Mechanistically, the uptake of apoptotic cells caused STAT6-phosphorylation and PPARγ-activation. This in turn caused anti-inflammatory polarization in macrophages, seen by enhanced Arg1 production. Using arginase-1 inhibitor-treatment and Arg1^{-/-} mice confirmed its important role to mediate ECP-mediated immunosuppression. Arg1 depletes arginine, which is essential for the function of activated T-cells. In addition to the indirect inhibition of T-cells through Arg1-induction, adiponectin can inhibit T-cell activation directly, which was confirmed by reduced pathogenic CD25-expression. Adoptive transfer of activated T-cells enhanced the severity of colitis, whereas adiponectin pre-treated T-cells did not. ECP increased the frequencies of less-exhausted splenic tolerogenic T-cells in colitis mice, which suggests reduced inflammatory activities for these cells because KLRG1 identifies antigen-experienced T-cells lacking proliferative capacity. BTLA and CD47 are known to be inhibitory molecules which reduce pro-inflammatory T-cell responses. The complex mechanism of action of ECP is summarized in the graphical abstract (FIG. S21).

Importantly, anti-tumor immunity remained intact in ECP-treated melanoma-bearing mice, while corticosteroids abrogated ICI-induced anti-tumor immune effects. Consistent with reduced anti-tumor activity, prednisolone diminished all major immune cell populations in mice. Notably, antigen-specific immune cell frequencies were reduced upon steroid application, thereby limiting anti-tumor immunity. It was further investigated how ECP suppresses ICI-induced colitis without affecting anti-melanoma immunity. It was found that ECP-treated apoptotic cells are phagocytosed locally in inflamed colon tissue, but not within the tumor microenvironment. Furthermore, upregulation of adiponectin and Arg1 was seen only in colitis, but not tumor tissue. Similarly, increased frequencies of tolerogenic, antigen-specific T-cells in the tumor microenvironment after ECP-treatment was not found. The tissue tropism of ECP-treated apoptotic cells can be explained by strong chemokine release in the inflamed intestinal tract, which attracts the cells, while such a chemokine-milieu might not be present in the tumor tissue. Of interest, ECP allowed for recall immunity if T-cells from ECP-treated donors were adoptively transferred into secondary recipients.

Similar to the findings in the mouse models, it was observed that ECP-treatment within a multicenter prospective clinical trial and a retrospective study led to clinical responses in 92% and 90.9% of irAE patients, respectively. Comparable to the pre-clinical observations, ECP induced adiponectin in the blood and intestinal tissues of irAE-patients, without toxic effects. All patients receiving ECP could reduce their immunosuppression regimens. The favorable safety profile of ECP observed in patients with irAE in this study is in agreement with the reported safety profile of patients treated with ECP for acute or cGVHD. Treatment of irAE patients with ECP in our prospective phase Ib/II trial revealed a complete remission rate of 100% for ICI-colitis patients at week 12 after the start of ECP which is higher than all reported response rates for ICI-colitis patients to date. The ORR across all irAEs was 92% at week 12 after the start of ECP. The high overall response rate was confirmed in the real-world cohort showing a clinical response rate of 90.9 percent (81.8% CR rate). The phase Ib/II study is the first prospective interventional controlled trial with defined analysis time points (week 6 and 12) for patients with ICI-induced colitis, hepatitis and dermatitis as no other currently published reports fulfill these criteria (Tables S6 and S7). Tables S6 and S7 are shown below.

TABLE S6
Clinical trials investigating treatment for ICI-colitis

| | | No. of | | |
|---|---|---|---|---|
| Treatment | Trial design | patients | Outcome measures | Outcome |
| Extracorporeal | Prospective; | 14 | Defined primary endpoint | Safety and efficacy |
| photopheresis | interventional; | | Response assessment | irAE improvement (≥1 grade |
| | multi-center | | | reduction) within 6/12 weeks |
| | | | ORR week 6/week 12 | 100%/100% |
| | | | CR week 12 colitis | 100% |
| | | | Steroid reduction | 100% |
| | | | Tumor progression | 40% |
| | | | Adverse events | Gr. 1-2: 50%, Gr. 3-4: 13% |
| Extracorporeal | Retrospective; | 11 | Defined primary endpoint | Not defined |
| photopheresis | multi-center | | Response assessment | irAE improvement after ECP |
| | | | | therapy |
| | | | ORR | 89% |
| | | | CR | 89% |
| | | | Steroid reduction | 100% |
| | | | Tumor progression | only partially known |
| | | | | 2 of 6 patients treated in FR |
| | | | Adverse events | only partially known |
| | | | | grade 1-2: |
| | | | | 8/8 patients treated in FR and ES |
| | | | | grade 3-4: |
| | | | | 4/8 patients treated in FR and ES |
| Tocilizumab | Prospective | 10 | Defined primary endpoint | Clinical improvement of colitis |
| | | | Response assessment | Colitis improvement (≥1 grade |
| | | | | reduction) within 8 weeks |
| | | | ORR | 80% |
| | | | CR | 0% |
| | | | Steroid reduction | Not described |
| | | | Tumor progression | 44% |
| | | | Adverse events | 80% any grade; 25% grade 3-4 |
| Vedolizumab | Retrospective; | 62 | Defined primary endpoint | Not defined |
| | observational | | Response assessment | Sustained resolution of colitis |
| | | | | to ≤grade 1 |
| | | | ORR (clinical remission) | 89% |
| | | | CR | No information |
| | | | Steroid reduction | Not described |
| | | | Tumor progression | 24% (before) vs. 54% (after) |

TABLE S7
Clinical trials investigating treatment for ICI-hepatitis

| | | No. of | | |
|---|---|---|---|---|
| Treatment | Trial design | patients | Outcome measures | Outcome |
| Extracorporeal | Prospective; | 14 | Defined primary endpoint | Safety and efficacy |
| photopheresis | interventional; | | Response assessment | irAE improvement (≥1 grade |
| | multi-center | | | reduction) within 6/12 weeks |
| | (EudraCT 2021- | | ORR week 6/week 12 | 88%/83% |
| | 002073-26) | | CR week 6/week 12 | 29%/33% |
| | | | Steroid reduction | 100% |
| | | | Tumor progression | 40% |
| | | | Adverse events | Grade 1-2: 75%, Grade 3-4: 0% |
| Extracorporeal | Retrospective; | 11 | Defined primary endpoint | Not defined |
| photopheresis | multi-center | | Response assessment | irAE improvement after ECP |
| | (clinicaltrials.gov: | | | therapy |
| | NCT05414552) | | ORR | 100% |
| | | | CR | 100% |
| | | | Steroid reduction | 100% |
| | | | Tumor progression | only partially known |
| | | | | 1 of 1 patient treated in FR |
| | | | Adverse events | only partially known |
| | | | | grade 1-2: |
| | | | | 1/1 patient treated in FR |
| | | | | grade 3-4: |
| | | | | 0/1 patient treated in FR |
| Mycophenolate | Retrospective | 8 | Defined primary endpoint | Efficacy and safety |
| mofetil | | | Response assessment | Improve of liver function tests to |
| | | | | grade 1 or less |
| | | | ORR | 50% |
| | | | CR | Not reported |
| | | | Steroid reduction | Not reported |
| | | | Tumor progression | Unclear |
| | | | Adverse events | 25% |
| Mycophenolate | Case Report | 1 | Response assessment | Improvement of liver injury |
| mofetil | | | Response | Improved liver injury within 13 |
| | | | | days |
| | | | Steroid reduction | Tapered from 60 to 10 mg/d |
| | | | Tumor progression | Stable disease |
| | | | Adverse events | Not reported |
| Mycophenolate | Case Report | 1 | Response assessment | Reduction of AST, ALT, ALP |
| mofetil | | | Response | Discharge of MMF after 68 days |
| | | | Steroid reduction | Tapered from 500 to 10 mg/d |
| | | | Tumor progression | Lymph node metastases, death at |
| | | | | 9.7 month post irAE hospitalization |
| | | | Adverse events | Not reported |
| Mycophenolate | Case Report | 1 | Response assessment | Reduction of bilirubin, AST, ALT |
| mofetil | | | Response | Liver function normalized within 3 |
| | | | | weeks |
| | | | Steroid reduction | Discontinuation after 1 year |
| | | | Tumor progression | Not reported |
| | | | Adverse events | Not reported |
| Tacrolimus | Case Report | 1 | Response assessment | Improvement of liver function |
| | | | Response | Near-resolution of liver function at |
| | | | | 12 months post presentation |
| | | | Steroid reduction | Cessation at 12 months |
| | | | Tumor progression | Partial response |
| | | | Adverse events | Not reported |
| Tacrolimus | Case Report | 2 | Response assessment | Improvement hepatitis |
| | | | Response | Decreasing ALT levels within 2-5 |
| | | | | days after start of treatment |
| | | | Steroid reduction | Cessation of prednisolone over 6 |
| | | | | months |
| | | | Tumor progression | Not reported |
| | | | Adverse events | Not reported |
| Anti- | Case Report | 1 | Response assessment | Improvement of liver function |
| Thymocyte | | | Response | Transaminase and lymphocyte |
| Globulin | | | | count reduction |
| | | | Steroid reduction | Weaned over 6 weeks |
| | | | Tumor progression | Not reported |
| | | | Adverse events | Fever, rigors, cytokine release |
| Anti- | Case Report | 1 | Response assessment | Improvement of liver function |
| Thymocyte | | | Response | Improvement of liver functions |
| Globulin | | | | within 12 days, ongoing until day |
| (ATG) and n- | | | | 72 |
| Acetylcysteine | | | | |
| (NAC) | | | Steroid reduction | Unclear |
| | | | Tumor progression | Not reported |
| | | | Adverse events | No infectious complications |
| Infliximab | Case Report | 1 | Response assessment | Improvement of ALT and Bilirubin |
| | | | | levels and liver inflammation |
| | | | Response | Partial response, reduction of ALT |
| | | | | and Bilirubin after 2 cycles |
| | | | Steroid reduction | Tapering to 30%, off steroid at 14 |
| | | | | months after last ICI |
| | | | Tumor progression | Stable liver metastases |
| | | | Adverse events | Not reported |
| Enteric-coated | Case Report | 1 | Response assessment | Improvement of liver function |
| budesonide | | | Response | Decline of ALT and AST; normal |
| | | | | after 1 month |
| | | | Steroid reduction | Not reported |
| | | | Tumor progression | Not reported |
| | | | Adverse events | Mild bilateral leg edema |

A prospective trial using interleukin-6 blockade by tocilizumab for ICI-induced colitis reported the best response at any time point during a period of 8 weeks after treatment start. A reduction in symptoms in 8 of 10 patients but no CR was reported. It was observed that ECP induced a 100% CR rate of ICI-colitis at week 12. A retrospective observational cohort study on ICI-colitis patients reported response rates of 89% and 88% to vedolizumab and infliximab, respectively. The response was counted as best response at any time point during the observational period, meaning that even patients that had a short lived response that was lost afterwards counted as responders, in contrast to the prospective trial in which response was determined at predefined time points. Furthermore, the study provided no information on the CR-rate. A retrospective case series of 28 patients with ICI-colitis refractory to steroids and/or infliximab who received vedolizumab reported an endoscopic remission rate of 29%. A retrospective analysis of 13 patients who were administered infliximab for ICI-colitis reported an ORR of 45% and CR of 31%. A retrospective analysis of 8 patients treated with infliximab for ICI colitis reported an ORR of 50% and CR rate of 38%. Overall, treatment of irAE patients with ECP in the prospective phase Ib/II trial revealed a complete remission rate of 100% for ICI-colitis patients at week 12 after start of ECP and an ORR across all irAEs of 92%. The high ORR was confirmed in a real-world cohort and showed a clinical response rate of 90.9 percent (81.8% CR rate).

In summary, ECP was identified as a novel immunomodulatory approach to control ICI-induced colitis and irAEs in mice and humans. This is the first study clarifying the underlying molecular mechanisms of ECP-treatment in controlled in vivo models. The studies showed in mice that ECP-treated leukocytes underwent apoptosis and were phagocytosed by intestinal macrophages, which in turn caused STAT6- and PPARγ-activation. This increased adiponectin-production in colitis tissue. It was found that that adiponectin enhanced arginase-1 production in myeloid cells, and both ECP and adiponectin reduced pro-inflammatory T-cell frequencies in mice. Tolerogenic T-cells were increased in the inflamed intestines but not in melanoma tissue, thereby leaving anti-tumor immunity unaffected. Corticosteroids reduced anti-tumor immunity.

These findings indicated that ECP can meet the unmet clinical need of a novel therapeutic approach to treat ICI-induced irAEs without negatively affecting anti-tumor immunity.

### REFERENCES

1. Weber, J. S., Hodi, F. S., Wolchok J D, Topalian S L, Schadendorf D, Larkin J, Sznol M, Long G V, Li H, Waxman I M, Jiang J, Robert C. Safety Profile of Nivolumab Monotherapy: A Pooled Analysis of Patients With Advanced Melanoma. J Clin Oncol. 35, 785-792 (2017).
2. Haanen, J., Obeid M, Spain L, Carbonnel F, Wang Y, Robert C, Lyon A R, Wick W, Kostine M, Peters S, Jordan K, Larkin J. Management of toxicities from immunotherapy: ESMO Clinical Practice Guideline for diagnosis, treatment and follow-up. Ann Oncol. 33, 1217-1238 (2022).
3. Schneider, B. J., Naidoo J, Santomasso B D, Lacchetti C, Adkins S, Anadkat M, Atkins M B, Brassil K J, Caterino J M, Chau I, Davies M J, Ernstoff M S, Fecher L, Ghosh M, Jaiyesimi I, Mammen J S, Naing A, Nastoupil L J, Phillips T, Porter L D, Reichner C A, Seigel C, Song J M, Spira A, Suarez-Almazor M, Swami U, Thompson J A, Vikas P, Wang Y, Weber J S, Funchain P, Bollin K. Management of Immune-Related Adverse Events in Patients Treated With Immune Checkpoint Inhibitor Therapy: ASCO Guideline Update. J Clin Oncol. 39, 4073-4126 (2021).
4. Luo, J., Beattie J A, Fuentes P, Rizvi H, Egger J V, Kern J A, Leung D Y M, Lacouture M E, Kris M G, Gambarin M, Santomasso B D, Faleck D M, Hellmann M D. Beyond steroids: immunosuppressants in steroid-refractory/resistant immune related adverse events. J Thorac Oncol. 16, 1759-1764 (2021).
5. Horvat, T. Z., Adel, N. G., Dang T O, Momtaz P, Postow M A, Callahan M K, Carvajal R D, Dickson M A, D'Angelo S P, Woo K M, Panageas K S, Wolchok J D, Chapman P B. Immune-related adverse events, need for systematic immunosuppression, and effects on survival and time to treatment failure in patients with melanoma treated with ipilimumab at Memorial Sloan Kettering Cancer Center. J Clin Oncol. 33, 3193-3198 (2015).
6. Marschner, D., Falk M, Javorniczky N R, Hanke-Müller K, Rawluk J, Schmitt-Graeff A, Simonetta F, Haring E, Dicks S, Ku M, Duquesne S, Aumann K, Rafei-Shamsabadi D, Meiss F, Marschner P, Boerries M, Negrin R S, Duyster J, Zeiser R, Köhler N. MicroRNA-146a regulates immune-related adverse events caused by immune checkpoint inhibitors. JCI Insight 5, 132334 (2020).
7. Postow, M. A., Sidlow R, Hellmann M D. Immune-Related Adverse Events Associated with Immune Checkpoint Blockade. N Eng J Med 378, 158-168 (2018).
8. Del Castillo, M., Romero F A, Argüello E, Kyi C, Postow M A, Redelman-Sidi G. The spectrum of serious infections among patients receiving immune checkpoint block-ade for the treatment of melanoma. Clin Infect Dis. 63, 1490-1493 (2016).
9. Montfort, A., Filleron T, Virazels M, Dufau C, Milhès J, Pages C, Olivier P, Ayyoub M, Mounier M, Lusque A, Brayer S, Delord J P, Andrieu-Abadie N, Levade T, Colacios C, Ségui B, Meyer N. Combining nivolumab and ipilimumab with infliximab or certolizumab in patients with advanced melanoma: first results of a phase 1b clinical trial. Clin Cancer Res. 27, 1037-1047 (2021).
10. Holmstroem, R. B., Nielsen O H, Jacobsen S, Riis L B, Theile S, Bjerrum J T, Vilmann P, Johansen J S, Boisen M K, Eefsen R H L, Marie Svane I, Nielsen D L, Chen I M. COLAR: open-label clinical study of IL-6 blockade with tocilizumab for the treatment of immune checkpoint inhibitor-induced colitis and arthritis. J Immunother Cancer. 9, e005111 (2022).
11. Zou, F., Faleck, D., Thomas A, Harris J, Satish D, Wang X, Charabaty A, Ernstoff M S, Glitza Oliva I C, Hanauer S, McQuade J, Obeid M, Shah A, Richards D M, Sharon E, Wolchok J, Thompson J, Wang Y. Efficacy and safety of vedolizumab and infliximab treatment for immune-mediated diarrhea and colitis in patients with cancer: a two-center observational study. J Immunother Cancer. 11, e003277 (2021).
12. Abu-Sbeih, H., Ali, F. S., Alsaadi D, Jennings J, Luo W, Gong Z, Richards D M, Charabaty A, Wang Y. Outcomes of vedolizumab therapy in patients with immune checkpoint inhibitor-induced colitis: a multi-center study. J Immunother Cancer. 6, 142 (2018).
13. Hillock, N. T., Heard, S., Kichenadasse G, Hill C L, Andrews J. Infliximab for ipilimumab-induced colitis: A series of 13 patients. Asia Pac J Clin Oncol. 5, e284-e290 (2017).
14. Kadokawa, Y., Takagi M, Yoshida T, Tatsumi A, Fujita K, Inoue T, Ohe S, Nakai Y, Yamamoto S, Otsuka T, Ishihara R, Isei T, Kumagai T, Nishimura K, Imamura F. Efficacy and safety of Infliximab for steroid-resistant immune-related adverse events: A retrospective study. Mol Clin Oncol. 4, 65 (2021).
15. Apostolova, P., Unger, S., Meiss, F., von Bubnoff, D., Aumann, K., Becher, B., Zeiser, R. Extracorporeal photopheresis for severe immune checkpoint inhibitor-induced autoimmune colitis. N Eng J Med 382, 294-296 (2020).
16. Gaillet, A., Delage, L., Wislez M, Goupil F, Mouthon L, Terrier B. Extracorporeal photopheresis and cyclophosphamide for cancer-associated systemic sclerosis worsening induced by immune checkpoint inhibitors: a case report. Clin Exp Rheumatol. 40, 2004-2005 (2022).
17. Scherer, P. E., Williams S, Fogliano M, Baldini G, Lodish H F. A novel serum protein similar to Clq, produced exclusively in adipocytes. J Biol Chem. 270, 26746-26749 (1995).
18. Polito, R., Nigro, E., Messina A, Monaco M L, Monda V, Scudiero O, Cibelli G, Valenzano A, Picciocchi E, Zammit C, Pisanelli D, Monda M, Cincione I R, Daniele A, Messina G. Adiponectin and Orexin-A as a Potential Immunity Link Between Adipose Tissue and Central Nervous System. Front Physiol. 9, 982 (2018).
19. Luo, N., Liu, J., Chung, B. H., Yang, Q., Klein, R. L., Garvey W T, Fu Y. Macrophage adiponectin expression improves insulin sensitivity and protects against inflammation and atherosclerosis. Diabetes 59, 791-799 (2010).
20. Perez-Ruiz, E., Minute L, Otano I, Alvarez M, Ochoa M C, Belsue V, de Andrea C, Rodriguez-Ruiz M E, Perez-Gracia J L, Marquez-Rodas I, Llacer C, Alvarez M, de Luque V, Molina C, Teijeira A, Berraondo P, Melero I. Prophylactic TNF blockade uncouples efficacy and toxicity in dual CTLA-4 and PD-1 immunotherapy. Nature 569, 428-432 (2019).
21. Beck, K. E., Blansfield, J. A., Tran, K. Q., Feldman, A. L., Hughes, M. S., Royal, R. E., et al. Enterocolitis in patients with cancer after antibody blockade of cytotoxic T-lymphocyte-associated antigen 4. J Clin Oncol. 24, 2283-2289 (2006).
22. Schwab, L., Goroncy, L., Palaniyandi, S., Gautam, S., Triantafyllopoulou, A., Mocsai, A, Reichardt, W., Karlsson, F. J. Radhakrishnan, S. V., Hanke, K., Schmitt-Graeff, A., Freudenberg, M., von Loewenich, F. D., Wolf, P., Leonhardt, F., Baxan, N., Pfeifer, D., Schmah, O., Schönle, A., Martin, S. F., Mertelsmann, R., Duyster, J, Finke, J., Prinz, M., Henneke, P., Häcker, H., Hildebrandt, G. C., Häcker, G., Zeiser, R. Neutrophil granulocytes recruited upon translocation of intestinal bacteria enhance GvHD via tissue damage. Nature medicine 20, 648-654 (2014).
23. Zeiser, R., Warnatz, K., Rosshart S, Sagar, Tanriver Y. GVHD, IBD, and primary immunodeficiencies: The gut as a target of immunopathology resulting from impaired immunity. Eur J Immunol. 52, 1406-1418 (2022).
24. Gardai, S. J., McPhillips, K. A., Frasch, S. C., Janssen, W. J. Starefeldt A, Murphy-Ullrich J E, et al. Cell-surface calreticulin initiates clearance of viable or apoptotic cells through trans-activation of LRP on the phagocyte. Cell 123, 321-334 (2005).
25. Yu, T., Gan S, Zhu Q, Dai D, Li N, Wang H, Chen X, Hou D, Wang Y, Pan Q, Xu J, Zhang X, Liu J, Pei S, Peng C, Wu P, Romano S, Mao C, Huang M, Zhu X, Shen K, Qin J, Xiao Y. Modulation of M2 macrophage polarization by the crosstalk between Stat6 and Trim24. Nat Commun. 10, 4353 (2019).
26. Orecchioni, M., Ghosheh, Y., Pramod, A. B., Ley K. Macrophage Polarization: Different Gene Signatures in M1(LPS+) vs. Classically and M2(LPS-) vs. Alternatively Activated Macrophages. Front Immunol. 10, 1084 (2019).
27. Bouhlel, M. A., Derudas B, Rigamonti E, Dièvart R, Brozek J, Haulon S, Zawadzki C, Jude B, Torpier G, Marx N, Staels B, Chinetti-Gbaguidi G. PPARgamma activation primes human monocytes into alternative M2 macrophages with anti-inflammatory properties. Cell Metab. 2, 137-143 (2007).
28. Highfill, S. L., Rodriguez, P. C., Zhou, Q., Goetz, C. A., Koehn, B. H., Veenstra, R., Taylor P A, Panoskaltsis-Mortari A, Serody J S, Munn D H, Tolar J, Ochoa A C, Blazar B R. Bone marrow myeloid-derived suppressor cells (MDSCs) inhibit graft-versus-host disease (GVHD) via an arginase-1-dependent mechanism that is up-regulated by interleukin-13. Blood 116, 5738-5747 (2010).
29. Gordon, S. Alternative activation of macrophages. Nat Rev Immunol. 1, 23-35 (2003).
30. Rubin, L. A., Kurman C C, Fritz M E, Biddison W E, Boutin B, et al. Soluble interleukin 2 receptors are released from activated human lymphoid cells in vitro. J Immunol. 135, 3172-3177 (1985).
31. Ross, S. H., Cantrell D A. Signaling and Function of Interleukin-2 in T Lymphocytes. Annu Rev Immunol. 36, 411-433 (2018).
32. Alfei, F., Kanev K, Hofmann M, Wu M, Ghoneim H E, Roelli P, Utzschneider D T, von Hoesslin M, Cullen J G, Fan Y, Eisenberg V, Wohlleber D, Steiger K, Merkler D, Delorenzi M, Knolle P A, Cohen C J, Thimme R, Youngblood B, Zehn D. TOX reinforces the phenotype and longevity of exhausted T cells in chronic viral infection. Nature 571, 265-269 (2019).
33. Voehringer, D., Koschella, M., Pircher, H. Lack of proliferative capacity of human effector and memory T cells expressing killer cell lectinlike receptor G1 (KLRG1). Blood 100, 3698-3702 (2002).
34. Watanabe, N., Gavrieli, M., Sedy, J. R., Yang, J., Fallarino F, Loftin S K, Hurchla M A, Zimmerman N, Sim J, Zang X, Murphy T L, Russell J H, Allison J P, Murphy K M. BTLA is a lymphocyte inhibitory receptor with similarities to CTLA-4 and PD-1. Nat Immunol. 4, 670-679 (2003).
35. Maas-Bauer, K., Kiote-Schmidt C, Bertz H, Apostolova P, Wäsch R, Ihorst G, Finke J, Zeiser R. Ruxolitinib-ECP combination treatment for refractory severe chronic graft-versus-host disease. Bone Marrow Transplant. 56, 909-916 (2021).
36. Zeiser, R., Polverelli, N., Ram R, Hashmi S K, Chakraverty R, Middeke J M, Musso M, Giebel S, Uzay A, Langmuir P, Hollaender N, Gowda M, Stefanelli T, Lee S J, Teshima T, Locatelli F, for the REACH3 Investigators. Ruxolitinib for Glucocorticoid-Refractory Chronic Graft-versus-Host Disease. N Eng J Med 385, 228-238 (2021).
37. Mastroianni, J., Stickel, N., Andrlova, H., Hanke K, Melchinger W, Duquesne S, Schmidt D, Falk M, Andrieux G, Pfeifer D, Dierbach H, Schmitt-Graeff A, Meiss F, Boerries M, Zeiser R. miR-146a Controls Immune Response in the Melanoma Microenvironment. Cancer Res 79, 183-195 (2019).
38. Berner, H. S., Lyngstadaas, S. P., Spahr A et al. Adiponectin and its receptors are expressed in bone-forming cells. Bone 35, 842-849 (2004).
39. Osborn, O., and Olefsky, J. M. The cellular and signaling networks linking the immune system and metabolism in disease. Nature medicine 18, 363-374 (2012).
40. Arita, Y., Kihara S, Ouchi N, Takahashi M, Maeda K, Miyagawa J, Hotta K, Shimomura I, Nakamura T, Miyaoka K, Kuriyama H, Nishida M, Yamashita S, Okubo K, Matsubara K, Muraguchi M, Ohmoto Y, Funahashi T, Matsuzawa Y. Paradoxical decrease of an adipose-specific protein, adiponectin, in obesity. Biochem Biophys Res Commun. 257, 79-83 (1999).
41. Lindsay, R. S., Funahashi T, Hanson R L, Matsuzawa Y, Tanaka S, Tataranni P A, Knowler W C, Krakoff J. Adiponectin and development of type 2 diabetes in the Pima Indian population. Lancet 360, 57-58 (2002).
42. Pang, T. T. L., Narendran P. The distribution of adiponectin receptors on human peripheral blood mononuclear cells. Ann N Y Acad Sci. 1150, 143-145 (2008).
43. Ohashi, K., Parker J L, Ouchi N, Higuchi A, Vita J A, Gokce N, Pedersen A A, Kalthoff C, Tullin S, Sams A, Summer R, Walsh K. Adiponectin promotes macrophage polarization toward an anti-inflammatory phenotype. J Biol Chem. 285, 6153-6160 (2010).
44. Kumada, M., Kihara S, Ouchi N, Kobayashi H, Okamoto Y, Ohashi K, Maeda K, Nagaretani H, Kishida K, Maeda N, Nagasawa A, Funahashi T, Matsuzawa Y.

Adiponectin specifically increased tissue inhibitor of metalloproteinase-1 through interleukin-10 expression in human macrophages. Circulation 109, 2046-2049 (2004).
45. Meacham, C. E., Jeffery E C, Burgess R J, Sivakumar C D, Arora M A, Stanley A M, Colby E M, Crane G M, Zhao Z, Morrison S J. Adiponectin receptors sustain haematopoietic stem cells throughout adulthood by protecting them from inflammation. Nat Cell Biol. 24, 697-707 (2022).
46. Maeda, N., Shimomura I, Kishida K, Nishizawa H, Matsuda M, Nagaretani H, Furuyama N, Kondo H, Takahashi M, Arita Y, Komuro R, Ouchi N, Kihara S, Tochino Y, Okutomi K, Horie M, Takeda S, Aoyama T, Funahashi T, Matsuzawa Y. Diet-induced insulin resistance in mice lacking adiponectin/ACRP30. Nature medicine 8, 731-737 (2002).
47. Xu, A., Wang, Y., Keshaw H, Xu L Y, Lam K S, Cooper G J. The fat-derived hormone adiponectin alleviates alcoholic and nonalcoholic fatty liver diseases in mice. The Journal of clinical investigation 112, 91-100 (2003).
48. Wang, L., Luo Y, Luo L, Wu D, Ding X, Zheng H, Wu H, Liu B, Yang X, Silva F, Wang C, Zhang X, Zheng X, Chen J, Brigman J, Mandell M, Zhou Z, Liu F, Yang X O, Liu M. Adiponectin restrains ILC2 activation by AMPK-mediated feedback inhibition of IL-33 signaling. J Exp Med. 218, e20191054 (2021).
49. Fioravanti, A., Tenti S., Bacarelli M. R., Damiani A., Li Gobbi F., Bandinelli F., Cheleschi S., Galeazzi M., Benucci M. Tocilizumab modulates serum levels of adiponectin and chemerin in patients with rheumatoid arthritis: Potential cardiovascular protective role of IL-6 inhibition. Clin. Exp. Rheumatol. 37, 293-300 (2019).
50. Choi, I. A., Sagawa A., Lee E. Y., Lee E. B., Song Y. W. Tocilizumab Increases body weight and serum adipokine levels in patients with rheumatoid arthritis independently of their treatment response: A retrospective cohort study. J. Korean Med. Sci. 35, e155 (2020).
51. Kobayashi, Y., Iwata A, Suzuki K, Suto A, Kawashima S, Saito Y, Owada T, Kobayashi M, Watanabe N, Nakajima H. B and T lymphocyte attenuator inhibits LPS-induced endotoxic shock by suppressing Toll-like receptor 4 signaling in innate immune cells. PNAS 110, 5121-5126 (2013).
52. Bouguermouh, S., Van V Q, Martel J, Gautier P, Rubio M, Sarfati M. CD47 expression on T cell is a self-control negative regulator of type 1 immune response. J Immunol. 180, 8073-8082 (2008).
53. Zeiser, R., Blazar, B. R. Acute Graft-versus-host disease-Biologic process, prevention, and therapy. N Eng J Med 377, 2167-2179 (2017).
54. Zeiser, R., Blazar, B. R. Pathophysiology of Chronic Graft-versus-Host Disease and Therapeutic Targets. N Eng J Med 377, 2565-2579 (2017).
55. Dahl, E. K., Abed O K, Kjeldsen J, Donia M, Svane I M, Dige A, Agnholt J S, Bjerrum J T, Seidelin J B. Safety and efficacy of infliximab and corticosteroid therapy in checkpoint inhibitor-induced colitis. Aliment Pharmacol Ther. 56, 1370-1382 (2022).
56. Alexander, J. L., Ibraheim H, Sheth B, Little J, Khan M S, Richards C, Hunter N, Chauhan D, Ratnakumaran R, McHugh K, Pinato D J, Nathan P, Choy J, Crusz S M, Furness A, Turajlic S, Pickering L, Larkin J, Teare J P, Papa S, Speight A, Sharma A, Powell N. Clinical outcomes of patients with corticosteroid refractory immune checkpoint inhibitor-induced enterocolitis treated with infliximab. J Immunother Cancer. 7, e002742 (2021).
57. Lesage, C., Longvert C, Prey S, Maanaoui S, Dréno B, Machet L, Zehou O, Kramkimel N, Jeudy G, Skowron F, Aubin F, Visseaux L, Mansard S, Dereure O, Lesage F X, Guillot B; French Group of Onco-Dermatology. Incidence and Clinical Impact of Anti-TNFα Treatment of Severe Immune Checkpoint Inhibitor-induced Colitis in Advanced Melanoma: The Mecolit Survey. J Immunother. 42, 175-179 (2019).
58. Mir, R., Shaw, H. M., Nathan P D. Mycophenolate mofetil alongside high-dose corticosteroids: optimizing the management of combination immune checkpoint inhibitor-induced colitis. Melanoma Res. 29, 102-106 (2019).
59. Zhang, E., Kiely, C., Sandanayake N, Tattersall S. Calcineurin inhibitors in steroid and anti-TNF-alpha refractory immune checkpoint inhibitor colitis. JGH Open. 5, 558-562 (2021).
60. Thomas, A. S., Ma W, Wang Y. Ustekinumab for Refractory Colitis Associated with Immune Checkpoint Inhibitors. N Eng J Med 384, 581-583 (2021).
61. Esfahani, K., Hudson, M., Batist G. Tofacitinib for Refractory Immune-Related Colitis from PD-1 Therapy. N Eng J Med 382, 2374-2375 (2020).
62. Kadokawa, Y., Inoue, S., Tatsumi A, Uchida M, Fujita K, Takagi M, Inoue T, Ohe S, Nakai Y, Otsuka T, Abe Y, Nakabori T, Isei T, Kumagai T, Nishimura K, Ohkawa K. Efficacy and safety of mycophenolate mofetil in treating immune-related hepatitis induced by immune checkpoint inhibitor use: A retrospective study. JGH Open. 7, 87-97 (2023).
63. Omori, G., Takada K, Murase K, Hayasaka N, Nakamura H, Iyama S, Ohnuma H, Miyanishi K, Fukuta F, Tanaka T, Masumori N, Kato J. Successful mycophenolate mofetil treatment of a patient with severe steroid-refractory hepatitis evoked by nivolumab plus ipilimumab treatment for relapsed bladder cancer. Clin Case Rep. 9, 654-659 (2020).
64 Nakano, K., Nishizawa M, Fukuda N, Urasaki T, Wang X, Mitani H, Takahashi S. Mycophenolate mofetil as a successful treatment of corticosteroid-resistant immune checkpoint inhibitor-induced hepatitis. Oxf Med Case Reports. 4, omaa027 (2020).
65. Ueno, M., Takabatake H, Hata A, Kayahara T, Morimoto Y, Notohara K, Mizuno M. Mycophenolate mofetil for immune checkpoint inhibitor-related hepatotoxicity relapsing during dose reduction of corticosteroid: A report of two cases and literature review. Cancer Rep (Hoboken) 9, e1624 (2022).
66. Ea, V., Ngu N L Y, Kua H W, Mishra G. Tacrolimus for the Management of Delayed Onset and Treatment-Refractory Immune-Related Hepatitis. ACG Case Rep J. 10, e01007 (2023).
67. McIlwaine, S., Cullen, A., Stratton L, Oladipo B, Cash J, Carser J, Braniff C. The use of tacrolimus in the management of checkpoint inhibitor immunotherapy-induced hepatitis. J R Coll Physicians Edinb. 52, 20-23 (2022).
68. Chmiel, K. D., Suan D, Liddle C, Nankivell B, Ibrahim R, Bautista C, Thompson J, Fulcher D, Kefford R. Resolution of severe ipilimumab-induced hepatitis after antithymocyte globulin therapy. J Clin Oncol 29, e237-240 (2011).
69. Motomura, D., Baetz, T., Grin A, Flemming J A. Severe Refractory Checkpoint Inhibitor-Related Hepatitis Reversed With Anti-Thymocyte Globulin and n-Acetylcysteine. Hepatology. 72, 2235-2238 (2020).
70. Corrigan, M., Haydon G, Thompson F, Rajoriya N, Peplow C L, Hubscher S G, Steven N, Hirschfield G M, Armstrong M J. Infliximab for the treatment of refractory immune-related hepatitis secondary to checkpoint inhibitors: A case report. JHEP Rep. 1, 66-69 (2019).
71. Eleftheriotis, G., Skopelitis, E. Immune-checkpoint inhibitor-associated grade 3 hepatotoxicity managed with enteric-coated budesonide monotherapy: A case report. Medicine (Baltimore). 101, e29473 (2022).
72. Dhomen, N., Reis-Filho, J. S., da Rocha Dias, S., Hayward, R., Savage, K., Delmas, V., Larue, L., Pritchard, C., Marais, R. Oncogenic Braf induces melanocyte senescence and melanoma in mice. Cancer Cell 15, 294-303 (2009).
73. Vidal, K., Grosjean, I., evillard, J. P., Gespach, C. & Kaiserlian, D. Immortalization of mouse intestinal epithelial cells by the SV40-large T gene. Phenotypic and immune characterization of the MODE-K cell line. J Immunol Methods 166, 63-73 (1993).
74. Johncilla, M., Misdraji, J., Pratt D S, Agoston A T, Lauwers G Y, Srivastava A, Doyle L A. Ipilimumab-associated Hepatitis: Clinicopathologic Characterization in a Series of 11 Cases. Am J Surg Pathol. 39, 1075-1084 (2015).
75. Gerlach, U., et al. Centrosome-, chromosomal-passenger- and cell-cycle-associated mRNAs are differentially regulated in the development of sporadic colorectal cancer. J Pathol 208, 462-472 (2006).
76. Hamarsheh, S., Groß O, Brummer T, Zeiser R. Immune modulatory effects of oncogenic KRAS in cancer. Nat Commun. 11, 5439 (2020).
77. Ritchie, M. E., Phipson, B., Wu, D., Hu, Y., Law, C. W., Shi, W., Smyth, G. K. limma powers differential expression analyses for RNA-sequencing and microarray studies. Nucleic Acids Res. 43, e47 (2015).
78. Zheng, G. X., Terry J M, Belgrader P, Ryvkin P, Bent Z W, Wilson R, Ziraldo S B, Wheeler T D, McDermott G P, Zhu J, Gregory M T, Shuga J, Montesclaros L, Underwood J G, Masquelier D A, Nishimura S Y, Schnall-Levin M, Wyatt P W, Hindson C M, Bharadwaj R, Wong A, Ness K D, Beppu L W, Deeg H J, McFarland C, Loeb K R, Valente W J, Ericson N G, Stevens E A, Radich J P, Mikkelsen T S, Hindson B J, Bielas J H. Massively parallel digital transcriptional profiling of single cells. Nat Commun. 8, 14049 (2017).
79. Hao, Y., et al. Integrated analysis of multimodal single-cell data. Cell 184, 3573-3587 e3529 (2021).
80. Ianevski, A., Giri A K, Aittokallio T. Fully-automated and ultra-fast cell-type identification using specific marker combinations from single-cell transcriptomic data. Nat Commun. 13, 1246. (2022).
81. Badia-I-Mompel, P., Vélez Santiago J, Braunger J, Geiss C, Dimitrov D, Müller-Dott S, Taus P, Dugourd A, Holland C H, Ramirez Flores R O, Saez-Rodriguez J. decoupleR: ensemble of computational methods to infer biological activities from omics data. Bioinform Adv. 2, vbac016 (2022).
82. Garcia-Alonso, L., Holland C H, Ibrahim M M, Turei D, Saez-Rodriguez J. Benchmark and integration of resources for the estimation of human transcription factor activities. Genome Res. 8, 1363-1375 (2019).
83. Schubert, M., Klinger B, Klünemann M, Sieber A, Uhlitz F, Sauer S, Garnett M J, Blüthgen N, Saez-Rodriguez J. Perturbation-response genes reveal signaling footprints in cancer gene expression. Nat Commun. 9, 20 (2018).
84 Stickel, N., Hanke, K., Marschner, D., Prinz, G., Köhler, M., Melchinger, W., Pfeifer, D., Schmitt-Graeff, A., Brummer, T., Heine, A., Brossart, P., Wolf, D., von Bubnoff, N., Finke, J., Duyster, J., Ferrara, J., Salzer, U., Zeiser, R. MicroRNA-146a reduces MHC-II expression via targeting JAK/STAT-signaling in dendritic cells after stem cell transplantation. Leukemia 31, 2732-2741 (2017).

1-21. (canceled)

22. A method of treating immune checkpoint blockade (ICB)-induced immune related adverse events (irAEs) in a subject, comprising administering a therapeutically effective amount of Adiponectin or Adiponectin Receptor Agonist (ARA) to the subject.

23. The method of claim 22, wherein the subject is receiving checkpoint blockade therapy.

24. The method of claim 22, wherein the therapeutically effective amount of Adiponectin or ARA is administered at least 1 hour before, during, and/or at least one hour after administration of checkpoint blockade therapy.

25. The method of claim 22, wherein the checkpoint blockade therapy comprises administration of at least one antibody.

26. The method of claim 25, wherein the at least one antibody is selected from the group comprising cytotoxic T-lymphocyte antigen 4 (CTLA-4), programmed cell death-1 (PD-1) and/or programmed cell death ligand-1 (PD-L1) or Lymphocyte-activation gene 3 (LAG-3).

27. The method of claim 22, wherein the irAEs affect the gastrointestinal tract, lungs, endocrine glands, skin, liver, or any combination thereof.

28. The method of claim 22, wherein the irAEs comprise an ICB-induced colitis.

29. The method of claim 28, wherein administering the therapeutically effective amount of Adiponectin or ARA to the subject reduces the ICB-induced colitis.

30. The method of claim 22, wherein administering the therapeutically effective amount of Adiponectin or ARA to the subject does not interfere with an anti-tumor response induced by the checkpoint blockade therapy.

31. A method for treating a subject with ICB-induced irAEs, the method comprising:
administering a combination of a therapeutically effective amount of Adiponectin or ARA with an anti-tumor immunotherapy comprising administering lymphocytes to the subject.

32. The method of claim 31, wherein the lymphocytes are prepared by:
a) obtaining a sample derived from an isolated blood sample of the subject,
b) adding a photosensitizing agent to the sample, and
c) subjecting the sample to irradiation.

33. The method of claim 32, wherein the photosensitizing agent is a psoralen reagent.

34. The method of claim 32, wherein the photosensitizing agent is 8-methoxypsoralen.

35. The method of claim 32, wherein the irradiation is ultraviolet A (UVA) irradiation.

36. The method of claim 31, wherein the anti-tumor immunotherapy is extracorporeal photopheresis.

37. The method of claim 31, wherein the lymphocytes are immunoregulatory T cells.

38. The method of claim 37, wherein the immunoregulatory T cells are prepared by:
a) obtaining a sample derived from an isolated blood sample of the subject,
b) adding a photosensitizing agent to the sample, and
c) subjecting the sample to irradiation.

39. The method of claim 32, wherein the photosensitizing agent is a psoralen reagent.

40. The method of claim 31, wherein the subject suffers from cancer.

41. The method of claim 31, wherein the irAEs comprise an ICB-induced colitis.


SEQUENCE LISTING (72 sequences)

SEQ ID NO: 1 - DNA, 19 bp, synthetic construct
  source 1..19: mol_type = other DNA
tatgatggctccactggta

SEQ ID NO: 2 - DNA, 20 bp, synthetic construct
  source 1..20: mol_type = other DNA
gagcatagccttgtccttct

SEQ ID NO: 3 - DNA, 18 bp, synthetic construct
  source 1..18: mol_type = other DNA
gcccgaaacgccgaatat

SEQ ID NO: 4 - DNA, 19 bp, synthetic construct
  source 1..19: mol_type = other DNA
ccgtggttcgtggctctct

SEQ ID NO: 5 - DNA, 22 bp, synthetic construct
  source 1..22: mol_type = other DNA
cgtgttgttggtggcactgtga

SEQ ID NO: 6 - DNA, 22 bp, synthetic construct
  source 1..22: mol_type = other DNA
ccacatcagtgttccaggagac

SEQ ID NO: 7 - DNA, 20 bp, synthetic construct
  source 1..20: mol_type = other DNA
ctccacccaagggaacttgt

SEQ ID NO: 8 - DNA, 20 bp, synthetic construct
  source 1..20: mol_type = other DNA
aggaccaagaagacctgcat

SEQ ID NO: 9 - DNA, 22 bp, synthetic construct
  source 1..22: mol_type = other DNA
tctgcctcagtttctcctggct

SEQ ID NO: 10 - DNA, 23 bp, synthetic construct
  source 1..23: mol_type = other DNA
gtaatagagccagggaacgaagc

SEQ ID NO: 11 - DNA, 20 bp, synthetic construct
  source 1..20: mol_type = other DNA
acacagagacgggcaacatt

SEQ ID NO: 12 - DNA, 20 bp, synthetic construct
  source 1..20: mol_type = other DNA
gaagagtcgggagacccctt

SEQ ID NO: 13 - DNA, 19 bp, synthetic construct
  source 1..19: mol_type = other DNA
cctatgccttacccatgct

SEQ ID NO: 14 - DNA, 19 bp, synthetic construct
  source 1..19: mol_type = other DNA
tttccaagtcctgatgctg

SEQ ID NO: 15 - DNA, 24 bp, synthetic construct
  source 1..24: mol_type = other DNA
ggaatctgcatgggcaacctgtgt

SEQ ID NO: 16 - DNA, 22 bp, synthetic construct
  source 1..22: mol_type = other DNA
agggtctacgtctcgcaagcca

SEQ ID NO: 17 - DNA, 20 bp, synthetic construct
  source 1..20: mol_type = other DNA
gaaagccgccatagtctgga

SEQ ID NO: 18 - DNA, 20 bp, synthetic construct
  source 1..20: mol_type = other DNA
ctcttcatagctgccggagg

SEQ ID NO: 19 - DNA, 22 bp, synthetic construct
  source 1..22: mol_type = other DNA
ggcggtggaatacaatgtgtcc

SEQ ID NO: 20 - DNA, 22 bp, synthetic construct
  source 1..22: mol_type = other DNA
agcaggtcaaggtgaacagctg

SEQ ID NO: 21 - DNA, 22 bp, synthetic construct
  source 1..22: mol_type = other DNA
gcgagtgacgacttcaacctgg

SEQ ID NO: 22 - DNA, 21 bp, synthetic construct
  source 1..21: mol_type = other DNA
cgtcctccaggtcgaagcctc

SEQ ID NO: 23 - DNA, 22 bp, synthetic construct
  source 1..22: mol_type = other DNA
tacgatcagcgcctcaatgcca

SEQ ID NO: 24 - DNA, 22 bp, synthetic construct
  source 1..22: mol_type = other DNA
agcaggaaaccagccactagct

SEQ ID NO: 25 - DNA, 22 bp, synthetic construct
  source 1..22: mol_type = other DNA
gcaagattgcctgtctctccag

SEQ ID NO: 26 - DNA, 22 bp, synthetic construct
  source 1..22: mol_type = other DNA
cttgagagtccagtgttccgtc

SEQ ID NO: 27 - DNA, 20 bp, synthetic construct
  source 1..20: mol_type = other DNA
agcctaagatgagcgcaagt

SEQ ID NO: 28 - DNA, 20 bp, synthetic construct
  source 1..20: mol_type = other DNA
ttactaggcagatggccaca

SEQ ID NO: 29 - DNA, 22 bp, synthetic construct
  source 1..22: mol_type = other DNA
ggcagtggcaagaccataagga

SEQ ID NO: 30 - DNA, 22 bp, synthetic construct
  source 1..22: mol_type = other DNA
catctctccgtggatagactgg

SEQ ID NO: 31 - DNA, 22 bp, synthetic construct
  source 1..22: mol_type = other DNA
tacagacgtgctcctggtaggt

SEQ ID NO: 32 - DNA, 22 bp, synthetic construct
  source 1..22: mol_type = other DNA
ccgagcatttccagtgccttct

SEQ ID NO: 33 - DNA, 22 bp, synthetic construct
  source 1..22: mol_type = other DNA
ggtgctgacaatgtgttggagc

SEQ ID NO: 34 - DNA, 22 bp, synthetic construct
  source 1..22: mol_type = other DNA
ggtgctgacaatgtgttggagc

SEQ ID NO: 35 - DNA, 22 bp, synthetic construct
  source 1..22: mol_type = other DNA
gcaaagaggtcccaggctacat

SEQ ID NO: 36 - DNA, 23 bp, synthetic construct
  source 1..23: mol_type = other DNA
cctgtaatcacgtcagaagtccc

SEQ ID NO: 37 - DNA, 22 bp, synthetic construct
  source 1..22: mol_type = other DNA
ccgatttgctgttcctcgcctt

SEQ ID NO: 38 - DNA, 22 bp, synthetic construct
  source 1..22: mol_type = other DNA
gacctgagcaatggcagtgatg

SEQ ID NO: 39 - DNA, 22 bp, synthetic construct
  source 1..22: mol_type = other DNA
acagcacctcagtgtcacgatg

SEQ ID NO: 40 - DNA, 22 bp, synthetic construct
  source 1..22: mol_type = other DNA
ttctgtgccacatcagccgtca

SEQ ID NO: 41 - DNA, 22 bp, synthetic construct
  source 1..22: mol_type = other DNA
tggagatgtcgcagactggctt

SEQ ID NO: 42 - DNA, 22 bp, synthetic construct
  source 1..22: mol_type = other DNA
gaggaatcaccttgccagcact

SEQ ID NO: 43 - DNA, 22 bp, synthetic construct
  source 1..22: mol_type = other DNA
gcttaggagtctgcctttgctg

SEQ ID NO: 44 - DNA, 22 bp, synthetic construct
  source 1..22: mol_type = other DNA
gtcaggtgaacctttgcggaca

SEQ ID NO: 45 - DNA, 22 bp, synthetic construct
  source 1..22: mol_type = other DNA
gaagtggaacggagggtccttt

SEQ ID NO: 46 - DNA, 22 bp, synthetic construct
  source 1..22: mol_type = other DNA
gtctggaactcgtaggtgacct

SEQ ID NO: 47 - DNA, 22 bp, synthetic construct
  source 1..22: mol_type = other DNA
catgcagcctatcctgagacct

SEQ ID NO: 48 - DNA, 22 bp, synthetic construct
  source 1..22: mol_type = other DNA
ggtcaggtttgcctcacacttc

SEQ ID NO: 49 - DNA, 22 bp, synthetic construct
  source 1..22: mol_type = other DNA
tacttcgggcagtctctgagtg

SEQ ID NO: 50 - DNA, 22 bp, synthetic construct
  source 1..22: mol_type = other DNA
atggttgcctccagtctcagca

SEQ ID NO: 51 - DNA, 22 bp, synthetic construct
  source 1..22: mol_type = other DNA
gtgtgaggaactggtcgcctat

SEQ ID NO: 52 - DNA, 22 bp, synthetic construct
  source 1..22: mol_type = other DNA
ccgttctctggtccaaccgata

SEQ ID NO: 53 - DNA, 22 bp, synthetic construct
  source 1..22: mol_type = other DNA
tgccaggatgaccttagtgtcg

SEQ ID NO: 54 - DNA, 22 bp, synthetic construct
  source 1..22: mol_type = other DNA
cagagtgactgtggttccgtag

SEQ ID NO: 55 - DNA, 22 bp, synthetic construct
  source 1..22: mol_type = other DNA
ctccagaaggtggctttgatgc

SEQ ID NO: 56 - DNA, 22 bp, synthetic construct
  source 1..22: mol_type = other DNA
gtgaaacccagcatccgtggaa

SEQ ID NO: 57 - DNA, 22 bp, synthetic construct
  source 1..22: mol_type = other DNA
ctttccgagagcaacatccagc

SEQ ID NO: 58 - DNA, 22 bp, synthetic construct
  source 1..22: mol_type = other DNA
gttgctggagtcgtcagacagt

SEQ ID NO: 59 - DNA, 22 bp, synthetic construct
  source 1..22: mol_type = other DNA
gtgagtgcgatgacttctcctg

SEQ ID NO: 60 - DNA, 22 bp, synthetic construct
  source 1..22: mol_type = other DNA
caggtgtcagtgcgtgtagtac

SEQ ID NO: 61 - DNA, 22 bp, synthetic construct
  source 1..22: mol_type = other DNA
acacaagctccagcagacgaag

SEQ ID NO: 62 - DNA, 22 bp, synthetic construct
  source 1..22: mol_type = other DNA
tccacctgcttctctgtcagct

SEQ ID NO: 63 - DNA, 22 bp, synthetic construct
  source 1..22: mol_type = other DNA
actcattcctggagcaaccgtg

SEQ ID NO: 64 - DNA, 23 bp, synthetic construct
  source 1..23: mol_type = other DNA
gctgtgaaagacaggttgagtcc

SEQ ID NO: 65 - DNA, 22 bp, synthetic construct
  source 1..22: mol_type = other DNA
tgatgtccgagtcaaccagacg

SEQ ID NO: 66 - DNA, 22 bp, synthetic construct
  source 1..22: mol_type = other DNA
actcctctgagaagccaagagc

SEQ ID NO: 67 - DNA, 22 bp, synthetic construct
  source 1..22: mol_type = other DNA
atgcttcaggctgccgtctgtg

SEQ ID NO: 68 - DNA, 22 bp, synthetic construct
  source 1..22: mol_type = other DNA
cagtttccgtcattcggcacca

SEQ ID NO: 69 - DNA, 23 bp, synthetic construct
  source 1..23: mol_type = other DNA
ccaaagccagtagcatcatggtc

SEQ ID NO: 70 - DNA, 22 bp, synthetic construct
  source 1..22: mol_type = other DNA
ggatggtgaagttggctacagg

SEQ ID NO: 71 - DNA, 22 bp, synthetic construct
  source 1..22: mol_type = other DNA
ggaagtgactgtctaccaccga

SEQ ID NO: 72 - DNA, 22 bp, synthetic construct
  source 1..22: mol_type = other DNA
ctcaggaagtgcttggtctctc